(19) United States
(12) Patent Application Publication
(10) Pub. No.: US 2024/0254557 A1
(43) Pub. Date: Aug. 1, 2024

(54) DIAGNOSTIC FOR SEPSIS ENDOTYPES AND/OR SEVERITY

(71) Applicant: THE UNIVERSITY OF BRITISH COLUMBIA, VANCOUVER (CA)

(72) Inventors: ROBERT E. HANCOCK, Vancouver (CA); ARJUN BAGHELA, Delta (CA); GABRIELA COHEN FREUE, Vancouver (CA)

(73) Assignee: THE UNIVERSITY OF BRITISH COLUMBIA, VANCOUVER, BC (CA)

(21) Appl. No.: 18/563,344

(22) PCT Filed: May 25, 2022
(86) PCT No.: PCT/CA2022/050831

§ 371 Date: Nov. 21, 2023

Related U.S. Application Data
(60) Provisional application No. 63/192,746, filed on May 25, 2021.

(51) Int. Cl. C12Q 1/6883; A61K 45/06; C12Q 1/6809; C12Q 1/686

(52) U.S. Cl. CPC C12Q 1/6883; A61K 45/06; C12Q 1/6809; C12Q 1/686; C12Q 2600/112; C12Q 2600/158

(57) ABSTRACT

The present disclosure relates to methods for classifying a subject into a sepsis mechanistic endotype as well as methods for predicting severity of sepsis in a subject. The methods can comprise use of a biological sample obtained from the subject at first clinical presentation. The classification of the subject into a sepsis mechanistic endotype and/or prediction of severity of sepsis may, for example, allow for treatment of sepsis using an approach suitable to the particular mechanistic endotype and/or severity.

### CROSS-REFERENCE TO RELATED APPLICATIONS

The present application claims the benefit of priority from co-pending U.S. provisional application No. 63/192,746 filed on May 25, 2021, the contents of which are incorporated herein by reference in their entirety.

### FIELD

The present disclosure relates to the field of biomarkers for sepsis. For example, the present disclosure relates to a unique set of DNA sequences that, may for example, enable the separation of sepsis patients into distinct mechanistic and/or clinically meaningful clusters, as well as the prediction of sepsis severity and mortality at first clinical presentation.

### BACKGROUND

Sepsis continues to be the major infection-related cause of death globally, leading to an estimated 19.7% of deaths (e.g., 11 million deaths in 2017) annually. More recently sepsis has been recognized to be the major cause of mortality in patients with severe Covid-19 infections. Despite advances in modem medicine including new antibiotics and vaccines, earlier recognition and best practice treatments, and efficient well-equipped intensive care units, there is a high rate of mortality, about 22%, that has remained little changed for decades.

Sepsis is described as a dysfunctional, life-threatening response to infection and is extremely common (estimated 48.9 million cases leading to 11 million annual deaths in 2017). Inter-individual clinical variability in the course of early sepsis can prevent clinicians from appropriately triaging patients for optimal treatment. Identifying gene expression signatures capturing specific host responses in ER and ICU patients may, for example, allow clinicians to identify the most at-risk groups of patients, provide early diagnostic certainty and enable appropriate use of antibiotics and development of disease-specific therapies, as well as identifying patients who likely do not need such intensive treatment, thus reducing costs and saving hospital resources.

Sepsis is notorious for the clinical heterogeneity observed in patients, who often demonstrate broad and fairly non-specific symptomology in the emergency room (ER), and can rapidly deteriorate thereafter. Therefore, it is difficult for clinicians to appropriately detect and triage sepsis patients early on in the disease course. The general doctrine that each hour's delay in initiating antibiotics costs lives, is still accepted by clinicians, often by initiating early treatment with potent antibiotics in the hope that the progression to sepsis is hindered. The problem is that some patients who will go on to severe sepsis are not recognized early enough while others that do not have sepsis will be treated incorrectly. The latter has the downside of contributing to the rise of antibiotic resistance, since broad-spectrum antibiotics are overused even in cases where there is no bacterial infection. Thus, a novel means of triaging sepsis patients is desirable, given the rapid deterioration and the societal repercussions of increased antibiotic resistance and health care costs.

Biomarkers for the diagnosis of sepsis have been proposed in U.S. Pat. Nos. 7,767,395; 8,029,982, U.S. Patent Application Publication No. 2011/0312521; U.S. Patent Application Publication No. 2011/0076685; U.S. Patent Application Publication No. 2020/0140948A1, International Patent Application Publication No. WO 2013/152047, International Patent Application Publication No. WO 2014/209238, International Patent Application Publication No. WO 2015/135071A1, International Patent Application No. WO 2018/146162A1, and International Patent Application Publication No. WO 2016/145426A1.

Blood transcriptomics has proven useful in obtaining systems-level representations of the responses dysregulated during sepsis. Using this method, several groups have identified, either in the ER or ICU, gene expression signatures that discriminate between sepsis and Systemic Inflammatory Response Syndrome (SIRS), or between patients who survive or succumb [Pena O M, et al. EBioMedicine. 2014; 1:64-71, doi:10.1016/j.ebiom.2014.10.003; McHugh L, et al. PLoS Medicine 2015; 12:e1001916 doi:10.1371/journal.pmed.1001916; Sweeney T E, et al. Science Transl. Med. 2015; 7:287ra71. doi:10.1126/scitranslmed.aaa5993; Scicluna B P, et al. Amer. J. Resp. Crit. Care Medi. 2015; 192:826-835. doi:10.1164/rccm.201502-03550C]. Nevertheless, these approaches typically lack sensitivity because of substantial heterogeneity in patients with similar outcomes that is not considered. This includes but is not limited to responses driven by individual genetic variation, demographic factors, the infection source and agent, appropriateness of therapeutic intervention, comorbidities including pre-existing immune-suppressive conditions, and/or epigenetics [Leligdowicz A, and Matthay M A. Critical Care 23:80, doi:10.1186/s13054-019-2372-2]. These shortcomings have led to sepsis being refrained as a condition comprised of several subgroups termed endotypes, which represent distinct biologically-driven and clinically-relevant groups of patients with varied severity and clinical outcomes, where endotypes are defined as subtypes of a condition, defined by distinct functional and/or pathobiological mechanisms. Specifically, endotypes may provide more sensitive markers enabling risk-stratification and opportunities for individualized therapies. However, endotypes in sepsis have only been characterized in patients with advanced disease, while early prognostication of endotype status is desirable.

Framing sepsis in the context of endotypes has the potential to identify dysregulation of biological processes common to subgroups, thus enabling endotype-specific treatment of patients in a specific host-directed (e.g., immunomodulatory) manner. Previous research identifying endotypes has shown that subgroups of patients exist in sepsis patients, particularly in those who present to intensive care units (ICU) [Scicluna B P et al. Lancet Resp. Med. 2017; 5:816-826. doi:10.1016/52213-2600(17)30294-1; Davenport E E, et al. The Lancet Resp. Med. 2016; 4:259-271. doi:10.1016/52213-2600(16)00046-1; Maslove D M, et al. Critical Care. 2012; 16:R183. doi:10.1186/cc11667; Sweeney T E, et al. Critical Care Med. 2018; 46:915-925. doi:10.1097/CCM.0000000000003084], but have not addressed patients just entering the emergency room (ER) at first clinical presentation.

The molecular responses determining endotype status have often been explained by the influence of several immune cell types, most notably neutrophils, monocytes, and T cell subsets that bear the features of immunosuppression [Hotchkiss R S et al. Lancet Infect. Dis. 2013; 13:260-8. doi:10.1016/S1473-3099(13)70001-X]. To date the published studies demonstrate generally that there are endotypes identifiable after sepsis has already been confirmed. However, this represents a stage in a patient's clinical course where prognosis is arguably less useful, since patients have already deteriorated and likely already require intensive care and antibiotics. Moreover, there is little consensus as to the make-up and nature of endotypes.

This background information is provided for the purpose of making known information believed by the applicant to be of possible relevance to the present invention. No admission is necessarily intended, nor should be construed, that any of the preceding information constitutes prior art against the present invention.

### SUMMARY

An objective of the present disclosure was to identify endotypes at first clinical presentation, where patients show broad clinical traits and final sepsis diagnoses are not established. Whole blood and clinical data profiles were collected from 115 patients in emergency rooms and 82 patients in one intensive care unit, and compared to 9 healthy controls from the same sources. ER patients were recruited into the study within two hours of admission if the attending clinician suspected possible sepsis and observed two or more systemic inflammatory response syndrome (SIRS) symptoms. Blood RNA-Seq transcriptomic profiles were analyzed to identify early mechanistic gene expression signatures useful for triage. Machine learning was used to uncover endotypes (subdivisions of the disease with distinct pathophysiological mechanisms and clinical responses) and to validate corresponding gene signatures with prognostic value. Patients with early sepsis exhibited evidence of five mechanistically distinct endotypes, namely Neutrophilic-Suppressive (NPS), Inflammatory (INF), Innate Host Defense (IHD), Interferon (IFN), and Adaptive (ADA) endotypes. Subsequently, a classification tool employing 88 genes was used to accurately predict endotype status in a validation cohort while another 247 showed suitable differential expression in the given endotypes to be useful in differentiation between endotypes. This included 82 ICU patients, of which 27 patients had Covid-19-mediated sepsis. Subsets of these 88 genes can be used, for example, to accurately identify specific endotypes (including those causing higher severity), through gene expression analysis of patient blood. Across all patients, the NPS and INF endotypes showed the worse prognosis, with higher organ dysfunction scores and severity. Furthermore, a predictive severity signature was demonstrated. This provides a method to triage a diverse spectrum of prospective pre-diagnosis sepsis patients in the emergency room (ER) into 5 mechanism-based endotypes based on the underlying molecular responses, and shows that endotypes are associated with specific clinical characteristics and outcomes. These endotypes remain detectable in the intensive care unit (ICU), indicating they are stable. The separation of patients into endotypes has prognostic value and can inform a physician regarding future severity, enabling only the worst afflicted patients to receive the most intensive treatments and driving the potential for personalized medicines. Furthermore, signatures predicting enhanced severity independent of endotype status are described.

Accordingly, the present disclosure includes a method for classifying a subject into a sepsis mechanistic endotype selected from neutrophilic-suppressive (NPS), inflammatory (INF), innate host defense (IHD), interferon (IFN) and adaptive (ADA) endotypes, the method comprising: (a) determining, in a biological sample from the subject, a level of expression for each of a plurality of genes, to provide a sample gene signature; and (b) comparing the sample gene signature with a reference gene signature to determine whether the subject has the sepsis mechanistic endotype, wherein the sample gene signature and reference gene signature comprise an NPS endotype sub-signature, an INF endotype sub-signature, an IHD endotype sub-signature, an IFN endotype sub-signature, an ADA endotype sub-signature or combinations thereof, wherein the NPS endotype sub-signature comprises genes selected from the group consisting of AGFG1, ARG1, ATP9A, ANXA3, EFNA1, GADD45A, GPR84, HPGD, IL1R1, KLF14, KREMEN1, MIR646HG, MLLT1, NSUN7, OLAH, ORM2, PCOLCE2, PFKFB2, SLC51A, TNFAIP8L3, ZDHHC19, ADAMTS3, AKR1C1, ALDH1A2, ALOX5AP, ALPL, AMPH, ANKRD55, BCL3, BTBD19, CA4, CD163L1, CD177, CD82, CST7, CYP19A1, CYSTM1, DAAM2, DGAT2, ECHDC3, ENTPD7, EXOSC4, FFAR3, FGF13, FSTL4, GALNT14, GRAMD1A, GRB10, GYG1, HPGD, IER3, IL18RAP, IL1R2, IL1RN, IRAG1-AS1, KCNE1B, KCNMA1, MCEMP1, MKNK1, MMP9, MSRA, NECAB1, NSMCE1-DT, OPLAH, PDGFC, PFKFB3, PHF24, PI3, PLIN4, PLIN5, PLK3, POR, PROK2, RFX2, RGL4, ROM1, S100A12, S100P, SEMA6B, SHROOM4, SLP1, SOCS3, SPATC1, SPDYA, SPINK8, SPP1, ST6GALNAC3, SYN2, TDRD9, TMEM120A, TMIGD3, TSPO, UPP1, and XCR1; wherein the INF endotype sub-signature comprises genes selected from the group consisting of BNIP3L, CA1, FAM83A, FECH, GLRX5, GYPA, IFIT1B, RHCE, RIOK3, RNF182, SLC6A19, SPTA1, THEM5, TLCD4, TSPAN5, TSPO2, ABCG2, ACHE, ACKR1, ACSL6, ADD2, AHSP, ALAS2, ALDH5A1, ANK1, ANKRD9, AQP1, ARHGEF12, ARHGEF37, ARL4A, ATP1B2, ATP1B2, BBOF1, BCAM, BCL2L1, BLVRB, BPGM, Clorf116, CA2, CISD2, CLIC2, CR1L, CR1L, CTNNAL1, CTSE, DCAF12, DMTN, DNAJC6, DPCD, DYRK3, EMID1, EPB42, ERFE, FAM210B, FAXDC2, FRMD4A, GMPR, GSPT1, GYPB, HBM, HEMGN, HEPACAM2, HMBS, IGF2BP2, ISCA1, ITLN1, KANK2, KCNH2, KDM7A-DT, KEL, KLC3, KLF1, KLHDC8A, KRT1, LRRC2, MAOA, MARCHF8, MBNL3, MFSD2B, MRC2, MXI1, MYL4, NFIX, NUDT4, OSBP2, PAGE2B, PBX1, PCDH1, PGF, PLEK2, PNP, PRDX2, PTPRF, RAPIGAP, RBM38, RFESD, RFESD, RGCC, RGS16, RHAG, RHD, RIPOR3, RNF175, RUNDC3A, SEC14L4, SELENBP1, SELENOP, SFRP2, SGIP1, SIAH2, SLC14A1, SLC1A5, SLC22A23, SLC2A1, SLC4A1, SLC6A8, SLC6A9, SLC7A5, SMIM5, SNCA, SOX6, SPTB, STRADB, TAL1, TENT5C, TFR2, TMCC2, TMOD1, TNS1, TRIM10, TRIM58, TSPAN7, TTC25, UBB, USP12, XK, YBX3, and YPEL4; wherein the IHD endotype sub-signature comprises genes selected from the group consisting of ABCA6, ADAM23, ALOX15, CACNA2D3, DYNC2H1, GPR34, GRAMD1C, LPL, MAP7, MIR155HG, PLCB1, SDC2, SIGLEC8, SPRED1, SLC16A14, SMPD3, TPPP3, TPRG1, ZNF600, ADGRD1, ANGPT1, GPR82, HDAC9, IL5RA, KLHDC1, PRSS33, PTGDR2, PTGFRN, TBC1D12, and TRIM2; wherein the IFN endotype sub-signature comprises genes selected from the group consisting of ANKRD22, APOL1, APOL4, BATF2, CARD17, CD274, EPSTI1, ETV7, GBP5, IDO1, IFITM3, P2RY14, PLEKHO1, RSAD2, SERPING1, TFEC, EXOC3L1, IRF7, OAS1, SEPTIN4, LY6E, and LAMP3; and wherein the ADA endotype sub-signature comprises genes selected from the group consisting of: CCL2, CDC45, CENPF, CLEC4F, GTSE1, IFI27, ISG15, KCTD14, KIF14, KIF15, KLHDC7B, LGALS3BP, OTOF, PDIA4, SIGLEC1, USP18, AGRN, CD38, CDCA7, CDT1, CTLA4, DHX58, EME1, FAM111B, HES4, IFI44L, IFIT3, IFNG-AS1, IL12RB2, IL4I1, KIF19, LAG3, MCM10, P2RY6, PACSIN1, PARM1, SAMD4A, SPATS2L, HERC5, TMPRSS3, TNFRSF13B, TSHR, and TTC21A.

In an embodiment, the reference gene signature represents a standard level of expression of the genes comprised therein and a difference between a sample endotype sub-signature and a reference endotype sub-signature indicates that the subject has the sepsis mechanistic endotype corresponding to that sub-signature.

In an embodiment, the sample gene signature and the reference gene signature comprise the NPS endotype sub-signature, the INF endotype sub-signature, the IHD endotype sub-signature, the IFN endotype sub-signature, and the ADA endotype sub-signature.

In an embodiment, the NPS endotype sub-signature comprises genes selected from the group consisting of AGFG1, ARG1, ATP9A, ANXA3, EFNA1, GADD45A, GPR84, HPGD, IL1R1, KLF14, KREMEN1, MIR646HG, MLLT1, NSUN7, OLAH, ORM2, PCOLCE2, PFKFB2, SLC51A, TNFAIP8L3, and ZDHHC19. In another embodiment, the NPS endotype sub-signature comprises: AGFG1, ARG1, ATP9A, ANXA3, EFNA1, GADD45A, GPR84, HPGD, IL1R1, KLF14, KREMEN1, MIR646HG, MLLT1, NSUN7, OLAH, ORM2, PCOLCE2, PFKFB2, SLC51A, TNFAIP8L3, and ZDHHC19.

In an embodiment, the INF endotype sub-signature comprises genes selected from the group consisting of BNIP3L, CA1, FAM83A, FECH, GLRX5, GYPA, IFIT1B, RHCE, RIOK3, RNF182, SLC6A19, SPTA1, THEM5, TLCD4, TSPAN5, and TSPO2. In another embodiment, the INF endotype sub-signature comprises: BNIP3L, CA1, FAM83A, FECH, GLRX5, GYPA, IFIT1B, RHCE, RIOK3, RNF182, SLC6A19, SPTA1, THEM5, TLCD4, TSPAN5, and TSPO2.

In an embodiment, the IHD endotype sub-signature comprises genes selected from the group consisting of: ABCA6, ADAM23, ALOX15, CACNA2D3, DYNC2H1, GPR34, GRAMD1C, LPL, MAP7, MIR155HG, PLCB1, SDC2, SIGLEC8, SPRED1, SLC16A14, SMPD3, TPPP3, TPRG1, and ZNF600. In another embodiment, the IHD endotype sub-signature comprises: ABCA6, ADAM23, ALOX15, CACNA2D3, DYNC2H1, GPR34, GRAMD1C, LPL, MAP7, MIR155HG, PLCB1, SDC2, SIGLEC8, SPRED1, SLC16A14, SMPD3, TPPP3, TPRG1, and ZNF600.

In an embodiment, the IFN endotype sub-signature comprises genes selected from the group consisting of ANKRD22, APOL1, APOL4, BATF2, CARD17, CD274, EPSTI1, ETV7, GBP5, IDO1, IFITM3, P2RY14, PLEKHO1, RSAD2, SERPING1, and TFEC. In another embodiment, the IFN endotype sub-signature comprises: ANKRD22, APOL1, APOL4, BATF2, CARD17, CD274, EPSTI1, ETV7, GBP5, IDO1, IFITM3, P2RY14, PLEKHO1, RSAD2, SERPING1, and TFEC.

In an embodiment, the ADA endotype sub-signature comprises genes selected from the group consisting of CCL2, CDC45, CENPF, CLEC4F, GTSE1, IFI27, ISG15, KCTD14, KIF14, KIF15, KLHDC7B, LGALS3BP, OTOF, PDIA4, SIGLEC1, and USP18. In another embodiment, the ADA endotype sub-signature comprises: CCL2, CDC45, CENPF, CLEC4F, GTSE1, IFI27, ISG15, KCTD14, KIF14, KIF15, KLHDC7B, LGALS3BP, OTOF, PDIA4, SIGLEC1 and USP18.

The present disclosure also includes a method for classifying a subject into a sepsis mechanistic endotype selected from neutrophilic-suppressive (NPS), inflammatory (INF), innate host defense (IHD), interferon (IFN) and adaptive (ADA) endotypes, the method comprising: (a) determining, in a biological sample from the subject, a level of expression for each of a plurality of genes, to provide a sample gene signature; and (b) comparing the sample gene signature with a reference gene signature to determine whether the subject has the sepsis mechanistic endotype, wherein the sample gene signature and reference gene signature comprise an NPS endotype signature pair, an INF endotype signature pair, an IHD endotype signature pair, an IFN endotype signature pair, an ADA endotype signature pair or combinations thereof, wherein the NPS endotype signature pair is selected from: GADD45A/EFNA1, EFNA1/MIR646HG, MIR646HG/KLF14, MLLT1/MIR646HG, ARG1/MLLT1, MLLT1/EFNA1, MLLT1/NSUN7, EFNA1/NSUN7, SLC51A/EFNA1, EFNA1/KLF14, ZDHHC19/EFNA1, EFNA1/AGFG1, NSUN7/KLF14, EFNA1/PFKFB2, MLLT1/KLF14, ADAMTS3/PCOLCE2, ADAMTS3/ZDHHC19, ADAMTS3/SLC51A, ADAMTS3/HPGD, ADAMTS3/SEMA6B, ADAMTS3/EFNA1, ADAMTS3/AGFG1, ADAMTS3/NSUN7, ADAMTS3/TNFAIP8L3, ADAMTS3/KREMEN1, ADAMTS3/ORM2, ADAMTS3/MIR646HG, ADAMTS3/KLF14, AGFG1/NSUN7, AGFG1/TNFAIP8L3, AGFG1/KREMEN1, AGFG1/ORM2, AGFG1/MIR646HG, AGFG1/KLF14, ANXA3/GPR84, ANXA3/OLAH, ANXA3/ADAMTS3, ANXA3/PCOLCE2, ANXA3/ZDHHC19, ANXA3/SLC51A, ANXA3/HPGD, ANXA3/SEMA6B, ANXA3/EFNA1, ANXA3/AGFG1, ANXA3/NSUN7, ANXA3/TNFAIP8L3, ANXA3/KREMEN1, ANXA3/ORM2, ANXA3/MIR646HG, ANXA3/KLF14, ARG1/PFKFB2, ARG1/MLLT1, ARG1/ANXA3, ARG1/GPR84, ARG1/OLAH, ARG1/ADAMTS3, ARG1/PCOLCE2, ARG1/ZDHHC19, ARG1/SLC51A, ARG1/HPGD, ARG1/SEMA6B, ARG1/EFNA1, ARG1/AGFG1, ARG1/NSUN7, ARG1/TNFAIP8L3, ARG1/KREMEN1, ARG1/ORM2, ARG1/MIR646HG, ARG1/KLF14, ATP9A/EPB41L4B, ATP9A/IL1R1, ATP9A/GADD45A, ATP9A/ARG1, ATP9A/PFKFB2, ATP9A/MLLT1, ATP9A/ANXA3, ATP9A/GPR84, ATP9A/OLAH, ATP9A/ADAMTS3, ATP9A/PCOLCE2, ATP9A/ZDHHC19, ATP9A/SLC51A, ATP9A/HPGD, ATP9A/SEMA6B, ATP9A/EFNA1, ATP9A/AGFG1, ATP9A/NSUN7, ATP9A/TNFAIP8L3, ATP9A/KREMEN1, ATP9A/ORM2, ATP9A/MIR646HG, ATP9A/KLF14, EFNA1/AGFG1, EFNA1/NSUN7, EFNA1/TNFAIP8L3, EFNA1/KREMEN1, EFNA1/ORM2, EFNA1/MIR646HG, EFNA1/KLF14, EPB41L4B/IL1R1, EPB41L4B/GADD45A, EPB41L4B/ARG1, EPB41L4B/PFKFB2, EPB41L4B/MLLT1, EPB41L4B/ANXA3, EPB41L4B/GPR84, EPB41L4B/OLAH, EPB41L4B/ADAMTS3, EPB41L4B/PCOLCE2, EPB41L4B/ZDHHC19, EPB41L4B/SLC51A, EPB41L4B/HPGD, EPB41L4B/SEMA6B, EPB41L4B/EFNA1, EPB41L4B/AGFG1I,EPB41L4B/NSUN7, EPB41L4B/TNFAIP8L3, EPB41L4B/KREMEN1, EPB41L4B/MIR646HG, EPB41L4B/KLF14, GADD45A/ARG1, GADD45A/PFKFB2, GADD45A/MLLT1, GADD45A/ANXA3, GADD45A/GPR84, GADD45A/OLAH, GADD45A/ADAMTS3, GADD45A/PCOLCE2, GADD45A/ZDHHC19, GADD45A/SLC51A, GADD45A/HPGD, GADD45A/SEMA6B, GADD45A/EFNA1, GADD45A/AGFG1, GADD45A/NSUN7, GADD45A/TNFAIP8L3, GADD45A/KREMEN1, GADD45A/ORM2, GADD45A/MIR646HG, GADD45A/KLF14, GPR84/OLAH, GPR84/ADAMTS3, GPR84/PCOLCE2, GPR84/ZDHHC19, GPR84/SLC51A, GPR84/HPGD, GPR84/SEMA6B, GPR84/EFNA1, GPR84/AGFG1, GPR84/NSUN7, GPR84/TNFAIP8L3, GPR84/KREMEN1, GPR84/ORM2, GPR84/MIR646HG, GPR84/KLF14, HPGD/SEMA6B, HPGD/EFNA1, HPGD/AGFG1, HPGD/NSUN7, HPGD/TNFAIP8L3, HPGD/KREMEN1, HPGD/ORM2, HPGD/MIR646HG, HPGD/KLF14, IL1R1/GADD45A, IL1R1/ARG1, IL1R1/PFKFB2, IL1R1/MLLT1, IL1R1/ANXA3, IL1R1/GPR84, IL1R1/OLAH, IL1R1/ADAMTS3, IL1R1/PCOLCE2, IL1R1/ZDHHC19, IL1R1/SLC51A, IL1R1/HPGD, IL1R1/SEMA6B, IL1R1/EFNA1, IL1R1/AGFG1, IL1R1/NSUN7, IL1R1/TNFAIP8L3, IL1R1/KREMEN1, IL1R1/ORM2, IL1R1/MIR646HG, IL1R1/KLF14, KREMEN1/ORM2, KREMEN1/MIR646HG, KREMEN1/KLF14, MIR646HG/KLF14, MLLT1/ANXA3, MLLT1/GPR84, MLLT1/OLAH, MLLT1/ADAMTS3, MLLT1/PCOLCE2, MLLT1/ZDHHC19, MLLT1/SLC51A, MLLT1/HPGD, MLLT1/SEMA6B, MLLT1/EFNA1, MLLT1/AGFG1, MLLT1/NSUN7, MLLT1/TNFAIP8L3, MLLT1/KREMEN1, MLLT1/ORM2, MLLT1/MIR646HG, MLLT1/KLF14, NSUN7/TNFAIP8L3, NSUN7/KREMEN1, NSUN7/ORM2, NSUN7/MIR646HG, NSUN7/KLF14, OLAH/ADAMTS3, OLAH/PCOLCE2, OLAH/ZDHHC19, OLAH/SLC51A, OLAH/HPGD, OLAH/SEMA6B, OLAH/EFNA1, OLAH/AGFG1, OLAH/NSUN7, OLAH/TNFAIP8L3, OLAH/KREMEN1, OLAH/ORM2, OLAH/MIR646HG, OLAH/KLF14, ORM2/MIR646HG, ORM2/KLF14, PCOLCE2/ZDHHC19, PCOLCE2/SLC51A, PCOLCE2/HPGD, PCOLCE2/SEMA6B, PCOLCE2/EFNA1, PCOLCE2/AGFG1, PCOLCE2/NSUN7, PCOLCE2/TNFAIP8L3, PCOLCE2/KREMEN1, PCOLCE2/ORM2, PCOLCE2/MIR646HG, PCOLCE2/KLF14, PFKFB2/MLLT1, PFKFB2/ANXA3, PFKFB2/GPR84, PFKFB2/OLAH, PFKFB2/ADAMTS3, PFKFB2/PCOLCE2, PFKFB2/ZDHHC19, PFKFB2/SLC51A, PFKFB2/HPGD, PFKFB2/SEMA6B, PFKFB2/EFNA1, PFKFB2/AGFG1, PFKFB2/NSUN7, PFKFB2/TNFAIP8L3, PFKFB2/KREMEN1, PFKFB2/ORM2, PFKFB2/MIR646HG, PFKFB2/KLF14, SEMA6B/EFNA1, SEMA6B/AGFG1, SEMA6B/NSUN7, SEMA6B/TNFAIP8L3, SEMA6B/KREMEN1, SEMA6B/ORM2, SEMA6B/MIR646HG, SEMA6B/KLF14, SLC51A/HPGD, SLC51A/SEMA6B, SLC51A/EFNA1, SLC51A/AGFG1, SLC51A/NSUN7, SLC51A/TNFAIP8L3, SLC51A/KREMEN1, SLC51A/ORM2, SLC51A/MIR646HG, SLC51A/KLF14, TNFAIP8L3/KREMEN1, TNFAIP8L3/ORM2, TNFAIP8L3/MIR646HG, TNFAIP8L3/KLF14, ZDHHC19/SLC51A, ZDHHC19/HPGD, ZDHHC19/SEMA6B, ZDHHC19/EFNA1, ZDHHC19/AGFG1, ZDHHC19/NSUN7, ZDHHC19/TNFAIP8L3, ZDHHC19/KREMEN1, ZDHHC19/ORM2, ZDHHC19/MIR646HG, and ZDHHC19/KLF14; wherein the INF endotype signature pair is selected from: FECH/TFEC, TFEC/IFIT1B, FECH/RNF182, IFIT1B/FECH, FECH/APOL4, FECH/GYPA, ITLN1/FECH, FECH/THEM5, IFIT1B/CA1, RHAG/FECH, FECH/FAM83A, RHCE/FECH, TFEC/CA1, SPTA1/FECH, ANKRD22/GLRX5, ANKRD22/GYPA, ANKRD22/IFIT1B, ANKRD22/ITLN1, ANKRD22/KLHDC8A, ANKRD22/RHCE, ANKRD22/RNF182, ANKRD22/SPTA1, ANKRD22/THEM5, ANKRD22/TSPAN5, APOL4/BNIP3L, APOL4/CA1, APOL4/DYRK3, APOL4/FAM83A, APOL4/GLRX5, APOL4/GYPA, APOL4/IFIT1B, APOL4/ITLN1, APOL4/KLHDC8A, APOL4/RHAG, APOL4/RHCE, APOL4/RIOK3, APOL4/RNF182, APOL4/SPTA1, APOL4/THEM5, APOL4/TLCD4, APOL4/TMCC2, APOL4/TSPAN5, APOL4/TSPO2, BNIP3L/ANKRD22, BNIP3L/CA1, BNIP3L/CARD17, BNIP3L/CD274, BNIP3L/DYRK3, BNIP3L/FAM83A, BNIP3L/GBP5, BNIP3L/GLRX5, BNIP3L/GYPA, BNIP3L/IFIT1B, BNIP3L/ITLN1, BNIP3L/KLHDC8A, BNIP3L/P2RY14, BNIP3L/RHAG, BNIP3L/RHCE, BNIP3L/RNF182, BNIP3L/SPTA1, BNIP3L/TFEC, BNIP3L/THEM5, BNIP3L/TLCD4, BNIP3L/TMCC2, BNIP3L/TSPAN5, BNIP3L/TSPO2, CA1/ANKRD22, CA1/CARD17, CA1/DYRK3, CA1/FAM83A, CA1/GBP5, CA1/GLRX5, CA1/GYPA, CA1/IFIT1B, CA1/ITLN1, CA1/KLHDC8A, CA1/P2RY14, CA1/RHCE, CA1/RNF182, CA1/SPTA1, CA1/THEM5, CA1/TLCD4, CA1/TSPAN5, CD274/CA1, CD274/DYRK3, CD274/FAM83A, CD274/GLRX5, CD274/GYPA, CD274/IFIT1B, CD274/ITLN1, CD274/KLHDC8A, CD274/RHCE, CD274/RNF182, CD274/SPTA1, CD274/THEM5, CD274/TLCD4, CD274/TMCC2, CD274/TSPAN5, DYRK3/ANKRD22, DYRK3/CARD17, DYRK3/FAM83A, DYRK3/GBP5, DYRK3/GLRX5, DYRK3/GYPA, DYRK3/IFIT1B, DYRK3/ITLN1, DYRK3/KLHDC8A, DYRK3/P2RY14, DYRK3/RHCE, DYRK3/RNF182, DYRK3/SPTA1, DYRK3/THEM5, DYRK3/TLCD4, DYRK3/TSPAN5, FAM83A/ANKRD22, FAM83A/CARD17, FAM83A/GBP5, FAM83A/GLRX5, FAM83A/GYPA, FAM83A/IFIT1B, FAM83A/ITLN1, FAM83A/KLHDC8A, FAM83A/P2RY14, FAM83A/RHCE, FAM83A/RNF182, FAM83A/SPTA1, FAM83A/THEM5, FAM83A/TLCD4, FAM83A/TSPAN5, FECH/ANKRD22, FECH/APOL4, FECH/BNIP3L, FECH/CA1, FECH/CARD17, FECH/CD274, FECH/DYRK3, FECH/FAM83A, FECH/GBP5, FECH/GLRX5, FECH/GYPA, FECH/IFIT1B, FECH/ITLN1, FECH/KLHDC8A, FECH/P2RY14, FECH/RHAG, FECH/RHCE, FECH/RIOK3, FECH/RNF182, FECH/SPTA1, FECH/TFEC, FECH/THEM5, FECH/TLCD4, FECH/TMCC2, FECH/TSPAN5, FECH/TSPO2, GBP5/GLRX5, GBP5/GYPA, GBP5/IFIT1B, GBP5/ITLN1, GBP5/KLHDC8A, GBP5/RHCE, GBP5/RNF182, GBP5/SPTA1, GBP5/THEM5, GBP5/TSPAN5, GLRX5/CARD17, GLRX5/IFIT1B, GLRX5/RHCE, GLRX5/THEM5, GYPA/CARD17, GYPA/GLRX5, GYPA/IFIT1B, GYPA/ITLN1, GYPA/P2RY14, GYPA/RHCE, GYPA/RNF182, GYPA/THEM5, IFIT1B/CARD17, ITLN1/CARD17, ITLN1/GLRX5, ITLN1/IFIT1B, ITLN1/RHCE, ITLN1/RNF182, ITLN1/THEM5, KLHDC8A/CARD17, KLHDC8A/GLRX5, KLHDC8A/GYPA, KLHDC8A/IFIT1B, KLHDC8A/ITLN1, KLHDC8A/P2RY14, KLHDC8A/RHCE, KLHDC8A/RNF182, KLHDC8A/SPTA1, KLHDC8A/THEM5, KLHDC8A/TSPAN5, P2RY14/GLRX5, P2RY14/IFIT1B, P2RY14/ITLN1, P2RY14/RHCE, P2RY14/RNF182, P2RY14/THEM5, RHAG/ANKRD22, RHAG/CA1, RHAG/CARD17, RHAG/CD274, RHAG/DYRK3, RHAG/FAM83A, RHAG/GBP5, RHAG/GLRX5, RHAG/GYPA, RHAG/IFIT1B, RHAG/ITLN1, RHAG/KLHDC8A, RHAG/P2RY14, RHAG/RHCE, RHAG/RNF182, RHAG/SPTA1, RHAG/THEM5, RHAG/TLCD4, RHAG/TMCC2, RHAG/TSPAN5, RHAG/TSPO2, RHCE/CARD17, RHCE/IFIT1B, RHCE/THEM5, RIOK3/ANKRD22, RIOK3/BNIP3L, RIOK3/CA1, RIOK3/CARD17, RIOK3/CD274, RIOK3/DYRK3, RIOK3/FAM83A, RIOK3/GBP5, RIOK3/GLRX5, RIOK3/GYPA, RIOK3/IFIT1B, RIOK3/ITLN1, RIOK3/KLHDC8A, RIOK3/P2RY14, RIOK3/RHAG, RIOK3/RHCE, RIOK3/RNF182, RIOK3/SPTA1, RIOK3/TFEC, RIOK3/THEM5, RIOK3/TLCD4, RIOK3/TMCC2, RIOK3/TSPAN5, RIOK3/TSPO2, RNF182/CARD17, RNF182/GLRX5, RNF182/IFIT1B, RNF182/RHCE, RNF182/THEM5, SPTA1/CARD17, SPTA1/GLRX5, SPTA1/GYPA, SPTA1/IFIT1B, SPTA1/ITLN1, SPTA1/P2RY14, SPTA1/RHCE, SPTA1/RNF182, SPTA1/THEM5, SPTA1/TSPAN5, TFEC/CA1, TFEC/DYRK3, TFEC/FAM83A, TFEC/GLRX5, TFEC/GYPA, TFEC/IFIT1B, TFEC/ITLN1, TFEC/KLHDC8A, TFEC/RHAG, TFEC/RHCE, TFEC/RNF182, TFEC/SPTA1, TFEC/THEM5, TFEC/TLCD4, TFEC/TMCC2, TFEC/TSPAN5, TFEC/TSPO2, THEM5/CARD17, THEM5/IFIT1B, TLCD4/ANKRD22, TLCD4/CARD17, TLCD4/GBP5, TLCD4/GLRX5, TLCD4/GYPA, TLCD4/IFIT1B, TLCD4/ITLN1, TLCD4/KLHDC8A, TLCD4/P2RY14, TLCD4/RHCE, TLCD4/RNF182, TLCD4/SPTA1, TLCD4/THEM5, TLCD4/TSPAN5, TMCC2/ANKRD22, TMCC2/CA1, TMCC2/CARD17, TMCC2/DYRK3, TMCC2/FAM83A, TMCC2/GBP5, TMCC2/GLRX5, TMCC2/GYPA, TMCC2/IFIT1B, TMCC2/ITLN1, TMCC2/KLHDC8A, TMCC2/P2RY14, TMCC2/RHCE, TMCC2/RNF182, TMCC2/SPTA1, TMCC2/THEM5, TMCC2/TLCD4, TMCC2/TSPAN5, TSPAN5/CARD17, TSPAN5/GLRX5, TSPAN5/GYPA, TSPAN5/IFIT1B, TSPAN5/ITLN1, TSPAN5/P2RY14, TSPAN5/RHCE, TSPAN5/RNF182, TSPAN5/THEM5, TSPO2/ANKRD22, TSPO2/CA1, TSPO2/CARD17, TSPO2/CD274, TSPO2/DYRK3, TSPO2/FAM83A, TSPO2/GBP5, TSPO2/GLRX5, TSPO2/GYPA, TSPO2/IFIT1B, TSPO2/ITLN1, TSPO2/KLHDC8A, TSPO2/P2RY14, TSPO2/RHCE, TSPO2/RNF182, TSPO2/SPTA1, TSPO2/THEM5, TSPO2/TLCD4, TSPO2/TMCC2, and TSPO2/TSPAN5; wherein the IHD endotype signature pair is selected from: MAP7/SPRED1, SPRED1/GPR34, IL5RA/SPRED1, SPRED1/TPRG1, HRK/SPRED1, SPRED1/PLCB1, TRIM2/SPRED1, SIGLEC8/SPRED1, SMPD3/SPRED1, SPRED1/ZNF600, SPRED1/SDC2, MAP7/GPR34, PRSS33/SPRED1, SPRED1/DYNC2H1, CACNA2D3/SPRED1, ADAM23/GPR34, ADAM23/MAP7, ADAM23/PLCB1, ADAM23/SPRED1, ALOX15/GPR34, ALOX15/PLCB1, ALOX15/SPRED1, BAALC/GPR34, BAALC/PLCB1, BAALC/SPRED1, CACNA2D3/DYNC2H1, CACNA2D3/GPR34, CACNA2D3/PLCB1, CACNA2D3/SPRED1, CACNA2D3/ZNF600, GPR34/DYNC2H1, GPR34/GRAMD1C, GPR34/PLCB1, GPR34/TPRG1, GPR34/ZNF600, GPR82/DYNC2H1, GPR82/GPR34, GPR82/GRAMD1C, GPR82/PLCB1, GPR82/TPRG1, GPR82/ZNF600, GRAMD1C/DYNC2H1, GRAMD1C/PLCB1, GRAMD1C/ZNF600, HRK/DYNC2H1, HRK/GPR34, HRK/MAP7, HRK/PLCB1, HRK/SPRED1, HRK/ZNF600, IL5RA/DYNC2H1, IL5RA/GPR34, IL5RA/PLCB1, IL5RA/SPRED1, IL5RA/TRIM2, MAP7/BAALC, MAP7/CACNA2D3, MAP7/DYNC2H1, MAP7/GPR34, MAP7/GPR82, MAP7/GRAMD1C, MAP7/PLCB1, MAP7/SPRED1, MAP7/TPRG1, MAP7/ZNF600, PLCB1/DYNC2H1, PLCB1/TPRG1, PLCB1/ZNF600, PRSS33/GPR34, PRSS33/PLCB1, PRSS33/SPRED1, SDC2/DYNC2H1, SDC2/GPR34, SDC2/PLCB1, SDC2/ZNF600, SIGLEC8/DYNC2H1, SIGLEC8/GPR34, SIGLEC8/MAP7, SIGLEC8/PLCB1, SIGLEC8/SPRED1, SIGLEC8/TRIM2, SMPD3/DYNC2H1, SMPD3/GPR34, SMPD3/MAP7, SMPD3/PLCB1, SMPD3/SPRED1, SMPD3/TRIM2, SPRED1/DYNC2H1, SPRED1/GPR34, SPRED1/GPR82, SPRED1/GRAMD1C, SPRED1/PLCB1, SPRED1/SDC2, SPRED1/TPRG1, SPRED1/ZNF600, TRIM2/CACNA2D3, TRIM2/DYNC2H1, TRIM2/GPR34, TRIM2/GPR82, TRIM2/GRAMD1C, TRIM2/HRK, TRIM2/MAP7, TRIM2/PLCB1, TRIM2/SDC2, TRIM2/SPRED1, TRIM2/TPRG1, and TRIM2/ZNF600; wherein the IFN endotype signature pair is selected from: ETV7/PLEKHO1, IFITM3/ETV7, ETV7/APOL1, BATF2/ETV7, PLEKHO1/BATF2, ETV7/EPSTI1, EPSTI1/BATF2, IFITM3/BATF2, USP18/EPSTI1, ETV7/SEPTIN4, ETV7/LAMP3, SERPING1/BATF2, LAMP3/BATF2, LAMP3/SERPING1, APOL1/BATF2, APOL1/CLEC4F, APOL1/EPSTI1, APOL1/EXOC3L1, APOL1/HES4, APOL1/IFITM3, APOL1/LY6E, APOL1/RSAD2, APOL1/SEPTIN4, APOL1/SERPING1, APOL1/TPPP3, BATF2/EXOC3L1, BATF2/HES4, CLEC4F/BATF2, CLEC4F/EXOC3L1, EPSTI1/BATF2, EPSTI1/CLEC4F, EPSTI1/EXOC3L1, EPSTI1/HES4, EPSTI1/IFITM3, EPSTI1/LY6E, EPSTI1/RSAD2, EPSTI1/SERPING1, EPSTI1/TPPP3, ETV7/APOL1, ETV7/BATF2, ETV7/CLEC4F, ETV7/EPSTI1, ETV7/EXOC3L1, ETV7/HES4, ETV7/IFITM3, ETV7/LAMP3, ETV7/LY6E, ETV7/PLEKHO1, ETV7/RSAD2, ETV7/SEPTIN4, ETV7/SERPING1, ETV7/TPPP3, EXOC3L1/HES4, IFITM3/SERPING1, IFITM3/CLEC4F, IFITM3/TPPP3, IFITM3/LY6E, IFITM3/EXOC3L1, IFITM3/HES4, LAMP3/APOL1, LAMP3/BATF2, LAMP3/CLEC4F, LAMP3/EPSTI1, LAMP3/EXOC3L1, LAMP3/HES4, LAMP3/IFITM3, LAMP3/LY6E, LAMP3/RSAD2, LAMP3/SEPTIN4, LAMP3/SERPING1, LAMP3/TPPP3, LY6E/BATF2, LY6E/EXOC3L1, PLEKHO1/APOL1, PLEKHO1/BATF2, PLEKHO1/EPSTI1, PLEKHO1/EXOC3L1, PLEKHO1/IFITM3, PLEKHO1/LAMP3, PLEKHO1/RSAD2, PLEKHO1/SEPTIN4, PLEKHO1/SERPING1, RSAD2/BATF2, RSAD2/CLEC4F, RSAD2/EXOC3L1, RSAD2/HES4, RSAD2/IFITM3, RSAD2/LY6E, RSAD2/SERPING1, RSAD2/TPPP3, SEPTIN4/BATF2, SEPTIN4/CLEC4F, SEPTIN4/EPSTI1, SEPTIN4/EXOC3L1, SEPTIN4/HES4, SEPTIN4/IFITM3, SEPTIN4/LY6E, SEPTIN4/RSAD2, SEPTIN4/SERPING1, SEPTIN4/TPPP3, SERPING1/BATF2, SERPING1/CLEC4F, SERPING1/EXOC3L1, SERPING1/HES4, SERPING1/LY6E, SERPING1/TPPP3, TPPP3/BATF2, and TPPP3/EXOC3L1; and wherein the ADA endotype signature pair is selected from: LGALS3BP/OTOF, LGALS3BP/IF127, LGALS3BP/KIF14, LGALS3BP/CENPF, GTSE1/LGALS3BP, LGALS3BP/KCTD14, LGALS3BP/PDIA4, LGALS3BP/TSHR, LGALS3BP/PLAAT2, OTOF/IF127, IGF1/LGALS3BP, CDC45/LGALS3BP, LGALS3BP/KIF15, LGALS3BP/IGLL5, LGALS3BP/MIXL, CAV1/LGALS3BP, CAV1/OTOF, CDC45/LGALS3BP, CDC45/OTOF, CENPF/KCTD14, GPRC5D/OTOF, GTSE1/LGALS3BP, GTSE1/OTOF, IGF1/LGALS3BP, IGF1/OTOF, KCTD14/KLHL14, KCTD14/PDIA4, KCTD14/TSHR, KIF14/KCTD14, LGALS3BP/CENPF, LGALS3BP/GPRC5D, LGALS3BP/IFI27, LGALS3BP/IGLL5, LGALS3BP/KCTD14, LGALS3BP/KIF14, LGALS3BP/KIF15, LGALS3BP/KLHL14, LGALS3BP/MIR155HG, LGALS3BP/MIXL1, LGALS3BP/OTOF, LGALS3BP/PDIA4, LGALS3BP/PLAAT2, LGALS3BP/SDC1, LGALS3BP/SLC16A14, LGALS3BP/TSHR, OTOF/CENPF, OTOF/IF127, OTOF/IGLL5, OTOF/KCTD14, OTOF/KIF14, OTOF/KIF15, OTOF/KLHL14, OTOF/MIR155HG, OTOF/MIXL1, OTOF/PDIA4, OTOF/PLAAT2, OTOF/SDC1, OTOF/SLC16A14, OTOF/TSHR, PLAAT2/KCTD14, TNFRSF17/LGALS3BP, and TNFRSF17/OTOF.

In an embodiment, the reference gene signature represents a standard level of expression of the genes comprised therein and a difference between a sample endotype signature pair and a reference endotype signature pair indicates that the subject has the sepsis mechanistic endotype corresponding to that signature pair.

In an embodiment, the sample gene signature and the reference gene signature comprise the NPS endotype signature pair, the INF endotype signature pair, the IHD endotype signature pair, the IFN endotype signature pair, and the ADA endotype signature pair.

In an embodiment, the NPS endotype signature pair is selected from: GADD45A/EFNA1, EFNA1/MIR646HG, MIR646HG/KLF14, MLLT1/MIR646HG, ARG1/MLLT1, MLLT1/EFNA1, MLLT1/NSUN7, EFNA1/NSUN7, SLC51A/EFNA1, EFNA1/KLF14, ZDHHC19/EFNA1, EFNA1/AGFG1, NSUN7/KLF14, EFNA1/PFKFB2, and MLLT1/KLF14. In another embodiment, the INF endotype signature pair is selected from: FECH/TFEC, TFEC/IFIT1B, FECH/RNF182, IFIT1B/FECH, FECH/APOL4, FECH/GYPA, ITLN1/FECH, FECH/THEM5, IFIT1B/CA1, RHAG/FECH, FECH/FAM83A, RHCE/FECH, TFEC/CA1, and SPTA1/FECH. In a further embodiment, the IHD endotype signature pair is selected from: MAP7/SPRED1, SPRED1/GPR34, IL5RA/SPRED1, SPRED1/TPRG1, HRK/SPRED1, SPRED1/PLCB1, TRIM2/SPRED1, SIGLEC8/SPRED1, SMPD3/SPRED1, SPRED1/ZNF600, SPRED1/SDC2, MAP7/GPR34, PRSS33/SPRED1, SPRED1/DYNC2H1, and CACNA2D3/SPRED1. In another embodiment, the IFN endotype signature pair is selected from: ETV7/PLEKHO1, IFITM3/ETV7, ETV7/APOL1, BATF2/ETV7, PLEKHO1/BATF2, ETV7/EPSTI1, EPSTI1/BATF2, IFITM3/BATF2, USP18/EPSTI1, ETV7/SEPTIN4, ETV7/LAMP3, SERPING1/BATF2, LAMP3/BATF2, and LAMP3/SERPING1. In an embodiment, the ADA endotype signature pair is selected from: LGALS3BP/OTOF, LGALS3BP/IFI27, LGALS3BP/KIF14, LGALS3BP/CENPF, GTSE1/LGALS3BP, LGALS3BP/KCTD14, LGALS3BP/PDIA4, LGALS3BP/TSHR, LGALS3BP/PLAAT2, OTOF/IF1I27, IGF1/LGALS3BP, CDC45/LGALS3BP, LGALS3BP/KIF15, LGALS3BP/IGLL5, and LGALS3BP/MIXL1.

The present disclosure also includes a method for predicting severity of sepsis in a subject, wherein the severity of the sepsis is selected from high severity sepsis, intermediate severity sepsis and low severity sepsis, the method comprising: (a) determining, in a biological sample from the subject, a level of expression for each of a plurality of genes, to provide a sample gene signature; and (b) comparing the sample gene signature with a reference gene signature to predict the severity of the sepsis in the subject, wherein high severity sepsis means a sequential organ failure assessment (SOFA) score of greater than or equal to 5, intermediate severity sepsis means a SOFA score of greater than or equal to 2 but less than 5, and low severity sepsis means a SOFA score of less than 2; and wherein the plurality of genes is selected from the group consisting of ABCA13, ADAMTS2, ADAMTS3, AK5, ANKRD22, ANKRD34B, ANLN, AQP1, ARG1, ARHGAP44, ARHGEF17, ASPM, ATP1B2, AURKA, AZU1, BAIAP3, BPI, Clorf226, CACNB4, CCL4L2, CCN3, CCNA1, CD177, CD24, CDK1, CDKN3, CEACAM6, CEACAM8, CENPA, CFH, CHDH, CHITI, CKAP2L, CLEC4C, CLEC4F, CLNK, COL17A1, CRISP2, CRISP3, CTSE, CTSG, CYP19A1, CYYR1, DEFA4, DENND2C, DEPDC1, DGKK, DLC1, DLGAP5, DNAH10, DOC2B, DSP, ELANE, ERG, FAM20A, FAM83A, FBN1, FFAR3, GOS2, GGT5, GLB1L2, GJB6, GPR84, GRAMD1C, GYPA, HBM, HMGB3, HP, HPGD, HRK, IGLL1, IL1R2, IL1RL1, INHBA, IQGAP3, ITGA7, ITGB4, KIF15, KIF20A, KLF14, LAMB3, LCN2, LGR4, LPL, LTF, MAFG, MERTK, METTL7B, MMP8, MMP9, MPO, MRC1, MROCKI, MS4A3, MS4A4A, NECAB1, NEIL3, NEK2, NRXN2, NUF2, OLAH, OLFM4, OLIG2, PCOLCE2, PCSK9, PHF24, PIGR, PLAAT2, PPARG, PRTN3, PTGES, PYCRI, RAB3IL1, RASGRF1, RETN, RHCE, RIPOR3, RPGRIP1, RRM2, S100A12, S100A8, SCN8A, SEMA6B, SERPINB10, SIGLEC8, SIL1, SLC16A1, SLC28A3, SLC39A8, SLC4A10, SLC51A, SLC6A19, SLC8A3, SLCO4A1, SMIMI, SMPDL3A, SPATC1, SPOP, SSBP2, TCN1, TCTEX1D1, TDRD9, TEAD2, TFRC, THBS1, TIMP3, TLN2, TMEM255A, TMEM45A, TNFAIP8L3, TNIP3, TROAP, TTK, VSIG4, WNT3, YPEL4, and ZDHHC19.

In an embodiment, the plurality of genes comprises CCL4L2, GPR84, HRK, MMP8, GGT5, and RASGRF1. In another embodiment, the plurality of genes is CCL4L2, GPR84, HRK, MMP8, GGT5, and RASGRF1.

In an embodiment, determining the level of expression comprises detecting nucleic acids encoded by each of the plurality of genes. In another embodiment, determining the level of expression comprises one or more of a polymerase chain reaction (PCR) amplification method, a non-PCR based amplification method, reverse transcriptase-(RT) PCR, Q-beta replicase amplification, ligase chain reaction, signal amplification (Ampliprobe), light cycling, differential display, Northern analysis, hybridization, microarray analysis, DNA sequencing, RNA sequencing (RNA-Seq), MassArray analysis and MALDI-TOF mass spectrometry. In a further embodiment, determining the level of expression comprises a polymerase chain reaction (PCR) amplification method. In another embodiment, determining the level of expression comprises RNA sequencing (RNA-Seq).

In an embodiment, the biological sample comprises sputum, blood, nasal brushings, throat swabs, urine, amniotic fluid, plasma, serum, saliva, semen, bone marrow, tissue or fine needle biopsy samples, stool, bronchoalveolar lavage fluid, cerebrospinal fluid, peritoneal fluid, pleural fluid, skin, or cells therefrom. In another embodiment, the biological sample comprises blood. In an embodiment, the biological sample has been obtained from the subject prior to admission in an intensive care unit. In another embodiment, the biological sample has been obtained from the subject at first clinical presentation. In a further embodiment, the biological sample has been obtained from the subject within the first day after entry into an intensive care unit.

The present disclosure also includes a use of one or more therapies that act specifically against a mechanism associated with a sepsis mechanistic endotype, for treatment of sepsis in a subject classified as having the sepsis mechanistic endotype by a method for classifying a subject into a sepsis mechanistic endotype of the present disclosure.

The present disclosure also includes one or more therapies that act specifically against a mechanism associated with a sepsis mechanistic endotype for use to treat sepsis in a subject classified as having the sepsis mechanistic endotype by a method for classifying a subject into a sepsis mechanistic endotype of the present disclosure.

The present disclosure also includes a use of an effective amount of one or more antibiotics for treatment of sepsis in a subject predicted as having high or intermediate severity sepsis by a method comprising: (a) determining, in a biological sample from the subject, a level of expression for each of a plurality of genes, to provide a sample gene signature; and (b) comparing the sample gene signature with a reference gene signature to predict the severity of the sepsis in the subject, wherein high severity sepsis means a sequential organ failure assessment (SOFA) score of greater than or equal to 5, and intermediate severity sepsis means a SOFA score of greater than or equal to 2 but less than 5; and wherein the plurality of genes is selected from the group consisting of ABCA13, ADAMTS2, ADAMTS3, AK5, ANKRD22, ANKRD34B, ANLN, AQP1, ARG1, ARHGAP44, ARHGEF17, ASPM, ATP1B2, AURKA, AZU1, BAIAP3, BPI, Clorf226, CACNB4, CCL4L2, CCN3, CCNA1, CD177, CD24, CDK1, CDKN3, CEACAM6, CEACAM8, CENPA, CFH, CHDH, CHIT1, CKAP2L, CLEC4C, CLEC4F, CLNK, COL17A1, CRISP2, CRISP3, CTSE, CTSG, CYP19A1, CYYR1, DEFA4, DENND2C, DEPDC1, DGKK, DLC1, DLGAP5, DNAH10, DOC2B, DSP, ELANE, ERG, FAM20A, FAM83A, FBN1, FFAR3, GOS2, GGT5, GLB1L2, GJB6, GPR84, GRAMD1C, GYPA, HBM, HMGB3, HP, HPGD, HRK, IGLL1, IL1R2, IL1RL1, INHBA, IQGAP3, ITGA7, ITGB4, KIF15, KIF20A, KLF14, LAMB3, LCN2, LGR4, LPL, LTF, MAFG, MERTK, METTL7B, MMP8, MMP9, MPO, MRC1, MROCKI, MS4A3, MS4A4A, NECAB1, NEIL3, NEK2, NRXN2, NUF2, OLAH, OLFM4, OLIG2, PCOLCE2, PCSK9, PHF24, PIGR, PLAAT2, PPARG, PRTN3, PTGES, PYCRI, RAB3IL1, RASGRF1, RETN, RHCE, RIPOR3, RPGRIP1, RRM2, S100A12, S100A8, SCN8A, SEMA6B, SERPINB10, SIGLEC8, SIL1, SLC16A1, SLC28A3, SLC39A8, SLC4A10, SLC51A, SLC6A19, SLC8A3, SLCO4A1, SMIM1, SMPDL3A, SPATC1, SPOP, SSBP2, TCN1, TCTEX1D1, TDRD9, TEAD2, TFRC, THBS1, TIMP3, TLN2, TMEM255A, TMEM45A, TNFAIP8L3, TNIP3, TROAP, TTK, VSIG4, WNT3, YPEL4, and ZDHHC19.

The present disclosure also includes one or more antibiotics for use to treat sepsis in a subject predicted as having high or intermediate severity sepsis by a method for predicting severity of sepsis comprising: (i) determining, in a biological sample from the subject, a level of expression for each of a plurality of genes, to provide a sample gene signature; and (ii) comparing the sample gene signature with a reference gene signature to predict the severity of the sepsis in the subject, wherein high severity sepsis means a sequential organ failure assessment (SOFA) score of greater than or equal to 5, and intermediate severity sepsis means a SOFA score of greater than or equal to 2 but less than 5; and wherein the plurality of genes is selected from the group consisting of ABCA13, ADAMTS2, ADAMTS3, AK5, ANKRD22, ANKRD34B, ANLN, AQP1, ARG1, ARHGAP44, ARHGEF17, ASPM, ATP1B2, AURKA, AZU1, BAIAP3, BPI, Clorf226, CACNB4, CCL4L2, CCN3, CCNA1, CD177, CD24, CDK1, CDKN3, CEACAM6, CEACAM8, CENPA, CFH, CHDH, CHIT1, CKAP2L, CLEC4C, CLEC4F, CLNK, COL17A1, CRISP2, CRISP3, CTSE, CTSG, CYP19A1, CYYR1, DEFA4, DENND2C, DEPDC1, DGKK, DLC1, DLGAP5, DNAH10, DOC2B, DSP, ELANE, ERG, FAM20A, FAM83A, FBN1, FFAR3, GOS2, GGT5, GLB1L2, GJB6, GPR84, GRAMD1C, GYPA, HBM, HMGB3, HP, HPGD, HRK, IGLL1, IL1R2, IL1RL1, INHBA, IQGAP3, ITGA7, ITGB4, KIF15, KIF20A, KLF14, LAMB3, LCN2, LGR4, LPL, LTF, MAFG, MERTK, METTL7B, MMP8, MMP9, MPO, MRC1, MROCKI, MS4A3, MS4A4A, NECAB1, NEIL3, NEK2, NRXN2, NUF2, OLAH, OLFM4, OLIG2, PCOLCE2, PCSK9, PHF24, PIGR, PLAAT2, PPARG, PRTN3, PTGES, PYCRI, RAB3IL1, RASGRF1, RETN, RHCE, RIPOR3, RPGRIP1, RRM2, S100A12, S100A8, SCN8A, SEMA6B, SERPINB10, SIGLEC8, SIL1, SLC16A1, SLC28A3, SLC39A8, SLC4A10, SLC51A, SLC6A19, SLC8A3, SLCO4A1, SMIM1, SMPDL3A, SPATC1, SPOP, SSBP2, TCN1, TCTEX1D1, TDRD9, TEAD2, TFRC, THBS1, TIMP3, TLN2, TMEM255A, TMEM45A, TNFAIP8L3, TNIP3, TROAP, TTK, VSIG4, WNT3, YPEL4, and ZDHHC19.

In an embodiment, the one or more antibiotics is one or a combination of a glycopeptide, a cephalosporin, a beta-lactam, a beta-lactamase inhibitor, a carbapenem, a quinolone, a fluoroquinolone, an aminoglycoside, a macrolide and a monobactam.

The present disclosure also includes a kit: (a) for classifying a subject into a sepsis mechanistic endotype selected from neutrophilic-suppressive (NPS), inflammatory (INF), innate host defense (IHD), interferon (IFN) and adaptive (ADA) endotypes, the kit comprising gene specific reagents, each of the gene specific reagents capable of detecting an expression product of (i) a respective one of a plurality of genes or complement thereof in an NPS endotype sub-signature, an INF endotype sub-signature, an IHD endotype sub-signature, an IFN endotype sub-signature, an ADA endotype sub-signature or combinations thereof, wherein the NPS endotype sub-signature, the INF endotype sub-signature, the IHD endotype sub-signature, the IFN endotype sub-signature, the ADA endotype sub-signature or combinations thereof are as described herein; or (ii) a respective one of a plurality of genes or complement thereof in an NPS endotype signature pair, an INF endotype signature pair, an IHD endotype signature pair, an IFN endotype signature pair, an ADA endotype signature pair or combinations thereof, wherein the NPS endotype signature pair, the INF endotype signature pair, the IHD endotype signature pair, the IFN endotype signature pair, the ADA endotype signature pair or combinations thereof are as described herein; or (b) for predicting severity of sepsis in a subject, wherein the severity of the sepsis is selected from high severity sepsis, intermediate severity sepsis and low severity sepsis, wherein high severity sepsis means a sequential organ failure assessment (SOFA) score of greater than or equal to 5, intermediate severity sepsis means a SOFA score of greater than or equal to 2 but less than 5, and low severity sepsis means a SOFA score ofless than 2, the kit comprising gene specific reagents, each of the gene specific reagents capable of detecting an expression product of a respective one of a plurality of genes as described herein or complement thereof, and optionally instructions for use.

The present disclosure also includes a method for identifying a candidate agent for the treatment of sepsis in a subject classified as having a sepsis mechanistic endotype selected from neutrophilic-suppressive (NPS), inflammatory (INF), innate host defense (IHD), interferon (IFN) and adaptive (ADA) endotypes, the method comprising: (a) contacting a cell having the sepsis endotype with a test agent, (b) determining the level of expression for each of a plurality of genes in the cell to provide an expression signature; (c) comparing the expression signature with a reference signature, wherein the reference signature represents the level of expression of the plurality of genes in a normal cell; and (d) selecting the test agent as a candidate agent for treatment of the sepsis when the expression signature substantially corresponds with the reference signature, wherein the expression signature and reference signature comprise: (a) an NPS endotype sub-signature for an NPS endotype cell, an INF endotype sub-signature for an INF endotype cell, an IHD endotype sub-signature for an IHD endotype cell, an IFN endotype sub-signature for an IFN endotype cell and an ADA endotype sub-signature for an ADA endotype cell, wherein the NPS endotype sub-signature, the INF endotype sub-signature, the IHD endotype sub-signature, the IFN endotype sub-signature, the ADA endotype sub-signature or combinations thereof are as described herein; or (b) an NPS endotype signature pair for an NPS endotype cell, an INF endotype signature pair for an INF endotype cell, an IHD endotype signature pair for an IHD endotype cell, an IFN endotype signature pair for an IFN endotype cell, and an ADA endotype signature pair for an ADA endotype cell, wherein the NPS endotype signature pair, the INF endotype signature pair, the IHD endotype signature pair, the IFN endotype signature pair, the ADA endotype signature pair or combinations thereof are as described herein.

In an embodiment is provided a method of detecting a sepsis mechanistic endotype, using endotype-specific gene signatures for the NPS, INF, IHD, IFN and ADA endotypes, in a biological sample obtained from a subject suspected of having sepsis, at risk of developing severe sepsis, at risk of organ failure, or having endotoxin tolerance, the method comprising detecting a level of expression, in a biological sample obtained from the subject, for each of a plurality of Endotoxin Tolerance Signature genes to provide a sample gene signature, wherein plurality of genes is selected from the group consisting of NPS signature: AGFG1, ARG1, ATP9A, ANXA3, EFNA1, GADD45A, GPR84, HPGD, IL1R1, KLF14, KREMEN1, MIR646HG, MLLT1, NSUN7, OLAH, ORM2, PCOLCE2, PFKFB2, SLC51A, TNFAIP8L3, ZDHHC19, ADAMTS3, AKR1C1, ALDH1A2, ALOX5AP, ALPL, AMPH, ANKRD55, BCL3, BTBD19, CA4, CD163L1, CD177, CD82, CST7, CYP19A1, CYSTM1, DAAM2, DGAT2, ECHDC3, ENTPD7, EXOSC4, FFAR3, FGF13, FSTL4, GALNT14, GRAMD1A, GRB10, GYG1, HPGD, IER3, IL18RAP, IL1R2, IL1RN, IRAG1-AS1, KCNE1B, KCNMA1, MCEMP1, MKNK1, MMP9, MSRA, NECAB1, NSMCE1-DT, OPLAH, PDGFC, PFKFB3, PHF24, PI3, PLIN4, PLIN5, PLK3, POR, PROK2, RFX2, RGL4, ROM1, S100A12, S100P, SEMA6B, SHROOM4, SLP1, SOCS3, SPATC1, SPDYA, SPINK8, SPP1, ST6GALNAC3, SYN2, TDRD9, TMEM120A, TMIGD3, TSPO, UPP1, XCR1; INF signature: BNIP3L, CA1, FAM83A, FECH, GLRX5, GYPA, IFIT1B, RHCE, RIOK3, RNF182, SLC6A19, SPTA1, THEM5, TLCD4, TSPAN5, TSPO2, ABCG2, ACHE, ACKR1, ACSL6, ADD2, AHSP, ALAS2, ALDH5A1, ANK1, ANKRD9, AQP1, ARHGEF12, ARHGEF37, ARL4A, ATP1B2, ATP1B2, BBOF1, BCAM, BCL2L1, BLVRB, BPGM, Clorf116, CA2, CISD2, CLIC2, CR1L, CR1L, CTNNAL1, CTSE, CTSE, DCAF12, DMTN, DNAJC6, DPCD, DYRK3, EMID1, EPB42, ERFE, FAM210B, FAXDC2, FRMD4A, GMPR, GSPT1, GYPB, HBM, HEMGN, HEPACAM2, HMBS, IGF2BP2, ISCA1, ITLN1, KANK2, KCNH2, KDM7A-DT, KEL, KLC3, KLF1, KLHDC8A, KRT1, LRRC2, MAOA, MAOA, MARCHF8, MBNL3, MFSD2B, MRC2, MXI1, MYL4, NFIX, NUDT4, OSBP2, PAGE2B, PBX1, PCDH1, PGF, PLEK2, PNP, PRDX2, PTPRF, RAPIGAP, RBM38, RFESD, RFESD, RGCC, RGS16, RHAG, RHD, RIPOR3, RNF175, RUNDC3A, SEC14L4, SELENBP1, SELENOP, SFRP2, SGIP1, SIAH2, SLC14A1, SLC1A5, SLC22A23, SLC2A1, SLC6A8, SLC6A9, SLC7A5, SMIM5, SNCA, SOX6, SPTB, STRADB, TAL1, TENT5C, TFR2, TMCC2, TMOD1, TNS1, TRIM10, TRIM58, TSPAN7, TTC25, UBB, USP12, XK, YBX3, YPEL4; IHD signature: ABCA6, ADAM23, ALOX15, CACNA2D3, DYNC2H1, GPR34, GRAMD1C, LPL, MAP7, MIR155HG, PLCB1, SDC2, SIGLEC8, SPRED1, SLC16A14, SMPD3, TPPP3, TPRG1, ZNF600, ADGRD1, ANGPT1, GPR82, HDAC9, IL5RA, KLHDC1, PRSS33, PTGDR2, PTGFRN, TBC1D12, TRIM2; IFN signature: ANKRD22, APOL1, APOL4, BATF2, CARD17, CD274, EPSTI1, ETV7, GBP5, IFITM3, P2RY14, PLEKHO1, RSAD2, SERPING1, TFEC, EXOC3L1, IRF7, OAS1, SEPTIN4, LY6E, LAMP3; ADA signature: CCL2, CDC45, CENPF, CLEC4F, GTSE1, IFI27, KCTD14, KIF14, KIF15, KLHDC7B, LGALS3BP, OTOF, PDIA4, SIGLEC1, AGRN, CD38, CDCA7, CDT1, CTLA4, DHX58, EME1, FAM111B, HES4, IFI44L, IFIT3, IFNG-AS1, IL12RB2, IL4I1, KIF19, LAG3, MCM10, P2RY6, PACSIN1, PARM1, SAMD4A, SPATS2L, HERC5, TMPRSS3, TNFRSF13B, TSHR, TTC21A, wherein an Endotype Signature gene signature is detected when the sample gene signature is different from a reference gene signature, wherein the reference gene signature represents a standard level of expression of each of the plurality of genes.

In another embodiment is provided a method of detecting a sepsis mechanistic endotype, using endotype-specific gene signatures for the NPS, INF, IHD, IFN and ADA endotypes, in a biological sample obtained from a subject suspected of having sepsis, at risk of developing severe sepsis, at risk of organ failure, or having endotoxin tolerance, the method comprising detecting a level of expression, in a biological sample obtained from the subject, for each of a plurality of Endotoxin Tolerance Signature genes to provide a sample gene signature, wherein plurality of genes is selected from the group consisting of NPS-selective: AGFG1, ARG1, ATP9A, ANXA3, EFNA1, GADD45A, GPR84, HPGD, IL1R1, KLF14, KREMEN1, MIR646HG, MLLT1, NSUN7, OLAH, ORM2, PCOLCE2, PFKFB2, SLC51A, TNFAIP8L3, ZDHHC19; INF-selective: BNIP3L, CA1, FAM83A, FECH, GLRX5, GYPA, IFIT1B, RHCE, RIOK3, RNF182, SLC6A19, SPTA1, THEM5, TLCD4, TSPAN5, TSPO2, IHD-selective: ABCA6, ADAM23, ALOX15, CACNA2D3, DYNC2H1, GPR34, GRAMD1C, LPL, MAP7, MIR155HG, PLCB1, SDC2, SIGLEC8, SPRED1, SLC16A14, SMPD3, TPPP3, TPRG1, ZNF600; IFN-selective: ANKRD22, APOL1, APOL4, BATF2, CARD17, CD274, EPSTI1, ETV7, GBP5, IFITM3, P2RY14, PLEKHO1, RSAD2, SERPING1, TFEC; ADA-selective: CCL2, CDC45, CENPF, CLEC4F, GTSE1, IFI27, KCTD14, KIF14, KIF15, KLHDC7B, LGALS3BP, OTOF, PDIA4, SIGLEC1.

In an embodiment, the difference between the sample gene signature and the reference gene signature is defined by a difference in expression of at least two of the plurality of genes in an expression change direction, at least 5 of the plurality of genes in an expression change direction, at least 10 of the plurality of genes in an expression change direction, at least 15 of the plurality of genes in an expression change direction, at least 20 of the plurality of genes in an expression change direction, at least 25 of the plurality of genes in an expression change direction, at least 30 of the plurality of genes in an expression change direction, or at least 31 of the plurality of genes in an expression change direction.

In another embodiment is provided a method of detecting a sepsis mechanistic endotype, using endotype-specific gene signatures for the NPS, INF, IHD, IFN and ADA endotypes, in a biological sample obtained from a subject suspected of having sepsis, at risk of developing severe sepsis, at risk of organfailure, or having endotoxin tolerance, the method comprising detecting a level of expression, in a biological sample obtained from the subject, for each of a plurality of Endotoxin Tolerance Signature genes to provide a sample gene signature, wherein plurality of genes is selected from the group consisting of two genes, and wherein the pair of genes are selected from the pairs comprising NPS signature pairs: GADD45A/EFNA1, EFNA1/MIR646HG, MIR646HG/KLF14, MLLT1/MIR646HG, ARG1/MLLT1, MLLT1/EFNA1, MLLT1/NSUN7, EFNA1/NSUN7, SLC51A/EFNA1, EFNA1/KLF14, ZDHHC19/EFNA1, EFNA1/AGFG1, NSUN7/KLF14, EFNA1/PFKFB2, MLLT1/KLF14. ADAMTS3/PCOLCE2; ADAMTS3/ZDHHC19; ADAMTS3/SLC51A; ADAMTS3/HPGD; ADAMTS3/SEMA6B; ADAMTS3/EFNA1; ADAMTS3/AGFG1; ADAMTS3/NSUN7; ADAMTS3/TNFAIP8L3; ADAMTS3/KREMEN1; ADAMTS3/ORM2; ADAMTS3/MIR646HG; ADAMTS3/KLF14; AGFG1/NSUN7; AGFG1/TNFAIP8L3; AGFG1/KREMEN1; AGFG1/ORM2; AGFG1/MIR646HG; AGFG1/KLF14; ANXA3/GPR84; ANXA3/OLAH; ANXA3/ADAMTS3; ANXA3/PCOLCE2; ANXA3/ZDHHC19; ANXA3/SLC51A; ANXA3/HPGD; ANXA3/SEMA6B; ANXA3/EFNA1; ANXA3/AGFG1; ANXA3/NSUN7; ANXA3/TNFAIP8L3; ANXA3/KREMEN1; ANXA3/ORM2; ANXA3/MIR646HG; ANXA3/KLF14; ARG1/PFKFB2; ARG1/MLLT1; ARG1/ANXA3; ARG1/GPR84; ARG1/OLAH; ARG1/ADAMTS3; ARG1/PCOLCE2; ARG1/ZDHHC19; ARG1/SLC51A; ARG1/HPGD; ARG1/SEMA6B; ARG1/EFNA1; ARG1/AGFG1; ARG1/NSUN7; ARG1/TNFAIP8L3; ARG1/KREMEN1; ARG1/ORM2; ARG1/MIR646HG; ARG1/KLF14; ATP9A/EPB41L4B; ATP9A/IL1R1; ATP9A/GADD45A; ATP9A/ARG1; ATP9A/PFKFB2; ATP9A/MLLT1; ATP9A/ANXA3; ATP9A/GPR84; ATP9A/OLAH; ATP9A/ADAMTS3; ATP9A/PCOLCE2; ATP9A/ZDHHC19; ATP9A/SLC51A; ATP9A/HPGD; ATP9A/EMA6B; ATP9A/EFNA1; ATP9A/AGFG1; ATP9A/NSUN7; ATP9A/TNFAIP8L3; ATP9A/KREMEN1; ATP9A/ORM2; ATP9A/MIR646HG; ATP9A/KLF14; EFNA1/AGFG1; EFNA1/NSUN7; EFNA1/TNFAIP8L3; EFNA1/KREMEN1; EFNA1/ORM2; EFNA1/MIR646HG; EFNA1/KLF14; EPB41L4B/IL1R1; EPB41L4B/GADD45A; EPB41L4B/ARG1; EPB41L4B/PFKFB2; EPB41L4B/LLT1; EPB41L4B/ANXA3; EPB41L4B/GPR84; EPB41L4B/OLAH; EPB41L4B/ADAMTS3; EPB41L4B/PCOLCE2; EPB41L4B/ZDHHC19; EPB41L4B/SLC51A; EPB41L4B/HPGD; EPB41L4B/SEMA6B; EPB41L4B/EFNA1; EPB41L4B/AGFG1; EPB41L4B/NSUN7; EPB41L4B/TNFAIP8L3; EPB41L4B/KREMEN1; EPB41L4B/MIR646HG; EPB41L4B/KLF14; GADD45A/ARG1; GADD45A/PFKFB2; GADD45A/MLLT1; GADD45A/ANXA3; GADD45A/GPR84; GADD45A/OLAH; GADD45A/ADAMTS3; GADD45A/PCOLCE2; GADD45A/ZDHHC19; GADD45A/SLC51A; GADD45A/HPGD; GADD45A/SEMA6B; GADD45A/EFNA1; GADD45A/AGFG1; GADD45A/NSUN7; GADD45A/TNFAIP8L3; GADD45A/KREMEN1; GADD45A/ORM2; GADD45A/MIR646HG; GADD45A/KLF14; GPR84/OLAH; GPR84/ADAMTS3; GPR84/PCOLCE2; GPR84/ZDHHC19; GPR84/SLC51A; GPR84/HPGD; GPR84/SEMA6B; GPR84/EFNA1; GPR84/AGFG1; GPR84/NSUN7; GPR84/TNFAIP8L3; GPR84/KREMEN1; GPR84/ORM2; GPR84/MIR646HG; GPR84/KLF14; HPGD/SEMA6B; HPGD/EFNA1; HPGD/AGFG1; HPGD/NSUN7; HPGD/TNFAIP8L3; HPGD/KREMEN1; HPGD/ORM2; HPGD/MIR646HG; HPGD/KLF14; IL1R1/GADD45A; IL1R1/ARG1; IL1R1/PFKFB2; IL1R1/MLLT1; IL1R1/ANXA3; IL1R1/GPR84; IL1R1/OLAH; IL1R1/ADAMTS3; IL1R1/PCOLCE2; IL1R1/ZDHHC19; IL1R1/SLC51A; IL1R1/HPGD; IL1R1/SEMA6B; IL1R1/EFNA1; IL1R1/AGFG1; IL1R1/NSUN7; IL1R1/TNFAIP8L3; IL1R1/KREMEN1; IL1R1/ORM2; IL1R1/MIR646HG; IL1R1/KLF14; KREMEN1/ORM2; KREMEN1/MIR646HG; KREMEN1/KLF14; MIR646HG/KLF14; MLLT1/ANXA3; MLLT1/GPR84; MLLT1/OLAH; MLLT1/ADAMTS3; MLLT1/PCOLCE2; MLLT1/ZDHHC19; MLLT1/SLC51A; MLLT1/HPGD; MLLT1/SEMA6B; MLLT1/EFNA1; MLLT1/AGFG1; MLLT1/SUN7; MLLT1/TNFAIP8L3; MLLT1/KREMEN1; MLLT1/ORM2; MLLT1/MIR646HG; MLLT1/KLF14; NSUN7/TNFAIP8L3; NSUN7/KREMEN1; NSUN7/ORM2; NSUN7/MIR646HG; NSUN7/KLF14; OLAH/ADAMTS3; OLAH/PCOLCE2; OLAH/ZDHHC19; OLAH/SLC51A; OLAH/HPGD; OLAH/SEMA6B; OLAH/EFNA1; OLAH/AGFG1; OLAH/NSUN7; OLAH/TNFAIP8L3; OLAH/KREMEN1; OLAH/ORM2; OLAH/MIR646HG; OLAH/KLF14; ORM2/MIR646HG; ORM2/KLF14; PCOLCE2/ZDHHC19; PCOLCE2/SLC51A; PCOLCE2/HPGD; PCOLCE2/SEMA6B; PCOLCE2/EFNA1; PCOLCE2/AGFG1; PCOLCE2/NSUN7; PCOLCE2/TNFAIP8L3; PCOLCE2/KREMEN1; PCOLCE2/ORM2; PCOLCE2/MIR646HG; PCOLCE2/KLF14; PFKFB2/MLLT1; PFKFB2/ANXA3; PFKFB2/GPR84; PFKFB2/OLAH; PFKFB2/ADAMTS3; PFKFB2/PCOLCE2; PFKFB2/ZDHHC19; PFKFB2/SLC51A; PFKFB2/HPGD; PFKFB2/SEMA6B; PFKFB2/EFNA1; PFKFB2/AGFG1; PFKFB2/NSUN7; PFKFB2/TNFAIP8L3; PFKFB2/KREMEN1; PFKFB2/ORM2; PFKFB2/MIR646HG; PFKFB2/KLF14; SEMA6B/EFNA1; SEMA6B/AGFG1; SEMA6B/NSUN7; SEMA6B/TNFAIP8L3; SEMA6B/KREMEN1; SEMA6B/ORM2; SEMA6B/MIR646HG; SEMA6B/KLF14; SLC51A/HPGD; SLC51A/SEMA6B; SLC51A/EFNA1; SLC51A/AGFG1; SLC51A/NSUN7; SLC51A/TNFAIP8L3; SLC51A/KREMEN1; SLC51A/ORM2; SLC51A/MIR646HG; SLC51A/KLF14; TNFAIP8L3/KREMEN1; TNFAIP8L3/ORM2; TNFAIP8L3/MIR646HG; TNFAIP8L3/KLF14; ZDHHC19/SLC51A; ZDHHC19/HPGD; ZDHHC19/SEMA6B; ZDHHC19/EFNA1; ZDHHC19/AGFG1; ZDHHC19/NSUN7; ZDHHC19/TNFAIP8L3; ZDHHC19/KREMEN1; ZDHHC19/ORM2; ZDHHC19/MIR646HG; ZDHHC19/KLF14; INF signature pairs: FECH/TFEC, TFEC/IFIT1B, FECH/RNF182, IFIT1B/FECH, FECH/APOL4, FECH/GYPA, ITLN1/FECH, FECH/THEM5, IFIT1B/CA1, RHAG/FECH, FECH/FAM83A, RHCE/FECH, TFEC/CA1, SPTA1/FECH, ANKRD22/GLRX5; ANKRD22/GYPA; ANKRD22/IFIT1B; ANKRD22/ITLN1; ANKRD22/KLHDC8A; ANKRD22/RHCE; ANKRD22/RNF182; ANKRD22/SPTA1; ANKRD22/THEM5; ANKRD22/TSPAN5; APOL4/BNIP3L; APOL4/CA1; APOL4/DYRK3; APOL4/FAM83A; APOL4/GLRX5; APOL4/GYPA; APOL4/IFIT1B; APOL4/ITLN1; APOL4/KLHDC8A; APOL4/RHAG; APOL4/RHCE; APOL4/RIOK3; APOL4/RNF182; APOL4/SPTA1; APOL4/THEM5; APOL4/TLCD4; APOL4/TMCC2; APOL4/TSPAN5; APOL4/TSPO2; BNIP3L/ANKRD22; BNIP3L/CA1; BNIP3L/CARD17; BNIP3L/CD274; BNIP3L/DYRK3; BNIP3L/FAM83A; BNIP3L/GBP5; BNIP3L/GLRX5; BNIP3L/GYPA; BNIP3L/IFIT1B; BNIP3L/ITLN1; BNIP3L/KLHDC8A; BNIP3L/P2RY14; BNIP3L/RHAG; BNIP3L/RHCE; BNIP3L/RNF182; BNIP3L/SPTA1; BNIP3L/TFEC; BNIP3L/THEM5; BNIP3L/TLCD4; BNIP3L/TMCC2; BNIP3L/TSPAN5; BNIP3L/TSPO2; CA1/ANKRD22; CA1/CARD17; CA1/DYRK3; CA1/FAM83A; CA1/GBP5; CA1/GLRX5; CA1/GYPA; CA1/IFIT1B; CA1/ITLN1; CA1/KLHDC8A; CA1/P2RY14; CA1/RHCE; CA1/RNF182; CA1/SPTA1; CA1/THEM5; CA1/TLCD4; CA1/TSPAN5; CD274/CA1; CD274/DYRK3; CD274/FAM83A; CD274/GLRX5; CD274/GYPA; CD274/IFIT1B; CD274/ITLN1; CD274/KLHDC8A; CD274/RHCE; CD274/RNF182; CD274/SPTA1; CD274/THEM5; CD274/TLCD4; CD274/TMCC2; CD274/TSPAN5; DYRK3/ANKRD22; DYRK3/CARD17; DYRK3/FAM83A; DYRK3/GBP5; DYRK3/GLRX5; DYRK3/GYPA; DYRK3/IFIT1B; DYRK3/ITLN1; DYRK3/KLHDC8A; DYRK3/P2RY14; DYRK3/RHCE; DYRK3/RNF182; DYRK3/SPTA1; DYRK3/THEM5; DYRK3/TLCD4; DYRK3/TSPAN5; FAM83A/ANKRD22; FAM83A/CARD17; FAM83A/GBP5; FAM83A/GLRX5; FAM83A/GYPA; FAM83A/IFIT1B; FAM83A/ITLN1; FAM83A/KLHDC8A; FAM83A/P2RY14; FAM83A/RHCE; FAM83A/RNF182; FAM83A/SPTA1; FAM83A/THEM5; FAM83A/TLCD4; FAM83A/TSPAN5; FECH/ANKRD22; FECH/APOL4; FECH/BNIP3L; FECH/CA1; FECH/CARD17; FECH/CD274; FECH/DYRK3; FECH/FAM83A; FECH/GBP5; FECH/GLRX5; FECH/GYPA; FECH/IFIT1B; FECH/ITLN1; FECH/KLHDC8A; FECH/P2RY14; FECH/RHAG; FECH/RHCE; FECH/RIOK3; FECH/RNF182; FECH/SPTA1; FECH/TFEC; FECH/THEM5; FECH/TLCD4; FECH/TMCC2; FECH/TSPAN5; FECH/TSPO2; GBP5/GLRX5; GBP5/GYPA; GBP5/IFIT1B; GBP5/ITLN1; GBP5/KLHDC8A; GBP5/RHCE; GBP5/RNF182; GBP5/SPTA1; GBP5/THEM5; GBP5/TSPAN5; GLRX5/CARD17; GLRX5/IFIT1B; GLRX5/RHCE; GLRX5/THEM5; GYPA/CARD17; GYPA/GLRX5; GYPA/IFIT1B; GYPA/ITLN1; GYPA/P2RY14; GYPA/RHCE; GYPA/RNF182; GYPA/THEM5; IFIT1B/CARD17; ITLN1/CARD17; ITLN1/GLRX5; ITLN1/IFIT1B; ITLN1/RHCE; ITLN1/RNF182; ITLN1/THEM5; KLHDC8A/CARD17; KLHDC8A/GLRX5; KLHDC8A/GYPA; KLHDC8A/IFIT1B; KLHDC8A/ITLN1; KLHDC8A/P2RY14; KLHDC8A/RHCE; KLHDC8A/RNF182; KLHDC8A/SPTA1; KLHDC8A/THEM5; KLHDC8A/TSPAN5; P2RY14/GLRX5; P2RY14/IFIT1B; P2RY14/ITLN1; P2RY14/RHCE; P2RY14/RNF182; P2RY14/THEM5; RHAG/ANKRD22; RHAG/CA1; RHAG/CARD17; RHAG/CD274; RHAG/DYRK3; RHAG/FAM83A; RHAG/GBP5; RHAG/GLRX5; RHAG/GYPA; RHAG/IFIT1B; RHAG/ITLN1; RHAG/KLHDC8A; RHAG/P2RY14; RHAG/RHCE; RHAG/RNF182; RHAG/SPTA1; RHAG/THEM5; RHAG/TLCD4; RHAG/TMCC2; RHAG/TSPAN5; RHAG/TSPO2; RHCE/CARD17; RHCE/IFIT1B; RHCE/THEM5; RIOK3/ANKRD22; RIOK3/BNIP3L; RIOK3/CA1; RIOK3/CARD17; RIOK3/CD274; RIOK3/DYRK3; RIOK3/FAM83A; RIOK3/GBP5; RIOK3/GLRX5; RIOK3/GYPA; RIOK3/IFIT1B; RIOK3/ITLN1; RIOK3/KLHDC8A; RIOK3/P2RY14; RIOK3/RHAG; RIOK3/RHCE; RIOK3/RNF182; RIOK3/SPTA1; RIOK3/TFEC; RIOK3/THEM5; RIOK3/TLCD4; RIOK3/TMCC2; RIOK3/TSPAN5; RIOK3/TSPO2; RNF182/CARD17; RNF182/GLRX5; RNF182/IFIT1B; RNF182/RHCE; RNF182/THEM5; SPTA1/CARD17; SPTA1/GLRX5; SPTA1/GYPA; SPTA1/IFIT1B; SPTA1/ITLN1; SPTA1/P2RY14; SPTA1/RHCE; SPTA1/RNF182; SPTA1/THEM5; SPTA1/TSPAN5; TFEC/CA1; TFEC/DYRK3; TFEC/FAM83A; TFEC/GLRX5; TFEC/GYPA; TFEC/IFIT1B; TFEC/ITLN1; TFEC/KLHDC8A; TFEC/RHAG; TFEC/RHCE; TFEC/RNF182; TFEC/SPTA1; TFEC/THEM5; TFEC/TLCD4; TFEC/TMCC2; TFEC/TSPAN5; TFEC/TSPO2; THEM5/CARD17; THEM5/IFIT1B; TLCD4/ANKRD22; TLCD4/CARD17; TLCD4/GBP5; TLCD4/GLRX5; TLCD4/GYPA; TLCD4/IFIT1B; TLCD4/ITLN1; TLCD4/KLHDC8A; TLCD4/P2RY14; TLCD4/RHCE; TLCD4/RNF182; TLCD4/SPTA1; TLCD4/THEM5; TLCD4/TSPAN5; TMCC2/ABCA6; TMCC2/ANKRD22; TMCC2/CA1; TMCC2/CARD17; TMCC2/DYRK3; TMCC2/FAM83A; TMCC2/GBP5; TMCC2/GLRX5; TMCC2/GYPA; TMCC2/IFIT1B; TMCC2/ITLN1; TMCC2/KLHDC8A; TMCC2/P2RY14; TMCC2/RHCE; TMCC2/RNF182; TMCC2/SPTA1; TMCC2/THEM5; TMCC2/TLCD4; TMCC2/TSPAN5; TSPAN5/CARD17; TSPAN5/GLRX5; TSPAN5/GYPA; TSPAN5/IFIT1B; TSPAN5/ITLN1; TSPAN5/P2RY14; TSPAN5/RHCE; TSPAN5/RNF182; TSPAN5/THEM5; TSPO2/ANKRD22; TSPO2/CA1; TSPO2/CARD17; TSPO2/CD274; TSPO2/DYRK3; TSPO2/FAM83A; TSPO2/GBP5; TSPO2/GLRX5; TSPO2/GYPA; TSPO2/IFIT1B; TSPO2/ITLN1; TSPO2/KLHDC8A; TSPO2/P2RY14; TSPO2/RHCE; TSPO2/RNF182; TSPO2/SPTA1; TSPO2/THEM5; TSPO2/TLCD4; TSPO2/TMCC2; TSPO2/TSPAN5. IHD signature pairs: MAP7/SPRED1, SPRED1/GPR34, IL5RA/SPRED1, SPRED1/TPRG1, HRK/SPRED1, SPRED1/PLCB1, TRIM2/SPRED1, SIGLEC8/SPRED1, SMPD3/SPRED1, SPRED1/ZNF600, SPRED1/SDC2, MAP7/GPR34, PRSS33/SPRED1, SPRED1/DYNC2H1, CACNA2D3/SPRED1, ADAM23/GPR34; ADAM23/MAP7; ADAM23/PLCB1; ADAM23/SPRED1; ALOX15/GPR34; ALOX15/PLCB1; ALOX15/SPRED1; BAALC/GPR34; BAALC/PLCB1; BAALC/SPRED1; CACNA2D3/DYNC2H1; CACNA2D3/GPR34; CACNA2D3/PLCB1; CACNA2D3/SPRED1; CACNA2D3/ZNF600; GPR34/DYNC2H1; GPR34/GRAMD1C; GPR34/PLCB1; GPR34/PRG1; GPR34/ZNF600; GPR82/DYNC2H1; GPR82/GPR34; GPR82/GRAMD1C; GPR82/PLCB1; GPR82/TPRG1; GPR82/ZNF600; GRAMD1C/DYNC2H1; GRAMD1C/PLCB1; GRAMD1C/ZNF600; HRK/DYNC2H1; HRK/GPR34; HRK/MAP7; HRK/PLCB1; HRK/SPRED1; HRK/ZNF600; IL5RA/DYNC2H1; IL5RA/GPR34; IL5RA/PLCB1; IL5RA/SPRED1; IL5RA/TRIM2; MAP7/BAALC; MAP7/CACNA2D3; MAP7/DYNC2H1; MAP7/GPR34; MAP7/GPR82; MAP7/GRAMD1C; MAP7/PLCB1; MAP7/SPRED1; MAP7/TPRG1; MAP7/ZNF600; PLCB1/DYNC2H1; PLCB1/TPRG1; PLCB1/ZNF600; PRSS33/GPR34; PRSS33/PLCB1; PRSS33/SPRED1; SDC2/DYNC2H1; SDC2/GPR34; SDC2/PLCB1; SDC2/ZNF600; SIGLEC8/DYNC2H1; SIGLEC8/GPR34; SIGLEC8/MAP7; SIGLEC8/PLCB1; SIGLEC8/SPRED1; SIGLEC8/TRIM2; SMPD3/DYNC2H1; SMPD3/GPR34; SMPD3/MAP7; SMPD3/PLCB1; SMPD3/SPRED1; SMPD3/TRIM2; SPRED1/DYNC2H1; SPRED1/GPR34; SPRED1/GPR82; SPRED1/GRAMD1C; SPRED1/PLCB1; SPRED1/SDC2; SPRED1/TPRG1; SPRED1/ZNF600; TRIM2/CACNA2D3; TRIM2/DYNC2H1; TRIM2/GPR34; TRIM2/GPR82; TRIM2/GRAMD1C; TRIM2/HRK; TRIM2/MAP7; TRIM2/PLCB1; TRIM2/SDC2; TRIM2/SPRED1; TRIM2/TPRG1; TRIM2/ZNF600; IFN signature pairs: ETV7/PLEKHO1, IFITM3/ETV7, ETV7/APOL1, BATF2/ETV7, PLEKHO1/BATF2, ETV7/EPSTI1, EPSTI1/BATF2, IFITM3/BATF2, ETV7/BATF2, USP18/EPSTI1, ETV7/SEPTIN4, ETV7/LAMP3, SERPING1/BATF2, LAMP3/BATF2, LAMP3/SERPING1, APOL1/BATF2; APOL1/CLEC4F; APOL1/EPSTI1; APOL1/EXOC3L1; APOL1/HES4; APOL1/IFITM3; APOL1/LY6E; APOL1/RSAD2; APOL1/SEPTIN4; APOL1/SERPING1; APOL1/TPPP3; BATF2/EXOC3L1; BATF2/HES4; CLEC4F/BATF2; CLEC4F/EXOC3L1; EPSTI1/BATF2; EPSTI1/CLEC4F; EPSTI1/EXOC3L1; EPSTI1/HES4; EPSTI1/IFITM3; EPSTI1/LY6E; EPSTI1/RSAD2; EPSTI1/SERPING1; EPSTI1/TPPP3; ETV7/APOL1; ETV7/BATF2; ETV7/CLEC4F; ETV7/EPSTI1; ETV7/EXOC3L1; ETV7/HES4; ETV7/IFITM3; ETV7/LAMP3; ETV7/LY6E; ETV7/PLEKHO1; ETV7/RSAD2; ETV7/SEPTIN4; ETV7/SERPING1; ETV7/TPPP3; EXOC3L1/HES4; LAMP3/APOL1; LAMP3/BATF2; LAMP3/CLEC4F; LAMP3/EPSTI1; LAMP3/EXOC3L1; LAMP3/HES4; LAMP3/IFITM3; LAMP3/LY6E; LAMP3/RSAD2; LAMP3/SEPTIN4; LAMP3/SERPING1; LAMP3/TPPP3; LY6E/BATF2; LY6E/EXOC3L1; PLEKHO1/APOL1; PLEKHO1/BATF2; PLEKHO1/EPSTI1; PLEKHO1/EXOC3L1; PLEKHO1/IFITM3; PLEKHO1/LAMP3; PLEKHO1/RSAD2; PLEKHO1/SEPTIN4; PLEKHO1/SERPING1; RSAD2/BATF2; RSAD2/CLEC4F; RSAD2/EXOC3L1; RSAD2/HES4; RSAD2/IFITM3; RSAD2/LY6E; RSAD2/SERPING1; RSAD2/TPPP3; SEPTIN4/BATF2; SEPTIN4/CLEC4F; SEPTIN4/EPSTI1; SEPTIN4/EXOC3L1; SEPTIN4/HES4; SEPTIN4/IFITM3; SEPTIN4/LGALS3BP; SEPTIN4/LY6E; SEPTIN4/OTOF; SEPTIN4/RSAD2; SEPTIN4/SERPING1; SEPTIN4/TPPP3; SERPING1/BATF2; SERPING1/CLEC4F; SERPING1/EXOC3L1; SERPING1/HES4; SERPING1/LY6E; SERPING1/TPPP3; TPPP3/BATF2; TPPP3/EXOC3L1; ADA signature pairs: LGALS3BP/OTOF, LGALS3BP/IFI27, LGALS3BP/KIF14, LGALS3BP/CENPF, GTSE1/LGALS3BP, LGALS3BP/KCTD14, LGALS3BP/PDIA4, LGALS3BP/TSHR, LGALS3BP/PLAAT2, OTOF/IF127, IGF1/LGALS3BP, CDC45/LGALS3BP, LGALS3BP/KIF15, LGALS3BP/IGLL5, LGALS3BP/MIXL, CAV1/LGALS3BP; CAV1/OTOF; CDC45/LGALS3BP; CDC45/OTOF; CENPF/KCTD14; GPRC5D/OTOF; GTSE1/LGALS3BP; GTSE1/OTOF; IGF1/LGALS3BP; IGF1/OTOF; KCTD14/KLHL14; KCTD14/PDIA4; KCTD14/TSHR; KIF14/KCTD14; LGALS3BP/CENPF; LGALS3BP/GPRC5D; LGALS3BP/IFI27; LGALS3BP/IGLL5; LGALS3BP/KCTD14; LGALS3BP/KIF14; LGALS3BP/KIF15; LGALS3BP/KLHL14; LGALS3BP/MIR155HG; LGALS3BP/MIXL1; LGALS3BP/OTOF; LGALS3BP/PDIA4; LGALS3BP/PLAAT2; LGALS3BP/SDC1; LGALS3BP/SLC16A14; LGALS3BP/TSHR; OTOF/CENPF; OTOF/IF127; OTOF/IGLL5; OTOF/KCTD14; OTOF/KIF14; OTOF/KIF15; OTOF/KLHL14; OTOF/MIR155HG; OTOF/MIXL1; OTOF/PDIA4; OTOF/PLAAT2; OTOF/SDC1; OTOF/SLC16A14; OTOF/TSHR; PLAAT2/KCTD14; TNFRSF17/LGALS3BP; TNFRSF17/OTOF; or wherein the preferred pairs may comprise NPS signature pairs: GADD45A/EFNA1, EFNA1/MIR646HG, MIR646HG/KLF14, MLLT1/MIR646HG, ARG1/MLLT1, MLLT1/EFNA1, MLLT1/NSUN7, EFNA1/NSUN7, SLC51A/EFNA1, EFNA1/KLF14, ZDHHC19/EFNA1, EFNA1/AGFG1, NSUN7/KLF14, EFNA1/PFKFB2, MLLT1/KLF14. INF signature pairs: FECH/TFEC, TFEC/IFIT1B, FECH/RNF182, IFIT1B/FECH, FECH/APOL4, FECH/GYPA, ITLN1/FECH, FECH/THEM5, IFIT1B/CA1, RHAG/FECH, FECH/FAM83A, RHCE/FECH, TFEC/CA1, SPTA1/FECH. IHD signature pairs: MAP7/SPRED1, SPRED1/GPR34, IL5RA/SPRED1, SPRED1/TPRG1, HRK/SPRED1, SPRED1/PLCB1, TRIM2/SPRED1, SIGLEC8/SPRED1, SMPD3/SPRED1, SPRED1/ZNF600, SPRED1/SDC2, MAP7/GPR34, PRSS33/SPRED1, SPRED1/DYNC2H1, CACNA2D3/SPRED1. IFN signature pairs: ETV7/PLEKHO1, IFITM3/ETV7, ETV7/APOL1, BATF2/ETV7, PLEKHO1/BATF2, ETV7/EPSTI1, EPSTI1/BATF2, IFITM3/BATF2, ETV7/BATF2, USP18/EPSTI1, ETV7/SEPTIN4, ETV7/LAMP3, SERPING1/BATF2, LAMP3/BATF2, LAMP3/SERPING1. ADA signature pairs: LGALS3BP/OTOF, LGALS3BP/IFI27, LGALS3BP/KIF14, LGALS3BP/CENPF, GTSE1/LGALS3BP, LGALS3BP/KCTD14, LGALS3BP/PDIA4, LGALS3BP/TSHR, LGALS3BP/PLAAT2, OTOF/IFI27, IGF1/LGALS3BP, CDC45/LGALS3BP, LGALS3BP/KIF15, LGALS3BP/IGLL5, LGALS3BP/MIXL1.

In another embodiment is provided a method of detecting a sepsis mechanistic severity gene signature in a biological sample obtained from a subject suspected of having sepsis, at risk of developing severe sepsis, at risk of organ failure, or having endotoxin tolerance, the method comprising detecting a level of expression, in a biological sample obtained from the subject, for each of a plurality of Endotoxin Tolerance Signature genes to provide a sample gene signature, wherein the plurality of genes is selected from the group consisting of ABCA13, ADAMTS2, ADAMTS3, AK5, ANKRD22, ANKRD34B, ANLN, AQP1, ARG1, ARHGAP44, ARHGEF17, ASPM, ATP1B2, AURKA, AZU1, BAIAP3, BPI, Clorf226, CACNB4, CCL4L2, CCN3, CCNA1, CD177, CD24, CDK1, CDKN3, CEACAM6, CEACAM8, CENPA, CFH, CHDH, CHIT1, CKAP2L, CLEC4C, CLEC4F, CLNK, COL17A1, CRISP2, CRISP3, CTSE, CTSG, CYP19A1, CYYR1, DEFA4, DENND2C, DEPDC1, DGKK, DLC1, DLGAP5, DNAH10, DOC2B, DSP, ELANE, ERG, FAM20A, FAM83A, FBN1, FFAR3, GOS2, GGT5, GLB1L2, GJB6, GPR84, GRAMD1C, GYPA, HBM, HMGB3, HP, HPGD, HRK, IGLL1, IL1R2, IL1RL1, INHBA, IQGAP3, ITGA7, ITGB4, KIF15, KIF20A, KLF14, LAMB3, LCN2, LGR4, LPL, LTF, MAFG, MERTK, METTL7B, MMP8, MMP9, MPO, MRC1, MROCKI, MS4A3, MS4A4A, NECAB1, NEIL3, NEK2, NRXN2, NUF2, OLAH, OLFM4, OLIG2, PCOLCE2, PCSK9, PHF24, PIGR, PLAAT2, PPARG, PRTN3, PTGES, PYCRI, RAB3IL1, RASGRF1, RETN, RHCE, RIPOR3, RPGRIP1, RRM2, S100A12, S100A8, SCN8A, SEMA6B, SERPINB10, SIGLEC8, SIL1, SLC16A1, SLC28A3, SLC39A8, SLC4A10, SLC51A, SLC6A19, SLC8A3, SLCO4A1, SMIM1, SMPDL3A, SPATC1, SPOP, SSBP2, TCN1, TCTEX1D1, TDRD9, TEAD2, TFRC, THBS1, TIMP3, TLN2, TMEM255A, TMEM45A, TNFAIP8L3, TNIP3, TROAP, TTK, VSIG4, WNT3, YPEL4, ZDHHC19, wherein the severity gene signature is detected when the sample gene signature is different from a reference gene signature, wherein the reference gene signature represents a standard level of expression of each of the plurality of genes.

In an embodiment, the difference between the sample gene signature and the reference gene signature is defined by a difference in expression of at least two of the plurality of genes in an expression change direction, at least 5 of the plurality of genes in an expression change direction, at least 10 of the plurality of genes in an expression change direction, at least 15 of the plurality of genes in an expression change direction, at least 20 of the plurality of genes in an expression change direction, at least 25 of the plurality of genes in an expression change direction, at least 30 of the plurality of genes in an expression change direction, or at least 31 of the plurality of genes in an expression change direction.

In another embodiment is provided a method of detecting a sepsis mechanistic severity gene signature in a biological sample obtained from a subject suspected of having sepsis, at risk of developing severe sepsis, at risk of organ failure, or having endotoxin tolerance, the method comprising detecting a level of expression, in a biological sample obtained from the subject, for each of a plurality of Endotoxin Tolerance Signature genes to provide a sample gene signature, wherein plurality of genes is selected from the group consisting of ADAMTS2, RETN, MMP8, GOS2, CYP19A1, OLAH, SLC6A19, TNFAIP8L3.

In another embodiment is provided a method of detecting a sepsis mechanistic severity gene signature in a biological sample obtained from a subject suspected of having sepsis, at risk of developing severe sepsis, at risk of organ failure, or having endotoxin tolerance, the method comprising detecting a level of expression, in a biological sample obtained from the subject, for each of a plurality of Endotoxin Tolerance Signature genes to provide a sample gene signature, wherein plurality of genes is selected from the group consisting of CCL4L2, GPR84, HRK, MMP8, GGT5, RASGRF1.

In an embodiment, detecting the level of expression comprises detecting nucleic acids encoded by each of the plurality of genes.

In another embodiment, detecting the level of expression comprises one or more of a polymerase chain reaction (PCR) amplification method, a non-PCR based amplification method, reverse transcriptase-(RT) PCR, Q-beta replicase amplification, ligase chain reaction, signal amplification (Ampliprobe), light cycling, differential display, Northern analysis, hybridization, microarray analysis, DNA sequencing, Ref-Seq, MassArray analysis and MALDI-TOF mass spectrometry.

In a further embodiment, determining the level of expression comprises isolating mRNA from the biological sample, reverse transcribing the mRNA to generate cDNA products and contacting the cDNA products with a microarray comprising a plurality of polynucleotide probes capable of hybridizing to a plurality of cDNAs that are complementary to a plurality of mRNAs expressed from the plurality of genes.

In another embodiment, the biological sample comprises blood, plasma, serum, tissue, amniotic fluid, saliva, urine, stool, bronchoalveolar lavage fluid, cerebrospinal fluid or skin cells.

In another embodiment is provided a method for treating sepsis in a subject, the method comprising: a) detecting a specific endotype gene signature for the subject according to a method as described herein, wherein a difference between the sample gene signature and the reference gene signature indicates that the subject has sepsis or is at risk of developing sepsis, and b) if the subject has sepsis or, is at risk of developing sepsis, administering to the subject an effective amount of one or more medicines that act specifically against the mechanisms associated with the endotype.

In another embodiment is provided a method for identifying a candidate agent for the treatment of sepsis, the method comprising: a) contacting a sepsis endotype cell with a test agent, b) detecting the level of expression for each of a plurality of signature genes from a specific endotype in the endotoxin tolerant cell to provide an expression signature, wherein the plurality of genes is selected from NPS signature: AGFG1, ARG1, ATP9A, ANXA3, EFNA1, GADD45A, GPR84, HPGD, IL1R1, KLF14, KREMEN1, MIR646HG, MLLT1, NSUN7, OLAH, ORM2, PCOLCE2, PFKFB2, SLC51A, TNFAIP8L3, ZDHHC19, ADAMTS3, AKR1C1, ALDH1A2, ALOX5AP, ALPL, AMPH, ANKRD55, BCL3, BTBD19, CA4, CD163L1, CD177, CD82, CST7, CYP19A1, CYSTM1, DAAM2, DGAT2, ECHDC3, ENTPD7, EXOSC4, FFAR3, FGF13, FSTL4, GALNT14, GRAMD1A, GRB10, GYG1, HPGD, IER3, IL18RAP, IL1R2, IL1RN, IRAG1-AS1, KCNE1B, KCNMA1, MCEMP1, MKNK1, MMP9, MSRA, NECAB1, NSMCE1-DT, OPLAH, PDGFC, PFKFB3, PHF24, PI3, PLIN4, PLIN5, PLK3, POR, PROK2, RFX2, RGL4, ROM1, S100A12, S100P, SEMA6B, SHROOM4, SLP1, SOCS3, SPATC1, SPDYA, SPINK8, SPP1, ST6GALNAC3, SYN2, TDRD9, TMEM120A, TMIGD3, TSPO, UPP1, XCR1; INF signature: BNIP3L, CA1, FAM83A, FECH, GLRX5, GYPA, IFIT1B, RHCE, RIOK3, RNF182, SLC6A19, SPTA1, THEM5, TLCD4, TSPAN5, TSPO2, ABCG2, ACHE, ACKR1, ACSL6, ADD2, AHSP, ALAS2, ALDH5A1, ANK1, ANKRD9, AQP1, ARHGEF12, ARHGEF37, ARL4A, ATP1B2, ATP1B2, BBOF1, BCAM, BCL2L1, BLVRB, BPGM, Clorf116, CA2, CISD2, CLIC2, CR1L, CR1L, CTNNAL1, CTSE, CTSE, DCAF12, DMTN, DNAJC6, DPCD, DYRK3, EMID1, EPB42, ERFE, FAM210B, FAXDC2, FRMD4A, GMPR, GSPT1, GYPB, HBM, HEMGN, HEPACAM2, HMBS, IGF2BP2, ISCA1, ITLN1, KANK2, KCNH2, KDM7A-DT, KEL, KLC3, KLF1, KLHDC8A, KRT1, LRRC2, MAOA, MAOA, MARCHF8, MBNL3, MFSD2B, MRC2, MXI1, MYL4, NFIX, NUDT4, OSBP2, PAGE2B, PBX1, PCDH1, PGF, PLEK2, PNP, PRDX2, PTPRF, RAPIGAP, RBM38, RFESD, RFESD, RGCC, RGS16, RHAG, RHD, RIPOR3, RNF175, RUNDC3A, SEC14L4, SELENBP1, SELENOP, SFRP2, SGIP1, SIAH2, SLC14A1, SLC1A5, SLC22A23, SLC2A1, SLC6A8, SLC6A9, SLC7A5, SMIM5, SNCA, SOX6, SPTB, STRADB, TAL1, TENT5C, TFR2, TMCC2, TMOD1, TNS1, TRIM10, TRIM58, TSPAN7, TTC25, UBB, USP12, XK, YBX3, YPEL4; IHD signature: ABCA6, ADAM23, ALOX15, CACNA2D3, DYNC2H1, GPR34, GRAMD1C, LPL, MAP7, MIR155HG, PLCB1, SDC2, SIGLEC8, SPRED1, SLC16A14, SMPD3, TPPP3, TPRG1, ZNF600, ADGRD1, ANGPT1, GPR82, HDAC9, IL5RA, KLHDC1, PRSS33, PTGDR2, PTGFRN, TBC1D12, TRIM2; IFN signature: ANKRD22, APOL1, APOL4, BATF2, CARD17, CD274, EPSTI1, ETV7, GBP5, IFITM3, P2RYl4, PLEKHO1, RSAD2, SERPING1, TFEC, EXOC3L1, IRF7, OAS1, SEPTIN4, LY6E, LAMP3; ADA signature: CCL2, CDC45, CENPF, CLEC4F, GTSE1, IFI27, KCTD14, KIF14, KIF15, KLHDC7B, LGALS3BP, OTOF, PDIA4, SIGLEC1, AGRN, CD38, CDCA7, CDT1, CTLA4, DHX58, EME1, FAM111B, HES4, IFI44L, IFIT3, IFNG-AS1, IL12RB2, IL4I1, KIF19, LAG3, MCM10, P2RY6, PACSIN1, PARM1, SAMD4A, SPATS2L, HERC5, TMPRSS3, TNFRSF13B, TSHR, TTC21A, and c) selecting the test agent as a candidate agent for treatment of sepsis when the expression signature substantially corresponds with the reference signature.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should rather be given the broadest interpretation consistent with the description as a whole.

### BRIEF DESCRIPTION OF THE DRAWINGS

The embodiments of the disclosure will now be described in greater detail with reference to the attached drawings, in which:

FIG. 1 shows a general scheme for collection of samples according to an example of the present disclosure. One hundred and fifteen (115) suspected sepsis patients were recruited within the first two hours of admission from two ERs. Similarly, 82 patients were recruited within the first day of ICU admission, with patients suspected of Covid-19 infection.

FIG. 2 shows the biological characterization of the Neutrophilic-Suppressive (NPS), Inflammatory (INF), Innate Host Defence (IHD), Interferon (IFN), and Adaptive (ADA) endotypes. After separation of patients by their blood transcriptome into 5 endotypes, functional enrichment was performed by identifying pathways overrepresented within the genes for each endotype for both up- and down-regulated differently expressed (DE) genes, comparing each endotype to healthy controls (Fold change ≥2; Benjamini-Hochberg adjusted P value≤0.01). The ratio of each dysregulated pathway was the total number of DE genes divided by the pathway size (total number of proteins in each pathway). We focused here on pathways annotated to adaptive immune, innate immune, and cytokine signaling processes.

FIG. 3 shows cell composition analysis of, from left to right in each chart: NPS, INF, IHD, IFN, and ADA endotypes for neutrophils (top left chart); monocytes (top right chart); CD4+ T cells (middle left chart); CD8+ T cells (middle right chart); and plasma B cells (lower left chart). Cell proportions were estimated by the cell composition deconvoluting program CIBERSORT for each endotype.

FIG. 4 shows clinical characterization of the NPS, INF, IHD, IFN, and ADA endotypes: selected clinical symptomology and outcomes and their distributions (top); and organ failure probability within 28 days of ER admission (bottom). Clinical measures were compared between clusters using non-parametric comparison of rank statistics (Kruskal-Wallis test) and Chi-square tests depending on variable type. Dunn's Posthoc tests for Kruskal-Wallis tests: #p<0.05 vs. IHD. * p<0.05 vs. IFN.+p<0.05 vs. ADA.\hat{ }p<0.05 vs. INF. The Kaplan Meier graph for 28-day organ failure free probability (lower probability means more organ failure) as a function of time was compared statistically using the log rank test.

FIGS. 5-9 show minimally connected first order protein:protein interaction networks (drawn using the program NetworkAnalyst) of the unique endotype gene expression signatures (DE in the given endotype but not in any other endotype) for the NPS (FIG. 5), INF (FIG. 6), IHD (FIG. 7), IFN (FIG. 8) and ADA (FIG. 9) endotypes.

FIG. 10 shows a heatmap depicting the expression of genes with respect to, from left to right: NPS, INF, IHD, IFN and ADA endotypes. Each of the darker blocks running up the diagonal represents the DE genes defining that particular endotype. These genes were identified by a multinomial regression model that relied on just 88 genes to predict endotype status. These are listed in Table 3.

FIG. 11 shows endotype classification of severe non-Covid and severe Covid-19 ICU sepsis patients. Shown is a heatmap depicting gene set variation analysis (GSVA) enrichment statistics in ICU patients for each endotype signature based on a subset of 40 genes from the list in Table 3.

FIG. 12 shows pathway enrichment of up- and down-regulated genes comparing ICU endotypes to healthy controls.

FIG. 13 shows the clinical characteristics (from left to right: ICU stay days, SOFA at 24 hours and SOFA at 72 hours) for severe non-Covid and severe Covid-19 ICU sepsis patients according to endotype, from left to right in each chart: NPS, INF, IHD and IFT (top row); Covid-19 positivity and mortality in the ICU validation cohort for predicted endotypes, from left to right in each chart: NPS, INF, RID and IFN (middle row); and survival probability within 28 days of admission depicted using a Kaplan Meier analysis (bottom). The P value is based on a log rank test.

FIG. 14 shows the pathway enrichment in severe non-Covid and severe Covid-19 ICU sepsis patients: Pathway enrichment of up- and down-regulated genes directly comparing Covid-19 positive to negative patients (top); and a heatmap depicting GSVA enrichment statistics in Covid-19 PCR positive and negative patients for two of the endotype signatures (bottom, partial rearrangement of FIG. 11).

FIG. 15 shows pathway enrichment for DE genes defining each severity group when compared to healthy controls (n=9). Functional enrichment was performed on up- and down-regulated DE genes separately.

### DETAILED DESCRIPTION

### I. Definitions

Unless otherwise indicated, the definitions and embodiments described in this and other sections are intended to be applicable to all embodiments and aspects of the disclosure herein described for which they would be understood to be suitable by a person skilled in the art.

As used herein, the terms “comprising” (and any form thereof, such as “comprise” and “comprises”), “having” (and any form thereof, such as “have” and “has”), “including” (and any form thereof, such as “include” and “includes”) and “containing” (and any form thereof, such as “contain” and “contains”) and grammatical variations thereof, are inclusive or open-ended and do not exclude additional, unrecited features, elements, components, groups, integers and/or steps. The term “consisting essentially of” as used herein, is intended to specify the presence of the stated features, elements, components, groups, integers and/or steps as well as those that do not materially affect the basic and novel characteristic(s) of these features, elements, components, groups, integers and/or steps. The term “consisting of” and its derivatives are intended to be close-ended terms that specify the presence of the stated features, elements, components, groups, integers and/or steps, and also exclude the presence of other unstated features, elements, components, groups, integers and/or steps.

As used herein, terms of degree such as “substantially”, “about” and “approximately” mean a reasonable amount of deviation of the modified term such that the end result is not significantly changed. These terms of degree should be construed as including a deviation of at least ±5% or ±10% of the modified term if this deviation would not negate the meaning of the term it modifies.

As used in this disclosure, the singular forms “a”, “an” and “the” include plural references unless the content clearly dictates otherwise.

The term “plurality” as used herein means more than one, for example, two or more, three or more, four or more, and the like.

The term “and/or” as used herein means that the listed items are present, or used, individually or in combination. In effect, this term means that “at least one of” or “one or more” of the listed items is present or used.

The term “sepsis” as used herein refers to a clinical response to a suspected or proven infection. Sepsis may be defined, for example, as including two or more of the following symptoms: tachypnea or tachycardia; leukocytosis or leukopenia; and hyperthermia or hypothermia, and may manifest as a complex infectious and immunological disorder. Sepsis may be complicated by organ failure leading to severe sepsis and may require admission to an intensive care unit (ICU) and carries a higher risk of severity and death.

The term “gene” as used herein refers to a nucleic acid sequence that comprises coding sequences necessary for producing a polypeptide or precursor. Control sequences that direct and/or control expression of the coding sequences may also be encompassed by the term “gene” in some instances. The polypeptide or precursor may be encoded by a full length coding sequence or by a portion of the coding sequence. A gene may contain one or more modifications in either the coding or the untranslated regions that could affect the biological activity or the chemical structure of the polypeptide or precursor, the rate of expression, or the manner of expression control. Such modifications include, but are not limited to, mutations, insertions, deletions, and substitutions of one or more nucleotides, including single nucleotide polymorphisms that occur naturally in the population. The gene may constitute an uninterrupted coding sequence or it may include one or more subsequences. The term “gene” as used herein includes variants of the genes identified in Tables 3-6 and 8.

The sequences of the genes listed herein can readily be obtained by one of skill in the art from publicly available databases, such as but not limited to the GenBank database maintained by the National Center for Biotechnology (NCBI), for example, by searching using the provided gene symbols. These gene symbols are recognized by databases including but not limited to HGNC, Entrez Gene, UniProtKB/Swiss-Prot, OMIM, GeneLoc, and/or Ensembl; all aliases listed herein are defined by the GeneCards database.

The terms “gene expression profile” or “gene signature” and the like as used herein, refer to a group of genes expressed by a particular cell or tissue type wherein expression of the genes taken together, or the differential expression of such genes, is indicative and/or predictive of a certain condition, such as sepsis.

The term “differential expression” as used herein refers to quantitative and/or qualitative differences in the expression of a gene or a protein in diseased tissue or cells versus, e.g., normal tissue or cells. For example, a differentially expressed gene may have its expression activated or completely inactivated in normal versus disease conditions, or may be up-regulated (over-expressed) or down-regulated (under-expressed) in a disease condition versus a normal condition. Stated another way, a gene or protein is differentially expressed when expression of the gene or protein occurs at a higher or lower level in the diseased tissues or cells of a subject (e.g., a human patient) relative to the level of its expression in the normal (disease-free) tissues or cells of the subject (e.g., the human patient) and/or control tissues or cells.

The term “nucleic acid” as used herein, refers to a molecule comprised of one or more nucleotides, for example, ribonucleotides, deoxyribonucleotides, or both. The term includes monomers and polymers of nucleotides, with the nucleotides being bound together, in the case of the polymers, in sequence, typically via 5′ to 3′ linkages, although alternative linkages are also contemplated in some embodiments. The nucleotide polymers may be single or double-stranded. The nucleotides may be naturally occurring or may be synthetically produced analogs that are capable of forming base-pair relationships with naturally occurring base pairs. Examples of non-naturally occurring bases that are capable of forming base-pairing relationships include, but are not limited to, aza and deaza pyrimidine analogs, aza and deaza purine analogs, and other heterocyclic base analogs, wherein one or more of the carbon and nitrogen atoms of the pyrimidine rings have been substituted by heteroatoms, e.g., oxygen, sulphur, selenium, phosphorus, and the like.

The term “corresponding to” and grammatical variations thereof as used herein with respect to a nucleic acid sequence indicates that the nucleic acid sequence is identical to all or a portion of a reference nucleic acid sequence. In contradistinction, the term “complementary to” is used herein to indicate that the nucleic acid sequence is identical to all or a portion of the complementary strand of the reference nucleic acid sequence. For illustration, the nucleic acid sequence “TATAC” corresponds to a reference sequence “TATAC” and is complementary to a reference sequence “GTATA.”

The term “subject” as used herein includes all members of the animal kingdom including mammals, and optionally refers to humans. In an embodiment, the subject is human.

The term “biological sample” refers to a sample obtained from a subject (e.g., a human patient) or from components (e.g., cells) of a subject. The sample may be of any relevant biological tissue or fluid. The sample may be a “clinical sample” which is a sample derived from a patient. Such samples include, but are not limited to, sputum, blood, blood cells (e.g., white cells), nasal brushings, throat swabs, urine, amniotic fluid, plasma, semen, bone marrow, and tissue or fine needle biopsy samples, urine, peritoneal fluid, and pleural fluid, or cells therefrom. Biological samples may also include sections of tissues such as frozen sections taken for histological purposes. A biological sample may also be referred to as a “patient sample.”

As used herein, the term “effective amount” and the like means an amount effective, at dosages and for periods of time necessary to achieve a desired result. For example, in the context of treating sepsis, an effective amount e.g. of the one or more therapies that act specifically against a mechanism associated with a sepsis mechanistic endotype is an amount that, for example, reduces the sepsis compared to the sepsis without administration of the one or more therapies that act specifically against a mechanism associated with a sepsis mechanistic endotype. Effective amounts may vary according to factors such as the disease state, age, sex, weight and/or species of the subject. The amount of a given therapy or combination thereof that will correspond to such an amount will vary depending upon various factors, such as the given therapy or combination thereof, the pharmaceutical formulation, the route of administration or use, the identity of the subject being treated, and the like, but can nevertheless be routinely determined by one skilled in the art.

The terms “to treat”, “treating” and “treatment” and the like as used herein and as is well understood in the art, means an approach for obtaining beneficial or desired results, including clinical results. Beneficial or desired clinical results include, but are not limited to alleviation or amelioration of one or more symptoms of sepsis, diminishment of the extent of the sepsis, stabilized (i.e., not worsening) of the sepsis, delay or slowing of the progression of the sepsis, and/or amelioration or palliation of the disease state of the sepsis.

It is contemplated that any embodiment discussed herein can be implemented with respect to any of the disclosed methods, uses or compositions of the invention, and vice versa.

### II. Methods and Uses

An object of the present disclosure was to identify endotypes at first clinical presentation, where patients show broad clinical traits and final sepsis diagnoses are not established. To achieve this, next generation RNA-Seq was used to perform accurate whole blood transcriptomics and clinical metadata was collected in a cohort consisting of ER patients. Unsupervised consensus clustering was used to identify five endotypes with robust mechanistic and clinical characteristics. Also recruited, on the first day of ICU admission, was a second cohort of severely ill patients some of whom had SARS-CoV-2 infection. Candidate gene-expression signatures identifying endotype status, and a gene expression signature predicting the onset of sepsis were validated for future clinical use.

Whole blood and clinical data profiles were collected from 115 patients in emergency rooms (ERs) from two different countries/continents (Netherlands and Canada) and 82 patients in one intensive care unit (ICU; Canada) and compared to 9 healthy controls from the same sources. ER patients were recruited into the study within two hours of admission if the attending clinician suspected possible sepsis and observed two or more systemic inflammatory response syndrome (SIRS) symptoms. Blood RNA-Seq transcriptomic profiles were analyzed to identify early mechanistic gene expression signatures useful for triage. Machine learning was used to uncover endotypes (subdivisions of the disease with distinct pathophysiological mechanisms and clinical responses) and to validate corresponding gene signatures with prognostic value. Patients with early sepsis exhibited evidence of five mechanistically distinct endotypes, namely Neutrophilic-Suppressive (NPS), Inflammatory (INF), Innate Host Defense (IHD), Interferon (IFN), and Adaptive (ADA) endotypes each of which was defined by a set of approximately 200 genes that were uniquely differentially expressed in patients with the given endotype but not in any of the other endotypes. Subsequently, a classification tool employing 88 genes was used to accurately predict endotype status in a validation cohort, while another 247 showed suitable differential expression in the given endotypes to be useful in differentiation between endotypes. This included 82 ICU patients from Toronto, Canada, of which 27 patients had Covid-19-mediated sepsis. Subsets of these 88 genes can be used, for example, to accurately identify specific endotypes (including those causing higher severity), through gene expression analysis of patient blood. Across all patients, the NPS and INF endotypes showed the worse prognosis, with higher organ dysfunction scores and severity. Furthermore, a predictive severity signature was demonstrated. This provides a method to triage a diverse spectrum of prospective pre-diagnosis sepsis patients in the emergency room (ER) into 5 mechanism-based endotypes based on the underlying molecular responses, and shows that endotypes are associated with specific clinical characteristics and outcomes. These endotypes remain detectable in the intensive care unit (ICU), indicating they are stable.

The separation of patients into endotypes has prognostic value and can inform a physician regarding future severity, enabling only the worst afflicted patients to receive the most intensive treatments and driving the potential for personalized medicines directed at treating the underlying mechanisms for the specific endotype that a patient fits into. Furthermore, signatures predicting enhanced severity independent of endotype status are described. Accordingly, the present disclosure includes methods comprising a unique set of DNA sequences that, for example, may enable the separation of sepsis patients into distinct mechanistic and/or clinically meaningful clusters and/or the prediction of mortality risk and/or sequential organ failure assessment (SOFA) score/organ failure, for example, at first clinical presentation.

Accordingly, the present disclosure includes a method for classifying a subject into a sepsis mechanistic endotype selected from neutrophilic-suppressive (NPS), inflammatory (INF), innate host defense (IHD), interferon (IFN) and adaptive (ADA) endotypes, the method comprising:
(a) determining, in a biological sample from the subject, a level of expression for each of a plurality of genes, to provide a sample gene signature; and
(b) comparing the sample gene signature with a reference gene signature to determine whether the subject has the sepsis mechanistic endotype,
wherein the sample gene signature and reference gene signature comprise an NPS endotype sub-signature, an INF endotype sub-signature, an IHD endotype sub-signature, an IFN endotype sub-signature, an ADA endotype sub-signature or combinations thereof,
   wherein the NPS endotype sub-signature comprises genes selected from the group consisting of AGFG1, ARG1, ATP9A, ANXA3, EFNA1, GADD45A, GPR84, HPGD, IL1R1, KLF14, KREMEN1, MIR646HG, MLLT1, NSUN7, OLAH, ORM2, PCOLCE2, PFKFB2, SLC51A, TNFAIP8L3, ZDHHC19, ADAMTS3, AKR1C1, ALDH1A2, ALOX5AP, ALPL, AMPH, ANKRD55, BCL3, BTBD19, CA4, CD163L1, CD177, CD82, CST7, CYP19A1, CYSTM1, DAAM2, DGAT2, ECHDC3, ENTPD7, EXOSC4, FFAR3, FGF13, FSTL4, GALNT14, GRAMD1A, GRB10, GYG1, HPGD, IER3, IL18RAP, IL1R2, IL1RN, IRAG1-AS1, KCNE1B, KCNMA1, MCEMP1, MKNK1, MMP9, MSRA, NECAB1, NSMCE1-DT, OPLAH, PDGFC, PFKFB3, PHF24, PI3, PLIN4, PLIN5, PLK3, POR, PROK2, RFX2, RGL4, ROM1, S100A12, S100P, SEMA6B, SHROOM4, SLP1, SOCS3, SPATC1, SPDYA, SPINK8, SPP1, ST6GALNAC3, SYN2, TDRD9, TMEM120A, TMIGD3, TSPO, UPP1, and XCR1;
   wherein the INF endotype sub-signature comprises genes selected from the group consisting of BNIP3L, CA1, FAM83A, FECH, GLRX5, GYPA, IFIT1B, RHCE, RIOK3, RNF182, SLC6A19, SPTA1, THEM5, TLCD4, TSPAN5, TSPO2, ABCG2, ACHE, ACKR1, ACSL6, ADD2, AHSP, ALAS2, ALDH5A1, ANK1, ANKRD9, AQP1, ARHGEF12, ARHGEF37, ARL4A, ATP1B2, ATP1B2, BBOF1, BCAM, BCL2L1, BLVRB, BPGM, Clorf116, CA2, CISD2, CLIC2, CR1L, CR1L, CTNNAL1, CTSE, DCAF12, DMTN, DNAJC6, DPCD, DYRK3, EMID1, EPB42, ERFE, FAM210B, FAXDC2, FRMD4A, GMPR, GSPT1, GYPB, HBM, HEMGN, HEPACAM2, HMBS, IGF2BP2, ISCA1, ITLN1, KANK2, KCNH2, KDM7A-DT, KEL, KLC3, KLF1, KLHDC8A, KRT1, LRRC2, MAOA, MARCHF8, MBNL3, MFSD2B, MRC2, MXI1, MYL4, NFIX, NUDT4, OSBP2, PAGE2B, PBX1, PCDH1, PGF, PLEK2, PNP, PRDX2, PTPRF, RAPIGAP, RBM38, RFESD, RFESD, RGCC, RGS16, RHAG, RHD, RIPOR3, RNF175, RUNDC3A, SEC14L4, SELENBP1, SELENOP, SFRP2, SGIP1, SIAH2, SLC14A1, SLC1A5, SLC22A23, SLC2A1, SLC4A1, SLC6A8, SLC6A9, SLC7A5, SMIM5, SNCA, SOX6, SPTB, STRADB, TAL1, TENT5C, TFR2, TMCC2, TMOD1, TNS1, TRIM10, TRIM58, TSPAN7, TTC25, UBB, USP12, XK, YBX3, and YPEL4;
   wherein the IHD endotype sub-signature comprises genes selected from the group consisting of: ABCA6, ADAM23, ALOX15, CACNA2D3, DYNC2H1, GPR34, GRAMD1C, LPL, MAP7, MIR155HG, PLCB1, SDC2, SIGLEC8, SPRED1, SLC16A14, SMPD3, TPPP3, TPRG1, ZNF600, ADGRD1, ANGPT1, GPR82, HDAC9, IL5RA, KLHDC1, PRSS33, PTGDR2, PTGFRN, TBC1D12, and TRIM2;
   wherein the IFN endotype sub-signature comprises genes selected from the group consisting of ANKRD22, APOL1, APOL4, BATF2, CARD17, CD274, EPSTI1, ETV7, GBP5, IDO1, IFITM3, P2RY14, PLEKHO1, RSAD2, SERPING1, TFEC, EXOC3L1, IRF7, OAS1, SEPTIN4, LY6E, and LAMP3; and
   wherein the ADA endotype sub-signature comprises genes selected from the group consisting of CCL2, CDC45, CENPF, CLEC4F, GTSE1, IFI27, ISG15, KCTD14, KIF14, KIF15, KLHDC7B, LGALS3BP, OTOF, PDIA4, SIGLEC1, USP18, AGRN, CD38, CDCA7, CDT1, CTLA4, DHX58, EME1, FAM111B, HES4, IFI44L, IFIT3, IFNG-AS1, IL12RB2, IL4I1, KIF19, LAG3, MCM10, P2RY6, PACSIN1, PARM1, SAMD4A, SPATS2L, HERC5, TMPRSS3, TNFRSF13B, TSHR, and TTC21A.

In an embodiment, the sample gene signature and the reference gene signature comprise, consist essentially of or consist of the NPS endotype sub-signature, the INF endotype sub-signature, the IHD endotype sub-signature, the IFN endotype sub-signature, and the ADA endotype sub-signature. In another embodiment, the sample gene signature and the reference gene signature comprise the NPS endotype sub-signature, the INF endotype sub-signature, the IHD endotype sub-signature, the IFN endotype sub-signature, and the ADA endotype sub-signature.

In some embodiments, NPS endotype sub-signature, INF endotype sub-signature, IHD endotype sub-signature, IFN endotype sub-signature, ADA endotype sub-signature or combinations thereof may include all genes listed herein in respect to the respective NPS endotype sub-signature, INF endotype sub-signature, IHD endotype sub-signature, IFN endotype sub-signature, ADA endotype sub-signature or combinations thereof. Alternatively, in some embodiments, the NPS endotype sub-signature, INF endotype sub-signature, IHD endotype sub-signature, IFN endotype sub-signature, ADA endotype sub-signature may include a single gene, a pair, or a multiple (e.g., three genes, four genes, five genes, six genes, seven genes, eight genes, nine genes, ten genes, etc.) that is a subset of the genes listed herein in respect to the NPS endotype sub-signature, INF endotype sub-signature, IHD endotype sub-signature, IFN endotype sub-signature, ADA endotype sub-signature. In an embodiment, the NPS endotype sub-signature, INF endotype sub-signature, IHD endotype sub-signature, IFN endotype sub-signature, ADA endotype sub-signature or combinations thereof may include all genes listed for the respective sub-signature or combinations thereofin the 88 gene signature of Table 3. In another embodiment, the NPS endotype sub-signature, INF endotype sub-signature, IHD endotype sub-signature, IFN endotype sub-signature, ADA endotype sub-signature or combinations thereof may include all genes listed for the respective sub-signature or combinations thereof in the subset of 40 genes from the list in Table 3, namely NSUN7, ATP9A, PFKFB2, ARG1, ANXA3, IL1R1, GADD45A, MLLT1, MIR646HG, AGFG1, KREMEN1, RIOK3, BNIP3L, TLCD4, SPTA1, TSPAN5, GLRX5, IFIT1B, ADAM23, MAP7, CACNA2D3, GPR34, GRAMD1C, PLCB1, DYNC2H1, TPRG1, ZNF600, TPPP3, PLEKHO1, APOL1, EPSTI1, RSAD2, IFITM3, SERPING1, GTSE1, CDC45, CENPF, KIF14, PDIA4, and KIF15.

In an embodiment, the NPS endotype sub-signature comprises genes selected from the group consisting of AGFG1, ARG1, ATP9A, ANXA3, EFNA1, GADD45A, GPR84, HPGD, IL1R1, KLF14, KREMEN1, MIR646HG, MLLT1, NSUN7, OLAH, ORM2, PCOLCE2, PFKFB2, SLC51A, TNFAIP8L3, and ZDHHC19. In another embodiment, the NPS endotype sub-signature consists essentially of genes selected from the group consisting of: AGFG1, ARG1, ATP9A, ANXA3, EFNA1, GADD45A, GPR84, HPGD, IL1R1, KLF14, KREMEN1, MIR646HG, MLLT1, NSUN7, OLAH, ORM2, PCOLCE2, PFKFB2, SLC51A, TNFAIP8L3, and ZDHHC19. In a further embodiment, the NPS endotype sub-signature consists of genes selected from the group consisting of: AGFG1, ARG1, ATP9A, ANXA3, EFNA1, GADD45A, GPR84, HPGD, IL1R1, KLF14, KREMEN1, MIR646HG, MLLT1, NSUN7, OLAH, ORM2, PCOLCE2, PFKFB2, SLC51A, TNFAIP8L3, and ZDHHC19. In another embodiment, the NPS endotype sub-signature comprises, consists essentially of or consists of 1, 2, 3, 4, 5, 6, 7, 8, 9, 10, 11, 12, 13, 14, 15, 16, 17, 18, 19, 20 or 21 genes selected from the group consisting of: AGFG1, ARG1, ATP9A, ANXA3, EFNA1, GADD45A, GPR84, HPGD, IL1R1, KLF14, KREMEN1, MIR646HG, MLLT1, NSUN7, OLAH, ORM2, PCOLCE2, PFKFB2, SLC51A, TNFAIP8L3, and ZDHHC19. In another embodiment, the NPS endotype sub-signature comprises, consists essentially of or consists of 1, 2, 3, 4, 5, 6, 7, 8, 9, 10 or 11 genes selected from the group consisting of: NSUN7, ATP9A, PFKFB2, ARG1, ANXA3, IL1R1, GADD45A, MLLT1, MIR646HG, AGFG1 and KREMEN1.

In an embodiment, the NPS endotype sub-signature comprises: AGFG1, ARG1, ATP9A, ANXA3, EFNA1, GADD45A, GPR84, HPGD, IL1R1, KLF14, KREMEN1, MIR646HG, MLLT1, NSUN7, OLAH, ORM2, PCOLCE2, PFKFB2, SLC51A, TNFAIP8L3, and ZDHHC19. In another embodiment of the present disclosure, the NPS endotype sub-signature consists essentially of AGFG1, ARG1, ATP9A, ANXA3, EFNA1, GADD45A, GPR84, HPGD, IL1R1, KLF14, KREMEN1, MIR646HG, MLLT1, NSUN7, OLAH, ORM2, PCOLCE2, PFKFB2, SLC51A, TNFAIP8L3, and ZDHHC19. In a further embodiment, the NPS endotype sub-signature consists of: AGFG1, ARG1, ATP9A, ANXA3, EFNA1, GADD45A, GPR84, HPGD, IL1R1, KLF14, KREMEN1, MIR646HG, MLLT1, NSUN7, OLAH, ORM2, PCOLCE2, PFKFB2, SLC51A, TNFAIP8L3, and ZDHHC19. In another embodiment, the NPS endotype sub-signature comprises: NSUN7, ATP9A, PFKFB2, ARG1, ANXA3, IL1R1, GADD45A, MLLT1, MIR646HG, AGFG1 and KREMEN1. In another embodiment, the NPS endotype sub-signature consists essentially of: NSUN7, ATP9A, PFKFB2, ARG1, ANXA3, IL1R1, GADD45A, MLLT1, MIR646HG, AGFG1 and KREMEN1. In another embodiment, the NPS endotype sub-signature consists of: NSUN7, ATP9A, PFKFB2, ARG1, ANXA3, IL1R1, GADD45A, MLLT1, MIR646HG, AGFG1 and KREMEN1.

In an embodiment, the INF endotype sub-signature comprises genes selected from the group consisting of BNIP3L, CA1, FAM83A, FECH, GLRX5, GYPA, IFIT1B, RHCE, RIOK3, RNF182, SLC6A19, SPTA1, THEM5, TLCD4, TSPAN5, and TSPO2. In another embodiment, the INF endotype sub-signature consists essentially of genes selected from the group consisting of BNIP3L, CA1, FAM83A, FECH, GLRX5, GYPA, IFIT1B, RHCE, RIOK3, RNF182, SLC6A19, SPTA1, THEM5, TLCD4, TSPAN5, and TSPO2. In a further embodiment, the INF endotype sub-signature consists of genes selected from the group consisting of BNIP3L, CA1, FAM83A, FECH, GLRX5, GYPA, IFIT1B, RHCE, RIOK3, RNF182, SLC6A19, SPTA1, THEM5, TLCD4, TSPAN5, and TSPO2. In another embodiment, the INF endotype sub-signature comprises, consists essentially of or consists of 1, 2, 3, 4, 5, 6, 7, 8, 9, 10, 11, 12, 13, 14, 15 or 16 genes selected from the group consisting of BNIP3L, CA1, FAM83A, FECH, GLRX5, GYPA, IFIT1B, RHCE, RIOK3, RNF182, SLC6A19, SPTA1, THEM5, TLCD4, TSPAN5, and TSPO2. In another embodiment, the INF endotype sub-signature comprises, consists essentially of or consists of 1, 2, 3, 4, 5, 6 or 7 genes selected from the group consisting of RIOK3, BNIP3L, TLCD4, SPTA1, TSPAN5, GLRX5, and IFIT1B.

In an embodiment, the INF endotype sub-signature comprises: BNIP3L, CA1, FAM83A, FECH, GLRX5, GYPA, IFIT1B, RHCE, RIOK3, RNF182, SLC6A19, SPTA1, THEM5, TLCD4, TSPAN5, and TSPO2. In another embodiment of the present disclosure, the INF endotype sub-signature consists essentially of BNIP3L, CA1, FAM83A, FECH, GLRX5, GYPA, IFIT1B, RHCE, RIOK3, RNF182, SLC6A19, SPTA1, THEM5, TLCD4, TSPAN5, and TSPO2. In a further embodiment, the INF endotype sub-signature consists of BNIP3L, CA1, FAM83A, FECH, GLRX5, GYPA, IFIT1B, RHCE, RIOK3, RNF182, SLC6A19, SPTA1, THEM5, TLCD4, TSPAN5, and TSPO2. In another embodiment, the INF endotype sub-signature comprises: RIOK3, BNIP3L, TLCD4, SPTA1, TSPAN5, GLRX5, and IFIT1B. In another embodiment, the INF endotype sub-signature consists essentially of RIOK3, BNIP3L, TLCD4, SPTA1, TSPAN5, GLRX5, and IFIT1B. In another embodiment, the INF endotype sub-signature consists of RIOK3, BNIP3L, TLCD4, SPTA1, TSPAN5, GLRX5, and IFIT1B.

In an embodiment, the IHD endotype sub-signature comprises genes selected from the group consisting of ABCA6, ADAM23, ALOX15, CACNA2D3, DYNC2H1, GPR34, GRAMD1C, LPL, MAP7, MIR155HG, PLCB1, SDC2, SIGLEC8, SPRED1, SLC16A14, SMPD3, TPPP3, TPRG1, and ZNF600. In another embodiment, the IHD endotype sub-signature consists essentially of genes selected from the group consisting of ABCA6, ADAM23, ALOX15, CACNA2D3, DYNC2H1, GPR34, GRAMD1C, LPL, MAP7, MIR155HG, PLCB1, SDC2, SIGLEC8, SPRED1, SLC16A14, SMPD3, TPPP3, TPRG1, and ZNF600. In a further embodiment, the IHD endotype sub-signature consists of genes selected from the group consisting of ABCA6, ADAM23, ALOX15, CACNA2D3, DYNC2H1, GPR34, GRAMD1C, LPL, MAP7, MIR155HG, PLCB1, SDC2, SIGLEC8, SPRED1, SLC16A14, SMPD3, TPPP3, TPRG1, and ZNF600. In another embodiment, the IHD endotype sub-signature comprises, consists essentially of or consists of 1, 2, 3, 4, 5, 6, 7, 8, 9, 10, 11, 12, 13, 14, 15, 16, 17, 18 or 19 genes selected from the group consisting of ABCA6, ADAM23, ALOX15, CACNA2D3, DYNC2H1, GPR34, GRAMD1C, LPL, MAP7, MIR155HG, PLCB1, SDC2, SIGLEC8, SPRED1, SLC16A14, SMPD3, TPPP3, TPRG1, and ZNF600. In another embodiment, the IHD endotype sub-signature comprises, consists essentially of or consists of 1, 2, 3, 4, 5, 6, 7, 8, 9 or 10 genes selected from the group consisting of ADAM23, MAP7, CACNA2D3, GPR34, GRAMD1C, PLCB1, DYNC2H1, TPRG1, ZNF600, and TPPP3.

In an embodiment, the IHD endotype sub-signature comprises: ABCA6, ADAM23, ALOX15, CACNA2D3, DYNC2H1, GPR34, GRAMD1C, LPL, MAP7, MIR155HG, PLCB1, SDC2, SIGLEC8, SPRED1, SLC16A14, SMPD3, TPPP3, TPRG1, and ZNF600. In another embodiment, the IHD endotype sub-signature consists essentially of ABCA6, ADAM23, ALOX15, CACNA2D3, DYNC2H1, GPR34, GRAMD1C, LPL, MAP7, MIR155HG, PLCB1, SDC2, SIGLEC8, SPRED1, SLC16A14, SMPD3, TPPP3, TPRG1, and ZNF600. In a further embodiment, the IHD endotype sub-signature consists of ABCA6, ADAM23, ALOX15, CACNA2D3, DYNC2H1, GPR34, GRAMD1C, LPL, MAP7, MIR155HG, PLCB1, SDC2, SIGLEC8, SPRED1, SLC16A14, SMPD3, TPPP3, TPRG1, and ZNF600. In another embodiment, the IHD endotype sub-signature comprises: ADAM23, MAP7, CACNA2D3, GPR34, GRAMD1C, PLCB1, DYNC2H1, TPRG1, ZNF600, and TPPP3. In another embodiment, the IHD endotype sub-signature consists essentially of ADAM23, MAP7, CACNA2D3, GPR34, GRAMD1C, PLCB1, DYNC2H1, TPRG1, ZNF600, and TPPP3. In another embodiment, the IHD endotype sub-signature consists of ADAM23, MAP7, CACNA2D3, GPR34, GRAMD1C, PLCB1, DYNC2H1, TPRG1, ZNF600, and TPPP3.

In an embodiment, the the IFN endotype sub-signature comprises genes selected from the group consisting of ANKRD22, APOL1, APOL4, BATF2, CARD17, CD274, EPSTI1, ETV7, GBP5, IDO1, IFITM3, P2RY14, PLEKHO1, RSAD2, SERPING1, and TFEC. In another embodiment, the IFN endotype sub-signature consists essentially of genes selected from the group consisting of ANKRD22, APOL1, APOL4, BATF2, CARD17, CD274, EPSTI1, ETV7, GBP5, IDO1, IFITM3, P2RY14, PLEKHO1, RSAD2, SERPING1, and TFEC. In a further embodiment, IFN endotype sub-signature consists of genes selected from the group consisting of ANKRD22, APOL1, APOL4, BATF2, CARD17, CD274, EPSTI1, ETV7, GBP5, IDO1, IFITM3, P2RY14, PLEKHO1, RSAD2, SERPING1, and TFEC. In another embodiment, the IFN endotype sub-signature comprises, consists essentially of or consists of 1, 2, 3, 4, 5, 6, 7, 8, 9, 10, 11, 12, 13, 14, 15 or 16 genes selected from the group consisting of ANKRD22, APOL1, APOL4, BATF2, CARD17, CD274, EPSTI1, ETV7, GBP5, IDO1, IFITM3, P2RY14, PLEKHO1, RSAD2, SERPING1, and TFEC. In another embodiment, the IFN endotype sub-signature comprises, consists essentially of or consists of 1, 2, 3, 4, 5 or 6 genes selected from the group consisting of PLEKHO1, APOL1, EPSTI1, RSAD2, IFITM3, and SERPING1.

In an embodiment, the IFN endotype sub-signature comprises: ANKRD22, APOL1, APOL4, BATF2, CARD17, CD274, EPSTI1, ETV7, GBP5, IDO1, IFITM3, P2RY14, PLEKHO1, RSAD2, SERPING1, and TFEC. In another embodiment, the IFN endotype sub-signature consists essentially of ANKRD22, APOL1, APOL4, BATF2, CARD17, CD274, EPSTI1, ETV7, GBP5, IDO1, IFITM3, P2RY14, PLEKHO1, RSAD2, SERPING1, and TFEC. In a further embodiment, the IFN endotype sub-signature consists of ANKRD22, APOL1, APOL4, BATF2, CARD17, CD274, EPSTI1, ETV7, GBP5, IDO1, IFITM3, P2RY14, PLEKHO1, RSAD2, SERPING1, and TFEC. In another embodiment, the IFN endotype sub-signature comprises: PLEKHO1, APOL1, EPSTI1, RSAD2, IFITM3, and SERPING1. In another embodiment, the IFN endotype sub-signature consists essentially of PLEKHO1, APOL1, EPSTI1, RSAD2, IFITM3, and SERPING1. In another embodiment, the IFN endotype sub-signature consists of PLEKHO1, APOL1, EPSTI1, RSAD2, IFITM3, and SERPING1.

In an embodiment, the ADA endotype sub-signature comprises genes selected from the group consisting of CCL2, CDC45, CENPF, CLEC4F, GTSE1, IFI27, ISGI5, KCTD14, KIF14, KIF15, KLHDC7B, LGALS3BP, OTOF, PDIA4, SIGLEC1, and USP18. In another embodiment, the ADA endotype sub-signature consists essentially of genes selected from the group consisting of CCL2, CDC45, CENPF, CLEC4F, GTSE1, IFI27, ISGI5, KCTD14, KIF14, KIF15, KLHDC7B, LGALS3BP, OTOF, PDIA4, SIGLEC1, and USP18. In a further embodiment, the ADA endotype sub-signature consists of genes selected from the group consisting of CCL2, CDC45, CENPF, CLEC4F, GTSE1, IFI27, ISGI5, KCTD14, KIF14, KIF15, KLHDC7B, LGALS3BP, OTOF, PDIA4, SIGLEC1, and USP18. In another embodiment, the ADA endotype sub-signature comprises, consists essentially of or consists of 1, 2, 3, 4, 5, 6, 7, 8, 9, 10, 11, 12, 13, 14, 15 or 16 genes selected from the group consisting of CCL2, CDC45, CENPF, CLEC4F, GTSE1, IFI27, ISGI5, KCTD14, KIF14, KIF15, KLHDC7B, LGALS3BP, OTOF, PDIA4, SIGLEC1, and USP18. In another embodiment, the ADA endotype sub-signature comprises, consists essentially of or consists of 1, 2, 3, 4, 5 or 6 genes selected from the group consisting of GTSE1, CDC45, CENPF, KIF14, PDIA4, and KIF15.

In an embodiment, the ADA endotype sub-signature comprises: CCL2, CDC45, CENPF, CLEC4F, GTSE1, IF127, ISG15, KCTD14, KIF14, KIF15, KLHDC7B, LGALS3BP, OTOF, PDIA4, SIGLEC1 and USP18. In another embodiment, the ADA endotype sub-signature consists essentially of: CCL2, CDC45, CENPF, CLEC4F, GTSE1, IF127, ISG15, KCTD14, KIF14, KIF15, KLHDC7B, LGALS3BP, OTOF, PDIA4, SIGLEC1 and USP18. In a further embodiment, the ADA endotype sub-signature consists of: CCL2, CDC45, CENPF, CLEC4F, GTSE1, IF127, ISG15, KCTD14, KIF14, KIF15, KLHDC7B, LGALS3BP, OTOF, PDIA4, SIGLEC1 and USP18. In another embodiment, the ADA endotype sub-signature comprises: GTSE1, CDC45, CENPF, KIF14, PDIA4, and KIF15. In another embodiment, the ADA endotype sub-signature consists essentially of: GTSE1, CDC45, CENPF, KIF14, PDIA4, and KIF15. In another embodiment, the ADA endotype sub-signature consists of: GTSE1, CDC45, CENPF, KIF14, PDIA4, and KIF15.

In an embodiment, the reference gene signature represents a standard level of expression of the genes comprised therein and a difference between a sample endotype sub-signature and a reference endotype sub-signature indicates that the subject has the sepsis mechanistic endotype corresponding to that sub-signature.

The present disclosure also includes a method for classifying a subject into a sepsis mechanistic endotype selected from neutrophilic-suppressive (NPS), inflammatory (INF), innate host defense (IHD), interferon (IFN) and adaptive (ADA) endotypes, the method comprising:
(a) determining, in a biological sample from the subject, a level of expression for each of a plurality of genes, to provide a sample gene signature; and
(b) comparing the sample gene signature with a reference gene signature to determine whether the subject has the sepsis mechanistic endotype,
wherein the sample gene signature and reference gene signature comprise an NPS endotype signature pair, an INF endotype signature pair, an IHD endotype signature pair, an IFN endotype signature pair, an ADA endotype signature pair or combinations thereof,
   wherein the NPS endotype signature pair is selected from: GADD45A/EFNA1, EFNA1/MIR646HG, MIR646HG/KLF14, MLLT1/MIR646HG, ARG1/MLLT1, MLLT1/EFNA1, MLLT1/NSUN7, EFNA1I/NSUN7, SLC51A/EFNA1, EFNA1/KLF14, ZDHHC19/EFNA1, EFNA1/AGFG1, NSUN7/KLF14, EFNA1/PFKFB2, MLLT1/KLF14, ADAMTS3/PCOLCE2, ADAMTS3/ZDHHC19, ADAMTS3/SLC51A, ADAMTS3/HPGD, ADAMTS3/SEMA6B, ADAMTS3/EFNA1, ADAMTS3/AGFG1, ADAMTS3/NSUN7, ADAMTS3/TNFAIP8L3, ADAMTS3/KREMEN1, ADAMTS3/ORM2, ADAMTS3/MIR646HG, ADAMTS3/KLF14, AGFG1/NSUN7, AGFG1/TNFAIP8L3, AGFG1/KREMEN1, AGFG1/ORM2, AGFG1/MIR646HG, AGFG1/KLF14, ANXA3/GPR84, ANXA3/OLAH, ANXA3/ADAMTS3, ANXA3/PCOLCE2, ANXA3/ZDHHC19, ANXA3/SLC51A, ANXA3/HPGD, ANXA3/SEMA6B, ANXA3/EFNA1, ANXA3/AGFG1, ANXA3/NSUN7, ANXA3/TNFAIP8L3, ANXA3/KREMEN1, ANXA3/ORM2, ANXA3/MIR646HG, ANXA3/KLF14, ARG1/PFKFB2, ARG1/MLLT1, ARG1/ANXA3, ARG1/GPR84, ARG1/OLAH, ARG1/ADAMTS3, ARG1/PCOLCE2, ARG1/ZDHHC19, ARG1/SLC51A, ARG1/HPGD, ARG1/SEMA6B, ARG1/EFNA1, ARG1/AGFG1, ARG1/NSUN7, ARG1/TNFAIP8L3, ARG1/KREMEN1, ARG1/ORM2, ARG1/MIR646HG, ARG1/KLF14, ATP9A/EPB41L4B, ATP9A/IL1R1, ATP9A/GADD45A, ATP9A/ARG1, ATP9A/PFKFB2, ATP9A/MLLT1, ATP9A/ANXA3, ATP9A/GPR84, ATP9A/OLAH, ATP9A/ADAMTS3, ATP9A/PCOLCE2, ATP9A/ZDHHC19, ATP9A/SLC51A, ATP9A/HPGD, ATP9A/SEMA6B, ATP9A/EFNA1, ATP9A/AGFG1, ATP9A/NSUN7, ATP9A/TNFAIP8L3, ATP9A/KREMEN1, ATP9A/ORM2, ATP9A/MIR646HG, ATP9A/KLF14, EFNA1/AGFG1, EFNA1/NSUN7, EFNA1/TNFAIP8L3, EFNA1/KREMEN1, EFNA1/ORM2, EFNA1/MIR646HG, EFNA1/KLF14, EPB41L4B/IL1R1, EPB41L4B/GADD45A, EPB41L4B/ARG1, EPB41L4B/PFKFB2, EPB41L4B/MLLT1, EPB41L4B/ANXA3, EPB41L4B/GPR84, EPB41L4B/OLAH, EPB41L4B/ADAMTS3, EPB41L4B/PCOLCE2, EPB41L4B/ZDHHC19, EPB41L4B/SLC51A, EPB41L4B/HPGD, EPB41L4B/SEMA6B, EPB41L4B/EFNA1, EPB41L4B/AGFG1I,EPB41L4B/NSUN7, EPB41L4B/TNFAIP8L3, EPB41L4B/KREMEN1, EPB41L4B/MIR646HG, EPB41L4B/KLF14, GADD45A/ARG1, GADD45A/PFKFB2, GADD45A/MLLT1, GADD45A/ANXA3, GADD45A/GPR84, GADD45A/OLAH, GADD45A/ADAMTS3, GADD45A/PCOLCE2, GADD45A/ZDHHC19, GADD45A/SLC51A, GADD45A/HPGD, GADD45A/SEMA6B, GADD45A/EFNA1, GADD45A/AGFG1, GADD45A/NSUN7, GADD45A/TNFAIP8L3, GADD45A/KREMEN1, GADD45A/ORM2, GADD45A/MIR646HG, GADD45A/KLF14, GPR84/OLAH, GPR84/ADAMTS3, GPR84/PCOLCE2, GPR84/ZDHHC19, GPR84/SLC51A, GPR84/HPGD, GPR84/SEMA6B, GPR84/EFNA1, GPR84/AGFG1, GPR84/NSUN7, GPR84/TNFAIP8L3, GPR84/KREMEN1, GPR84/ORM2, GPR84/MIR646HG, GPR84/KLF14, HPGD/SEMA6B, HPGD/EFNA1, HPGD/AGFG1, HPGD/NSUN7, HPGD/TNFAIP8L3, HPGD/KREMEN1, HPGD/ORM2, HPGD/MIR646HG, HPGD/KLF14, IL1R1/GADD45A, ILIR1/ARG1, IL1R1/PFKFB2, IL1R1/MLLT1, IL1R1/ANXA3, IL1R1/GPR84, IL1R1/OLAH, IL1R1/ADAMTS3, IL1R1/PCOLCE2, IL1R1/ZDHHC19, IL1R1/SLC51A, IL1R1/HPGD, IL1R1/SEMA6B, IL1R1/EFNA1, IL1R1/AGFG1, IL1R1/NSUN7, IL1R1/TNFAIP8L3, IL1R1/KREMEN1, IL1R1/ORM2, IL1R1/MIR646HG, IL1R1/KLF14, KREMEN1/ORM2, KREMEN1/MIR646HG, KREMEN1/KLF14, MIR646HG/KLF14, MLLT1/ANXA3, MLLT1/GPR84, MLLT1/OLAH, MLLT1/ADAMTS3, MLLT1/PCOLCE2, MLLT1/ZDHHC19, MLLT1/SLC51A, MLLT1/HPGD, MLLT1/SEMA6B, MLLT1/EFNA1, MLLT1/AGFG1, MLLT1/NSUN7, MLLT1/TNFAIP8L3, MLLT1/KREMEN1, MLLT1/ORM2, MLLT1/MIR646HG, MLLT1/KLF14, NSUN7/TNFAIP8L3, NSUN7/KREMEN1, NSUN7/ORM2, NSUN7/MIR646HG, NSUN7/KLF14, OLAH/ADAMTS3, OLAH/PCOLCE2, OLAH/ZDHHC19, OLAH/SLC51A, OLAH/HPGD, OLAH/SEMA6B, OLAH/EFNA1, OLAH/AGFG1, OLAH/NSUN7, OLAH/TNFAIP8L3, OLAH/KREMEN1, OLAH/ORM2, OLAH/MIR646HG, OLAH/KLF14, ORM2/MIR646HG, ORM2/KLF14, PCOLCE2/ZDHHC19, PCOLCE2/SLC51A, PCOLCE2/HPGD, PCOLCE2/SEMA6B, PCOLCE2/EFNA1, PCOLCE2/AGFG1, PCOLCE2/NSUN7, PCOLCE2/TNFAIP8L3, PCOLCE2/KREMEN1, PCOLCE2/ORM2, PCOLCE2/MIR646HG, PCOLCE2/KLF14, PFKFB2/MLLT1, PFKFB2/ANXA3, PFKFB2/GPR84, PFKFB2/OLAH, PFKFB2/ADAMTS3, PFKFB2/PCOLCE2, PFKFB2/ZDHHC19, PFKFB2/SLC51A, PFKFB2/HPGD, PFKFB2/SEMA6B, PFKFB2/EFNA1, PFKFB2/AGFG1, PFKFB2/NSUN7, PFKFB2/TNFAIP8L3, PFKFB2/KREMEN1, PFKFB2/ORM2, PFKFB2/MIR646HG, PFKFB2/KLF14, SEMA6B/EFNA1, SEMA6B/AGFG1, SEMA6B/NSUN7, SEMA6B/TNFAIP8L3, SEMA6B/KREMEN1, SEMA6B/ORM2, SEMA6B/MIR646HG, SEMA6B/KLF14, SLC51A/HPGD, SLC51A/SEMA6B, SLC51A/EFNA1, SLC51A/AGFG1, SLC51A/NSUN7, SLC51A/TNFAIP8L3, SLC51A/KREMEN1, SLC51A/ORM2, SLC51A/MIR646HG, SLC51A/KLF14, TNFAIP8L3/KREMEN1, TNFAIP8L3/ORM2, TNFAIP8L3/MIR646HG, TNFAIP8L3/KLF14, ZDHHC19/SLC51A, ZDHHC19/HPGD, ZDHHC19/SEMA6B, ZDHHC19/EFNA1, ZDHHC19/AGFG1, ZDHHC19/NSUN7, ZDHHC19/TNFAIP8L3, ZDHHC19/KREMEN1, ZDHHC19/ORM2, ZDHHC19/MIR646HG, and ZDHHC19/KLF14;
   wherein the INF endotype signature pair is selected from: FECH/TFEC, TFEC/IFIT1B, FECH/RNF182, IFIT1B/FECH, FECH/APOL4, FECH/GYPA, ITLN1/FECH, FECH/THEM5, IFIT1B/CA1, RHAG/FECH, FECH/FAM83A, RHCE/FECH, TFEC/CA1, SPTA1/FECH, ANKRD22/GLRX5, ANKRD22/GYPA, ANKRD22/IFIT1B, ANKRD22/ITLN1, ANKRD22/KLHDC8A, ANKRD22/RHCE, ANKRD22/RNF182, ANKRD22/SPTA1, ANKRD22/THEM5, ANKRD22/TSPAN5, APOL4/BNIP3L, APOL4/CA1, APOL4/DYRK3, APOL4/FAM83A, APOL4/GLRX5, APOL4/GYPA, APOL4/IFIT1B, APOL4/ITLN1, APOL4/KLHDC8A, APOL4/RHAG, APOL4/RHCE, APOL4/RIOK3, APOL4/RNF182, APOL4/SPTA1, APOL4/THEM5, APOL4/TLCD4, APOL4/TMCC2, APOL4/TSPAN5, APOL4/TSPO2, BNIP3L/ANKRD22, BNIP3L/CA1, BNIP3L/CARD17, BNIP3L/CD274, BNIP3L/DYRK3, BNIP3L/FAM83A, BNIP3L/GBP5, BNIP3L/GLRX5, BNIP3L/GYPA, BNIP3L/IFIT1B, BNIP3L/ITLN1, BNIP3L/KLHDC8A, BNIP3L/P2RY14, BNIP3L/RHAG, BNIP3L/RHCE, BNIP3L/RNF182, BNIP3L/SPTA1, BNIP3L/TFEC, BNIP3L/THEM5, BNIP3L/TLCD4, BNIP3L/TMCC2, BNIP3L/TSPAN5, BNIP3L/TSPO2, CA1/ANKRD22, CA1/CARD17, CA1/DYRK3, CA1/FAM83A, CA1/GBP5, CA1/GLRX5, CA1/GYPA, CA1/IFIT1B, CA1/ITLN1, CA1/KLHDC8A, CA1/P2RY14, CA1/RHCE, CA1/RNF182, CA1/SPTA1, CA1/THEM5, CA1/TLCD4, CA1/TSPAN5, CD274/CA1, CD274/DYRK3, CD274/FAM83A, CD274/GLRX5, CD274/GYPA, CD274/IFIT1B, CD274/ITLN1, CD274/KLHDC8A, CD274/RHCE, CD274/RNF182, CD274/SPTA1, CD274/THEM5, CD274/TLCD4, CD274/TMCC2, CD274/TSPAN5, DYRK3/ANKRD22, DYRK3/CARD17, DYRK3/FAM83A, DYRK3/GBP5, DYRK3/GLRX5, DYRK3/GYPA, DYRK3/IFIT1B, DYRK3/ITLN1, DYRK3/KLHDC8A, DYRK3/P2RY14, DYRK3/RHCE, DYRK3/RNF182, DYRK3/SPTA1, DYRK3/THEM5, DYRK3/TLCD4, DYRK3/TSPAN5, FAM83A/ANKRD22, FAM83A/CARD17, FAM83A/GBP5, FAM83A/GLRX5, FAM83A/GYPA, FAM83A/IFIT1B, FAM83A/ITLN1, FAM83A/KLHDC8A, FAM83A/P2RY14, FAM83A/RHCE, FAM83A/RNF182, FAM83A/SPTA1, FAM83A/THEM5, FAM83A/TLCD4, FAM83A/TSPAN5, FECH/ANKRD22, FECH/APOL4, FECH/BNIP3L, FECH/CA1, FECH/CARD17, FECH/CD274, FECH/DYRK3, FECH/FAM83A, FECH/GBP5, FECH/GLRX5, FECH/GYPA, FECH/IFIT1B, FECH/ITLN1, FECH/KLHDC8A, FECH/P2RY14, FECH/RHAG, FECH/RHCE, FECH/RIOK3, FECH/RNF182, FECH/SPTA1, FECH/TFEC, FECH/THEM5, FECH/TLCD4, FECH/TMCC2, FECH/TSPAN5, FECH/TSPO2, GBP5/GLRX5, GBP5/GYPA, GBP5/IFIT1B, GBP5/ITLN1, GBP5/KLHDC8A, GBP5/RHCE, GBP5/RNF182, GBP5/SPTA1, GBP5/THEM5, GBP5/TSPAN5, GLRX5/CARD17, GLRX5/IFIT1B, GLRX5/RHCE, GLRX5/THEM5, GYPA/CARD17, GYPA/GLRX5, GYPA/IFIT1B, GYPA/ITLN1, GYPA/P2RY14, GYPA/RHCE, GYPA/RNF182, GYPA/THEM5, IFIT1B/CARD17, ITLN1/CARD17, ITLN1/GLRX5, ITLN1/IFIT1B, ITLN1/RHCE, ITLN1/RNF182, ITLN1/THEM5, KLHDC8A/CARD17, KLHDC8A/GLRX5, KLHDC8A/GYPA, KLHDC8A/IFIT1B, KLHDC8A/ITLN1, KLHDC8A/P2RY14, KLHDC8A/RHCE, KLHDC8A/RNF182, KLHDC8A/SPTA1, KLHDC8A/THEM5, KLHDC8A/TSPAN5, P2RY14/GLRX5, P2RY14/IFIT1B, P2RY14/ITLN1, P2RY14/RHCE, P2RY14/RNF182, P2RY14/THEM5, RHAG/ANKRD22, RHAG/CA1, RHAG/CARD17, RHAG/CD274, RHAG/DYRK3, RHAG/FAM83A, RHAG/GBP5, RHAG/GLRX5, RHAG/GYPA, RHAG/IFIT1B, RHAG/ITLN1, RHAG/KLHDC8A, RHAG/P2RY14, RHAG/RHCE, RHAG/RNF182, RHAG/SPTA1, RHAG/THEM5, RHAG/TLCD4, RHAG/TMCC2, RHAG/TSPAN5, RHAG/TSPO2, RHCE/CARD17, RHCE/IFIT1B, RHCE/THEM5, RIOK3/ANKRD22, RIOK3/BNIP3L, RIOK3/CA1, RIOK3/CARD17, RIOK3/CD274, RIOK3/DYRK3, RIOK3/FAM83A, RIOK3/GBP5, RIOK3/GLRX5, RIOK3/GYPA, RIOK3/IFIT1B, RIOK3/ITLN1, RIOK3/KLHDC8A, RIOK3/P2RY14, RIOK3/RHAG, RIOK3/RHCE, RIOK3/RNF182, RIOK3/SPTA1, RIOK3/TFEC, RIOK3/THEM5, RIOK3/TLCD4, RIOK3/TMCC2, RIOK3/TSPAN5, RIOK3/TSPO2, RNF182/CARD17, RNF182/GLRX5, RNF182/IFIT1B, RNF182/RHCE, RNF182/THEM5, SPTA1/CARD17, SPTA1/GLRX5, SPTA1/GYPA, SPTA1/IFIT1B, SPTA1/ITLN1, SPTA1/P2RY14, SPTA1/RHCE, SPTA1/RNF182, SPTA1/THEM5, SPTA1/TSPAN5, TFEC/CA1, TFEC/DYRK3, TFEC/FAM83A, TFEC/GLRX5, TFEC/GYPA, TFEC/IFIT1B, TFEC/ITLN1, TFEC/KLHDC8A, TFEC/RHAG, TFEC/RHCE, TFEC/RNF182, TFEC/SPTA1, TFEC/THEM5, TFEC/TLCD4, TFEC/TMCC2, TFEC/TSPAN5, TFEC/TSPO2, THEM5/CARD17, THEM5/IFIT1B, TLCD4/ANKRD22, TLCD4/CARD17, TLCD4/GBP5, TLCD4/GLRX5, TLCD4/GYPA, TLCD4/IFIT1B, TLCD4/ITLN1, TLCD4/KLHDC8A, TLCD4/P2RY14, TLCD4/RHCE, TLCD4/RNF182, TLCD4/SPTA1, TLCD4/THEM5, TLCD4/TSPAN5, TMCC2/ANKRD22, TMCC2/CA1, TMCC2/CARD17, TMCC2/DYRK3, TMCC2/FAM83A, TMCC2/GBP5, TMCC2/GLRX5, TMCC2/GYPA, TMCC2/IFIT1B, TMCC2/ITLN1, TMCC2/KLHDC8A, TMCC2/P2RY14, TMCC2/RHCE, TMCC2/RNF182, TMCC2/SPTA1, TMCC2/THEM5, TMCC2/TLCD4, TMCC2/TSPAN5, TSPAN5/CARD17, TSPAN5/GLRX5, TSPAN5/GYPA, TSPAN5/IFIT1B, TSPAN5/ITLN1, TSPAN5/P2RY14, TSPAN5/RHCE, TSPAN5/RNF182, TSPAN5/THEM5, TSPO2/ANKRD22, TSPO2/CA1, TSPO2/CARD17, TSPO2/CD274, TSPO2/DYRK3, TSPO2/FAM83A, TSPO2/GBP5, TSPO2/GLRX5, TSPO2/GYPA, TSPO2/IFIT1B, TSPO2/ITLN1, TSPO2/KLHDC8A, TSPO2/P2RY14, TSPO2/RHCE, TSPO2/RNF182, TSPO2/SPTA1, TSPO2/THEM5, TSPO2/TLCD4, TSPO2/TMCC2, and TSPO2/TSPAN5;
   wherein the IHD endotype signature pair is selected from: MAP7/SPRED1, SPRED1/GPR34, IL5RA/SPRED1, SPRED1/TPRG1, HRK/SPRED1, SPRED1/PLCB1, TRIM2/SPRED1, SIGLEC8/SPRED1, SMPD3/SPRED1, SPRED1/ZNF600, SPRED1/SDC2, MAP7/GPR34, PRSS33/SPRED1, SPRED1/DYNC2H1, CACNA2D3/SPRED1, ADAM23/GPR34, ADAM23/MAP7, ADAM23/PLCB1, ADAM23/SPRED1, ALOX15/GPR34, ALOX15/PLCB1, ALOX15/SPRED1, BAALC/GPR34, BAALC/PLCB1, BAALC/SPRED1, CACNA2D3/DYNC2H1, CACNA2D3/GPR34, CACNA2D3/PLCB1, CACNA2D3/SPRED1, CACNA2D3/ZNF600, GPR34/DYNC2H1, GPR34/GRAMD1C, GPR34/PLCB1, GPR34/TPRG1, GPR34/ZNF600, GPR82/DYNC2H1, GPR82/GPR34, GPR82/GRAMD1C, GPR82/PLCB1, GPR82/TPRG1, GPR82/ZNF600, GRAMD1C/DYNC2H1, GRAMD1C/PLCB1, GRAMD1C/ZNF600, HRK/DYNC2H1, HRK/GPR34, HRK/MAP7, HRK/PLCB1, HRK/SPRED1, HRK/ZNF600, IL5RA/DYNC2H1, IL5RA/GPR34, IL5RA/PLCB1, IL5RA/SPRED1, IL5RA/TRIM2, MAP7/BAALC, MAP7/CACNA2D3, MAP7/DYNC2H1, MAP7/GPR34, MAP7/GPR82, MAP7/GRAMD1C, MAP7/PLCB1, MAP7/SPRED1, MAP7/TPRG1, MAP7/ZNF600, PLCB1/DYNC2H1, PLCB1/TPRG1, PLCB1/ZNF600, PRSS33/GPR34, PRSS33/PLCB1, PRSS33/SPRED1, SDC2/DYNC2H1, SDC2/GPR34, SDC2/PLCB1, SDC2/ZNF600, SIGLEC8/DYNC2H1, SIGLEC8/GPR34, SIGLEC8/MAP7, SIGLEC8/PLCB1, SIGLEC8/SPRED1, SIGLEC8/TRIM2, SMPD3/DYNC2H1, SMPD3/GPR34, SMPD3/MAP7, SMPD3/PLCB1, SMPD3/SPRED1, SMPD3/TRIM2, SPRED1/DYNC2H1, SPRED1/GPR34, SPRED1/GPR82, SPRED1/GRAMDIC, SPRED1/PLCB1, SPRED1/SDC2, SPRED1/TPRG1, SPRED1/ZNF600, TRIM2/CACNA2D3, TRIM2/DYNC2H1, TRIM2/GPR34, TRIM2/GPR82, TRIM2/GRAMD1C, TRIM2/HRK, TRIM2/MAP7, TRIM2/PLCB1, TRIM2/SDC2, TRIM2/SPRED1, TRIM2/TPRG1, and TRIM2/ZNF600;
   wherein the IFN endotype signature pair is selected from: ETV7/PLEKHO1, IFITM3/ETV7, ETV7/APOL1, BATF2/ETV7, PLEKHO1/BATF2, ETV7/EPSTI1, EPSTI1/BATF2, IFITM3/BATF2, USP18/EPSTI1, ETV7/SEPTIN4, ETV7/LAMP3, SERPING1/BATF2, LAMP3/BATF2, LAMP3/SERPING1, APOL1/BATF2, APOL1/CLEC4F, APOL1/EPSTI1, APOL1/EXOC3L1, APOL1/HES4, APOL1/IFITM3, APOL1/LY6E, APOL1/RSAD2, APOL1/SEPTIN4, APOL1/SERPING1, APOL1/TPPP3, BATF2/EXOC3L1, BATF2/HES4, CLEC4F/BATF2, CLEC4F/EXOC3L1, EPSTI1/BATF2, EPSTI1/CLEC4F, EPSTI1/EXOC3L1, EPSTI1/HES4, EPSTI1/IFITM3, EPSTI1/LY6E, EPSTI1/RSAD2, EPSTI1/SERPING1, EPSTI1/TPPP3, ETV7/APOL1, ETV7/BATF2, ETV7/CLEC4F, ETV7/EPSTI1, ETV7/EXOC3L1, ETV7/HES4, ETV7/IFITM3, ETV7/LAMP3, ETV7/LY6E, ETV7/PLEKHO1, ETV7/RSAD2, ETV7/SEPTIN4, ETV7/SERPING1, ETV7/TPPP3, EXOC3L1/HES4, IFITM3/SERPING1, IFITM3/CLEC4F, IFITM3/TPPP3, IFITM3/LY6E, IFITM3/EXOC3L1, IFITM3/HES4, LAMP3/APOL1, LAMP3/BATF2, LAMP3/CLEC4F, LAMP3/EPSTI1, LAMP3/EXOC3L1, LAMP3/HES4, LAMP3/IFITM3, LAMP3/LY6E, LAMP3/RSAD2, LAMP3/SEPTIN4, LAMP3/SERPING1, LAMP3/TPPP3, LY6E/BATF2, LY6E/EXOC3L1, PLEKHO1/APOL1, PLEKHO1/BATF2, PLEKHO1/EPSTI1, PLEKHO1/EXOC3L1, PLEKHO1/IFITM3, PLEKHO1/LAMP3, PLEKHO1/RSAD2, PLEKHO1/SEPTIN4, PLEKHO1/SERPING1, RSAD2/BATF2, RSAD2/CLEC4F, RSAD2/EXOC3L1, RSAD2/HES4, RSAD2/IFITM3, RSAD2/LY6E, RSAD2/SERPING1, RSAD2/TPPP3, SEPTIN4/BATF2, SEPTIN4/CLEC4F, SEPTIN4/EPSTI1, SEPTIN4/EXOC3L1, SEPTIN4/HES4, SEPTIN4/IFITM3, SEPTIN4/LY6E, SEPTIN4/RSAD2, SEPTIN4/SERPING1, SEPTIN4/TPPP3, SERPING1/BATF2, SERPING1/CLEC4F, SERPING1/EXOC3L1, SERPING1/HES4, SERPING1/LY6E, SERPING1/TPPP3, TPPP3/BATF2, and TPPP3/EXOC3L1; and wherein the ADA endotype signature pair is selected from: LGALS3BP/OTOF, LGALS3BP/IFI27, LGALS3BP/KIF14, LGALS3BP/CENPF, GTSE1/LGALS3BP, LGALS3BP/KCTD14, LGALS3BP/PDIA4, LGALS3BP/TSHR, LGALS3BP/PLAAT2, OTOF/IFI27, IGF1/LGALS3BP, CDC45/LGALS3BP, LGALS3BP/KIF15, LGALS3BP/IGLL5, LGALS3BP/MIXL, CAV1/LGALS3BP, CAV1/OTOF, CDC45/LGALS3BP, CDC45/OTOF, CENPF/KCTD14, GPRC5D/OTOF, GTSE1/LGALS3BP, GTSE1/OTOF, IGF1/LGALS3BP, IGF1/OTOF, KCTD14/KLHL14, KCTD14/PDIA4, KCTD14/TSHR, KIF14/KCTD14, LGALS3BP/CENPF, LGALS3BP/GPRC5D, LGALS3BP/IFI27, LGALS3BP/IGLL5, LGALS3BP/KCTD14, LGALS3BP/KIF14, LGALS3BP/KIF15, LGALS3BP/KLHL14, LGALS3BP/MIR155HG, LGALS3BP/MIXL1, LGALS3BP/OTOF, LGALS3BP/PDIA4, LGALS3BP/PLAAT2, LGALS3BP/SDC1, LGALS3BP/SLC16A14, LGALS3BP/TSHR, OTOF/CENPF, OTOF/IFI27, OTOF/IGLL5, OTOF/KCTD14, OTOF/KIF14, OTOF/KIF15, OTOF/KLHL14, OTOF/MIR155HG, OTOF/MIXL1, OTOF/PDIA4, OTOF/PLAAT2, OTOF/SDC1, OTOF/SLC16A14, OTOF/TSHR, PLAAT2/KCTD14, TNFRSF17/LGALS3BP, and TNFRSF17/OTOF.

In an embodiment, the sample gene signature and the reference gene signature comprise, consist essentially of or consist of the NPS endotype signature pair, the INF endotype signature pair, the IHD endotype signature pair, the IFN endotype signature pair, and the ADA endotype signature pair. In another embodiment, the sample gene signature and the reference gene signature comprise the NPS endotype signature pair, the INF endotype signature pair, the IHD endotype signature pair, the IFN endotype signature pair, and the ADA endotype signature pair.

In an embodiment, the NPS endotype signature pair is selected from: GADD45A/EFNA1, EFNA1/MIR646HG, MIR646HG/KLF14, MLLT1/MIR646HG, ARG1/MLLT1, MLLT1/EFNA1, MLLT1/NSUN7, EFNA1/NSUN7, SLC51A/EFNA1, EFNA1/KLF14, ZDHHC19/EFNA1, EFNA1/AGFG1, NSUN7/KLF14, EFNA1/PFKFB2, and MLLT1/KLF14. In another embodiment, the INF endotype signature pair is selected from: FECH/TFEC, TFEC/IFIT1B, FECH/RNF182, IFIT1B/FECH, FECH/APOL4, FECH/GYPA, ITLN1/FECH, FECH/THEM5, IFIT1B/CA1, RHAG/FECH, FECH/FAM83A, RHCE/FECH, TFEC/CA1, and SPTA1/FECH. In a further embodiment, the IHD endotype signature pair is selected from: MAP7/SPRED1, SPRED1/GPR34, IL5RA/SPRED1, SPRED1/TPRG1, HRK/SPRED1, SPRED1/PLCB1, TRIM2/SPRED1, SIGLEC8/SPRED1, SMPD3/SPRED1, SPRED1/ZNF600, SPRED1/SDC2, MAP7/GPR34, PRSS33/SPRED1, SPRED1/DYNC2H1, and CACNA2D3/SPRED1. In another embodiment, the IFN endotype signature pair is selected from: ETV7/PLEKHO1, IFITM3/ETV7, ETV7/APOL1, BATF2/ETV7, PLEKHO1/BATF2, ETV7/EPSTI1, EPSTI1/BATF2, IFITM3/BATF2, USP18/EPSTI1, ETV7/SEPTIN4, ETV7/LAMP3, SERPING1/BATF2, LAMP3/BATF2, and LAMP3/SERPING1. In a further embodiment, the ADA endotype signature pair is selected from: LGALS3BP/OTOF, LGALS3BP/IFI27, LGALS3BP/KIF14, LGALS3BP/CENPF, GTSE1/LGALS3BP, LGALS3BP/KCTD14, LGALS3BP/PDIA4, LGALS3BP/TSHR, LGALS3BP/PLAAT2, OTOF/IF1I27, IGF1/LGALS3BP, CDC45/LGALS3BP, LGALS3BP/KIF15, LGALS3BP/IGLL5, and LGALS3BP/MIXL1.

In an embodiment, the reference gene signature represents a standard level of expression of the genes comprised therein and a difference between a sample endotype signature pair and a reference endotype signature pair indicates that the subject has the sepsis mechanistic endotype corresponding to that signature pair.

The present disclosure also includes a method for predicting severity of sepsis in a subject, wherein the severity of the sepsis is selected from high severity sepsis, intermediate severity sepsis and low severity sepsis, the method comprising:
(a) determining, in a biological sample from the subject, a level of expression for each of a plurality of genes, to provide a sample gene signature; and
(b) comparing the sample gene signature with a reference gene signature to determine the severity of the sepsis in the subject,
wherein high severity sepsis means a sequential organ failure assessment (SOFA) score of greater than or equal to 5, intermediate severity sepsis means a SOFA score of greater than or equal to 2 but less than 5, and low severity sepsis means a SOFA score of less than 2; and
   wherein the plurality of genes is selected from the group consisting of ABCA13, ADAMTS2, ADAMTS3, AK5, ANKRD22, ANKRD34B, ANLN, AQP1, ARG1, ARHGAP44, ARHGEF17, ASPM, ATP1B2, AURKA, AZU1, BAIAP3, BPI, Clorf226, CACNB4, CCL4L2, CCN3, CCNA1, CD177, CD24, CDK1, CDKN3, CEACAM6, CEACAM8, CENPA, CFH, CHDH, CHIT1, CKAP2L, CLEC4C, CLEC4F, CLNK, COL17A1, CRISP2, CRISP3, CTSE, CTSG, CYP19A1, CYYR1, DEFA4, DENND2C, DEPDC1, DGKK, DLC1, DLGAP5, DNAH10, DOC2B, DSP, ELANE, ERG, FAM20A, FAM83A, FBN1, FFAR3, GOS2, GGT5, GLB1L2, GJB6, GPR84, GRAMD1C, GYPA, HBM, HMGB3, HP, HPGD, HRK, IGLL1, IL1R2, IL1RL1, INHBA, IQGAP3, ITGA7, ITGB4, KIF15, KIF20A, KLF14, LAMB3, LCN2, LGR4, LPL, LTF, MAFG, MERTK, METTL7B, MMP8, MMP9, MPO, MRC1, MROCKI, MS4A3, MS4A4A, NECAB1, NEIL3, NEK2, NRXN2, NUF2, OLAH, OLFM4, OLIG2, PCOLCE2, PCSK9, PHF24, PIGR, PLAAT2, PPARG, PRTN3, PTGES, PYCRI, RAB3IL1, RASGRF1, RETN, RHCE, RIPOR3, RPGRIP1, RRM2, S100A12, S100A8, SCN8A, SEMA6B, SERPINB10, SIGLEC8, SIL1, SLC16A1, SLC28A3, SLC39A8, SLC4A10, SLC51A, SLC6A19, SLC8A3, SLCO4A1, SMIMI, SMPDL3A, SPATC1, SPOP, SSBP2, TCN1, TCTEX1D1, TDRD9, TEAD2, TFRC, THBS1, TIMP3, TLN2, TMEM255A, TMEM45A, TNFAIP8L3, TNIP3, TROAP, TTK, VSIG4, WNT3, YPEL4, and ZDHHC19.

In an embodiment of the method for predicting severity of sepsis in a subject, the plurality of genes comprises, consists essentially of or consists of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156 or 157 genes selected from the group consisting of ABCA13, ADAMTS2, ADAMTS3, AK5, ANKRD22, ANKRD34B, ANLN, AQP1, ARG1, ARHGAP44, ARHGEF17, ASPM, ATP1B2, AURKA, AZU1, BAIAP3, BPI, Clorf226, CACNB4, CCL4L2, CCN3, CCNA1, CD177, CD24, CDK1, CDKN3, CEACAM6, CEACAM8, CENPA, CFH, CHDH, CHITI, CKAP2L, CLEC4C, CLEC4F, CLNK, COL17A1, CRISP2, CRISP3, CTSE, CTSG, CYP19A1, CYYR1, DEFA4, DENND2C, DEPDC1, DGKK, DLC1, DLGAP5, DNAH10, DOC2B, DSP, ELANE, ERG, FAM20A, FAM83A, FBN1, FFAR3, GOS2, GGT5, GLB1L2, GJB6, GPR84, GRAMD1C, GYPA, HBM, HMGB3, HP, HPGD, HRK, IGLL1, IL1R2, IL1RL1, INHBA, IQGAP3, ITGA7, ITGB4, KIF15, KIF20A, KLF14, LAMB3, LCN2, LGR4, LPL, LTF, MAFG, MERTK, METTL7B, MMP8, MMP9, MPO, MRC1, MROCKI, MS4A3, MS4A4A, NECAB1, NEIL3, NEK2, NRXN2, NUF2, OLAH, OLFM4, OLIG2, PCOLCE2, PCSK9, PHF24, PIGR, PLAAT2, PPARG, PRTN3, PTGES, PYCRI, RAB3IL1, RASGRF1, RETN, RHCE, RIPOR3, RPGRIP1, RRM2, S100A12, S100A8, SCN8A, SEMA6B, SERPINB10, SIGLEC8, SILl, SLC16A1, SLC28A3, SLC39A8, SLC4A10, SLC51A, SLC6A19, SLC8A3, SLCO4A1, SMIMI, SMPDL3A, SPATC1, SPOP, SSBP2, TCN1, TCTEX1D1, TDRD9, TEAD2, TFRC, THBS1, TIMP3, TLN2, TMEM255A, TMEM45A, TNFAIP8L3, TNIP3, TROAP, TTK, VSIG4, WNT3, YPEL4, and ZDHHC19.

In an embodiment of the method for predicting severity of sepsis in a subject, the plurality of genes comprises, consists essentially of or consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, or 73 genes selected from the group consisting of AK5, ANKRD22, ARHGEF17, ASPM, ATP1B2, AURKA, BAIAP3, Clorf226, CACNB4, CCL4L2, CCN3, CD177, CD24, CENPA, CFH, CHDH, CHITI, CKAP2L, CLEC4C, DENND2C, DLGAP5, DNAH10, DSP, FAM20A, FBN1, GOS2, GGT5, GLB1L2, GPR84, GRAMD1C, HBM, HMGB3, HP, HRK, IQGAP3, ITGB4, KIF15, LAMB3, LCN2, LPL, LTF, MAFG, MERTK, MMP8, MMP9, MRC1, MS4A4A, NRXN2, NUF2, PHF24, PTGES, PYCRI, RAB3IL1, RETN, RPGRIP1, RRM2, SCN8A, SERPINB10, SIL1, SLC16A1, SLC39A8, SLC4A10, SLC6A19, SLC8A3, SMIM1, SPATC1, SPOP, SSBP2, TCTEX1D1, TEAD2, TLN2, TMEM255A, and TMEM45A. In another embodiment, the plurality of genes comprises, consists essentially of or consists of CCL4L2, GPR84, HRK, MMP8, GGT5, RASGRF1. In another embodiment, the plurality of genes comprises CCL4L2, GPR84, HRK, MMP8, GGT5, and RASGRF1. In a further embodiment, the plurality of genes is CCL4L2, GPR84, HRK, MMP8, GGT5, and RASGRF1.

The biological sample can comprise any suitable biological sample, the selection of which can be readily made by a person skilled in the art. In an embodiment, the biological sample comprises sputum, blood, nasal brushings, throat swabs, urine, amniotic fluid, plasma, serum, saliva, semen, bone marrow, tissue or fine needle biopsy samples, stool, bronchoalveolar lavage fluid, cerebrospinal fluid, peritoneal fluid, pleural fluid, skin, or cells therefrom. In another embodiment, the biological sample comprises blood.

Determining the level of expression for the plurality of genes can comprise any suitable method, the selection of which can be made by a person skilled in the art. For example, the expression of the genes may be determined by detection of an expression product of each gene. The expression product may be, for example, RNA, cDNA prepared from RNA and/or protein. In an embodiment, the expression product is cDNA prepared from RNA. When the expression product is RNA or cDNA, the entire sequence of the gene may be detected, or any definitive portion of the gene, for example, a sequence of 10 nucleotides or more, may be detected. Methods of detecting and quantifying expression of genes are well-known in the art and include the use of detectably labelled polynucleotide probes, antibodies, aptamers, and the like. In an embodiment, detecting the level of expression comprises detecting nucleic acids encoded by each of the plurality of genes. In an embodiment, determining the level of expression comprises one or more of a polymerase chain reaction (PCR) amplification method, a non-PCR based amplification method, reverse transcriptase-(RT) PCR, Q-beta replicase amplification, ligase chain reaction, signal amplification (Ampliprobe), light cycling, differential display, Northern analysis, hybridization, microarray analysis, DNA sequencing, RNA-Seq, MassArray analysis and MALDI-TOF mass spectrometry. In another embodiment, determining the level of expression comprises a polymerase chain reaction (PCR) amplification method or reverse transcriptase-(RT) PCR. In another embodiment, determining the level of expression comprises a polymerase chain reaction (PCR) amplification method. In another embodiment, determining the level of expression comprises reverse transcriptase-(RT) PCR. In another embodiment, determining the level of expression comprises RNA sequencing (RNA-Seq). In an embodiment, prior to RNA-Seq, the method comprises extracting total RNA from the biological sample by any suitable method followed by preparation of cDNA libraries via any suitable method. The selection of suitable methods and means for extracting total RNA and preparation of cDNA libraries can be readily selected by a person skilled in the art.

In certain embodiments, determining the level of expression comprises the use of detectably labelled polynucleotides. The methods may further comprise one or more of isolation of nucleic acids from the biological sample, purification of the nucleic acids, reverse transcription of RNA, and/or nucleic acid amplification. In some embodiments, the polynucleotide probes used to determine the level of expression may be immobilized on a solid support, for example, as an array or microarray allowing for more rapid processing of the sample. Methods of preparing arrays and microarrays are well known in the art. In addition, a number of standard microarrays are available commercially that include probes for detecting some of the genes identified herein and thus may be suitable for use in these methods. For example, Affymetrix U133 GeneChip™ arrays (Affymetrix, Inc., Santa Clara, CA), Agilent Technologies genomic cDNA microarrays (Santa Clara, CA), and arrays available from Illumina, Inc. (San Diego, CA). These arrays have probe sets for the whole human genome immobilized on a chip, and can be used to determine up- and down-regulation of genes in test samples. Custom-made arrays and microarrays for detecting pre-selected genes are also available commercially from a number of companies. Instruments and reagents for performing gene expression analysis are commercially available (for example, the Affymetrix GeneChip™ System). In some embodiments, the expression data obtained from the analysis may then be input into an appropriate database for further analysis if necessary or desired. In some embodiments, the determining the level of expression comprises, after conversion to cDNAs, the use of Matrix-assisted laser desorption/ionization—time of flight (MALDI-TOF) mass spectrometry using, for example the Sequenom MassARRAY® system (see, for example, Kricka L J. Clin Chem 1999; 45:453-458).

The expression of certain genes known as “housekeeping genes”, “reference genes”, or “control genes” may also be determined in the biological sample as a means of ensuring the veracity of the expression profile. Such genes are genes that are consistently expressed in many tissue types, including cancerous and normal tissues, and thus are useful to normalize gene expression profiles. Determining the expression of housekeeping genes, reference genes, or control genes in parallel with the plurality of genes, may, for example, provide further assurance that the techniques used for determination of the gene expression profile are working properly. Appropriate housekeeping genes (also referred to herein as reference genes and control genes) can be readily selected by the skilled person.

The levels of expression determined are compared to a suitable reference gene signature, which may, for example, be corresponding levels of expression in a biological sample from a healthy individual, e.g., in embodiments wherein the reference gene signature represents a standard level of expression of the genes. The comparison may include, for example, a visual inspection and/or an arithmetic or statistical comparison of measurements and may take into account expression of any reference genes. Suitable methods of comparison to determine differences in expression levels of genes are well known in the art. In an embodiment, the comparison comprises use of a trained model/classification tool.

In an embodiment, the biological sample has been obtained from the subject prior to admission in an intensive care unit (ICU). In an embodiment, the biological sample has been obtained from the subject at first clinical presentation. In a further embodiment, the biological sample has been obtained from the subject within about 2 hours of admission into an emergency room. In another embodiment, the sample has been obtained from the subject within the first day after entry into an intensive care unit (ICU).

In the examples of the present disclosure, the clear associations between endotypes and clinical symptomology and outcomes indicated that the sepsis mechanistic endotypes represent, for example, a useful tool to prognosticate patients, while their underlying mechanistic differences indicate the potential for personalized therapy. Identification of individual mechanisms in sepsis, for example has the additional benefit that it can be used to guide physicians in treating patients based on the individual features of their type of sepsis.

Accordingly, the present disclosure also includes a method of treating sepsis, the method comprising: (a) classifying a subject into a sepsis mechanistic endotype selected from neutrophilic-suppressive (NPS), inflammatory (INF), innate host defense (IHD), interferon (IFN) and adaptive (ADA) endotypes by a method for classifying a subject into a sepsis mechanistic endotype of the present disclosure; and (b) administering to the subject, an effective amount of one or more therapies that act specifically against a mechanism associated with the sepsis mechanistic endotype. The present disclosure also includes a use of one or more therapies that act specifically against a mechanism associated with a sepsis mechanistic endotype selected from neutrophilic-suppressive (NPS), inflammatory (INF), innate host defense (IHD), interferon (IFN) and adaptive (ADA) endotypes, for treatment of sepsis in a subject classified as having the sepsis mechanistic endotype by a method for classifying a subject into a sepsis mechanistic endotype of the present disclosure. The present disclosure also includes a use of one or more therapies that act specifically against a mechanism associated with a sepsis mechanistic endotype selected from neutrophilic-suppressive (NPS), inflammatory (INF), innate host defense (IHD), interferon (IFN) and adaptive (ADA) endotypes, for preparation of a medicament for treatment of sepsis in a subject classified as having the sepsis mechanistic endotype by a method for classifying a subject into a sepsis mechanistic endotype of the present disclosure. The present disclosure further includes one or more therapies that act specifically against a mechanism associated with a sepsis mechanistic endotype selected from neutrophilic-suppressive (NPS), inflammatory (INF), innate host defense (IHD), interferon (IFN) and adaptive (ADA) endotypes for use to treat sepsis in a subject classified as having the sepsis mechanistic endotype by a method for classifying a subject into a sepsis mechanistic endotype of the present disclosure. The method for classifying a subject into a sepsis mechanistic endotype of the present disclosure can comprise: (a) determining, in a biological sample from the subject, a level of expression for each of a plurality of genes, to provide a sample gene signature; and (b) comparing the sample gene signature with a reference gene signature to determine whether the subject has the sepsis mechanistic endotype, wherein the sample gene signature and reference gene signature comprise an NPS endotype sub-signature, an INF endotype sub-signature, an IHD endotype sub-signature, an IFN endotype sub-signature, an ADA endotype sub-signature or combinations thereof, wherein the NPS endotype sub-signature comprises genes selected from the group consisting of: AGFG1, ARG1, ATP9A, ANXA3, EFNA1, GADD45A, GPR84, HPGD, IL1R1, KLF14, KREMEN1, MIR646HG, MLLT1, NSUN7, OLAH, ORM2, PCOLCE2, PFKFB2, SLC51A, TNFAIP8L3, ZDHHC19, ADAMTS3, AKR1C1, ALDH1A2, ALOX5AP, ALPL, AMPH, ANKRD55, BCL3, BTBD19, CA4, CD163L1, CD177, CD82, CST7, CYP19A1, CYSTM1, DAAM2, DGAT2, ECHDC3, ENTPD7, EXOSC4, FFAR3, FGF13, FSTL4, GALNT14, GRAMD1A, GRB10, GYG1, HPGD, IER3, IL18RAP, IL1R2, IL1RN, IRAG1-AS1, KCNE1B, KCNMA1, MCEMP1, MKNK1, MMP9, MSRA, NECAB1, NSMCE1-DT, OPLAH, PDGFC, PFKFB3, PHF24, PI3, PLIN4, PLIN5, PLK3, POR, PROK2, RFX2, RGL4, ROM1, S100A12, S100P, SEMA6B, SHROOM4, SLP1, SOCS3, SPATC1, SPDYA, SPINK8, SPP1, ST6GALNAC3, SYN2, TDRD9, TMEM120A, TMIGD3, TSPO, UPP1, and XCR1; wherein the INF endotype sub-signature comprises genes selected from the group consisting of: BNIP3L, CA1, FAM83A, FECH, GLRX5, GYPA, IFIT1B, RHCE, RIOK3, RNF182, SLC6A19, SPTA1, THEM5, TLCD4, TSPAN5, TSPO2, ABCG2, ACHE, ACKR1, ACSL6, ADD2, AHSP, ALAS2, ALDH5A1, ANK1, ANKRD9, AQP1, ARHGEF12, ARHGEF37, ARL4A, ATP1B2, ATP1B2, BBOF1, BCAM, BCL2L1, BLVRB, BPGM, Clorf116, CA2, CISD2, CLIC2, CR1L, CR1L, CTNNAL1, CTSE, DCAF12, DMTN, DNAJC6, DPCD, DYRK3, EMID1, EPB42, ERFE, FAM210B, FAXDC2, FRMD4A, GMPR, GSPT1, GYPB, HBM, HEMGN, HEPACAM2, HMBS, IGF2BP2, ISCA1, ITLN1, KANK2, KCNH2, KDM7A-DT, KEL, KLC3, KLF1, KLHDC8A, KRT1, LRRC2, MAOA, MARCHF8, MBNL3, MFSD2B, MRC2, MXI1, MYL4, NFIX, NUDT4, OSBP2, PAGE2B, PBX1, PCDH1, PGF, PLEK2, PNP, PRDX2, PTPRF, RAP1GAP, RBM38, RFESD, RFESD, RGCC, RGS16, RHAG, RHD, RIPOR3, RNF175, RUNDC3A, SEC14L4, SELENBP1, SELENOP, SFRP2, SGIP1, SIAH2, SLC14A1, SLC1A5, SLC22A23, SLC2A1, SLC4A1, SLC6A8, SLC6A9, SLC7A5, SMIM5, SNCA, SOX6, SPTB, STRADB, TAL1, TENT5C, TFR2, TMCC2, TMOD1, TNS1, TRIM10, TRIM58, TSPAN7, TTC25, UBB, USP12, XK, YBX3, and YPEL4; wherein the IHD endotype sub-signature comprises genes selected from the group consisting of: ABCA6, ADAM23, ALOX15, CACNA2D3, DYNC2H1, GPR34, GRAMD1C, LPL, MAP7, MIR155HG, PLCB1, SDC2, SIGLEC8, SPRED1, SLC16A14, SMPD3, TPPP3, TPRG1, ZNF600, ADGRD1, ANGPT1, GPR82, HDAC9, IL5RA, KLHDC1, PRSS33, PTGDR2, PTGFRN, TBC1D12, and TRIM2; wherein the IFN endotype sub-signature comprises genes selected from the group consisting of: ANKRD22, APOL1, APOL4, BATF2, CARD17, CD274, EPSTI1, ETV7, GBP5, IDO1, IFITM3, P2RYl4, PLEKHO1, RSAD2, SERPING1, TFEC, EXOC3L1, IRF7, OAS1, SEPTIN4, LY6E, and LAMP3; and wherein the ADA endotype sub-signature comprises genes selected from the group consisting of: CCL2, CDC45, CENPF, CLEC4F, GTSE1, IFI27, ISG15, KCTD14, KIF14, KIF15, KLHDC7B, LGALS3BP, OTOF, PDIA4, SIGLEC1, USP18, AGRN, CD38, CDCA7, CDT1, CTLA4, DHX58, EME1, FAM111B, HES4, IFI44L, IFIT3, IFNG-AS1, IL12RB2, IL4I1, KIF19, LAG3, MCM10, P2RY6, PACSIN1, PARM1, SAMD4A, SPATS2L, HERC5, TMPRSS3, TNFRSF13B, TSHR, and TTC21A and/or comprise: (a) determining, in a biological sample from the subject, a level of expression for each of a plurality of genes, to provide a sample gene signature; and (b) comparing the sample gene signature with a reference gene signature to determine whether the subject has the sepsis mechanistic endotype, wherein the sample gene signature and reference gene signature comprise an NPS endotype signature pair, an INF endotype signature pair, an IHD endotype signature pair, an IFN endotype signature pair, an ADA endotype signature pair or combinations thereof, wherein the NPS endotype signature pair is selected from: GADD45A/EFNA1, EFNA1/MIR646HG, MIR646HG/KLF14, MLLT1/MIR646HG, ARG1/MLLT1, MLLT1/EFNA1, MLLT1/NSUN7, EFNA1/NSUN7, SLC51A/EFNA1, EFNA1/KLF14, ZDHHC19/EFNA1, EFNA1/AGFG1, NSUN7/KLF14, EFNA1/PFKFB2, MLLT1/KLF14, ADAMTS3/PCOLCE2, ADAMTS3/ZDHHC19, ADAMTS3/SLC51A, ADAMTS3/HPGD, ADAMTS3/SEMA6B, ADAMTS3/EFNA1, ADAMTS3/AGFG1, ADAMTS3/NSUN7, ADAMTS3/TNFAIP8L3, ADAMTS3/KREMEN1, ADAMTS3/ORM2, ADAMTS3/MIR646HG, ADAMTS3/KLF14, AGFG1/NSUN7, AGFG1/TNFAIP8L3, AGFG1/KREMEN1, AGFG1/ORM2, AGFG1/MIR646HG, AGFG1/KLF14, ANXA3/GPR84, ANXA3/OLAH, ANXA3/ADAMTS3, ANXA3/PCOLCE2, ANXA3/ZDHHC19, ANXA3/SLC51A, ANXA3/HPGD, ANXA3/SEMA6B, ANXA3/EFNA1, ANXA3/AGFG1, ANXA3/NSUN7, ANXA3/TNFAIP8L3, ANXA3/KREMEN1, ANXA3/ORM2, ANXA3/MIR646HG, ANXA3/KLF14, ARG1/PFKFB2, ARG1/MLLT1, ARG1/ANXA3, ARG1/GPR84, ARG1/OLAH, ARG1/ADAMTS3, ARG1/PCOLCE2, ARG1/ZDHHC19, ARG1/SLC51A, ARG1/HPGD, ARG1/SEMA6B, ARG1/EFNA1, ARG1/AGFG1, ARG1/NSUN7, ARG1/TNFAIP8L3, ARG1/KREMEN1, ARG1/ORM2, ARG1/MIR646HG, ARG1/KLF14, ATP9A/EPB41L4B, ATP9A/IL1R1, ATP9A/GADD45A, ATP9A/ARG1, ATP9A/PFKFB2, ATP9A/MLLT1, ATP9A/ANXA3, ATP9A/GPR84, ATP9A/OLAH, ATP9A/ADAMTS3, ATP9A/PCOLCE2, ATP9A/ZDHHC19, ATP9A/SLC51A, ATP9A/HPGD, ATP9A/SEMA6B, ATP9A/EFNA1, ATP9A/AGFG1, ATP9A/NSUN7, ATP9A/TNFAIP8L3, ATP9A/KREMEN1, ATP9A/ORM2, ATP9A/MIR646HG, ATP9A/KLF14, EFNA1/AGFG1, EFNA1/NSUN7, EFNA1/TNFAIP8L3, EFNA1/KREMEN1, EFNA1/ORM2, EFNA1/MIR646HG, EFNA1/KLF14, EPB41L4B/IL1R1, EPB41L4B/GADD45A, EPB41L4B/ARG1, EPB41L4B/PFKFB2, EPB41L4B/MLLT1, EPB41L4B/ANXA3, EPB41L4B/GPR84, EPB41L4B/OLAH, EPB41L4B/ADAMTS3, EPB41L4B/PCOLCE2, EPB41L4B/ZDHHC19, EPB41L4B/SLC51A, EPB41L4B/HPGD, EPB41L4B/SEMA6B, EPB41L4B/EFNA1, EPB41L4B/AGFG1, EPB41L4B/NSUN7, EPB41L4B/TNFAIP8L3, EPB41L4B/KREMEN1, EPB41L4B/MIR646HG, EPB41L4B/KLF14, GADD45A/ARG1, GADD45A/PFKFB2, GADD45A/MLLT1, GADD45A/ANXA3, GADD45A/GPR84, GADD45A/OLAH, GADD45A/ADAMTS3, GADD45A/PCOLCE2, GADD45A/ZDHHC19, GADD45A/SLC51A, GADD45A/HPGD, GADD45A/SEMA6B, GADD45A/EFNA1, GADD45A/AGFG1, GADD45A/NSUN7, GADD45A/TNFAIP8L3, GADD45A/KREMEN1, GADD45A/ORM2, GADD45A/MIR646HG, GADD45A/KLF14, GPR84/OLAH, GPR84/ADAMTS3, GPR84/PCOLCE2, GPR84/ZDHHC19, GPR84/SLC51A, GPR84/HPGD, GPR84/SEMA6B, GPR84/EFNA1, GPR84/AGFG1, GPR84/NSUN7, GPR84/TNFAIP8L3, GPR84/KREMEN1, GPR84/ORM2, GPR84/MIR646HG, GPR84/KLF14, HPGD/SEMA6B, HPGD/EFNA1, HPGD/AGFG1, HPGD/NSUN7, HPGD/TNFAIP8L3, HPGD/KREMEN1, HPGD/ORM2, HPGD/MIR646HG, HPGD/KLF14, IL1R1/GADD45A, IL1R1/ARG1, IL1R1/PFKFB2, IL1R1/MLLT1, IL1R1/ANXA3, IL1R1/GPR84, IL1R1/OLAH, IL1R1/ADAMTS3, IL1R1/PCOLCE2, IL1R1/ZDHHC19, IL1R1/SLC51A, IL1R1/HPGD, IL1R1/SEMA6B, IL1R1/EFNA1, IL1R1/AGFG1, IL1R1/NSUN7, IL1R1/TNFAIP8L3, IL1R1/KREMEN1, IL1R1/ORM2, IL1R1/MIR646HG, IL1R1/KLF14, KREMEN1/ORM2, KREMEN1/MIR646HG, KREMEN1/KLF14, MIR646HG/KLF14, MLLT1/ANXA3, MLLT1/GPR84, MLLT1/OLAH, MLLT1/ADAMTS3, MLLT1/PCOLCE2, MLLT1/ZDHHC19, MLLT1/SLC51A, MLLT1/HPGD, MLLT1/SEMA6B, MLLT1/EFNA1, MLLT1/AGFG1, MLLT1/NSUN7, MLLT1/TNFAIP8L3, MLLT1/KREMEN1, MLLT1/ORM2, MLLT1/MIR646HG, MLLT1/KLF14, NSUN7/TNFAIP8L3, NSUN7/KREMEN1, NSUN7/ORM2, NSUN7/MIR646HG, NSUN7/KLF14, OLAH/ADAMTS3, OLAH/PCOLCE2, OLAH/ZDHHC19, OLAH/SLC51A, OLAH/HPGD, OLAH/SEMA6B, OLAH/EFNA1, OLAH/AGFG1, OLAH/NSUN7, OLAH/TNFAIP8L3, OLAH/KREMEN1, OLAH/ORM2, OLAH/MIR646HG, OLAH/KLF14, ORM2/MIR646HG, ORM2/KLF14, PCOLCE2/ZDHHC19, PCOLCE2/SLC51A, PCOLCE2/HPGD, PCOLCE2/SEMA6B, PCOLCE2/EFNA1, PCOLCE2/AGFG1, PCOLCE2/NSUN7, PCOLCE2/TNFAIP8L3, PCOLCE2/KREMEN1, PCOLCE2/ORM2, PCOLCE2/MIR646HG, PCOLCE2/KLF14, PFKFB2/MLLT1, PFKFB2/ANXA3, PFKFB2/GPR84, PFKFB2/OLAH, PFKFB2/ADAMTS3, PFKFB2/PCOLCE2, PFKFB2/ZDHHC19, PFKFB2/SLC51A, PFKFB2/HPGD, PFKFB2/SEMA6B, PFKFB2/EFNA1, PFKFB2/AGFG1, PFKFB2/NSUN7, PFKFB2/TNFAIP8L3, PFKFB2/KREMEN1, PFKFB2/ORM2, PFKFB2/MIR646HG, PFKFB2/KLF14, SEMA6B/EFNA1, SEMA6B/AGFG1, SEMA6B/NSUN7, SEMA6B/TNFAIP8L3, SEMA6B/KREMEN1, SEMA6B/ORM2, SEMA6B/MIR646HG, SEMA6B/KLF14, SLC51A/HPGD, SLC51A/SEMA6B, SLC51A/EFNA1, SLC51A/AGFG1, SLC51A/NSUN7, SLC51A/TNFAIP8L3, SLC51A/KREMEN1, SLC51A/ORM2, SLC51A/MIR646HG, SLC51A/KLF14, TNFAIP8L3/KREMEN1, TNFAIP8L3/ORM2, TNFAIP8L3/MIR646HG, TNFAIP8L3/KLF14, ZDHHC19/SLC51A, ZDHHC19/HPGD, ZDHHC19/SEMA6B, ZDHHC19/EFNA1, ZDHHC19/AGFG1, ZDHHC19/NSUN7, ZDHHC19/TNFAIP8L3, ZDHHC19/KREMEN1, ZDHHC19/ORM2, ZDHHC19/MIR646HG, and ZDHHC19/KLF14; wherein the INF endotype signature pair is selected from: FECH/TFEC, TFEC/IFIT1B, FECH/RNF182, IFIT1B/FECH, FECH/APOL4, FECH/GYPA, ITLN1/FECH, FECH/THEM5, IFIT1B/CA1, RHAG/FECH, FECH/FAM83A, RHCE/FECH, TFEC/CA1, SPTA1/FECH, ANKRD22/GLRX5, ANKRD22/GYPA, ANKRD22/IFIT1B, ANKRD22/ITLN1, ANKRD22/KLHDC8A, ANKRD22/RHCE, ANKRD22/RNF182, ANKRD22/SPTA1, ANKRD22/THEM5, ANKRD22/TSPAN5, APOL4/BNIP3L, APOL4/CA1, APOL4/DYRK3, APOL4/FAM83A, APOL4/GLRX5, APOL4/GYPA, APOL4/IFIT1B, APOL4/ITLN1, APOL4/KLHDC8A, APOL4/RHAG, APOL4/RHCE, APOL4/RIOK3, APOL4/RNF182, APOL4/SPTA1, APOL4/THEM5, APOL4/TLCD4, APOL4/TMCC2, APOL4/TSPAN5, APOL4/TSPO2, BNIP3L/ANKRD22, BNIP3L/CA1, BNIP3L/CARD17, BNIP3L/CD274, BNIP3L/DYRK3, BNIP3L/FAM83A, BNIP3L/GBP5, BNIP3L/GLRX5, BNIP3L/GYPA, BNIP3L/IFIT1B, BNIP3L/ITLN1, BNIP3L/KLHDC8A, BNIP3L/P2RY14, BNIP3L/RHAG, BNIP3L/RHCE, BNIP3L/RNF182, BNIP3L/SPTA1, BNIP3L/TFEC, BNIP3L/THEM5, BNIP3L/TLCD4, BNIP3L/TMCC2, BNIP3L/TSPAN5, BNIP3L/TSPO2, CA1/ANKRD22, CA1/CARD17, CA1/DYRK3, CA1/FAM83A, CA1/GBP5, CA1/GLRX5, CA1/GYPA, CA1/IFIT1B, CA1/ITLN1, CA1/KLHDC8A, CA1/P2RY14, CA1/RHCE, CA1/RNF182, CA1/SPTA1, CA1/THEM5, CA1/TLCD4, CA1/TSPAN5, CD274/CA1, CD274/DYRK3, CD274/FAM83A, CD274/GLRX5, CD274/GYPA, CD274/IFIT1B, CD274/ITLN1, CD274/KLHDC8A, CD274/RHCE, CD274/RNF182, CD274/SPTA1, CD274/THEM5, CD274/TLCD4, CD274/TMCC2, CD274/TSPAN5, DYRK3/ANKRD22, DYRK3/CARD17, DYRK3/FAM83A, DYRK3/GBP5, DYRK3/GLRX5, DYRK3/GYPA, DYRK3/IFIT1B, DYRK3/ITLN1, DYRK3/KLHDC8A, DYRK3/P2RY14, DYRK3/RHCE, DYRK3/RNF182, DYRK3/SPTA1, DYRK3/THEM5, DYRK3/TLCD4, DYRK3/TSPAN5, FAM83A/ANKRD22, FAM83A/CARD17, FAM83A/GBP5, FAM83A/GLRX5, FAM83A/GYPA, FAM83A/IFIT1B, FAM83A/ITLN1, FAM83A/KLHDC8A, FAM83A/P2RY14, FAM83A/RHCE, FAM83A/RNF182, FAM83A/SPTA1, FAM83A/THEM5, FAM83A/TLCD4, FAM83A/TSPAN5, FECH/ANKRD22, FECH/APOL4, FECH/BNIP3L, FECH/CA1, FECH/CARD17, FECH/CD274, FECH/DYRK3, FECH/FAM83A, FECH/GBP5, FECH/GLRX5, FECH/GYPA, FECH/IFIT1B, FECH/ITLN1, FECH/KLHDC8A, FECH/P2RY14, FECH/RHAG, FECH/RHCE, FECH/RIOK3, FECH/RNF182, FECH/SPTA1, FECH/TFEC, FECH/THEM5, FECH/TLCD4, FECH/TMCC2, FECH/TSPAN5, FECH/TSPO2, GBP5/GLRX5, GBP5/GYPA, GBP5/IFIT1B, GBP5/ITLN1, GBP5/KLHDC8A, GBP5/RHCE, GBP5/RNF182, GBP5/SPTA1, GBP5/THEM5, GBP5/TSPAN5, GLRX5/CARD17, GLRX5/IFIT1B, GLRX5/RHCE, GLRX5/THEM5, GYPA/CARD17, GYPA/GLRX5, GYPA/IFIT1B, GYPA/ITLN1, GYPA/P2RY14, GYPA/RHCE, GYPA/RNF182, GYPA/THEM5, IFIT1B/CARD17, ITLN1/CARD17, ITLN1/GLRX5, ITLN1/IFIT1B, ITLN1/RHCE, ITLN1/RNF182, ITLN1/THEM5, KLHDC8A/CARD17, KLHDC8A/GLRX5, KLHDC8A/GYPA, KLHDC8A/IFIT1B, KLHDC8A/ITLN1, KLHDC8A/P2RY14, KLHDC8A/RHCE, KLHDC8A/RNF182, KLHDC8A/SPTA1, KLHDC8A/THEM5, KLHDC8A/TSPAN5, P2RY14/GLRX5, P2RY14/IFIT1B, P2RY14/ITLN1, P2RY14/RHCE, P2RY14/RNF182, P2RY14/THEM5, RHAG/ANKRD22, RHAG/CA1, RHAG/CARD17, RHAG/CD274, RHAG/DYRK3, RHAG/FAM83A, RHAG/GBP5, RHAG/GLRX5, RHAG/GYPA, RHAG/IFIT1B, RHAG/ITLN1, RHAG/KLHDC8A, RHAG/P2RY14, RHAG/RHCE, RHAG/RNF182, RHAG/SPTA1, RHAG/THEM5, RHAG/TLCD4, RHAG/TMCC2, RHAG/TSPAN5, RHAG/TSPO2, RHCE/CARD17, RHCE/IFIT1B, RHCE/THEM5, RIOK3/ANKRD22, RIOK3/BNIP3L, RIOK3/CA1, RIOK3/CARD17, RIOK3/CD274, RIOK3/DYRK3, RIOK3/FAM83A, RIOK3/GBP5, RIOK3/GLRX5, RIOK3/GYPA, RIOK3/IFIT1B, RIOK3/ITLN1, RIOK3/KLHDC8A, RIOK3/P2RY14, RIOK3/RHAG, RIOK3/RHCE, RIOK3/RNF182, RIOK3/SPTA1, RIOK3/TFEC, RIOK3/THEM5, RIOK3/TLCD4, RIOK3/TMCC2, RIOK3/TSPAN5, RIOK3/TSPO2, RNF182/CARD17, RNF182/GLRX5, RNF182/IFIT1B, RNF182/RHCE, RNF182/THEM5, SPTA1/CARD17, SPTA1/GLRX5, SPTA1/GYPA, SPTA1/IFIT1B, SPTA1/ITLN1, SPTA1/P2RY14, SPTA1/RHCE, SPTA1/RNF182, SPTA1/THEM5, SPTA1/TSPAN5, TFEC/CA1, TFEC/DYRK3, TFEC/FAM83A, TFEC/GLRX5, TFEC/GYPA, TFEC/IFIT1B, TFEC/ITLN1, TFEC/KLHDC8A, TFEC/RHAG, TFEC/RHCE, TFEC/RNF182, TFEC/SPTA1, TFEC/THEM5, TFEC/TLCD4, TFEC/TMCC2, TFEC/TSPAN5, TFEC/TSPO2, THEM5/CARD17, THEM5/IFIT1B, TLCD4/ANKRD22, TLCD4/CARD17, TLCD4/GBP5, TLCD4/GLRX5, TLCD4/GYPA, TLCD4/IFIT1B, TLCD4/ITLN1, TLCD4/KLHDC8A, TLCD4/P2RY14, TLCD4/RHCE, TLCD4/RNF182, TLCD4/SPTA1, TLCD4/THEM5, TLCD4/TSPAN5, TMCC2/ANKRD22, TMCC2/CA1, TMCC2/CARD17, TMCC2/DYRK3, TMCC2/FAM83A, TMCC2/GBP5, TMCC2/GLRX5, TMCC2/GYPA, TMCC2/IFIT1B, TMCC2/ITLN1, TMCC2/KLHDC8A, TMCC2/P2RY14, TMCC2/RHCE, TMCC2/RNF182, TMCC2/SPTA1, TMCC2/THEM5, TMCC2/TLCD4, TMCC2/TSPAN5, TSPAN5/CARD17, TSPAN5/GLRX5, TSPAN5/GYPA, TSPAN5/IFIT1B, TSPAN5/ITLN1, TSPAN5/P2RY14, TSPAN5/RHCE, TSPAN5/RNF182, TSPAN5/THEM5, TSPO2/ANKRD22, TSPO2/CA1, TSPO2/CARD17, TSPO2/CD274, TSPO2/DYRK3, TSPO2/FAM83A, TSPO2/GBP5, TSPO2/GLRX5, TSPO2/GYPA, TSPO2/IFIT1B, TSPO2/ITLN1, TSPO2/KLHDC8A, TSPO2/P2RY14, TSPO2/RHCE, TSPO2/RNF182, TSPO2/SPTA1, TSPO2/THEM5, TSPO2/TLCD4, TSPO2/TMCC2, and TSPO2/TSPAN5; wherein the IHD endotype signature pair is selected from: MAP7/SPRED1, SPRED1/GPR34, IL5RA/SPRED1, SPRED1/TPRG1, HRK/SPRED1, SPRED1/PLCB1, TRIM2/SPRED1, SIGLEC8/SPRED1, SMPD3/SPRED1, SPRED1/ZNF600, SPRED1/SDC2, MAP7/GPR34, PRSS33/SPRED1, SPRED1/DYNC2H1, CACNA2D3/SPRED1, ADAM23/GPR34, ADAM23/MAP7, ADAM23/PLCB1, ADAM23/SPRED1, ALOX15/GPR34, ALOX15/PLCB1, ALOX15/SPRED1, BAALC/GPR34, BAALC/PLCB1, BAALC/SPRED1, CACNA2D3/DYNC2H1, CACNA2D3/GPR34, CACNA2D3/PLCB1, CACNA2D3/SPRED1, CACNA2D3/ZNF600, GPR34/DYNC2H1, GPR34/GRAMD1C, GPR34/PLCB1, GPR34/TPRG1, GPR34/ZNF600, GPR82/DYNC2H1, GPR82/GPR34, GPR82/GRAMD1C, GPR82/PLCB1, GPR82/TPRG1, GPR82/ZNF600, GRAMD1C/DYNC2H1, GRAMD1C/PLCB1, GRAMD1C/ZNF600, HRK/DYNC2H1, HRK/GPR34, HRK/MAP7, HRK/PLCB1, HRK/SPRED1, HRK/ZNF600, IL5RA/DYNC2H1, IL5RA/GPR34, IL5RA/PLCB1, IL5RA/SPRED1, IL5RA/TRIM2, MAP7/BAALC, MAP7/CACNA2D3, MAP7/DYNC2H1, MAP7/GPR34, MAP7/GPR82, MAP7/GRAMD1C, MAP7/PLCB1, MAP7/SPRED1, MAP7/TPRG1, MAP7/ZNF600, PLCB1/DYNC2H1, PLCB1/TPRG1, PLCB1/ZNF600, PRSS33/GPR34, PRSS33/PLCB1, PRSS33/SPRED1, SDC2/DYNC2H1, SDC2/GPR34, SDC2/PLCB1, SDC2/ZNF600, SIGLEC8/DYNC2H1, SIGLEC8/GPR34, SIGLEC8/MAP7, SIGLEC8/PLCB1, SIGLEC8/SPRED1, SIGLEC8/TRIM2, SMPD3/DYNC2H1, SMPD3/GPR34, SMPD3/MAP7, SMPD3/PLCB1, SMPD3/SPRED1, SMPD3/TRIM2, SPRED1/DYNC2H1, SPRED1/GPR34, SPRED1/GPR82, SPRED1/GRAMD1C, SPRED1/PLCB1, SPRED1/SDC2, SPRED1/TPRG1, SPRED1/ZNF600, TRIM2/CACNA2D3, TRIM2/DYNC2H1, TRIM2/GPR34, TRIM2/GPR82, TRIM2/GRAMD1C, TRIM2/HRK, TRIM2/MAP7, TRIM2/PLCB1, TRIM2/SDC2, TRIM2/SPRED1, TRIM2/TPRG1, and TRIM2/ZNF600; wherein the IFN endotype signature pair is selected from: ETV7/PLEKHO1, IFITM3/ETV7, ETV7/APOL1, BATF2/ETV7, PLEKHO1/BATF2, ETV7/EPSTI1, EPSTI1/BATF2, IFITM3/BATF2, USP18/EPSTI1, ETV7/SEPTIN4, ETV7/LAMP3, SERPING1/BATF2, LAMP3/BATF2, LAMP3/SERPING1, APOL1/BATF2, APOL1/CLEC4F, APOL1/EPSTI1, APOL1/EXOC3L1, APOL1/HES4, APOL1/IFITM3, APOL1/LY6E, APOL1/RSAD2, APOL1/SEPTIN4, APOL1/SERPING1, APOL1/TPPP3, BATF2/EXOC3L1, BATF2/HES4, CLEC4F/BATF2, CLEC4F/EXOC3L1, EPSTI1/BATF2, EPSTI1/CLEC4F, EPSTI1/EXOC3L1, EPSTI1/HES4, EPSTI1/IFITM3, EPSTI1/LY6E, EPSTI1/RSAD2, EPSTI1/SERPING1, EPSTI1/TPPP3, ETV7/APOL1, ETV7/BATF2, ETV7/CLEC4F, ETV7/EPSTI1, ETV7/EXOC3L1, ETV7/HES4, ETV7/IFITM3, ETV7/LAMP3, ETV7/LY6E, ETV7/PLEKHO1, ETV7/RSAD2, ETV7/SEPTIN4, ETV7/SERPING1, ETV7/TPPP3, EXOC3L1/HES4, IFITM3/SERPING1, IFITM3/CLEC4F, IFITM3/TPPP3, IFITM3/LY6E, IFITM3/EXOC3L1, IFITM3/HES4, LAMP3/APOL1, LAMP3/BATF2, LAMP3/CLEC4F, LAMP3/EPSTI1, LAMP3/EXOC3L1, LAMP3/HES4, LAMP3/IFITM3, LAMP3/LY6E, LAMP3/RSAD2, LAMP3/SEPTIN4, LAMP3/SERPING1, LAMP3/TPPP3, LY6E/BATF2, LY6E/EXOC3L1, PLEKHO1/APOL1, PLEKHO1/BATF2, PLEKHO1/EPSTI1, PLEKHO1/EXOC3L1, PLEKHO1/IFITM3, PLEKHO1/LAMP3, PLEKHO1/RSAD2, PLEKHO1/SEPTIN4, PLEKHO1/SERPING1, RSAD2/BATF2, RSAD2/CLEC4F, RSAD2/EXOC3L1, RSAD2/HES4, RSAD2/IFITM3, RSAD2/LY6E, RSAD2/SERPING1, RSAD2/TPPP3, SEPTIN4/BATF2, SEPTIN4/CLEC4F, SEPTIN4/EPSTI1, SEPTIN4/EXOC3L1, SEPTIN4/HES4, SEPTIN4/IFITM3, SEPTIN4/LY6E, SEPTIN4/RSAD2, SEPTIN4/SERPING1, SEPTIN4/TPPP3, SERPING1/BATF2, SERPING1/CLEC4F, SERPING1/EXOC3L1, SERPING1/HES4, SERPING1/LY6E, SERPING1/TPPP3, TPPP3/BATF2, and TPPP3/EXOC3L1; and wherein the ADA endotype signature pair is selected from: LGALS3BP/OTOF, LGALS3BP/IFI27, LGALS3BP/KIF14, LGALS3BP/CENPF, GTSE1/LGALS3BP, LGALS3BP/KCTD14, LGALS3BP/PDIA4, LGALS3BP/TSHR, LGALS3BP/PLAAT2, OTOF/IFI27, IGF1/LGALS3BP, CDC45/LGALS3BP, LGALS3BP/KIF15, LGALS3BP/IGLL5, LGALS3BP/MIXL, CAV1/LGALS3BP, CAV1/OTOF, CDC45/LGALS3BP, CDC45/OTOF, CENPF/KCTD14, GPRC5D/OTOF, GTSE1/LGALS3BP, GTSE1/OTOF, IGF1/LGALS3BP, IGF1/OTOF, KCTD14/KLHL14, KCTD14/PDIA4, KCTD14/TSHR, KIF14/KCTD14, LGALS3BP/CENPF, LGALS3BP/GPRC5D, LGALS3BP/IFI27, LGALS3BP/IGLL5, LGALS3BP/KCTD14, LGALS3BP/KIF14, LGALS3BP/KIF15, LGALS3BP/KLHL14, LGALS3BP/MIR155HG, LGALS3BP/MIXL1, LGALS3BP/OTOF, LGALS3BP/PDIA4, LGALS3BP/PLAAT2, LGALS3BP/SDC1, LGALS3BP/SLC16A14, LGALS3BP/TSHR, OTOF/CENPF, OTOF/IFI27, OTOF/IGLL5, OTOF/KCTD14, OTOF/KIF14, OTOF/KIF15, OTOF/KLHL14, OTOF/MIR155HG, OTOF/MIXL1, OTOF/PDIA4, OTOF/PLAAT2, OTOF/SDC1, OTOF/SLC16A14, OTOF/TSHR, PLAAT2/KCTD14, TNFRSF17/LGALS3BP, and TNFRSF17/OTOF. It will be appreciated by a person skilled in the art that embodiments for such methods for classifying a subject into a sepsis mechanistic endotype in such methods of treatment and uses can be varied as described herein for the methods for classifying a subject into a sepsis mechanistic endotype.

The one or more therapies that act specifically against a mechanism associated with the sepsis mechanistic endotype are any suitable therapies that act specifically against a mechanism associated with the particular sepsis mechanistic endotype, the selection of which can be readily made by a person skilled in the art having regard to the present disclosure.

For example, a person skilled in the art would readily appreciate that stimulating part of the immune system that is lacking in patients with the NPS endotype may be useful to treat subjects classified as having the NPS endotype. In an embodiment, the treatment of subjects classified as having the NPS endotype comprises a treatment that reverses cellular reprogramming and/or boosts T-cell function. In another embodiment, the treatment that reverses cellular reprogramming is interferon gamma (IFN-γ).

A person skilled in the art would also readily appreciate that because the INF endotype is inflammatory, an anti-inflammatory therapy may be useful to treat subjects classified as having the INF endotype whereas this would not be indicated, for example, for subjects classified as having the NPS endotype, which demonstrates an early immunosuppressive character. In an embodiment, the treatment of subjects classified as having the INF endotype comprises treatment with an anti-inflammatory therapy. In another embodiment, the anti-inflammatory therapy is a glucocorticoid or a monoclonal antibody against TNF-α.

A person skilled in the art would also readily appreciate that because the IHD and ADA endotypes demonstrate neutropenia, treatment with one or more therapies useful for treating neutropenia, such as granulocyte macrophage colony-stimulating factor (GM-CSF) therapy may be useful in treating subjects classified as having the IHD and/or ADA endotypes. In an embodiment, the treatment of subjects classified as having the IHD or ADA endotypes comprises treatment with a therapy useful for treating neutropenia. In another embodiment, the therapy useful for treating neutropenia is granulocyte macrophage colony-stimulating factor (GM-CSF).

A person skilled in the art would also readily appreciate that because the INF and IHD endotypes demonstrate turn on of reactive oxygen species (ROS) production, anti-oxidant therapy may be useful in treating subjects classified as having the INF and/or IHD endotypes. In an embodiment, the treatment of subjects classified as having the INF or IHD endotypes comprises treatment with anti-oxidant therapy.

Given the repercussions of increased antibiotic resistance and health care costs, triaging sepsis patients prior to treatment with antibiotics may also be desirable.

Accordingly, the present disclosure also includes a a method of treating sepsis in a subject predicted as having high or intermediate severity sepsis, the method comprising: (a) predicting that the subject has high or intermediate severity sepsis by a method comprising: (i) determining, in a biological sample from the subject, a level of expression for each of a plurality of genes, to provide a sample gene signature; and (ii) comparing the sample gene signature with a reference gene signature to predict the severity of the sepsis in the subject, wherein high severity sepsis means a sequential organ failure assessment (SOFA) score of greater than or equal to 5, and intermediate severity sepsis means a SOFA score of greater than or equal to 2 but less than 5; and wherein the plurality of genes is selected from the group consisting of ABCA13, ADAMTS2, ADAMTS3, AK5, ANKRD22, ANKRD34B, ANLN, AQP1, ARG1, ARHGAP44, ARHGEF17, ASPM, ATP1B2, AURKA, AZU1, BAIAP3, BPI, Clorf226, CACNB4, CCL4L2, CCN3, CCNA1, CD177, CD24, CDK1, CDKN3, CEACAM6, CEACAM8, CENPA, CFH, CHDH, CHITI, CKAP2L, CLEC4C, CLEC4F, CLNK, COL17A1, CRISP2, CRISP3, CTSE, CTSG, CYP19A1, CYYR1, DEFA4, DENND2C, DEPDC1, DGKK, DLC1, DLGAP5, DNAH10, DOC2B, DSP, ELANE, ERG, FAM20A, FAM83A, FBN1, FFAR3, GOS2, GGT5, GLB1L2, GJB6, GPR84, GRAMD1C, GYPA, HBM, HMGB3, HP, HPGD, HRK, IGLL1, IL1R2, IL1RL1, INHBA, IQGAP3, ITGA7, ITGB4, KIF15, KIF20A, KLF14, LAMB3, LCN2, LGR4, LPL, LTF, MAFG, MERTK, METTL7B, MMP8, MMP9, MPO, MRC1, MROCKI, MS4A3, MS4A4A, NECAB1, NEIL3, NEK2, NRXN2, NUF2, OLAH, OLFM4, OLIG2, PCOLCE2, PCSK9, PHF24, PIGR, PLAAT2, PPARG, PRTN3, PTGES, PYCRI, RAB3IL1, RASGRF1, RETN, RHCE, RIPOR3, RPGRIP1, RRM2, S100A12, S100A8, SCN8A, SEMA6B, SERPINB10, SIGLEC8, SIL1, SLC16A1, SLC28A3, SLC39A8, SLC4A10, SLC51A, SLC6A19, SLC8A3, SLCO4A1, SMIM1, SMPDL3A, SPATC1, SPOP, SSBP2, TCN1, TCTEX1D1, TDRD9, TEAD2, TFRC, THBS1, TIMP3, TLN2, TMEM255A, TMEM45A, TNFAIP8L3, TNIP3, TROAP, TTK, VSIG4, WNT3, YPEL4, and ZDHHC19; and (b) administering an effective amount of one or more antibiotics to the subject. The present disclosure also includes a use of an effective amount of one or more antibiotics for treatment of sepsis in a subject predicted as having high or intermediate severity sepsis by a method comprising: (a) determining, in a biological sample from the subject, a level of expression for each of a plurality of genes, to provide a sample gene signature; and (b) comparing the sample gene signature with a reference gene signature to predict the severity of the sepsis in the subject, wherein high severity sepsis means a sequential organ failure assessment (SOFA) score of greater than or equal to 5, and intermediate severity sepsis means a SOFA score of greater than or equal to 2 but less than 5; and wherein the plurality of genes is selected from the group consisting of ABCA13, ADAMTS2, ADAMTS3, AK5, ANKRD22, ANKRD34B, ANLN, AQP1, ARG1, ARHGAP44, ARHGEF17, ASPM, ATP1B2, AURKA, AZU1, BAIAP3, BPI, Clorf226, CACNB4, CCL4L2, CCN3, CCNA1, CD177, CD24, CDK1, CDKN3, CEACAM6, CEACAM8, CENPA, CFH, CHDH, CHIT1, CKAP2L, CLEC4C, CLEC4F, CLNK, COL17A1, CRISP2, CRISP3, CTSE, CTSG, CYP19A1, CYYR1, DEFA4, DENND2C, DEPDC1, DGKK, DLC1, DLGAP5, DNAH10, DOC2B, DSP, ELANE, ERG, FAM20A, FAM83A, FBN1, FFAR3, GOS2, GGT5, GLB1L2, GJB6, GPR84, GRAMD1C, GYPA, HBM, HMGB3, HP, HPGD, HRK, IGLL1, IL1R2, IL1RL1, INHBA, IQGAP3, ITGA7, ITGB4, KIF15, KIF20A, KLF14, LAMB3, LCN2, LGR4, LPL, LTF, MAFG, MERTK, METTL7B, MMP8, MMP9, MPO, MRC1, MROCKI, MS4A3, MS4A4A, NECAB1, NEIL3, NEK2, NRXN2, NUF2, OLAH, OLFM4, OLIG2, PCOLCE2, PCSK9, PHF24, PIGR, PLAAT2, PPARG, PRTN3, PTGES, PYCRI, RAB3IL1, RASGRF1, RETN, RHCE, RIPOR3, RPGRIP1, RRM2, S100A12, S100A8, SCN8A, SEMA6B, SERPINB10, SIGLEC8, SIL1, SLC16A1, SLC28A3, SLC39A8, SLC4A10, SLC51A, SLC6A19, SLC8A3, SLCO4A1, SMIM1, SMPDL3A, SPATC1, SPOP, SSBP2, TCN1, TCTEX1D1, TDRD9, TEAD2, TFRC, THBS1, TIMP3, TLN2, TMEM255A, TMEM45A, TNFAIP8L3, TNIP3, TROAP, TTK, VSIG4, WNT3, YPEL4, and ZDHHC19. The present disclosure also includes a use of an effective amount of one or more antibiotics for preparation of a medicament for treatment of sepsis in a subject predicted as having high or intermediate severity sepsis by a method comprising: (a) determining, in a biological sample from the subject, a level of expression for each of a plurality of genes, to provide a sample gene signature; and (b) comparing the sample gene signature with a reference gene signature to predict the severity of the sepsis in the subject, wherein high severity sepsis means a sequential organ failure assessment (SOFA) score of greater than or equal to 5, and intermediate severity sepsis means a SOFA score of greater than or equal to 2 but less than 5; and wherein the plurality of genes is selected from the group consisting of ABCA13, ADAMTS2, ADAMTS3, AK5, ANKRD22, ANKRD34B, ANLN, AQP1, ARG1, ARHGAP44, ARHGEF17, ASPM, ATP1B2, AURKA, AZU1, BAIAP3, BPI, Clorf226, CACNB4, CCL4L2, CCN3, CCNA1, CD177, CD24, CDK1, CDKN3, CEACAM6, CEACAM8, CENPA, CFH, CHDH, CHIT1, CKAP2L, CLEC4C, CLEC4F, CLNK, COL17A1, CRISP2, CRISP3, CTSE, CTSG, CYP19A1, CYYR1, DEFA4, DENND2C, DEPDC1, DGKK, DLC1, DLGAP5, DNAH10, DOC2B, DSP, ELANE, ERG, FAM20A, FAM83A, FBN1, FFAR3, GOS2, GGT5, GLB1L2, GJB6, GPR84, GRAMD1C, GYPA, HBM, HMGB3, HP, HPGD, HRK, IGLL1, IL1R2, IL1RL1, INHBA, IQGAP3, ITGA7, ITGB4, KIF15, KIF20A, KLF14, LAMB3, LCN2, LGR4, LPL, LTF, MAFG, MERTK, METTL7B, MMP8, MMP9, MPO, MRC1, MROCKI, MS4A3, MS4A4A, NECAB1, NEIL3, NEK2, NRXN2, NUF2, OLAH, OLFM4, OLIG2, PCOLCE2, PCSK9, PHF24, PIGR, PLAAT2, PPARG, PRTN3, PTGES, PYCRI, RAB3IL1, RASGRF1, RETN, RHCE, RIPOR3, RPGRIP1, RRM2, S100A12, S100A8, SCN8A, SEMA6B, SERPINB10, SIGLEC8, SIL1, SLC16A1, SLC28A3, SLC39A8, SLC4A10, SLC51A, SLC6A19, SLC8A3, SLCO4A1, SMIM1, SMPDL3A, SPATC1, SPOP, SSBP2, TCN1, TCTEX1D1, TDRD9, TEAD2, TFRC, THBS1, TIMP3, TLN2, TMEM255A, TMEM45A, TNFAIP8L3, TNIP3, TROAP, TTK, VSIG4, WNT3, YPEL4, and ZDHHC19. The present disclosure further includes one or more antibiotics for use to treat sepsis in a subject predicted as having high or intermediate severity sepsis by a method for predicting severity of sepsis comprising: (i) determining, in a biological sample from the subject, a level of expression for each of a plurality of genes, to provide a sample gene signature; and (ii) comparing the sample gene signature with a reference gene signature to predict the severity of the sepsis in the subject, wherein high severity sepsis means a sequential organ failure assessment (SOFA) score of greater than or equal to 5, and intermediate severity sepsis means a SOFA score of greater than or equal to 2 but less than 5; and wherein the plurality of genes is selected from the group consisting of ABCA13, ADAMTS2, ADAMTS3, AK5, ANKRD22, ANKRD34B, ANLN, AQP1, ARG1, ARHGAP44, ARHGEF17, ASPM, ATP1B2, AURKA, AZU1, BAIAP3, BPI, Clorf226, CACNB4, CCL4L2, CCN3, CCNA1, CD177, CD24, CDK1, CDKN3, CEACAM6, CEACAM8, CENPA, CFH, CHDH, CHITI, CKAP2L, CLEC4C, CLEC4F, CLNK, COL17A1, CRISP2, CRISP3, CTSE, CTSG, CYP19A1, CYYR1, DEFA4, DENND2C, DEPDC1, DGKK, DLC1, DLGAP5, DNAH10, DOC2B, DSP, ELANE, ERG, FAM20A, FAM83A, FBN1, FFAR3, GOS2, GGT5, GLB1L2, GJB6, GPR84, GRAMD1C, GYPA, HBM, HMGB3, HP, HPGD, HRK, IGLL1, IL1R2, IL1RL1, INHBA, IQGAP3, ITGA7, ITGB4, KIF15, KIF20A, KLF14, LAMB3, LCN2, LGR4, LPL, LTF, MAFG, MERTK, METTL7B, MMP8, MMP9, MPO, MRC1, MROCKI, MS4A3, MS4A4A, NECAB1, NEIL3, NEK2, NRXN2, NUF2, OLAH, OLFM4, OLIG2, PCOLCE2, PCSK9, PHF24, PIGR, PLAAT2, PPARG, PRTN3, PTGES, PYCRI, RAB3IL1, RASGRF1, RETN, RHCE, RIPOR3, RPGRIP1, RRM2, S100A12, S100A8, SCN8A, SEMA6B, SERPINB10, SIGLEC8, SIL1, SLC16A1, SLC28A3, SLC39A8, SLC4A10, SLC51A, SLC6A19, SLC8A3, SLCO4A1, SMIMI, SMPDL3A, SPATC1, SPOP, SSBP2, TCN1, TCTEX1D1, TDRD9, TEAD2, TFRC, THBS1, TIMP3, TLN2, TMEM255A, TMEM45A, TNFAIP8L3, TNIP3, TROAP, TTK, VSIG4, WNT3, YPEL4, and ZDHHC19. In an embodiment, the sepsis is high severity sepsis. In another embodiment of the present disclosure, the sepsis is intermediate severity sepsis. It will be appreciated by a person skilled in the art that embodiments of such a method for predicting that the subject has high or intermediate severity sepsis can be varied, as appropriate, as described herein for the embodiments of the method for predicting severity of sepsis in a subject, wherein the severity of the sepsis is selected from high severity sepsis, intermediate severity sepsis and low severity sepsis.

Examples of suitable antibiotics for treating sepsis include, but are not limited to, glycopeptides (such as vancomycin, oritavancin or telavancin) cephalosporins (such as ceftriaxone, cefotaxime, or cefepime), beta-lactams/beta-lactamase inhibitors (such as piperacillin-tazobactam, or ticarcillin-clavulanate), carbapenems (such as imipenem or meropenem), quinolones and fluoroquinolones (such as ciprofloxacin, moxifloxacin or levofloxacin), aminoglycosides (such as gentamicin, tobramycin or amikacin), macrolides (such as azithromycin, clarithromycin or erythromycin) and monobactams (such as aztreonam), and various combinations thereof. Typically, combinations comprise antibiotics from different classes. In an embodiment, the one or more antibiotics is one or a combination of a glycopeptide, a cephalosporin, a beta-lactam, a beta-lactamase inhibitor, a carbapenem, a quinolone, a fluoroquinolone, an aminoglycoside, a macrolide and a monobactam.

The one or more therapies that act specifically against a mechanism associated with a sepsis mechanistic endotype or one or more antibiotics can be administered to a subject or used in a variety of forms depending on the selected route of administration or use, as will be understood by those skilled in the art, and which may depend, for example, on the particular therapy, antibiotic or combination thereof. In an embodiment, the one or more therapies that act specifically against a mechanism associated with a sepsis mechanistic endotype or one or more antibiotics are administered to the subject, or used, by oral (including buccal) or parenteral (including intravenous, intraperitoneal, subcutaneous, intramuscular, transepithelial, nasal, intrapulmonary, intrathecal, rectal, topical, patch, pump and transdermal) administration or use and the one or more therapies that act specifically against a mechanism associated with a sepsis mechanistic endotype or one or more antibiotics formulated accordingly. For example, the one or more therapies that act specifically against a mechanism associated with a sepsis mechanistic endotype or one or more antibiotics are administered or used in an injection, in a spray, in a tablet/caplet, in a powder, topically, in a gel, in drops, by a patch, by an implant, by a slow release pump or by any other suitable method of administration or use, the selection of which can be made by a person skilled in the art.

Treatment methods or uses comprise administering to a subject or use of an effective amount of one or more therapies that act specifically against a mechanism associated with a sepsis mechanistic endotype or one or more antibiotics, as the case may be, optionally consisting of a single administration or use, or alternatively comprising a series of administrations or uses. The length of the treatment period or use depends on a variety of factors, such as the severity of the sepsis, the age of the subject, the identity of the one or more therapies that act specifically against a mechanism associated with a sepsis mechanistic endotype or one or more antibiotics, and/or a combination thereof. It will also be appreciated that the effective amount of a therapy, antibiotic or combination thereof used for the treatment or use may increase or decrease over the course of a particular treatment regime or use. Changes in dosage may result and become apparent by standard diagnostic assays known in the art. In an embodiment, the one or more therapies that act specifically against a mechanism associated with a sepsis mechanistic endotype or one or more antibiotics, as the case may be, are administered or used in an amount and for duration sufficient to treat the subject.

The one or more therapies that act specifically against a mechanism associated with a sepsis mechanistic endotype or one or more antibiotics may be administered or used alone or in combination (i.e., a combination of therapies or a combination of antibiotics, as the case may be). When administered or used in combination, it is an embodiment that the combination of therapies or combination of antibiotics, as the case may be, are administered or used contemporaneously. As used herein the term “contemporaneous” in reference to administration of two substances to a subject or use means providing each of the two substances so that they are both biologically active in the individual at the same time. The exact details of the administration or use will depend on the pharmacokinetics of the two substances in the presence of each other, and can include administering or using the two substances within a few hours of each other, or even administering or using one substance within 24 hours of administration or use of the other, if the pharmacokinetics are suitable. Design of suitable dosing regimens is routine for one skilled in the art. In particular embodiments, two substances will be administered or used substantially simultaneously, i.e., within minutes of each other, or in a single composition that contains both substances. It is a further embodiment that a combination of the two substances is administered to a subject or used in a non-contemporaneous fashion.

The dosage of the one or more therapies that act specifically against a mechanism associated with a sepsis mechanistic endotype or one or more antibiotics can vary depending on many factors such as pharmacodynamic properties, the mode of administration or use, the age, health and weight of the subject, the frequency of the treatment or use and the type of concurrent treatment or use, if any, and the clearance rate in the subject. One of skill in the art can determine the appropriate dosage for a particular therapy, antibiotic or combination thereof.

### III. Further Aspects and Embodiments

The present disclosure also includes a method for identifying a candidate agent for the treatment of sepsis in a subject classified as having a sepsis mechanistic endotype selected from neutrophilic-suppressive (NPS), inflammatory (INF), innate host defense (IHD), interferon (IFN) and adaptive (ADA) endotypes, the method comprising: (a) contacting a cell having the sepsis endotype with a test agent, (b) determining the level of expression for each of a plurality of genes in the cell to provide an expression signature; (c) comparing the expression signature with a reference signature, wherein the reference signature represents the level of expression of the plurality of genes in a normal cell; and (d) selecting the test agent as a candidate agent for treatment of the sepsis when the expression signature substantially corresponds with the reference signature, wherein the expression signature and reference signature comprise an NPS endotype sub-signature for an NPS endotype cell, an INF endotype sub-signature for an INF endotype cell, an IHD endotype sub-signature for an IHD endotype cell, an IFN endotype sub-signature for an IFN endotype cell and an ADA endotype sub-signature for an ADA endotype cell, wherein the NPS endotype sub-signature comprises genes selected from the group consisting of AGFG1, ARG1, ATP9A, ANXA3, EFNA1, GADD45A, GPR84, HPGD, IL1R1, KLF14, KREMEN1, MIR646HG, MLLT1, NSUN7, OLAH, ORM2, PCOLCE2, PFKFB2, SLC51A, TNFAIP8L3, ZDHHC19, ADAMTS3, AKR1C1, ALDH1A2, ALOX5AP, ALPL, AMPH, ANKRD55, BCL3, BTBD19, CA4, CD163L1, CD177, CD82, CST7, CYP19A1, CYSTM1, DAAM2, DGAT2, ECHDC3, ENTPD7, EXOSC4, FFAR3, FGF13, FSTL4, GALNT14, GRAMD1A, GRB10, GYG1, HPGD, IER3, IL18RAP, IL1R2, IL1RN, IRAG1-AS1, KCNE1B, KCNMA1, MCEMP1, MKNK1, MMP9, MSRA, NECAB1, NSMCE1-DT, OPLAH, PDGFC, PFKFB3, PHF24, PI3, PLIN4, PLIN5, PLK3, POR, PROK2, RFX2, RGL4, ROM1, S100A12, S100P, SEMA6B, SHROOM4, SLP1, SOCS3, SPATC1, SPDYA, SPINK8, SPP1, ST6GALNAC3, SYN2, TDRD9, TMEM120A, TMIGD3, TSPO, UPP1, and XCR1; wherein the INF endotype sub-signature comprises genes selected from the group consisting of BNIP3L, CA1, FAM83A, FECH, GLRX5, GYPA, IFIT1B, RHCE, RIOK3, RNF182, SLC6A19, SPTA1, THEM5, TLCD4, TSPAN5, TSPO2, ABCG2, ACHE, ACKR1, ACSL6, ADD2, AHSP, ALAS2, ALDH5A1, ANK1, ANKRD9, AQP1, ARHGEF12, ARHGEF37, ARL4A, ATP1B2, ATP1B2, BBOF1, BCAM, BCL2L1, BLVRB, BPGM, Clorf116, CA2, CISD2, CLIC2, CR1L, CR1L, CTNNAL1, CTSE, DCAF12, DMTN, DNAJC6, DPCD, DYRK3, EMID1, EPB42, ERFE, FAM210B, FAXDC2, FRMD4A, GMPR, GSPT1, GYPB, HBM, HEMGN, HEPACAM2, HMBS, IGF2BP2, ISCA1, ITLN1, KANK2, KCNH2, KDM7A-DT, KEL, KLC3, KLF1, KLHDC8A, KRT1, LRRC2, MAOA, MARCHF8, MBNL3, MFSD2B, MRC2, MXI1, MYL4, NFIX, NUDT4, OSBP2, PAGE2B, PBX1, PCDH1, PGF, PLEK2, PNP, PRDX2, PTPRF, RAPIGAP, RBM38, RFESD, RFESD, RGCC, RGS16, RHAG, RHD, RIPOR3, RNF175, RUNDC3A, SEC14L4, SELENBP1, SELENOP, SFRP2, SGIP1, SIAH2, SLC14A1, SLC1A5, SLC22A23, SLC2A1, SLC4A1, SLC6A8, SLC6A9, SLC7A5, SMIM5, SNCA, SOX6, SPTB, STRADB, TAL1, TENT5C, TFR2, TMCC2, TMOD1, TNS1, TRIM10, TRIM58, TSPAN7, TTC25, UBB, USP12, XK, YBX3, and YPEL4; wherein the IHD endotype sub-signature comprises genes selected from the group consisting of ABCA6, ADAM23, ALOX15, CACNA2D3, DYNC2H1, GPR34, GRAMD1C, LPL, MAP7, MIR155HG, PLCB1, SDC2, SIGLEC8, SPRED1, SLC16A14, SMPD3, TPPP3, TPRG1, ZNF600, ADGRD1, ANGPT1, GPR82, HDAC9, IL5RA, KLHDC1, PRSS33, PTGDR2, PTGFRN, TBC1D12, and TRIM2; wherein the IFN endotype sub-signature comprises genes selected from the group consisting of ANKRD22, APOL1, APOL4, BATF2, CARD17, CD274, EPSTI1, ETV7, GBP5, IDO1, IFITM3, P2RY14, PLEKHO1, RSAD2, SERPING1, TFEC, EXOC3L1, IRF7, OAS1, SEPTIN4, LY6E, and LAMP3; and wherein the ADA endotype sub-signature comprises genes selected from the group consisting of CCL2, CDC45, CENPF, CLEC4F, GTSE1, IFI27, ISG15, KCTD14, KIF14, KIF15, KLHDC7B, LGALS3BP, OTOF, PDIA4, SIGLEC1, USP18, AGRN, CD38, CDCA7, CDT1, CTLA4, DHX58, EME1, FAM111B, HES4, IFI44L, IFIT3, IFNG-AS1, IL12RB2, IL4I1, KIF19, LAG3, MCM10, P2RY6, PACSIN1, PARM1, SAMD4A, SPATS2L, HERC5, TMPRSS3, TNFRSF13B, TSHR, and TTC21A.

The present disclosure also includes a method for identifying a candidate agent for the treatment of sepsis in a subject classified as having a sepsis mechanistic endotype selected from neutrophilic-suppressive (NPS), inflammatory (INF), innate host defense (IHD), interferon (IFN) and adaptive (ADA) endotypes, the method comprising: (a) contacting a cell having the sepsis endotype with a test agent, (b) determining the level of expression for each of a plurality of genes in the cell to provide an expression signature; (c) comparing the expression signature with a reference signature, wherein the reference signature represents the level of expression of the plurality of genes in a normal cell; and (d) selecting the test agent as a candidate agent for treatment of the sepsis when the expression signature substantially corresponds with the reference signature, wherein the expression signature and reference signature comprise an NPS endotype signature pair for an NPS endotype cell, an INF endotype signature pair for an INF endotype cell, an IHD endotype signature pair for an IHD endotype cell, an IFN endotype signature pair for an IFN endotype cell, and an ADA endotype signature pair for an ADA endotype cell, wherein the NPS endotype signature pair is selected from: GADD45A/EFNA1, EFNA1/MIR646HG, MIR646HG/KLF14, MLLT1/MIR646HG, ARG1/MLLT1, MLLT1/EFNA1, MLLT1/NSUN7, EFNA1/NSUN7, SLC51A/EFNA1, EFNA1/KLF14, ZDHHC19/EFNA1, EFNA1/AGFG1, NSUN7/KLF14, EFNA1/PFKFB2, MLLT1/KLF14, ADAMTS3/PCOLCE2, ADAMTS3/ZDHHC19, ADAMTS3/SLC51A, ADAMTS3/HPGD, ADAMTS3/SEMA6B, ADAMTS3/EFNA1, ADAMTS3/AGFG1, ADAMTS3/NSUN7, ADAMTS3/TNFAIP8L3, ADAMTS3/KREMEN1, ADAMTS3/ORM2, ADAMTS3/MIR646HG, ADAMTS3/KLF14, AGFG1/NSUN7, AGFG1/TNFAIP8L3, AGFG1/KREMEN1, AGFG1/ORM2, AGFG1/MIR646HG, AGFG1/KLF14, ANXA3/GPR84, ANXA3/OLAH, ANXA3/ADAMTS3, ANXA3/PCOLCE2, ANXA3/ZDHHC19, ANXA3/SLC51A, ANXA3/HPGD, ANXA3/SEMA6B, ANXA3/EFNA1, ANXA3/AGFG1, ANXA3/NSUN7, ANXA3/TNFAIP8L3, ANXA3/KREMEN1, ANXA3/ORM2, ANXA3/MIR646HG, ANXA3/KLF14, ARG1/PFKFB2, ARG1/MLLT1, ARG1/ANXA3, ARG1/GPR84, ARG1/OLAH, ARG1/ADAMTS3, ARG1/PCOLCE2, ARG1/ZDHHC19, ARG1/SLC51A, ARG1/HPGD, ARG1/SEMA6B, ARG1/EFNA1, ARG1/AGFG1, ARG1/NSUN7, ARG1/TNFAIP8L3, ARG1/KREMEN1, ARG1/ORM2, ARG1/MIR646HG, ARG1/KLF14, ATP9A/EPB41L4B, ATP9A/IL1R1, ATP9A/GADD45A, ATP9A/ARG1, ATP9A/PFKFB2, ATP9A/MLLT1, ATP9A/ANXA3, ATP9A/GPR84, ATP9A/OLAH, ATP9A/ADAMTS3, ATP9A/PCOLCE2, ATP9A/ZDHHC19, ATP9A/SLC51A, ATP9A/HPGD, ATP9A/SEMA6B, ATP9A/EFNA1, ATP9A/AGFG1, ATP9A/NSUN7, ATP9A/TNFAIP8L3, ATP9A/KREMEN1, ATP9A/ORM2, ATP9A/MIR646HG, ATP9A/KLF14, EFNA1/AGFG1, EFNA1/NSUN7, EFNA1/TNFAIP8L3, EFNA1/KREMEN1, EFNA1/ORM2, EFNA1/MIR646HG, EFNA1/KLF14, EPB41L4B/IL1R1, EPB41L4B/GADD45A, EPB41L4B/ARG1, EPB41L4B/PFKFB2, EPB41L4B/MLLT1, EPB41L4B/ANXA3, EPB41L4B/GPR84, EPB41L4B/OLAH, EPB41L4B/ADAMTS3, EPB41L4B/PCOLCE2, EPB41L4B/ZDHHC19, EPB41L4B/SLC51A, EPB41L4B/HPGD, EPB41L4B/SEMA6B, EPB41L4B/EFNA1, EPB41L4B/AGFG1I,EPB41L4B/NSUN7, EPB41L4B/TNFAIP8L3, EPB41L4B/KREMEN1, EPB41L4B/MIR646HG, EPB41L4B/KLF14, GADD45A/ARG1, GADD45A/PFKFB2, GADD45A/MLLT1, GADD45A/ANXA3, GADD45A/GPR84, GADD45A/OLAH, GADD45A/ADAMTS3, GADD45A/PCOLCE2, GADD45A/ZDHHC19, GADD45A/SLC51A, GADD45A/HPGD, GADD45A/SEMA6B, GADD45A/EFNA1, GADD45A/AGFG1, GADD45A/NSUN7, GADD45A/TNFAIP8L3, GADD45A/KREMEN1, GADD45A/ORM2, GADD45A/MIR646HG, GADD45A/KLF14, GPR84/OLAH, GPR84/ADAMTS3, GPR84/PCOLCE2, GPR84/ZDHHC19, GPR84/SLC51A, GPR84/HPGD, GPR84/SEMA6B, GPR84/EFNA1, GPR84/AGFG1, GPR84/NSUN7, GPR84/TNFAIP8L3, GPR84/KREMEN1, GPR84/ORM2, GPR84/MIR646HG, GPR84/KLF14, HPGD/SEMA6B, HPGD/EFNA1, HPGD/AGFG1, HPGD/NSUN7, HPGD/TNFAIP8L3, HPGD/KREMEN1, HPGD/ORM2, HPGD/MIR646HG, HPGD/KLF14, IL1R1/GADD45A, ILIR1/ARG1, IL1R1/PFKFB2, IL1R1/MLLT1, IL1R1/ANXA3, IL1R1/GPR84, IL1R1/OLAH, IL1R1/ADAMTS3, IL1R1/PCOLCE2, IL1R1/ZDHHC19, IL1R1/SLC51A, IL1R1/HPGD, IL1R1/SEMA6B, IL1R1/EFNA1, IL1R1/AGFG1, IL1R1/NSUN7, IL1R1/TNFAIP8L3, IL1R1/KREMEN1, IL1R1/ORM2, IL1R1/MIR646HG, IL1R1/KLF14, KREMEN1/ORM2, KREMEN1/MIR646HG, KREMEN1/KLF14, MIR646HG/KLF14, MLLT1/ANXA3, MLLT1/GPR84, MLLT1/OLAH, MLLT1/ADAMTS3, MLLT1/PCOLCE2, MLLT1/ZDHHC19, MLLT1/SLC51A, MLLT1/HPGD, MLLT1/SEMA6B, MLLT1/EFNA1, MLLT1/AGFG1, MLLT1/NSUN7, MLLT1/TNFAIP8L3, MLLT1/KREMEN1, MLLT1/ORM2, MLLT1/MIR646HG, MLLT1/KLF14, NSUN7/TNFAIP8L3, NSUN7/KREMEN1, NSUN7/ORM2, NSUN7/MIR646HG, NSUN7/KLF14, OLAH/ADAMTS3, OLAH/PCOLCE2, OLAH/ZDHHC19, OLAH/SLC51A, OLAH/HPGD, OLAH/SEMA6B, OLAH/EFNA1, OLAH/AGFG1, OLAH/NSUN7, OLAH/TNFAIP8L3, OLAH/KREMEN1, OLAH/ORM2, OLAH/MIR646HG, OLAH/KLF14, ORM2/MIR646HG, ORM2/KLF14, PCOLCE2/ZDHHC19, PCOLCE2/SLC51A, PCOLCE2/HPGD, PCOLCE2/SEMA6B, PCOLCE2/EFNA1, PCOLCE2/AGFG1, PCOLCE2/NSUN7, PCOLCE2/TNFAIP8L3, PCOLCE2/KREMEN1, PCOLCE2/ORM2, PCOLCE2/MIR646HG, PCOLCE2/KLF14, PFKFB2/MLLT1, PFKFB2/ANXA3, PFKFB2/GPR84, PFKFB2/OLAH, PFKFB2/ADAMTS3, PFKFB2/PCOLCE2, PFKFB2/ZDHHC19, PFKFB2/SLC51A, PFKFB2/HPGD, PFKFB2/SEMA6B, PFKFB2/EFNA1, PFKFB2/AGFG1, PFKFB2/NSUN7, PFKFB2/TNFAIP8L3, PFKFB2/KREMEN1, PFKFB2/ORM2, PFKFB2/MIR646HG, PFKFB2/KLF14, SEMA6B/EFNA1, SEMA6B/AGFG1, SEMA6B/NSUN7, SEMA6B/TNFAIP8L3, SEMA6B/KREMEN1, SEMA6B/ORM2, SEMA6B/MIR646HG, SEMA6B/KLF14, SLC51A/HPGD, SLC51A/SEMA6B, SLC51A/EFNA1, SLC51A/AGFG1, SLC51A/NSUN7, SLC51A/TNFAIP8L3, SLC51A/KREMEN1, SLC51A/ORM2, SLC51A/MIR646HG, SLC51A/KLF14, TNFAIP8L3/KREMEN1, TNFAIP8L3/ORM2, TNFAIP8L3/MIR646HG, TNFAIP8L3/KLF14, ZDHHC19/SLC51A, ZDHHC19/HPGD, ZDHHC19/SEMA6B, ZDHHC19/EFNA1, ZDHHC19/AGFG1, ZDHHC19/NSUN7, ZDHHC19/TNFAIP8L3, ZDHHC19/KREMEN1, ZDHHC19/ORM2, ZDHHC19/MIR646HG, and ZDHHC19/KLF14; wherein the INF endotype signature pair is selected from: FECH/TFEC, TFEC/IFIT1B, FECH/RNF182, IFIT1B/FECH, FECH/APOL4, FECH/GYPA, ITLN1/FECH, FECH/THEM5, IFIT1B/CA1, RHAG/FECH, FECH/FAM83A, RHCE/FECH, TFEC/CA1, SPTA1/FECH, ANKRD22/GLRX5, ANKRD22/GYPA, ANKRD22/IFIT1B, ANKRD22/ITLN1, ANKRD22/KLHDC8A, ANKRD22/RHCE, ANKRD22/RNF182, ANKRD22/SPTA1, ANKRD22/THEM5, ANKRD22/TSPAN5, APOL4/BNIP3L, APOL4/CA1, APOL4/DYRK3, APOL4/FAM83A, APOL4/GLRX5, APOL4/GYPA, APOL4/IFIT1B, APOL4/ITLN1, APOL4/KLHDC8A, APOL4/RHAG, APOL4/RHCE, APOL4/RIOK3, APOL4/RNF182, APOL4/SPTA1, APOL4/THEM5, APOL4/TLCD4, APOL4/TMCC2, APOL4/TSPAN5, APOL4/TSPO2, BNIP3L/ANKRD22, BNIP3L/CA1, BNIP3L/CARD17, BNIP3L/CD274, BNIP3L/DYRK3, BNIP3L/FAM83A, BNIP3L/GBP5, BNIP3L/GLRX5, BNIP3L/GYPA, BNIP3L/IFIT1B, BNIP3L/ITLN1, BNIP3L/KLHDC8A, BNIP3L/P2RY14, BNIP3L/RHAG, BNIP3L/RHCE, BNIP3L/RNF182, BNIP3L/SPTA1, BNIP3L/TFEC, BNIP3L/THEM5, BNIP3L/TLCD4, BNIP3L/TMCC2, BNIP3L/TSPAN5, BNIP3L/TSPO2, CA1/ANKRD22, CA1/CARD17, CA1/DYRK3, CA1/FAM83A, CA1/GBP5, CA1/GLRX5, CA1/GYPA, CA1/IFIT1B, CA1/ITLN1, CA1/KLHDC8A, CA1/P2RY14, CA1/RHCE, CA1/RNF182, CA1/SPTA1, CA1/THEM5, CA1/TLCD4, CA1/TSPAN5, CD274/CA1, CD274/DYRK3, CD274/FAM83A, CD274/GLRX5, CD274/GYPA, CD274/IFIT1B, CD274/ITLN1, CD274/KLHDC8A, CD274/RHCE, CD274/RNF182, CD274/SPTA1, CD274/THEM5, CD274/TLCD4, CD274/TMCC2, CD274/TSPAN5, DYRK3/ANKRD22, DYRK3/CARD17, DYRK3/FAM83A, DYRK3/GBP5, DYRK3/GLRX5, DYRK3/GYPA, DYRK3/IFIT1B, DYRK3/ITLN1, DYRK3/KLHDC8A, DYRK3/P2RY14, DYRK3/RHCE, DYRK3/RNF182, DYRK3/SPTA1, DYRK3/THEM5, DYRK3/TLCD4, DYRK3/TSPAN5, FAM83A/ANKRD22, FAM83A/CARD17, FAM83A/GBP5, FAM83A/GLRX5, FAM83A/GYPA, FAM83A/IFIT1B, FAM83A/ITLN1, FAM83A/KLHDC8A, FAM83A/P2RY14, FAM83A/RHCE, FAM83A/RNF182, FAM83A/SPTA1, FAM83A/THEM5, FAM83A/TLCD4, FAM83A/TSPAN5, FECH/ANKRD22, FECH/APOL4, FECH/BNIP3L, FECH/CA1, FECH/CARD17, FECH/CD274, FECH/DYRK3, FECH/FAM83A, FECH/GBP5, FECH/GLRX5, FECH/GYPA, FECH/IFIT1B, FECH/ITLN1, FECH/KLHDC8A, FECH/P2RY14, FECH/RHAG, FECH/RHCE, FECH/RIOK3, FECH/RNF182, FECH/SPTA1, FECH/TFEC, FECH/THEM5, FECH/TLCD4, FECH/TMCC2, FECH/TSPAN5, FECH/TSPO2, GBP5/GLRX5, GBP5/GYPA, GBP5/IFIT1B, GBP5/ITLN1, GBP5/KLHDC8A, GBP5/RHCE, GBP5/RNF182, GBP5/SPTA1, GBP5/THEM5, GBP5/TSPAN5, GLRX5/CARD17, GLRX5/IFIT1B, GLRX5/RHCE, GLRX5/THEM5, GYPA/CARD17, GYPA/GLRX5, GYPA/IFIT1B, GYPA/ITLN1, GYPA/P2RY14, GYPA/RHCE, GYPA/RNF182, GYPA/THEM5, IFIT1B/CARD17, ITLN1/CARD17, ITLN1/GLRX5, ITLN1/IFIT1B, ITLN1/RHCE, ITLN1/RNF182, ITLN1/THEM5, KLHDC8A/CARD17, KLHDC8A/GLRX5, KLHDC8A/GYPA, KLHDC8A/IFIT1B, KLHDC8A/ITLN1, KLHDC8A/P2RY14, KLHDC8A/RHCE, KLHDC8A/RNF182, KLHDC8A/SPTA1, KLHDC8A/THEM5, KLHDC8A/TSPAN5, P2RY14/GLRX5, P2RY14/IFIT1B, P2RY14/ITLN1, P2RY14/RHCE, P2RY14/RNF182, P2RY14/THEM5, RHAG/ANKRD22, RHAG/CA1, RHAG/CARD17, RHAG/CD274, RHAG/DYRK3, RHAG/FAM83A, RHAG/GBP5, RHAG/GLRX5, RHAG/GYPA, RHAG/IFIT1B, RHAG/ITLN1, RHAG/KLHDC8A, RHAG/P2RY14, RHAG/RHCE, RHAG/RNF182, RHAG/SPTA1, RHAG/THEM5, RHAG/TLCD4, RHAG/TMCC2, RHAG/TSPAN5, RHAG/TSPO2, RHCE/CARD17, RHCE/IFIT1B, RHCE/THEM5, RIOK3/ANKRD22, RIOK3/BNIP3L, RIOK3/CA1, RIOK3/CARD17, RIOK3/CD274, RIOK3/DYRK3, RIOK3/FAM83A, RIOK3/GBP5, RIOK3/GLRX5, RIOK3/GYPA, RIOK3/IFIT1B, RIOK3/ITLN1, RIOK3/KLHDC8A, RIOK3/P2RY14, RIOK3/RHAG, RIOK3/RHCE, RIOK3/RNF182, RIOK3/SPTA1, RIOK3/TFEC, RIOK3/THEM5, RIOK3/TLCD4, RIOK3/TMCC2, RIOK3/TSPAN5, RIOK3/TSPO2, RNF182/CARD17, RNF182/GLRX5, RNF182/IFIT1B, RNF182/RHCE, RNF182/THEM5, SPTA1/CARD17, SPTA1/GLRX5, SPTA1/GYPA, SPTA1/IFIT1B, SPTA1/ITLN1, SPTA1/P2RY14, SPTA1/RHCE, SPTA1/RNF182, SPTA1/THEM5, SPTA1/TSPAN5, TFEC/CA1, TFEC/DYRK3, TFEC/FAM83A, TFEC/GLRX5, TFEC/GYPA, TFEC/IFIT1B, TFEC/ITLN1, TFEC/KLHDC8A, TFEC/RHAG, TFEC/RHCE, TFEC/RNF182, TFEC/SPTA1, TFEC/THEM5, TFEC/TLCD4, TFEC/TMCC2, TFEC/TSPAN5, TFEC/TSPO2, THEM5/CARD17, THEM5/IFIT1B, TLCD4/ANKRD22, TLCD4/CARD17, TLCD4/GBP5, TLCD4/GLRX5, TLCD4/GYPA, TLCD4/IFIT1B, TLCD4/ITLN1, TLCD4/KLHDC8A, TLCD4/P2RY14, TLCD4/RHCE, TLCD4/RNF182, TLCD4/SPTA1, TLCD4/THEM5, TLCD4/TSPAN5, TMCC2/ANKRD22, TMCC2/CA1, TMCC2/CARD17, TMCC2/DYRK3, TMCC2/FAM83A, TMCC2/GBP5, TMCC2/GLRX5, TMCC2/GYPA, TMCC2/IFIT1B, TMCC2/ITLN1, TMCC2/KLHDC8A, TMCC2/P2RY14, TMCC2/RHCE, TMCC2/RNF182, TMCC2/SPTA1, TMCC2/THEM5, TMCC2/TLCD4, TMCC2/TSPAN5, TSPAN5/CARD17, TSPAN5/GLRX5, TSPAN5/GYPA, TSPAN5/IFIT1B, TSPAN5/ITLN1, TSPAN5/P2RY14, TSPAN5/RHCE, TSPAN5/RNF182, TSPAN5/THEM5, TSPO2/ANKRD22, TSPO2/CA1, TSPO2/CARD17, TSPO2/CD274, TSPO2/DYRK3, TSPO2/FAM83A, TSPO2/GBP5, TSPO2/GLRX5, TSPO2/GYPA, TSPO2/IFIT1B, TSPO2/ITLN1, TSPO2/KLHDC8A, TSPO2/P2RY14, TSPO2/RHCE, TSPO2/RNF182, TSPO2/SPTA1, TSPO2/THEM5, TSPO2/TLCD4, TSPO2/TMCC2, and TSPO2/TSPAN5; wherein the IHD endotype signature pair is selected from: MAP7/SPRED1, SPRED1/GPR34, IL5RA/SPRED1, SPRED1/TPRG1, HRK/SPRED1, SPRED1/PLCB1, TRIM2/SPRED1, SIGLEC8/SPRED1, SMPD3/SPRED1, SPRED1/ZNF600, SPRED1/SDC2, MAP7/GPR34, PRSS33/SPRED1, SPRED1/DYNC2H1, CACNA2D3/SPRED1, ADAM23/GPR34, ADAM23/MAP7, ADAM23/PLCB1, ADAM23/SPRED1, ALOX15/GPR34, ALOX15/PLCB1, ALOX15/SPRED1, BAALC/GPR34, BAALC/PLCB1, BAALC/SPRED1, CACNA2D3/DYNC2H1, CACNA2D3/GPR34, CACNA2D3/PLCB1, CACNA2D3/SPRED1, CACNA2D3/ZNF600, GPR34/DYNC2H1, GPR34/GRAMD1C, GPR34/PLCB1, GPR34/TPRG1, GPR34/ZNF600, GPR82/DYNC2H1, GPR82/GPR34, GPR82/GRAMD1C, GPR82/PLCB1, GPR82/TPRG1, GPR82/ZNF600, GRAMD1C/DYNC2H1, GRAMD1C/PLCB1, GRAMD1C/ZNF600, HRK/DYNC2H1, HRK/GPR34, HRK/MAP7, HRK/PLCB1, HRK/SPRED1, HRK/ZNF600, IL5RA/DYNC2H1, IL5RA/GPR34, IL5RA/PLCB1, IL5RA/SPRED1, IL5RA/TRIM2, MAP7/BAALC, MAP7/CACNA2D3, MAP7/DYNC2H1, MAP7/GPR34, MAP7/GPR82, MAP7/GRAMD1C, MAP7/PLCB1, MAP7/SPRED1, MAP7/TPRG1, MAP7/ZNF600, PLCB1/DYNC2H1, PLCB1/TPRG1, PLCB1/ZNF600, PRSS33/GPR34, PRSS33/PLCB1, PRSS33/SPRED1, SDC2/DYNC2H1, SDC2/GPR34, SDC2/PLCB1, SDC2/ZNF600, SIGLEC8/DYNC2H1, SIGLEC8/GPR34, SIGLEC8/MAP7, SIGLEC8/PLCB1, SIGLEC8/SPRED1, SIGLEC8/TRIM2, SMPD3/DYNC2H1, SMPD3/GPR34, SMPD3/MAP7, SMPD3/PLCB1, SMPD3/SPRED1, SMPD3/TRIM2, SPRED1/DYNC2H1, SPRED1/GPR34, SPRED1/GPR82, SPRED1/GRAMD1C, SPRED1/PLCB1, SPRED1/SDC2, SPRED1/TPRG1, SPRED1/ZNF600, TRIM2/CACNA2D3, TRIM2/DYNC2H1, TRIM2/GPR34, TRIM2/GPR82, TRIM2/GRAMD1C, TRIM2/HRK, TRIM2/MAP7, TRIM2/PLCB1, TRIM2/SDC2, TRIM2/SPRED1, TRIM2/TPRG1, and TRIM2/ZNF600; wherein the IFN endotype signature pair is selected from: ETV7/PLEKHO1, IFITM3/ETV7, ETV7/APOL1, BATF2/ETV7, PLEKHO1/BATF2, ETV7/EPSTI1, EPSTI1/BATF2, IFITM3/BATF2, USP18/EPSTI1, ETV7/SEPTIN4, ETV7/LAMP3, SERPING1/BATF2, LAMP3/BATF2, LAMP3/SERPING1, APOL1/BATF2, APOL1/CLEC4F, APOL1/EPSTI1, APOL1/EXOC3L1, APOL1/HES4, APOL1/IFITM3, APOL1/LY6E, APOL1/RSAD2, APOL1/SEPTIN4, APOL1/SERPING1, APOL1/TPPP3, BATF2/EXOC3L1, BATF2/HES4, CLEC4F/BATF2, CLEC4F/EXOC3L1, EPSTI1/BATF2, EPSTI1/CLEC4F, EPSTI1/EXOC3L1, EPSTI1/HES4, EPSTI1/IFITM3, EPSTI1/LY6E, EPSTI1/RSAD2, EPSTI1/SERPING1, EPSTI1/TPPP3, ETV7/APOL1, ETV7/BATF2, ETV7/CLEC4F, ETV7/EPSTI1, ETV7/EXOC3L1, ETV7/HES4, ETV7/IFITM3, ETV7/LAMP3, ETV7/LY6E, ETV7/PLEKHO1, ETV7/RSAD2, ETV7/SEPTIN4, ETV7/SERPING1, ETV7/TPPP3, EXOC3L1/HES4, IFITM3/SERPING1, IFITM3/CLEC4F, IFITM3/TPPP3, IFITM3/LY6E, IFITM3/EXOC3L1, IFITM3/HES4, LAMP3/APOL1, LAMP3/BATF2, LAMP3/CLEC4F, LAMP3/EPSTI1, LAMP3/EXOC3L1, LAMP3/HES4, LAMP3/IFITM3, LAMP3/LY6E, LAMP3/RSAD2, LAMP3/SEPTIN4, LAMP3/SERPING1, LAMP3/TPPP3, LY6E/BATF2, LY6E/EXOC3L1, PLEKHO1/APOL1, PLEKHO1/BATF2, PLEKHO1/EPSTI1, PLEKHO1/EXOC3L1, PLEKHO1/IFITM3, PLEKHO1/LAMP3, PLEKHO1/RSAD2, PLEKHO1/SEPTIN4, PLEKHO1/SERPING1, RSAD2/BATF2, RSAD2/CLEC4F, RSAD2/EXOC3L1, RSAD2/HES4, RSAD2/IFITM3, RSAD2/LY6E, RSAD2/SERPING1, RSAD2/TPPP3, SEPTIN4/BATF2, SEPTIN4/CLEC4F, SEPTIN4/EPSTI1, SEPTIN4/EXOC3L1, SEPTIN4/HES4, SEPTIN4/IFITM3, SEPTIN4/LY6E, SEPTIN4/RSAD2, SEPTIN4/SERPING1, SEPTIN4/TPPP3, SERPING1/BATF2, SERPING1/CLEC4F, SERPING1/EXOC3L1, SERPING1/HES4, SERPING1/LY6E, SERPING1/TPPP3, TPPP3/BATF2, and TPPP3/EXOC3L1; and wherein the ADA endotype signature pair is selected from: LGALS3BP/OTOF, LGALS3BP/IF127, LGALS3BP/KIF14, LGALS3BP/CENPF, GTSE1/LGALS3BP, LGALS3BP/KCTD14, LGALS3BP/PDIA4, LGALS3BP/TSHR, LGALS3BP/PLAAT2, OTOF/IF127, IGF1/LGALS3BP, CDC45/LGALS3BP, LGALS3BP/KIF15, LGALS3BP/IGLL5, LGALS3BP/MIXL, CAV1/LGALS3BP, CAV1/OTOF, CDC45/LGALS3BP, CDC45/OTOF, CENPF/KCTD14, GPRC5D/OTOF, GTSE1/LGALS3BP, GTSE1/OTOF, IGF1/LGALS3BP, IGF1/OTOF, KCTD14/KLHL14, KCTD14/PDIA4, KCTD14/TSHR, KIF14/KCTD14, LGALS3BP/CENPF, LGALS3BP/GPRC5D, LGALS3BP/IFI27, LGALS3BP/IGLL5, LGALS3BP/KCTD14, LGALS3BP/KIF14, LGALS3BP/KIF15, LGALS3BP/KLHL14, LGALS3BP/MIR155HG, LGALS3BP/MIXL1, LGALS3BP/OTOF, LGALS3BP/PDIA4, LGALS3BP/PLAAT2, LGALS3BP/SDC1, LGALS3BP/SLC16A14, LGALS3BP/TSHR, OTOF/CENPF, OTOF/IF127, OTOF/IGLL5, OTOF/KCTD14, OTOF/KIF14, OTOF/KIF15, OTOF/KLHL14, OTOF/MIR155HG, OTOF/MIXL1, OTOF/PDIA4, OTOF/PLAAT2, OTOF/SDC1, OTOF/SLC16A14, OTOF/TSHR, PLAAT2/KCTD14, TNFRSF17/LGALS3BP, and TNFRSF17/OTOF.

The present disclosure also includes a kit for classifying a subject into a sepsis mechanistic endotype selected from neutrophilic-suppressive (NPS), inflammatory (INF), innate host defense (IHD), interferon (IFN) and adaptive (ADA) endotypes, the kit comprising gene specific reagents, each of the gene specific reagents capable of detecting an expression product of a respective one of a plurality of genes or complement thereof in an NPS endotype sub-signature, an INF endotype sub-signature, an IHD endotype sub-signature, an IFN endotype sub-signature, an ADA endotype sub-signature or combinations thereof, and optionally instructions for use, wherein the NPS endotype sub-signature comprises genes selected from the group consisting of AGFG1, ARG1, ATP9A, ANXA3, EFNA1, GADD45A, GPR84, HPGD, IL1R1, KLF14, KREMEN1, MIR646HG, MLLT1, NSUN7, OLAH, ORM2, PCOLCE2, PFKFB2, SLC51A, TNFAIP8L3, ZDHHC19, ADAMTS3, AKR1C1, ALDH1A2, ALOX5AP, ALPL, AMPH, ANKRD55, BCL3, BTBD19, CA4, CD163L1, CD177, CD82, CST7, CYP19A1, CYSTM1, DAAM2, DGAT2, ECHDC3, ENTPD7, EXOSC4, FFAR3, FGF13, FSTL4, GALNT14, GRAMD1A, GRB10, GYG1, HPGD, IER3, IL18RAP, IL1R2, IL1RN, IRAG1-AS1, KCNE1B, KCNMA1, MCEMP1, MKNK1, MMP9, MSRA, NECAB1, NSMCE1-DT, OPLAH, PDGFC, PFKFB3, PHF24, PI3, PLIN4, PLIN5, PLK3, POR, PROK2, RFX2, RGL4, ROM1, S100A12, S100P, SEMA6B, SHROOM4, SLP1, SOCS3, SPATC1, SPDYA, SPINK8, SPP1, ST6GALNAC3, SYN2, TDRD9, TMEM120A, TMIGD3, TSPO, UPP1, and XCR1; wherein the INF endotype sub-signature comprises genes selected from the group consisting of BNIP3L, CA1, FAM83A, FECH, GLRX5, GYPA, IFIT1B, RHCE, RIOK3, RNF182, SLC6A19, SPTA1, THEM5, TLCD4, TSPAN5, TSPO2, ABCG2, ACHE, ACKR1, ACSL6, ADD2, AHSP, ALAS2, ALDH5A1, ANK1, ANKRD9, AQP1, ARHGEF12, ARHGEF37, ARL4A, ATP1B2, ATP1B2, BBOF1, BCAM, BCL2L1, BLVRB, BPGM, Clorf116, CA2, CISD2, CLIC2, CR1L, CR1L, CTNNAL1, CTSE, DCAF12, DMTN, DNAJC6, DPCD, DYRK3, EMID1, EPB42, ERFE, FAM210B, FAXDC2, FRMD4A, GMPR, GSPT1, GYPB, HBM, HEMGN, HEPACAM2, HMBS, IGF2BP2, ISCA1, ITLN1, KANK2, KCNH2, KDM7A-DT, KEL, KLC3, KLF1, KLHDC8A, KRT1, LRRC2, MAOA, MARCHF8, MBNL3, MFSD2B, MRC2, MXI1, MYL4, NFIX, NUDT4, OSBP2, PAGE2B, PBX1, PCDH1, PGF, PLEK2, PNP, PRDX2, PTPRF, RAP1GAP, RBM38, RFESD, RFESD, RGCC, RGS16, RHAG, RHD, RIPOR3, RNF175, RUNDC3A, SEC14L4, SELENBP1, SELENOP, SFRP2, SGIP1, SIAH2, SLC14A1, SLC1A5, SLC22A23, SLC2A1, SLC4A1, SLC6A8, SLC6A9, SLC7A5, SMIM5, SNCA, SOX6, SPTB, STRADB, TAL1, TENT5C, TFR2, TMCC2, TMOD1, TNS1, TRIM10, TRIM58, TSPAN7, TTC25, UBB, USP12, XK, YBX3, and YPEL4; wherein the IHD endotype sub-signature comprises genes selected from the group consisting of ABCA6, ADAM23, ALOX15, CACNA2D3, DYNC2H1, GPR34, GRAMD1C, LPL, MAP7, MIR155HG, PLCB1, SDC2, SIGLEC8, SPRED1, SLC16A14, SMPD3, TPPP3, TPRG1, ZNF600, ADGRD1, ANGPT1, GPR82, HDAC9, IL5RA, KLHDC1, PRSS33, PTGDR2, PTGFRN, TBC1D12, and TRIM2; wherein the IFN endotype sub-signature comprises genes selected from the group consisting of ANKRD22, APOL1, APOL4, BATF2, CARD17, CD274, EPSTI1, ETV7, GBP5, IDO1, IFITM3, P2RY14, PLEKHO1, RSAD2, SERPING1, TFEC, EXOC3L1, IRF7, OAS1, SEPTIN4, LY6E, and LAMP3; and wherein the ADA endotype sub-signature comprises genes selected from the group consisting of CCL2, CDC45, CENPF, CLEC4F, GTSE1, IFI27, ISG15, KCTD14, KIF14, KIF15, KLHDC7B, LGALS3BP, OTOF, PDIA4, SIGLEC1, USP18, AGRN, CD38, CDCA7, CDT1, CTLA4, DHX58, EME1, FAM111B, HES4, IFI44L, IFIT3, IFNG-AS1, IL12RB2, IL4I1, KIF19, LAG3, MCM10, P2RY6, PACSIN1, PARM1, SAMD4A, SPATS2L, HERC5, TMPRSS3, TNFRSF13B, TSHR, and TTC21A.

The present disclosure also includes a kit for classifying a subject into a sepsis mechanistic endotype selected from neutrophilic-suppressive (NPS), inflammatory (INF), innate host defense (IHD), interferon (IFN) and adaptive (ADA) endotypes, the kit comprising gene specific reagents, each of the gene specific reagents capable of detecting an expression product of a respective one of a plurality of genes or complement thereof in an NPS endotype signature pair, an INF endotype signature pair, an IHD endotype signature pair, an IFN endotype signature pair, an ADA endotype signature pair or combinations thereof, and optionally instructions for use, wherein the NPS endotype signature pair is selected from: GADD45A/EFNA1, EFNA1/MIR646HG, MIR646HG/KLF14, MLLT1/MIR646HG, ARG1/MLLT1, MLLT1/EFNA1, MLLT1/NSUN7, EFNA1/NSUN7, SLC51A/EFNA1, EFNA1/KLF14, ZDHHC19/EFNA1, EFNA1/AGFG1, NSUN7/KLF14, EFNA1/PFKFB2, MLLT1/KLF14, ADAMTS3/PCOLCE2, ADAMTS3/ZDHHC19, ADAMTS3/SLC51A, ADAMTS3/HPGD, ADAMTS3/SEMA6B, ADAMTS3/EFNA1, ADAMTS3/AGFG1, ADAMTS3/NSUN7, ADAMTS3/TNFAIP8L3, ADAMTS3/KREMEN1, ADAMTS3/ORM2, ADAMTS3/MIR646HG, ADAMTS3/KLF14, AGFG1/NSUN7, AGFG1/TNFAIP8L3, AGFG1/KREMEN1, AGFG1/ORM2, AGFG1/MIR646HG, AGFG1/KLF14, ANXA3/GPR84, ANXA3/OLAH, ANXA3/ADAMTS3, ANXA3/PCOLCE2, ANXA3/ZDHHC19, ANXA3/SLC51A, ANXA3/HPGD, ANXA3/SEMA6B, ANXA3/EFNA1, ANXA3/AGFG1, ANXA3/NSUN7, ANXA3/TNFAIP8L3, ANXA3/KREMEN1, ANXA3/ORM2, ANXA3/MIR646HG, ANXA3/KLF14, ARG1/PFKFB2, ARG1/MLLT1, ARG1/ANXA3, ARG1/GPR84, ARG1/OLAH, ARG1/ADAMTS3, ARG1/PCOLCE2, ARG1/ZDHHC19, ARG1/SLC51A, ARG1/HPGD, ARG1/SEMA6B, ARG1/EFNA1, ARG1/AGFG1, ARG1/NSUN7, ARG1/TNFAIP8L3, ARG1/KREMEN1, ARG1/ORM2, ARG1/MIR646HG, ARG1/KLF14, ATP9A/EPB41L4B, ATP9A/IL1R1, ATP9A/GADD45A, ATP9A/ARG1, ATP9A/PFKFB2, ATP9A/MLLT1, ATP9A/ANXA3, ATP9A/GPR84, ATP9A/OLAH, ATP9A/ADAMTS3, ATP9A/PCOLCE2, ATP9A/ZDHHC19, ATP9A/SLC51A, ATP9A/HPGD, ATP9A/SEMA6B, ATP9A/EFNA1, ATP9A/AGFG1, ATP9A/NSUN7, ATP9A/TNFAIP8L3, ATP9A/KREMEN1, ATP9A/ORM2, ATP9A/MIR646HG, ATP9A/KLF14, EFNA1/AGFG1, EFNA1/NSUN7, EFNA1/TNFAIP8L3, EFNA1/KREMEN1, EFNA1/ORM2, EFNA1/MIR646HG, EFNA1/KLF14, EPB41L4B/IL1R1, EPB41L4B/GADD45A, EPB41L4B/ARG1, EPB41L4B/PFKFB2, EPB41L4B/MLLT1, EPB41L4B/ANXA3, EPB41L4B/GPR84, EPB41L4B/OLAH, EPB41L4B/ADAMTS3, EPB41L4B/PCOLCE2, EPB41L4B/ZDHHC19, EPB41L4B/SLC51A, EPB41L4B/HPGD, EPB41L4B/SEMA6B, EPB41L4B/EFNA1, EPB41L4B/AGFG1I,EPB41L4B/NSUN7, EPB41L4B/TNFAIP8L3, EPB41L4B/KREMEN1, EPB41L4B/MIR646HG, EPB41L4B/KLF14, GADD45A/ARG1, GADD45A/PFKFB2, GADD45A/MLLT1, GADD45A/ANXA3, GADD45A/GPR84, GADD45A/OLAH, GADD45A/ADAMTS3, GADD45A/PCOLCE2, GADD45A/ZDHHC19, GADD45A/SLC51A, GADD45A/HPGD, GADD45A/SEMA6B, GADD45A/EFNA1, GADD45A/AGFG1, GADD45A/NSUN7, GADD45A/TNFAIP8L3, GADD45A/KREMEN1, GADD45A/ORM2, GADD45A/MIR646HG, GADD45A/KLF14, GPR84/OLAH, GPR84/ADAMTS3, GPR84/PCOLCE2, GPR84/ZDHHC19, GPR84/SLC51A, GPR84/HPGD, GPR84/SEMA6B, GPR84/EFNA1, GPR84/AGFG1, GPR84/NSUN7, GPR84/TNFAIP8L3, GPR84/KREMEN1, GPR84/ORM2, GPR84/MIR646HG, GPR84/KLF14, HPGD/SEMA6B, HPGD/EFNA1, HPGD/AGFG1, HPGD/NSUN7, HPGD/TNFAIP8L3, HPGD/KREMEN1, HPGD/ORM2, HPGD/MIR646HG, HPGD/KLF14, IL1R1/GADD45A, IL1R1/ARG1, IL1R1/PFKFB2, IL1R1/MLLT1, IL1R1/ANXA3, IL1R1/GPR84, IL1R1/OLAH, IL1R1/ADAMTS3, IL1R1/PCOLCE2, IL1R1/ZDHHC19, IL1R1/SLC51A, IL1R1/HPGD, IL1R1/SEMA6B, IL1R1/EFNA1, IL1R1/AGFG1, IL1R1/NSUN7, IL1R1/TNFAIP8L3, IL1R1/KREMEN1, IL1R1/ORM2, IL1R1/MIR646HG, IL1R1/KLF14, KREMEN1/ORM2, KREMEN1/MIR646HG, KREMEN1/KLF14, MIR646HG/KLF14, MLLT1/ANXA3, MLLT1/GPR84, MLLT1/OLAH, MLLT1/ADAMTS3, MLLT1/PCOLCE2, MLLT1/ZDHHC19, MLLT1/SLC51A, MLLT1/HPGD, MLLT1/SEMA6B, MLLT1/EFNA1, MLLT1/AGFG1, MLLT1/NSUN7, MLLT1/TNFAIP8L3, MLLT1/KREMEN1, MLLT1/ORM2, MLLT1/MIR646HG, MLLT1/KLF14, NSUN7/TNFAIP8L3, NSUN7/KREMEN1, NSUN7/ORM2, NSUN7/MIR646HG, NSUN7/KLF14, OLAH/ADAMTS3, OLAH/PCOLCE2, OLAH/ZDHHC19, OLAH/SLC51A, OLAH/HPGD, OLAH/SEMA6B, OLAH/EFNA1, OLAH/AGFG1, OLAH/NSUN7, OLAH/TNFAIP8L3, OLAH/KREMEN1, OLAH/ORM2, OLAH/MIR646HG, OLAH/KLF14, ORM2/MIR646HG, ORM2/KLF14, PCOLCE2/ZDHHC19, PCOLCE2/SLC51A, PCOLCE2/HPGD, PCOLCE2/SEMA6B, PCOLCE2/EFNA1, PCOLCE2/AGFG1, PCOLCE2/NSUN7, PCOLCE2/TNFAIP8L3, PCOLCE2/KREMEN1, PCOLCE2/ORM2, PCOLCE2/MIR646HG, PCOLCE2/KLF14, PFKFB2/MLLT1, PFKFB2/ANXA3, PFKFB2/GPR84, PFKFB2/OLAH, PFKFB2/ADAMTS3, PFKFB2/PCOLCE2, PFKFB2/ZDHHC19, PFKFB2/SLC51A, PFKFB2/HPGD, PFKFB2/SEMA6B, PFKFB2/EFNA1, PFKFB2/AGFG1, PFKFB2/NSUN7, PFKFB2/TNFAIP8L3, PFKFB2/KREMEN1, PFKFB2/ORM2, PFKFB2/MIR646HG, PFKFB2/KLF14, SEMA6B/EFNA1, SEMA6B/AGFG1, SEMA6B/NSUN7, SEMA6B/TNFAIP8L3, SEMA6B/KREMEN1, SEMA6B/ORM2, SEMA6B/MIR646HG, SEMA6B/KLF14, SLC51A/HPGD, SLC51A/SEMA6B, SLC51A/EFNA1, SLC51A/AGFG1, SLC51A/NSUN7, SLC51A/TNFAIP8L3, SLC51A/KREMEN1, SLC51A/ORM2, SLC51A/MIR646HG, SLC51A/KLF14, TNFAIP8L3/KREMEN1, TNFAIP8L3/ORM2, TNFAIP8L3/MIR646HG, TNFAIP8L3/KLF14, ZDHHC19/SLC51A, ZDHHC19/HPGD, ZDHHC19/SEMA6B, ZDHHC19/EFNA1, ZDHHC19/AGFG1, ZDHHC19/NSUN7, ZDHHC19/TNFAIP8L3, ZDHHC19/KREMEN1, ZDHHC19/ORM2, ZDHHC19/MIR646HG, and ZDHHC19/KLF14; wherein the INF endotype signature pair is selected from: FECH/TFEC, TFEC/IFIT1B, FECH/RNF182, IFIT1B/FECH, FECH/APOL4, FECH/GYPA, ITLN1/FECH, FECH/THEM5, IFIT1B/CA1, RHAG/FECH, FECH/FAM83A, RHCE/FECH, TFEC/CA1, SPTA1/FECH, ANKRD22/GLRX5, ANKRD22/GYPA, ANKRD22/IFIT1B, ANKRD22/ITLN1, ANKRD22/KLHDC8A, ANKRD22/RHCE, ANKRD22/RNF182, ANKRD22/SPTA1, ANKRD22/THEM5, ANKRD22/TSPAN5, APOL4/BNIP3L, APOL4/CA1, APOL4/DYRK3, APOL4/FAM83A, APOL4/GLRX5, APOL4/GYPA, APOL4/IFIT1B, APOL4/ITLN1, APOL4/KLHDC8A, APOL4/RHAG, APOL4/RHCE, APOL4/RIOK3, APOL4/RNF182, APOL4/SPTA1, APOL4/THEM5, APOL4/TLCD4, APOL4/TMCC2, APOL4/TSPAN5, APOL4/TSPO2, BNIP3L/ANKRD22, BNIP3L/CA1, BNIP3L/CARD17, BNIP3L/CD274, BNIP3L/DYRK3, BNIP3L/FAM83A, BNIP3L/GBP5, BNIP3L/GLRX5, BNIP3L/GYPA, BNIP3L/IFIT1B, BNIP3L/ITLN1, BNIP3L/KLHDC8A, BNIP3L/P2RY14, BNIP3L/RHAG, BNIP3L/RHCE, BNIP3L/RNF182, BNIP3L/SPTA1, BNIP3L/TFEC, BNIP3L/THEM5, BNIP3L/TLCD4, BNIP3L/TMCC2, BNIP3L/TSPAN5, BNIP3L/TSPO2, CA1/ANKRD22, CA1/CARD17, CA1/DYRK3, CA1/FAM83A, CA1/GBP5, CA1/GLRX5, CA1/GYPA, CA1/IFIT1B, CA1/ITLN1, CA1/KLHDC8A, CA1/P2RY14, CA1/RHCE, CA1/RNF182, CA1/SPTA1, CA1/THEM5, CA1/TLCD4, CA1/TSPAN5, CD274/CA1, CD274/DYRK3, CD274/FAM83A, CD274/GLRX5, CD274/GYPA, CD274/IFIT1B, CD274/ITLN1, CD274/KLHDC8A, CD274/RHCE, CD274/RNF182, CD274/SPTA1, CD274/THEM5, CD274/TLCD4, CD274/TMCC2, CD274/TSPAN5, DYRK3/ANKRD22, DYRK3/CARD17, DYRK3/FAM83A, DYRK3/GBP5, DYRK3/GLRX5, DYRK3/GYPA, DYRK3/IFIT1B, DYRK3/ITLN1, DYRK3/KLHDC8A, DYRK3/P2RY14, DYRK3/RHCE, DYRK3/RNF182, DYRK3/SPTA1, DYRK3/THEM5, DYRK3/TLCD4, DYRK3/TSPAN5, FAM83A/ANKRD22, FAM83A/CARD17, FAM83A/GBP5, FAM83A/GLRX5, FAM83A/GYPA, FAM83A/IFIT1B, FAM83A/ITLN1, FAM83A/KLHDC8A, FAM83A/P2RY14, FAM83A/RHCE, FAM83A/RNF182, FAM83A/SPTA1, FAM83A/THEM5, FAM83A/TLCD4, FAM83A/TSPAN5, FECH/ANKRD22, FECH/APOL4, FECH/BNIP3L, FECH/CA1, FECH/CARD17, FECH/CD274, FECH/DYRK3, FECH/FAM83A, FECH/GBP5, FECH/GLRX5, FECH/GYPA, FECH/IFIT1B, FECH/ITLN1, FECH/KLHDC8A, FECH/P2RY14, FECH/RHAG, FECH/RHCE, FECH/RIOK3, FECH/RNF182, FECH/SPTA1, FECH/TFEC, FECH/THEM5, FECH/TLCD4, FECH/TMCC2, FECH/TSPAN5, FECH/TSPO2, GBP5/GLRX5, GBP5/GYPA, GBP5/IFIT1B, GBP5/ITLN1, GBP5/KLHDC8A, GBP5/RHCE, GBP5/RNF182, GBP5/SPTA1, GBP5/THEM5, GBP5/TSPAN5, GLRX5/CARD17, GLRX5/IFIT1B, GLRX5/RHCE, GLRX5/THEM5, GYPA/CARD17, GYPA/GLRX5, GYPA/IFIT1B, GYPA/ITLN1, GYPA/P2RY14, GYPA/RHCE, GYPA/RNF182, GYPA/THEM5, IFIT1B/CARD17, ITLN1/CARD17, ITLN1/GLRX5, ITLN1/IFIT1B, ITLN1/RHCE, ITLN1/RNF182, ITLN1/THEM5, KLHDC8A/CARD17, KLHDC8A/GLRX5, KLHDC8A/GYPA, KLHDC8A/IFIT1B, KLHDC8A/ITLN1, KLHDC8A/P2RY14, KLHDC8A/RHCE, KLHDC8A/RNF182, KLHDC8A/SPTA1, KLHDC8A/THEM5, KLHDC8A/TSPAN5, P2RY14/GLRX5, P2RY14/IFIT1B, P2RY14/ITLN1, P2RY14/RHCE, P2RY14/RNF182, P2RY14/THEM5, RHAG/ANKRD22, RHAG/CA1, RHAG/CARD17, RHAG/CD274, RHAG/DYRK3, RHAG/FAM83A, RHAG/GBP5, RHAG/GLRX5, RHAG/GYPA, RHAG/IFIT1B, RHAG/ITLN1, RHAG/KLHDC8A, RHAG/P2RY14, RHAG/RHCE, RHAG/RNF182, RHAG/SPTA1, RHAG/THEM5, RHAG/TLCD4, RHAG/TMCC2, RHAG/TSPAN5, RHAG/TSPO2, RHCE/CARD17, RHCE/IFIT1B, RHCE/THEM5, RIOK3/ANKRD22, RIOK3/BNIP3L, RIOK3/CA1, RIOK3/CARD17, RIOK3/CD274, RIOK3/DYRK3, RIOK3/FAM83A, RIOK3/GBP5, RIOK3/GLRX5, RIOK3/GYPA, RIOK3/IFIT1B, RIOK3/ITLN1, RIOK3/KLHDC8A, RIOK3/P2RY14, RIOK3/RHAG, RIOK3/RHCE, RIOK3/RNF182, RIOK3/SPTA1, RIOK3/TFEC, RIOK3/THEM5, RIOK3/TLCD4, RIOK3/TMCC2, RIOK3/TSPAN5, RIOK3/TSPO2, RNF182/CARD17, RNF182/GLRX5, RNF182/IFIT1B, RNF182/RHCE, RNF182/THEM5, SPTA1/CARD17, SPTA1/GLRX5, SPTA1/GYPA, SPTA1/IFIT1B, SPTA1/ITLN1, SPTA1/P2RY14, SPTA1/RHCE, SPTA1/RNF182, SPTA1/THEM5, SPTA1/TSPAN5, TFEC/CA1, TFEC/DYRK3, TFEC/FAM83A, TFEC/GLRX5, TFEC/GYPA, TFEC/IFIT1B, TFEC/ITLN1, TFEC/KLHDC8A, TFEC/RHAG, TFEC/RHCE, TFEC/RNF182, TFEC/SPTA1, TFEC/THEM5, TFEC/TLCD4, TFEC/TMCC2, TFEC/TSPAN5, TFEC/TSPO2, THEM5/CARD17, THEM5/IFIT1B, TLCD4/ANKRD22, TLCD4/CARD17, TLCD4/GBP5, TLCD4/GLRX5, TLCD4/GYPA, TLCD4/IFIT1B, TLCD4/ITLN1, TLCD4/KLHDC8A, TLCD4/P2RY14, TLCD4/RHCE, TLCD4/RNF182, TLCD4/SPTA1, TLCD4/THEM5, TLCD4/TSPAN5, TMCC2/ANKRD22, TMCC2/CA1, TMCC2/CARD17, TMCC2/DYRK3, TMCC2/FAM83A, TMCC2/GBP5, TMCC2/GLRX5, TMCC2/GYPA, TMCC2/IFIT1B, TMCC2/ITLN1, TMCC2/KLHDC8A, TMCC2/P2RY14, TMCC2/RHCE, TMCC2/RNF182, TMCC2/SPTA1, TMCC2/THEM5, TMCC2/TLCD4, TMCC2/TSPAN5, TSPAN5/CARD17, TSPAN5/GLRX5, TSPAN5/GYPA, TSPAN5/IFIT1B, TSPAN5/ITLN1, TSPAN5/P2RY14, TSPAN5/RHCE, TSPAN5/RNF182, TSPAN5/THEM5, TSPO2/ANKRD22, TSPO2/CA1, TSPO2/CARD17, TSPO2/CD274, TSPO2/DYRK3, TSPO2/FAM83A, TSPO2/GBP5, TSPO2/GLRX5, TSPO2/GYPA, TSPO2/IFIT1B, TSPO2/ITLN1, TSPO2/KLHDC8A, TSPO2/P2RY14, TSPO2/RHCE, TSPO2/RNF182, TSPO2/SPTA1, TSPO2/THEM5, TSPO2/TLCD4, TSPO2/TMCC2, and TSPO2/TSPAN5; wherein the IHD endotype signature pair is selected from: MAP7/SPRED1, SPRED1/GPR34, IL5RA/SPRED1, SPRED1/TPRG1, HRK/SPRED1, SPRED1/PLCB1, TRIM2/SPRED1, SIGLEC8/SPRED1, SMPD3/SPRED1, SPRED1/ZNF600, SPRED1/SDC2, MAP7/GPR34, PRSS33/SPRED1, SPRED1/DYNC2H1, CACNA2D3/SPRED1, ADAM23/GPR34, ADAM23/MAP7, ADAM23/PLCB1, ADAM23/SPRED1, ALOX15/GPR34, ALOX15/PLCB1, ALOX15/SPRED1, BAALC/GPR34, BAALC/PLCB1, BAALC/SPRED1, CACNA2D3/DYNC2H1, CACNA2D3/GPR34, CACNA2D3/PLCB1, CACNA2D3/SPRED1, CACNA2D3/ZNF600, GPR34/DYNC2H1, GPR34/GRAMD1C, GPR34/PLCB1, GPR34/TPRG1, GPR34/ZNF600, GPR82/DYNC2H1, GPR82/GPR34, GPR82/GRAMD1C, GPR82/PLCB1, GPR82/TPRG1, GPR82/ZNF600, GRAMD1C/DYNC2H1, GRAMD1C/PLCB1, GRAMD1C/ZNF600, HRK/DYNC2H1, HRK/GPR34, HRK/MAP7, HRK/PLCB1, HRK/SPRED1, HRK/ZNF600, IL5RA/DYNC2H1, IL5RA/GPR34, IL5RA/PLCB1, IL5RA/SPRED1, IL5RA/TRIM2, MAP7/BAALC, MAP7/CACNA2D3, MAP7/DYNC2H1, MAP7/GPR34, MAP7/GPR82, MAP7/GRAMD1C, MAP7/PLCB1, MAP7/SPRED1, MAP7/TPRG1, MAP7/ZNF600, PLCB1/DYNC2H1, PLCB1/TPRG1, PLCB1/ZNF600, PRSS33/GPR34, PRSS33/PLCB1, PRSS33/SPRED1, SDC2/DYNC2H1, SDC2/GPR34, SDC2/PLCB1, SDC2/ZNF600, SIGLEC8/DYNC2H1, SIGLEC8/GPR34, SIGLEC8/MAP7, SIGLEC8/PLCB1, SIGLEC8/SPRED1, SIGLEC8/TRIM2, SMPD3/DYNC2H1, SMPD3/GPR34, SMPD3/MAP7, SMPD3/PLCB1, SMPD3/SPRED1, SMPD3/TRIM2, SPRED1/DYNC2H1, SPRED1/GPR34, SPRED1/GPR82, SPRED1/GRAMD1C, SPRED1/PLCB1, SPRED1/SDC2, SPRED1/TPRG1, SPRED1/ZNF600, TRIM2/CACNA2D3, TRIM2/DYNC2H1, TRIM2/GPR34, TRIM2/GPR82, TRIM2/GRAMD1C, TRIM2/HRK, TRIM2/MAP7, TRIM2/PLCB1, TRIM2/SDC2, TRIM2/SPRED1, TRIM2/TPRG1, and TRIM2/ZNF600; wherein the IFN endotype signature pair is selected from: ETV7/PLEKHO1, IFITM3/ETV7, ETV7/APOL1, BATF2/ETV7, PLEKHO1/BATF2, ETV7/EPSTI1, EPSTI1/BATF2, IFITM3/BATF2, USP18/EPSTI1, ETV7/SEPTIN4, ETV7/LAMP3, SERPING1/BATF2, LAMP3/BATF2, LAMP3/SERPING1, APOL1/BATF2, APOL1/CLEC4F, APOL1/EPSTI1, APOL1/EXOC3L1, APOL1/HES4, APOL1/IFITM3, APOL1/LY6E, APOL1/RSAD2, APOL1/SEPTIN4, APOL1/SERPING1, APOL1/TPPP3, BATF2/EXOC3L1, BATF2/HES4, CLEC4F/BATF2, CLEC4F/EXOC3L1, EPSTI1/BATF2, EPSTI1/CLEC4F, EPSTI1/EXOC3L1, EPSTI1/HES4, EPSTI1/IFITM3, EPSTI1/LY6E, EPSTI1/RSAD2, EPSTI1/SERPING1, EPSTI1/TPPP3, ETV7/APOL1, ETV7/BATF2, ETV7/CLEC4F, ETV7/EPSTI1, ETV7/EXOC3L1, ETV7/HES4, ETV7/IFITM3, ETV7/LAMP3, ETV7/LY6E, ETV7/PLEKHO1, ETV7/RSAD2, ETV7/SEPTIN4, ETV7/SERPING1, ETV7/TPPP3, EXOC3L1/HES4, IFITM3/SERPING1, IFITM3/CLEC4F, IFITM3/TPPP3, IFITM3/LY6E, IFITM3/EXOC3L1, IFITM3/HES4, LAMP3/APOL1, LAMP3/BATF2, LAMP3/CLEC4F, LAMP3/EPSTI1, LAMP3/EXOC3L1, LAMP3/HES4, LAMP3/IFITM3, LAMP3/LY6E, LAMP3/RSAD2, LAMP3/SEPTIN4, LAMP3/SERPING1, LAMP3/TPPP3, LY6E/BATF2, LY6E/EXOC3L1, PLEKHO1/APOL1, PLEKHO1/BATF2, PLEKHO1/EPSTI1, PLEKHO1/EXOC3L1, PLEKHO1/IFITM3, PLEKHO1/LAMP3, PLEKHO1/RSAD2, PLEKHO1/SEPTIN4, PLEKHO1/SERPING1, RSAD2/BATF2, RSAD2/CLEC4F, RSAD2/EXOC3L1, RSAD2/HES4, RSAD2/IFITM3, RSAD2/LY6E, RSAD2/SERPING1, RSAD2/TPPP3, SEPTIN4/BATF2, SEPTIN4/CLEC4F, SEPTIN4/EPSTI1, SEPTIN4/EXOC3L1, SEPTIN4/HES4, SEPTIN4/IFITM3, SEPTIN4/LY6E, SEPTIN4/RSAD2, SEPTIN4/SERPING1, SEPTIN4/TPPP3, SERPING1/BATF2, SERPING1/CLEC4F, SERPING1/EXOC3L1, SERPING1/HES4, SERPING1/LY6E, SERPING1/TPPP3, TPPP3/BATF2, and TPPP3/EXOC3L1; and wherein the ADA endotype signature pair is selected from: LGALS3BP/OTOF, LGALS3BP/IF127, LGALS3BP/KIF14, LGALS3BP/CENPF, GTSE1/LGALS3BP, LGALS3BP/KCTD14, LGALS3BP/PDIA4, LGALS3BP/TSHR, LGALS3BP/PLAAT2, OTOF/IF127, IGF1/LGALS3BP, CDC45/LGALS3BP, LGALS3BP/KIF15, LGALS3BP/IGLL5, LGALS3BP/MIXL, CAV1/LGALS3BP, CAV1/OTOF, CDC45/LGALS3BP, CDC45/OTOF, CENPF/KCTD14, GPRC5D/OTOF, GTSE1/LGALS3BP, GTSE1/OTOF, IGF1/LGALS3BP, IGF1/OTOF, KCTD14/KLHL14, KCTD14/PDIA4, KCTD14/TSHR, KIF14/KCTD14, LGALS3BP/CENPF, LGALS3BP/GPRC5D, LGALS3BP/IFI27, LGALS3BP/IGLL5, LGALS3BP/KCTD14, LGALS3BP/KIF14, LGALS3BP/KIF15, LGALS3BP/KLHL14, LGALS3BP/MIR155HG, LGALS3BP/MIXL1, LGALS3BP/OTOF, LGALS3BP/PDIA4, LGALS3BP/PLAAT2, LGALS3BP/SDC1, LGALS3BP/SLC16A14, LGALS3BP/TSHR, OTOF/CENPF, OTOF/IF127, OTOF/IGLL5, OTOF/KCTD14, OTOF/KIF14, OTOF/KIF15, OTOF/KLHL14, OTOF/MIR155HG, OTOF/MIXL1, OTOF/PDIA4, OTOF/PLAAT2, OTOF/SDC1, OTOF/SLC16A14, OTOF/TSHR, PLAAT2/KCTD14, TNFRSF17/LGALS3BP, and TNFRSF17/OTOF.

The present disclosure also includes a kit for predicting severity of sepsis in a subject, wherein the severity of the sepsis is selected from high severity sepsis, intermediate severity sepsis and low severity sepsis, wherein high severity sepsis means a sequential organ failure assessment (SOFA) score of greater than or equal to 5, intermediate severity sepsis means a SOFA score of greater than or equal to 2 but less than 5, and low severity sepsis means a SOFA score of less than 2, the kit comprising gene specific reagents, each of the gene specific reagents capable of detecting an expression product of a respective one of a plurality of genes or complement thereof selected from the group consisting of ABCA13, ADAMTS2, ADAMTS3, AK5, ANKRD22, ANKRD34B, ANLN, AQP1, ARG1, ARHGAP44, ARHGEF17, ASPM, ATP1B2, AURKA, AZUl, BAIAP3, BPI, Clorf226, CACNB4, CCL4L2, CCN3, CCNA1, CD177, CD24, CDK1, CDKN3, CEACAM6, CEACAM8, CENPA, CFH, CHDH, CHIT1, CKAP2L, CLEC4C, CLEC4F, CLNK, COL17A1, CRISP2, CRISP3, CTSE, CTSG, CYP19A1, CYYR1, DEFA4, DENND2C, DEPDC1, DGKK, DLC1, DLGAP5, DNAH10, DOC2B, DSP, ELANE, ERG, FAM20A, FAM83A, FBN1, FFAR3, GOS2, GGT5, GLB1L2, GJB6, GPR84, GRAMD1C, GYPA, HBM, HMGB3, HP, HPGD, HRK, IGLL1, IL1R2, IL1RL1, INHBA, IQGAP3, ITGA7, ITGB4, KIF15, KIF20A, KLF14, LAMB3, LCN2, LGR4, LPL, LTF, MAFG, MERTK, METTL7B, MMP8, MMP9, MPO, MRC1, MROCKI, MS4A3, MS4A4A, NECAB1, NEIL3, NEK2, NRXN2, NUF2, OLAH, OLFM4, OLIG2, PCOLCE2, PCSK9, PHF24, PIGR, PLAAT2, PPARG, PRTN3, PTGES, PYCRI, RAB3IL1, RASGRF1, RETN, RHCE, RIPOR3, RPGRIP1, RRM2, S100A12, S100A8, SCN8A, SEMA6B, SERPINB10, SIGLEC8, SIL1, SLC16A1, SLC28A3, SLC39A8, SLC4A10, SLC51A, SLC6A19, SLC8A3, SLCO4A1, SMIMI, SMPDL3A, SPATC1, SPOP, SSBP2, TCN1, TCTEX1D1, TDRD9, TEAD2, TFRC, THBS1, TIMP3, TLN2, TMEM255A, TMEM45A, TNFAIP8L3, TNIP3, TROAP, TTK, VSIG4, WNT3, YPEL4, and ZDHHC19; and optionally instructions for use.

In an embodiment, the gene specific reagent is a gene specific probe that is capable of detecting the expression product (e.g., nucleic acid or protein) or the complement of a nucleic acid expression product for example, wherein detection of the nucleic acid or compliment thereof is subsequent to a suitable methodology for amplification. In an embodiment, the methodology for amplification comprises a polymerase chain reaction (PCR) amplification method or reverse transcriptase-(RT) PCR. Polynucleotide primers for reverse transcription of mRNA encoded by the gene, and/or for amplification of a nucleic acid sequence from the gene or from cDNA prepared from the gene encoded mRNA may also be provided in the kit.

In some embodiments, the kit comprises, consists essentially or consists of a microarray that comprises a plurality of the gene specific probes that are polynucleotides immobilized onto a solid support. In an embodiment, the microarray further comprises control polynucleotide probes specific for control sequences, such as housekeeping genes.

In an embodiment, the kit optionally further includes one or more other reagents for conducting a biological procedure, such as but not limited to buffers, salts, enzymes, enzyme co-factors, substrates, detection reagents, washing reagents, and combinations thereof. Additional components, such as buffers and solutions for the isolation and/or treatment of a test sample, may also be included in the kit. In a further embodiment, the kit additionally comprises one or more control sequences or samples. In some embodiments, one or more of the components of the kit are lyophilized and the kit further comprises reagents suitable for the reconstitution of the lyophilized component(s).

The various components of the kit are typically provided in suitable containers. In some embodiments, the container may itself be a suitable vessel for carrying out the biological procedure, for example, a microtitre plate. Where appropriate, the kit may also optionally contain reaction vessels, mixing vessels and other components that facilitate the preparation of reagents or a test sample, or the carrying out of the biological procedure. In some embodiments, the kit further includes one or more instruments for assisting with obtaining a test sample, such as but not limited to a syringe, pipette, forceps, or combinations thereof.

In some embodiments, reagents comprised in the kit and/or their containers may be color-coded to facilitate their use. When reagents are color-coded, addition of one reagent to another in a particular step may, for example, result in a change in the color of the mixture, thus providing an indication that the step was carried out.

In an embodiment, the kit contains instructions for use, which may be provided in any suitable format such as but not limited to in paper form, in computer-readable form, and/or in the form of directions or instructions for accessing a website. In another embodiment, the kit further comprises computer readable media comprising software, and/or directions or instructions for accessing a website that provides software, for example, to assist in the interpretation of results obtained from using the kit.

It will be appreciated by a person skilled in the art that embodiments for such methods for identifying a candidate agent for the treatment of sepsis and/or kits can also be varied, as appropriate, as described herein for the corresponding embodiments in the methods for classifying a subject into a sepsis mechanistic endotype and/or methods for predicting severity of sepsis in a subject, as the case may be.

### Endotype Specific Gene Signatures

GENES WITH OVERALL DISCRIMINATIVE SIGNATURE (MOST ALSO WORK IN PAIRS). NPS: AGFG1, ARG1, ATP9A, ANXA3, EFNA1, GADD45A, GPR84, HPGD, IL1R1, KLF14, KREMEN1, MIR646HG, MLLT1, NSUN7, OLAH, ORM2, PCOLCE2, PFKFB2, SLC51A, TNFAIP8L3, ZDHHC19. INF: BNIP3L, CA1, FAM83A, FECH, GLRX5, GYPA, IFIT1B, RHCE, RIOK3, RNF182, SLC6A19, SPTA1, THEM5, TLCD4, TSPAN5, TSPO2. IHD: ABCA6, ADAM23, ALOX15, CACNA2D3, DYNC2H1, GPR34, GRAMD1C, LPL, MAP7, MIR155HG, PLCB1, SDC2, SIGLEC8, SPRED1, SLC16A14, SMPD3, TPPP3, TPRG1, ZNF600. IFN: ANKRD22, APOL1, APOL4, BATF2, CARD17, CD274, EPSTI1, ETV7, GBP5, IFITM3, P2RY14, PLEKHO1, RSAD2, SERPING1, TFEC. ADA: CCL2, CDC45, CENPF, CLEC4F, GTSE1, IFI27, KCTD14, KIF14, KIF15, KLHDC7B, LGALS3BP, OTOF, PDIA4, SIGLEC1,

OTHER GENES WITH ENDOTYPE DIAGNOSTIC POTENTIAL: NPS: ADAMTS3, AKR1C1, ALDH1A2, ALOX5AP, ALPL, AMPH, ANKRD55, BCL3, BTBD19, CA4, CD163L1, CD177, CD82, CST7, CYP19A1, CYSTM1, DAAM2, DGAT2, ECHDC3, ENTPD7, EXOSC4, FFAR3, FGF13, FSTL4, GALNT14, GRAMD1A, GRB10, GYG1, HPGD, IER3, IL18RAP, IL1R2, IL1RN, IRAG1-AS1, KCNE1B, KCNMA1, MCEMP1, MKNK1, MMP9, MSRA, NECAB1, NSMCE1-DT, OPLAH, PDGFC, PFKFB3, PHF24, PI3, PLIN4, PLIN5, PLK3, POR, PROK2, RFX2, RGL4, ROM1, S100A12, S100P, SEMA6B, SHROOM4, SLP1, SOCS3, SPATC1, SPDYA, SPINK8, SPP1, ST6GALNAC3, SYN2, TDRD9, TMEM120A, TMIGD3, TSPO, UPP1, XCR1. INF: ABCG2, ACHE, ACKR1, ACSL6, ADD2, AHSP, ALAS2, ALDH5A1, ANK1, ANKRD9, AQP1, ARHGEF12, ARHGEF37, ARL4A, ATP1B2, ATP1B2, BBOF1, BCAM, BCL2L1, BLVRB, BPGM, Clorf116, CA2, CISD2, CLIC2, CR1L, CR1L, CTNNAL1, CTSE, CTSE, DCAF12, DMTN, DNAJC6, DPCD, DYRK3, EMID1, EPB42, ERFE, FAM210B, FAXDC2, FRMD4A, GMPR, GSPT1, GYPB, HBM, HEMGN, HEPACAM2, HMBS, IGF2BP2, ISCA1, ITLN1, KANK2, KCNH2, KDM7A-DT, KEL, KLC3, KLF1, KLHDC8A, KRT1, LRRC2, MAOA, MAOA, MARCHF8, MBNL3, MFSD2B, MRC2, MXI1, MYL4, NFIX, NUDT4, OSBP2, PAGE2B, PBX1, PCDH1, PGF, PLEK2, PNP, PRDX2, PTPRF, RAPIGAP, RBM38, RFESD, RFESD, RGCC, RGS16, RHAG, RHD, RIPOR3, RNF175, RUNDC3A, SEC14L4, SELENBP1, SELENOP, SFRP2, SGIP1, SIAH2, SLC14A1, SLC1A5, SLC22A23, SLC2A1, SLC6A8, SLC6A9, SLC7A5, SMIM5, SNCA, SOX6, SPTB, STRADB, TAL1, TENT5C, TFR2, TMCC2, TMOD1, TNS1, TRIM10, TRIM58, TSPAN7, TTC25, UBB, USP12, XK, YBX3, YPEL4. IHD: ADGRD1, ANGPT1, GPR82, HDAC9, IL5RA, KLHDC1, PRSS33, PTGDR2, PTGFRN, TBC1D12, TRIM2. IFN: EXOC3L1, IRF7, OAS1, SEPTIN4, LY6E, LAMP3. ADA: AGRN, CD38, CDCA7, CDT1, CTLA4, DHX58, EME1, FAM111B, HES4, IFI44L, IFIT3, IFNG-AS1, IL12RB2, IL4I1, KIF19, LAG3, MCM10, P2RY6, PACSIN1, PARM1, SAMD4A, SPATS2L, HERC5, TMPRSS3, TNFRSF13B, TSHR, TTC21A.

PREFERRED EMBODIMENTS. GENE PAIRS: NPS vs. Rest: GADD45A/EFNA1, EFNA1/MIR646HG, MIR646HG/KLF14, MLLT1/MIR646HG, ARG1/MLLT1, MLLT1/EFNA1, MLLT1/NSUN7, EFNA1/NSUN7, SLC51A/EFNA1, EFNA1/KLF14, ZDHHC19/EFNA1, EFNA1/AGFG1, NSUN7/KLF14, EFNA1/PFKFB2, MLLT1/KLF14. INF vs. Rest: FECH/TFEC, TFEC/IFIT1B, FECH/RNF182, IFIT1B/FECH, FECH/APOL4, FECH/GYPA, ITLN1/FECH, FECH/THEM5, IFIT1B/CA1, RHAG/FECH, FECH/FAM83A, RHCE/FECH, TFEC/CA1, SPTA1/FECH. IHD vs. Rest: MAP7/SPRED1, SPRED1/GPR34, IL5RA/SPRED1, SPRED1/TPRG1, HRK/SPRED1, SPRED1/PLCB1, TRIM2/SPRED1, SIGLEC8/SPRED1, SMPD3/SPRED1, SPRED1/ZNF600, SPRED1/SDC2, MAP7/GPR34, PRSS33/SPRED1, SPRED1/DYNC2H1, CACNA2D3/SPRED1. IFN vs. Rest: ETV7/PLEKHO1, IFITM3/ETV7, ETV7/APOL1, BATF2/ETV7, PLEKHO1/BATF2, ETV7/EPSTI1, EPSTI1/BATF2, IFITM3/BATF2, ETV7/BATF2, USP18/EPSTI1, ETV7/SEPTIN4, ETV7/LAMP3, SERPING1/BATF2, LAMP3/BATF2, LAMP3/SERPING1. ADA vs. Rest: LGALS3BP/OTOF, LGALS3BP/IF127, LGALS3BP/KIF14, LGALS3BP/CENPF, GTSE1/LGALS3BP, LGALS3BP/KCTD14, LGALS3BP/PDIA4, LGALS3BP/TSHR, LGALS3BP/PLAAT2, OTOF/IF127, IGF1/LGALS3BP, CDC45/LGALS3BP, LGALS3BP/KIF15, LGALS3BP/IGLL5, LGALS3BP/MIXL1

ADDITIONAL GENE PAIRS (AUC>0.75): NPS: ADAMTS3/PCOLCE2; ADAMTS3/ZDHHC19; ADAMTS3/SLC51A; ADAMTS3/HPGD; ADAMTS3/SEMA6B; ADAMTS3/EFNA1; ADAMTS3/AGFG1; ADAMTS3/NSUN7; ADAMTS3/TNFAIP8L3; ADAMTS3/KREMEN1; ADAMTS3/ORM2; ADAMTS3/MIR646HG; ADAMTS3/KLF14; AGFG1/NSUN7; AGFG1/TNFAIP8L3; AGFG1/KREMEN1; AGFG1/ORM2; AGFG1/MIR646HG; AGFG1/KLF14; ANXA3/GPR84; ANXA3/OLAH; ANXA3/ADAMTS3; ANXA3/PCOLCE2; ANXA3/ZDHHC19; ANXA3/SLC51A; ANXA3/HPGD; ANXA3/SEMA6B; ANXA3/EFNA1; ANXA3/AGFG1; ANXA3/NSUN7; ANXA3/TNFAIP8L3; ANXA3/KREMEN1; ANXA3/ORM2; ANXA3/MIR646HG; ANXA3/KLF14; ARG1/PFKFB2; ARG1/MLLT1; ARG1/ANXA3; ARG1/GPR84; ARG1/OLAH; ARG1/ADAMTS3; ARG1/PCOLCE2; ARG1/ZDHHC19; ARG1/SLC51A; ARG1/HPGD; ARG1/SEMA6B; ARG1/EFNA1; ARG1/AGFG1; ARG1/NSUN7; ARG1/TNFAIP8L3; ARG1/KREMEN1; ARG1/ORM2; ARG1/MIR646HG; ARG1/KLF14; ATP9A/EPB41L4B; ATP9A/IL1R1; ATP9A/GADD45A; ATP9A/ARG1; ATP9A/PFKFB2; ATP9A/MLLT1; ATP9A/ANXA3; ATP9A/GPR84; ATP9A/OLAH; ATP9A/ADAMTS3; ATP9A/PCOLCE2; ATP9A/ZDHHC19; ATP9A/SLC51A; ATP9A/HPGD; ATP9A/EMA6B; ATP9A/EFNA1; ATP9A/AGFG1; ATP9A/NSUN7; ATP9A/TNFAIP8L3; ATP9A/KREMEN1; ATP9A/ORM2; ATP9A/MIR646HG; ATP9A/KLF14; EFNA1/AGFG1; EFNA1/NSUN7; EFNA1/TNFAIP8L3; EFNA1/KREMEN1; EFNA1/ORM2; EFNA1/MIR646HG; EFNA1/KLF14; EPB41L4B/IL1R1; EPB41L4B/GADD45A; EPB41L4B/ARG1; EPB41L4B/PFKFB2; EPB41L4B/LLT1; EPB41L4B/ANXA3; EPB41L4B/GPR84; EPB41L4B/OLAH; EPB41L4B/ADAMTS3; EPB41L4B/PCOLCE2; EPB41L4B/ZDHHC19; EPB41L4B/SLC51A; EPB41L4B/HPGD; EPB41L4B/SEMA6B; EPB41L4B/EFNA1; EPB41L4B/AGFG1; EPB41L4B/NSUN7; EPB41L4B/TNFAIP8L3; EPB41L4B/KREMEN1; EPB41L4B/MIR646HG; EPB41L4B/KLF14; GADD45A/ARG1; GADD45A/PFKFB2; GADD45A/MLLT1; GADD45A/ANXA3; GADD45A/GPR84; GADD45A/OLAH; GADD45A/ADAMTS3; GADD45A/PCOLCE2; GADD45A/ZDHHC19; GADD45A/SLC51A; GADD45A/HPGD; GADD45A/SEMA6B; GADD45A/EFNA1; GADD45A/AGFG1; GADD45A/NSUN7; GADD45A/TNFAIP8L3; GADD45A/KREMEN1; GADD45A/ORM2; GADD45A/MIR646HG; GADD45A/KLF14; GPR84/OLAH; GPR84/ADAMTS3; GPR84/PCOLCE2; GPR84/ZDHHC19; GPR84/SLC51A; GPR84/HPGD; GPR84/SEMA6B; GPR84/EFNA1; GPR84/AGFG1; GPR84/NSUN7; GPR84/TNFAIP8L3; GPR84/KREMEN1; GPR84/ORM2; GPR84/MIR646HG; GPR84/KLF14; HPGD/SEMA6B; HPGD/EFNA1; HPGD/AGFG1; HPGD/NSUN7; HPGD/TNFAIP8L3; HPGD/KREMEN1; HPGD/ORM2; HPGD/MIR646HG; HPGD/KLF14; IL1R1/GADD45A; IL1R1/ARG1; IL1R1/PFKFB2; IL1R1/MLLT1; IL1R1/ANXA3; IL1R1/GPR84; IL1R1/OLAH; IL1R1/ADAMTS3; IL1R1/PCOLCE2; IL1R1/ZDHHC19; IL1R1/SLC51A; IL1R1/HPGD; IL1R1/SEMA6B; IL1R1/EFNA1; IL1R1/AGFG1; IL1R1/NSUN7; IL1R1/TNFAIP8L3; IL1R1/KREMEN1; IL1R1/ORM2; IL1R1/MIR646HG; IL1R1/KLF14; KREMEN1/ORM2; KREMEN1/MIR646HG; KREMEN1/KLF14; MIR646HG/KLF14; MLLT1/ANXA3; MLLT1/GPR84; MLLT1/OLAH; MLLT1/ADAMTS3; MLLT1/PCOLCE2; MLLT1/ZDHHC19; MLLT1/SLC51A; MLLT1/HPGD; MLLT1/SEMA6B; MLLT1/EFNA1; MLLT1/AGFG1; MLLT1/SUN7; MLLT1/TNFAIP8L3; MLLT1/KREMEN1; MLLT1/ORM2; MLLT1/MIR646HG; MLLT1/KLF14; NSUN7/TNFAIP8L3; NSUN7/KREMEN1; NSUN7/ORM2; NSUN7/MIR646HG; NSUN7/KLF14; OLAH/ADAMTS3; OLAH/PCOLCE2; OLAH/ZDHHC19; OLAH/SLC51A; OLAH/HPGD; OLAH/SEMA6B; OLAH/EFNA1; OLAH/AGFG1; OLAH/NSUN7; OLAH/TNFAIP8L3; OLAH/KREMEN1; OLAH/ORM2; OLAH/MIR646HG; OLAH/KLF14; ORM2/MIR646HG; ORM2/KLF14; PCOLCE2/ZDHHC19; PCOLCE2/SLC51A; PCOLCE2/HPGD; PCOLCE2/SEMA6B; PCOLCE2/EFNA1; PCOLCE2/AGFG1; PCOLCE2/NSUN7; PCOLCE2/TNFAIP8L3; PCOLCE2/KREMEN1; PCOLCE2/ORM2; PCOLCE2/MIR646HG; PCOLCE2/KLF14; PFKFB2/MLLT1; PFKFB2/ANXA3; PFKFB2/GPR84; PFKFB2/OLAH; PFKFB2/ADAMTS3; PFKFB2/PCOLCE2; PFKFB2/ZDHHC19; PFKFB2/SLC51A; PFKFB2/HPGD; PFKFB2/SEMA6B; PFKFB2/EFNA1; PFKFB2/AGFG1; PFKFB2/NSUN7; PFKFB2/TNFAIP8L3; PFKFB2/KREMEN1; PFKFB2/ORM2; PFKFB2/MIR646HG; PFKFB2/KLF14; SEMA6B/EFNA1; SEMA6B/AGFG1; SEMA6B/NSUN7; SEMA6B/TNFAIP8L3; SEMA6B/KREMEN1; SEMA6B/ORM2; SEMA6B/MIR646HG; SEMA6B/KLF14; SLC51A/HPGD; SLC51A/SEMA6B; SLC51A/EFNA1; SLC51A/AGFG1; SLC51A/NSUN7; SLC51A/TNFAIP8L3; SLC51A/KREMEN1; SLC51A/ORM2; SLC51A/MIR646HG; SLC51A/KLF14; TNFAIP8L3/KREMEN1; TNFAIP8L3/ORM2; TNFAIP8L3/MIR646HG; TNFAIP8L3/KLF14; ZDHHC19/SLC51A; ZDHHC19/HPGD; ZDHHC19/SEMA6B; ZDHHC19/EFNA1; ZDHHC19/AGFG1; ZDHHC19/NSUN7; ZDHHC19/TNFAIP8L3; ZDHHC19/KREMEN1; ZDHHC19/ORM2; ZDHHC19/MIR646HG; ZDHHC19/KLF14; INF: ANKRD22/GLRX5; ANKRD22/GYPA; ANKRD22/IFIT1B; ANKRD22/ITLN1; ANKRD22/KLHDC8A; ANKRD22/RHCE; ANKRD22/RNF182; ANKRD22/SPTA1; ANKRD22/THEM5; ANKRD22/TSPAN5; APOL4/BNIP3L; APOL4/CA1; APOL4/DYRK3; APOL4/FAM83A; APOL4/GLRX5; APOL4/GYPA; APOL4/IFIT1B; APOL4/ITLN1; APOL4/KLHDC8A; APOL4/RHAG; APOL4/RHCE; APOL4/RIOK3; APOL4/RNF182; APOL4/SPTA1; APOL4/THEM5; APOL4/TLCD4; APOL4/TMCC2; APOL4/TSPAN5; APOL4/TSPO2; BNIP3L/ANKRD22; BNIP3L/CA1; BNIP3L/CARD17; BNIP3L/CD274; BNIP3L/DYRK3; BNIP3L/FAM83A; BNIP3L/GBP5; BNIP3L/GLRX5; BNIP3L/GYPA; BNIP3L/IFIT1B; BNIP3L/ITLN1; BNIP3L/KLHDC8A; BNIP3L/P2RY14; BNIP3L/RHAG; BNIP3L/RHCE; BNIP3L/RNF182; BNIP3L/SPTA1; BNIP3L/TFEC; BNIP3L/THEM5; BNIP3L/TLCD4; BNIP3L/TMCC2; BNIP3L/TSPAN5; BNIP3L/TSPO2; CA1/ANKRD22; CA1/CARD17; CA1/DYRK3; CA1/FAM83A; CA1/GBP5; CA1/GLRX5; CA1/GYPA; CA1/IFIT1B; CA1/ITLN1; CA1/KLHDC8A; CA1/P2RY14; CA1/RHCE; CA1/RNF182; CA1/SPTA1; CA1/THEM5; CA1/TLCD4; CA1/TSPAN5; CD274/CA1; CD274/DYRK3; CD274/FAM83A; CD274/GLRX5; CD274/GYPA; CD274/IFIT1B; CD274/ITLN1; CD274/KLHDC8A; CD274/RHCE; CD274/RNF182; CD274/SPTA1; CD274/THEM5; CD274/TLCD4; CD274/TMCC2; CD274/TSPAN5; DYRK3/ANKRD22; DYRK3/CARD17; DYRK3/FAM83A; DYRK3/GBP5; DYRK3/GLRX5; DYRK3/GYPA; DYRK3/IFIT1B; DYRK3/ITLN1; DYRK3/KLHDC8A; DYRK3/P2RY14; DYRK3/RHCE; DYRK3/RNF182; DYRK3/SPTA1; DYRK3/THEM5; DYRK3/TLCD4; DYRK3/TSPAN5; FAM83A/ANKRD22; FAM83A/CARD17; FAM83A/GBP5; FAM83A/GLRX5; FAM83A/GYPA; FAM83A/IFIT1B; FAM83A/ITLN1; FAM83A/KLHDC8A; FAM83A/P2RY14; FAM83A/RHCE; FAM83A/RNF182; FAM83A/SPTA1; FAM83A/THEM5; FAM83A/TLCD4; FAM83A/TSPAN5; FECH/ANKRD22; FECH/APOL4; FECH/BNIP3L; FECH/CA1; FECH/CARD17; FECH/CD274; FECH/DYRK3; FECH/FAM83A; FECH/GBP5; FECH/GLRX5; FECH/GYPA; FECH/IFIT1B; FECH/ITLN1; FECH/KLHDC8A; FECH/P2RY14; FECH/RHAG; FECH/RHCE; FECH/RIOK3; FECH/RNF182; FECH/SPTA1; FECH/TFEC; FECH/THEM5; FECH/TLCD4; FECH/TMCC2; FECH/TSPAN5; FECH/TSPO2; GBP5/GLRX5; GBP5/GYPA; GBP5/IFIT1B; GBP5/ITLN1; GBP5/KLHDC8A; GBP5/RHCE; GBP5/RNF182; GBP5/SPTA1; GBP5/THEM5; GBP5/TSPAN5; GLRX5/CARD17; GLRX5/IFIT1B; GLRX5/RHCE; GLRX5/THEM5; GYPA/CARD17; GYPA/GLRX5; GYPA/IFIT1B; GYPA/ITLN1; GYPA/P2RY14; GYPA/RHCE; GYPA/RNF182; GYPA/THEM5; IFIT1B/CARD17; ITLN1/CARD17; ITLN1/GLRX5; ITLN1/IFIT1B; ITLN1/RHCE; ITLN1/RNF182; ITLN1/THEM5; KLHDC8A/CARD17; KLHDC8A/GLRX5; KLHDC8A/GYPA; KLHDC8A/IFIT1B; KLHDC8A/ITLN1; KLHDC8A/P2RY14; KLHDC8A/RHCE; KLHDC8A/RNF182; KLHDC8A/SPTA1; KLHDC8A/THEM5; KLHDC8A/TSPAN5; P2RY14/GLRX5; P2RY14/IFIT1B; P2RY14/ITLN1; P2RY14/RHCE; P2RY14/RNF182; P2RY14/THEM5; RHAG/ANKRD22; RHAG/CA1; RHAG/CARD17; RHAG/CD274; RHAG/DYRK3; RHAG/FAM83A; RHAG/GBP5; RHAG/GLRX5; RHAG/GYPA; RHAG/IFIT1B; RHAG/ITLN1; RHAG/KLHDC8A; RHAG/P2RY14; RHAG/RHCE; RHAG/RNF182; RHAG/SPTA1; RHAG/THEM5; RHAG/TLCD4; RHAG/TMCC2; RHAG/TSPAN5; RHAG/TSPO2; RHCE/CARD17; RHCE/IFIT1B; RHCE/THEM5; RIOK3/ANKRD22; RIOK3/BNIP3L; RIOK3/CA1; RIOK3/CARD17; RIOK3/CD274; RIOK3/DYRK3; RIOK3/FAM83A; RIOK3/GBP5; RIOK3/GLRX5; RIOK3/GYPA; RIOK3/IFIT1B; RIOK3/ITLN1; RIOK3/KLHDC8A; RIOK3/P2RY14; RIOK3/RHAG; RIOK3/RHCE; RIOK3/RNF182; RIOK3/SPTA1; RIOK3/TFEC; RIOK3/THEM5; RIOK3/TLCD4; RIOK3/TMCC2; RIOK3/TSPAN5; RIOK3/TSPO2; RNF182/CARD17; RNF182/GLRX5; RNF182/IFIT1B; RNF182/RHCE; RNF182/THEM5; SPTA1/CARD17; SPTA1/GLRX5; SPTA1/GYPA; SPTA1/IFIT1B; SPTA1/ITLN1; SPTA1/P2RY14; SPTA1/RHCE; SPTA1/RNF182; SPTA1/THEM5; SPTA1/TSPAN5; TFEC/CA1; TFEC/DYRK3; TFEC/FAM83A; TFEC/GLRX5; TFEC/GYPA; TFEC/IFIT1B; TFEC/ITLN1; TFEC/KLHDC8A; TFEC/RHAG; TFEC/RHCE; TFEC/RNF182; TFEC/SPTA1; TFEC/THEM5; TFEC/TLCD4; TFEC/TMCC2; TFEC/TSPAN5; TFEC/TSPO2; THEM5/CARD17; THEM5/IFIT1B; TLCD4/ANKRD22; TLCD4/CARD17; TLCD4/GBP5; TLCD4/GLRX5; TLCD4/GYPA; TLCD4/IFIT1B; TLCD4/ITLN1; TLCD4/KLHDC8A; TLCD4/P2RY14; TLCD4/RHCE; TLCD4/RNF182; TLCD4/SPTA1; TLCD4/THEM5; TLCD4/TSPAN5; TMCC2/ABCA6; TMCC2/ANKRD22; TMCC2/CA1; TMCC2/CARD17; TMCC2/DYRK3; TMCC2/FAM83A; TMCC2/GBP5; TMCC2/GLRX5; TMCC2/GYPA; TMCC2/IFIT1B; TMCC2/ITLN1; TMCC2/KLHDC8A; TMCC2/P2RY14; TMCC2/RHCE; TMCC2/RNF182; TMCC2/SPTA1; TMCC2/THEM5; TMCC2/TLCD4; TMCC2/TSPAN5; TSPAN5/CARD17; TSPAN5/GLRX5; TSPAN5/GYPA; TSPAN5/IFIT1B; TSPAN5/ITLN1; TSPAN5/P2RY14; TSPAN5/RHCE; TSPAN5/RNF182; TSPAN5/THEM5; TSPO2/ANKRD22; TSPO2/CA1; TSPO2/CARD17; TSPO2/CD274; TSPO2/DYRK3; TSPO2/FAM83A; TSPO2/GBP5; TSPO2/GLRX5; TSPO2/GYPA; TSPO2/IFIT1B; TSPO2/ITLN1; TSPO2/KLHDC8A; TSPO2/P2RY14; TSPO2/RHCE; TSPO2/RNF182; TSPO2/SPTA1; TSPO2/THEM5; TSPO2/TLCD4; TSPO2/TMCC2; TSPO2/TSPAN5; IHD: ADAM23/GPR34; ADAM23/MAP7; ADAM23/PLCB1; ADAM23/SPRED1; ALOX15/GPR34; ALOX15/PLCB1; ALOX15/SPRED1; BAALC/GPR34; BAALC/PLCB1; BAALC/SPRED1; CACNA2D3/DYNC2H1; CACNA2D3/GPR34; CACNA2D3/PLCB1; CACNA2D3/SPRED1; CACNA2D3/ZNF600; GPR34/DYNC2H1; GPR34/GRAMD1C; GPR34/PLCB1; GPR34/PRG1; GPR34/ZNF600; GPR82/DYNC2H1; GPR82/GPR34; GPR82/GRAMD1C; GPR82/PLCB1; GPR82/TPRG1; GPR82/ZNF600; GRAMD1C/DYNC2H1; GRAMD1C/PLCB1; GRAMD1C/ZNF600; HRK/DYNC2H1; HRK/GPR34; HRK/MAP7; HRK/PLCB1; HRK/SPRED1; HRK/ZNF600; IL5RA/DYNC2H1; IL5RA/GPR34; IL5RA/PLCB1; IL5RA/SPRED1; IL5RA/TRIM2; MAP7/BAALC; MAP7/CACNA2D3; MAP7/DYNC2H1; MAP7/GPR34; MAP7/GPR82; MAP7/GRAMD1C; MAP7/PLCB1; MAP7/SPRED1; MAP7/TPRG1; MAP7/ZNF600; PLCB1/DYNC2H1; PLCB1/TPRG1; PLCB1/ZNF600; PRSS33/GPR34; PRSS33/PLCB1; PRSS33/SPRED1; SDC2/DYNC2H1; SDC2/GPR34; SDC2/PLCB1; SDC2/ZNF600; SIGLEC8/DYNC2H1; SIGLEC8/GPR34; SIGLEC8/MAP7; SIGLEC8/PLCB1; SIGLEC8/SPRED1; SIGLEC8/TRIM2; SMPD3/DYNC2H1; SMPD3/GPR34; SMPD3/MAP7; SMPD3/PLCB1; SMPD3/SPRED1; SMPD3/TRIM2; SPRED1/DYNC2H1; SPRED1/GPR34; SPRED1/GPR82; SPRED1/GRAMD1C; SPRED1/PLCB1; SPRED1/SDC2; SPRED1/TPRG1; SPRED1/ZNF600; TRIM2/CACNA2D3; TRIM2/DYNC2H1; TRIM2/GPR34; TRIM2/GPR82; TRIM2/GRAMD1C; TRIM2/HRK; TRIM2/MAP7; TRIM2/PLCB1; TRIM2/SDC2; TRIM2/SPRED1; TRIM2/TPRG1; TRIM2/ZNF600; IFN: APOL1/BATF2; APOL1/CLEC4F; APOL1/EPSTI1; APOL1/EXOC3L1; APOL1/HES4; APOL1/IFITM3; APOL1/LY6E; APOL1/RSAD2; APOL1/SEPTIN4; APOL1/SERPING1; APOL1/TPPP3; BATF2/EXOC3L1; BATF2/HES4; CLEC4F/BATF2; CLEC4F/EXOC3L1; EPSTI1/BATF2; EPSTI1/CLEC4F; EPSTI1/EXOC3L1; EPSTI1/HES4; EPSTI1/IFITM3; EPSTI1/LY6E; EPSTI1/RSAD2; EPSTI1/SERPING1; EPSTI1/TPPP3; ETV7/APOL1; ETV7/BATF2; ETV7/CLEC4F; ETV7/EPSTI1; ETV7/EXOC3L1; ETV7/HES4; ETV7/IFITM3; ETV7/LAMP3; ETV7/LY6E; ETV7/PLEKHO1; ETV7/RSAD2; ETV7/SEPTIN4; ETV7/SERPING1; ETV7/TPPP3; EXOC3L1/HES4; LAMP3/APOL1; LAMP3/BATF2; LAMP3/CLEC4F; LAMP3/EPSTI1; LAMP3/EXOC3L1; LAMP3/HES4; LAMP3/IFITM3; LAMP3/LY6E; LAMP3/RSAD2; LAMP3/SEPTIN4; LAMP3/SERPING1; LAMP3/TPPP3; LY6E/BATF2; LY6E/EXOC3L1; PLEKHO1/APOL1; PLEKHO1/BATF2; PLEKHO1/EPSTI1; PLEKHO1/EXOC3L1; PLEKHO1/IFITM3; PLEKHO1/LAMP3; PLEKHO1/RSAD2; PLEKHO1/SEPTIN4; PLEKHO1/SERPING1; RSAD2/BATF2; RSAD2/CLEC4F; RSAD2/EXOC3L1; RSAD2/HES4; RSAD2/IFITM3; RSAD2/LY6E; RSAD2/SERPING1; RSAD2/TPPP3; SEPTIN4/BATF2; SEPTIN4/CLEC4F; SEPTIN4/EPSTI1; SEPTIN4/EXOC3L1; SEPTIN4/HES4; SEPTIN4/IFITM3; SEPTIN4/LGALS3BP; SEPTIN4/LY6E; SEPTIN4/OTOF; SEPTIN4/RSAD2; SEPTIN4/SERPING1; SEPTIN4/TPPP3; SERPING1/BATF2; SERPING1/CLEC4F; SERPING1/EXOC3L1; SERPING1/HES4; SERPING1/LY6E; SERPING1/TPPP3; TPPP3/BATF2; TPPP3/EXOC3L1; ADA: CAV1/LGALS3BP; CAV1/OTOF; CDC45/LGALS3BP; CDC45/OTOF; CENPF/KCTD14; GPRC5D/OTOF; GTSE1/LGALS3BP; GTSE1/OTOF; IGF1/LGALS3BP; IGF1/OTOF; KCTD14/KLHL14; KCTD14/PDIA4; KCTD14/TSHR; KIF14/KCTD14; LGALS3BP/CENPF; LGALS3BP/GPRC5D; LGALS3BP/IFI27; LGALS3BP/IGLL5; LGALS3BP/KCTD14; LGALS3BP/KIF14; LGALS3BP/KIF15; LGALS3BP/KLHL14; LGALS3BP/MIR155HG; LGALS3BP/MIXL1; LGALS3BP/OTOF; LGALS3BP/PDIA4; LGALS3BP/PLAAT2; LGALS3BP/SDC1; LGALS3BP/SLC16A14; LGALS3BP/TSHR; OTOF/CENPF; OTOF/IFI27; OTOF/IGLL5; OTOF/KCTD14; OTOF/KIF14; OTOF/KIF15; OTOF/KLHL14; OTOF/MIR155HG; OTOF/MIXL1; OTOF/PDIA4; OTOF/PLAAT2; OTOF/SDC1; OTOF/SLC16A14; OTOF/TSHR; PLAAT2/KCTD14; TNFRSF17/LGALS3BP; TNFRSF17/OTOF;

GENE SEVERITY SIGNATURE: ABCA13, ADAMTS2, ADAMTS3, AK5, ANKRD22, ANKRD34B, ANLN, AQP1, ARG1, ARHGAP44, ARHGEF17, ASPM, ATP1B2, AURKA, AZUl, BAIAP3, BPI, Clorf226, CACNB4, CCL4L2, CCN3, CCNA1, CD177, CD24, CDK1, CDKN3, CEACAM6, CEACAM8, CENPA, CFH, CHDH, CHITI, CKAP2L, CLEC4C, CLEC4F, CLNK, COL17A1, CRISP2, CRISP3, CTSE, CTSG, CYP19A1, CYYR1, DEFA4, DENND2C, DEPDC1, DGKK, DLC1, DLGAP5, DNAH10, DOC2B, DSP, ELANE, ERG, FAM20A, FAM83A, FBN1, FFAR3, GOS2, GGT5, GLB1L2, GJB6, GPR84, GRAMD1C, GYPA, HBM, HMGB3, HP, HPGD, HRK, IGLL1, IL1R2, ILIRL1, INHBA, IQGAP3, ITGA7, ITGB4, KIF15, KIF20A, KLF14, LAMB3, LCN2, LGR4, LPL, LTF, MAFG, MERTK, METTL7B, MMP8, MMP9, MPO, MRC1, MROCKI, MS4A3, MS4A4A, NECAB1, NEIL3, NEK2, NRXN2, NUF2, OLAH, OLFM4, OLIG2, PCOLCE2, PCSK9, PHF24, PIGR, PLAAT2, PPARG, PRTN3, PTGES, PYCRI, RAB3IL1, RASGRF1, RETN, RHCE, RIPOR3, RPGRIP1, RRM2, S100A12, S100A8, SCN8A, SEMA6B, SERPINB10, SIGLEC8, SIL1, SLC16A1, SLC28A3, SLC39A8, SLC4A10, SLC51A, SLC6A19, SLC8A3, SLCO4A1, SMIMI, SMPDL3A, SPATC1, SPOP, SSBP2, TCN1, TCTEX1D1, TDRD9, TEAD2, TFRC, THBS1, TIMP3, TLN2, TMEM255A, TMEM45A, TNFAIP8L3, TNIP3, TROAP, TTK, VSIG4, WNT3, YPEL4, ZDHHC19

REDUCED GENE SEVERITY SIGNATURES. (1) CCL4L2, GPR84, HRK, MMP8, GGT5, RASGRF1; (2) ADAMTS2, RETN, MMP8, GOS2, CYP19A1, OLAH, SLC6A19, TNFAIP8L3.

The following non-limiting examples are illustrative of the present disclosure:

### Examples

### I. Methods

### (a) Study Design and Clinical Data Collection

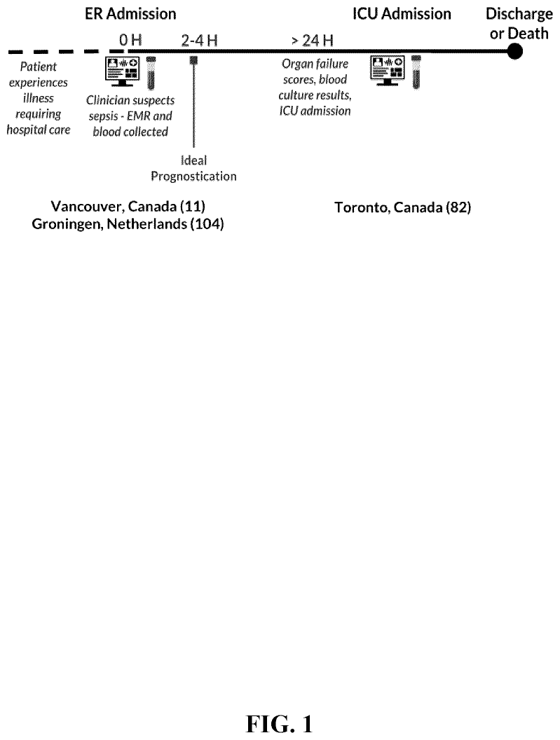

FIG. 1 shows the general study design. We enrolled adult patients (>18 years of age) with suspected sepsis, under ethical approval, within two hours of emergency room (ER) admission. Ethics approvals were obtained for all sequencing and bioinformatics studies, carried out in a manner blinded to patient identity. Suspicion of sepsis was based on the attending physician's informed opinion, with patients meeting Sepsis-3 criteria [Singer et al. 2016] or showing at least two SIRS criteria [Bone R C et al. Chest. 1992; 101:1644-1655. doi:10.1378/chest.101.6.1644] and suspicion of infection. Patients were excluded if death was impending (within 12 hours), if blood collection was unattainable, or consent was withheld. Enrollment included a full spectrum of individuals who might be suspected of being pre-septic, and while aware of the possibility that early therapy can strongly influence outcomes for such patients, we made no attempt to correct for treatments that might influence outcome measures since we were interested in the underlying mechanisms.

To enable retrospective association with gene expression data, various clinical metadata were collected at triage and within the 72 hours following ER admission, including demographics, ER test results reflecting signs of inflammation and infection, treatment data, and severity and outcome-specific clinical data, including measures of organ failure. In total 115 patients were recruited from ERs in Groningen, Netherlands (104) and Vancouver, Canada (11; sample collection limited by the SARS-CoV-2 pandemic). In addition, we recruited a cohort of 82 patients admitted to a Toronto, Canada tertiary care ICU with suspicion of pulmonary sepsis with and/or without Covid-19. Of these, 27 were confirmed to be infected with SARS-CoV-2 by subsequent viral PCR. Healthy control samples were also obtained from those who were either surgical controls or healthy volunteers (n=9).

(b) Blood collection and RNA-Seq

During usual ER blood sample collections for suspected sepsis patients, an additional 3 ml of blood was collected for RNA-Seq. Blood was collected in PAXgene™ Blood RNA tubes (BD Biosciences; San Jose, CA, USA) to ensure stabilization of intracellular RNA. After freezing, these were transported to Vancouver where RNA isolation and sample preparation was performed according to an established standard operating procedure (SOP) used in the REW Hancock lab (Lee, A. H. et al. Nature Communications 2019; 10(1):1092). Total RNA was extracted using PAXgene Blood RNA Kit (Qiagen; Germantown, MD, USA). Quantification and quality measures of total RNA were obtained using an Agilent™ 2100 Bioanalyzer (Agilent; Santa Clara, USA). Poly-adenylated RNA was captured using NEBNext™ Poly(A) mRNA Magnetic Isolation Module (NEB; Ipswich, USA). cDNA libraries were prepared using the KAPATm Total RNA HyperPrep Kit (Roche; Basel, Switzerland). RNA-Seq was then performed on Illumina™ Hi-Seq instrument using single read runs of 150 base-pair long sequence reads (excluding adapter/index sequences). Samples with RNA Integrity Numbers (RIN) below 6.5 were not sequenced. A standard data processing protocol was used [Lee A H et al. Nature Comm. 2019,10:1092. doi.org/10.1038/s41467-019-08794-x], including quality control using fastqc (v0.11.7) and multiqc (v1.6), alignment to the human genome (Ensembl GRCh38.92) using STAR (v2.6.0a), and read count assessments using htseq-count (v0.11.0). Finally, globin genes and genes with fewer than 10 counts were removed from count tables, and samples with fewer than one million total counts were not further analyzed. A variance stabilizing transformation (VST) was performed to render counts homoscedastic and normalized for varying library sizes. Following transformation, technical variation due to sequencing batch was removed using ComBat within the Surrogate Variable Analysis R package (3.30.1). Gene expression from the discovery and validation cohorts were treated independently of each other prior to VST normalization and batch correction to avoid signal leakage (also referred to as train-test leakage).

### (c) Cluster Validation and Consensus Clustering

In identifying subgroups or endotypes of a disease, one is trying to uncover genetic or clinical features that are common to a given subset of patients. The assumption is that patients with similar profiles share underlying mechanisms and outcomes and thus should be identified and treated accordingly. Cluster analysis or unsupervised data mining is the task of dividing a set of samples into a defined number of groups. Samples in the same group are more similar to samples in the same group when compared to samples in other groups.

Determining the optimal number of subgroups or clusters in a given disease, or more generally a data matrix is complex. In the absence of extensive domain knowledge and to avoid subjective decision making, we employed, to determine the number of sepsis endotypes, various empirical validity metrics, including consensus clustering cumulative distribution functions [Wilkerson M D, Hayes D N. Bioinformatics Appl. Note. 2010; 26:1572-1573. doi:10.1093/bioinformatics/btq170], Gap Statistic, Silhouette, Connectivity. We evaluated k values ranging from two to ten and applied validation metrics to determine the optimal number of clusters present. Further, to minimize the impact of noisy and irrelevant genes on the clustering results, we ranked genes by variance (mean absolute deviation) and performed cluster validation on the clustering inputs made up of the top 10 to 100 percent of genes (examining the top 10%, 25%, 50%, 75%, and 100% of genes). The optimal number of clusters was recorded for each clustering input for each cluster validation metric. Ultimately, the clustering input and k cluster value that were stable using the fewest input genes, were selected. We found that k cluster values of five and two were optimal using input genes for the top 10% of genes. To more broadly explore the diversity in transcriptional responses and underlying mechanisms, and enable further biological and clinical characterization, we selected 5 clusters.

Consensus k-medoids clustering was used to cluster patients on the basis of gene expression profiles. Consensus clustering, also referred to as ensemble clustering, is an algorithm that performs repeated clustering on subsamples of a portion of samples and genes. K-medoids with Manhattan distance was used for clustering since it is more robust to outliers, which are common in high-throughput omics data, when compared to other methods such as hierarchical clustering. Consensus clustering provided a consensus of the repeated clustering, which was robust to sampling variability. The consensus was represented as a consensus matrix, where each element is the fraction of times that two samples clustered together. Thus, a perfectly stable consensus matrix would consist of a matrix of 1's and 0's. The stability of the consensus matrix was quantified by CDF and area-under-the-CDF (AUCDF) plots.

### (d) Bioinformatics and Statistical Analysis

To determine the separation of clusters based on global gene expression, principal component analysis (PCA), differential expression relative to healthy controls, and functional pathway enrichment were explored. Dysregulated genes were identified in each endotype relative to healthy controls using DESeq2-based differential expression [Love M I et al. Genome Bio. 2014; 15:550. doi:10.1186/s13059-014-0550-8]. Endotypes were compared to a set of healthy control patients, with a separate set held out for the validation cohorts. A gene was considered differentially expressed (DE) if its absolute value fold change was >2 with an adjusted P value≤0.01. Functional characterization of differential expression was performed using an overrepresentation analysis (or enrichment analysis) of Reactome pathways, using the ReactomePA package [Fabregat A, et al. Nucleic Acids Res. 2018. doi:10.1093/nar/gkxl 132]. A pathway was considered enriched if its adjusted P value≤0.01. The pathway ratio represented the number of differentially expressed genes from that pathway relative to the total number of genes in the pathway. Cell proportions were estimated using the program CIBERSORT [https://cibersort.stanford.edu/; see, for example: Newman et al., 2015], since measured cell counts and differentials were not collected for several patients. Clinical measurements/variables were compared between clusters using non-parametric comparison of rank statistics (Kruskal-Wallis tests) and Chi-square tests depending on the variable type. The endotypes were compared in the context of impending severity and outcomes, as measured by sequential organ failure assessment (SOFA scores measured 24 and 72 hours post admission, length of hospital stay, treatments, ICU admission and mortality.

Subsequently gene expression signatures were identified by comparing global gene expression profiles between endotypes using differential expression analysis (top 200 differentially expressed genes when comparing each endotype to all others). This reflected the unique biological character of each endotype as revealed by plotting the gene expression differences onto protein:protein interaction (PPI) networks using NetworkAnalyst [Zhou G, et al. Nucleic Acids Res. 2019,47(W1):W234-W241]. Since protein:protein interactions (physical, metabolic or regulatory interactions) reveal the functionally-based interactions of individual proteins, the formation of tight and discrete networks indicated strong mechanistic differences between individual endotypes. In addition, gene set variation analysis (GSVA, an unsupervised method that calculates per sample enrichment scores as a function of gene expression inside and outside the gene set) [Hanzelmann S, et al. BMC Bioinformatics. 2013; 14:7. doi:10.1186/1471-2105-14-7] was used to assess the enrichment of the 200-gene signatures in each patient, which demonstrated that the signature corresponding to a single associated endotype was highly enriched in each of the classified patients. A classification scheme was derived for the endotype model using a supervised machine learning model, namely multinomial regression with least absolute shrinkage and selection operator (LASSO) [Tibshirani R. J. Royal Statistical Soc. B 2011:73:273-282] regularization. The trained multinomial endotype model was applied to each patient's gene expression profile (using the genes and model parameters) in the ICU validation cohorts to predict endotype status.

For some patients, the overall predicted prognoses for any given endotype was not accurate based on patient SOFA scores, ICU admission, and mortality. While not wishing to be limited by theory, this could reflect rapid and successful treatments like antibiotics to prevent further progression, or patient genetic background, or existing conditions that might influence deterioration. To this end, we characterized severity groups in a fashion similar to the endotypes, and built logistic regression models to predict severity groups. The goal was to identify gene signatures relevant to current definitions of sepsis. The Third International consensus definition of sepsis (Sepsis-3; Singer et al. 2016) includes SOFA as a proxy for organ dysfunction and sepsis, with increases in the SOFA score reflecting increases in mortality risk. The ER and ICU patients were combined to obtain a wide range of patient severity, based on SOFA scores measured 24 hours post ER/ICU admission. Patients were assigned to High (24-hour post ER/ICU admission SOFA scores >5; n=60), Intermediate (SOFA≥2 and <5; n=67), and Low (SOFA <2, n=67) severity groups. Similar to endotype characterization, gene expression profiles of severity groups were compared to healthy controls and to each other using differential expression and pathway enrichment.

### II. Results and Discussion

(a) Stratification of ER Patients with Suspicion of Sepsis into Five Clusters

Endotypes are subgroups of a condition, wherein each endotype is defined by distinct biological mechanisms, and they are clinically relevant. Several clinical trials have been unsuccessful in identifying biomarkers specific to sepsis [Marshall J C. Trends Molec. Med. 2014; 20:195-203. doi:10.1016/j.molmed.2014.01.007] while not wishing to be limited by theory, likely because the presence of heterogeneous subgroups is ignored. Results to date, largely driven by analysis of patient metadata and microarray transcriptomic studies, have indicated specific endotypes that have significantly higher severity scores and progress to worse outcomes; therefore, identifying endotype status early may permit aggressive interventions to prevent further progression of sepsis. Accordingly, the primary motivation for identifying endotypes in sepsis, particularly in its earliest stages, has been to dissect the heterogeneous molecular responses at play. Including a broad spectrum of patients also allowed us to identify possible molecular features differentiating sepsis and SIRS, which show similar early symptomology. Endotypes offer an insight into the specific molecular dysregulation occurring, enabling specific prognostic markers and therapeutic options to prevent deterioration.

We hypothesized that robust gene expression endotypes existed within suspected sepsis patients that are associated with disease severity and outcomes. Accordingly, a multi-cohort blood RNA-Seq study was performed on patients at first clinical presentation for whom the physician suspected the possibility of sepsis. Initial analyses to identify stable clusters of patients with similar transcriptomic profiles (endotypes) were performed with a cohort of 115 patients suspected of sepsis at first clinical presentation (within 2 hours of ER admission), originating from collaborating ERs in Netherlands and Canada (Table 1).

At the time of sampling, within two hours of emergency room (ER) admission, their quick Sequential Organ Failure Assessment (qSOFA) scores, which provided an early assessment of organ failure, tended to be modest, and as a cohort these patients demonstrated a full range from mild to relatively severe (range 0-3). Examining the demographic and clinical parameters of these patients (Table 1), the average age was 60 years (range 20-96), average SOFA scores measured 24 and 72 hours post admission were 2.0±0.18 (range 0-10) and 1.0±0.20 (range 0-11) respectively, and the average length of hospital stay was 8.1 days. Hospital mortality was relatively moderate at 13.9%, cf. the global mortality of 23% in sepsis [Rudd et al., 2020], consistent with the concept that around 50% of patients with prospective sepsis are subsequently found to have acquired more severe sepsis. The clinical heterogeneity observed within the cohorts exemplifies the need to accurately triage and prognosticate suspected sepsis patients. Cluster validation metrics were used to identify the optimal k value for clustering, namely 5 clusters according to Consensus Cluster CDF (examining the Top 10%, 25%, 50%, 75% and 100% of DE genes), and the minimal set of genes that should be used as input to a clustering algorithm.

### (b) The Endotype Model Provides Mechanistic Signatures of Early Sepsis

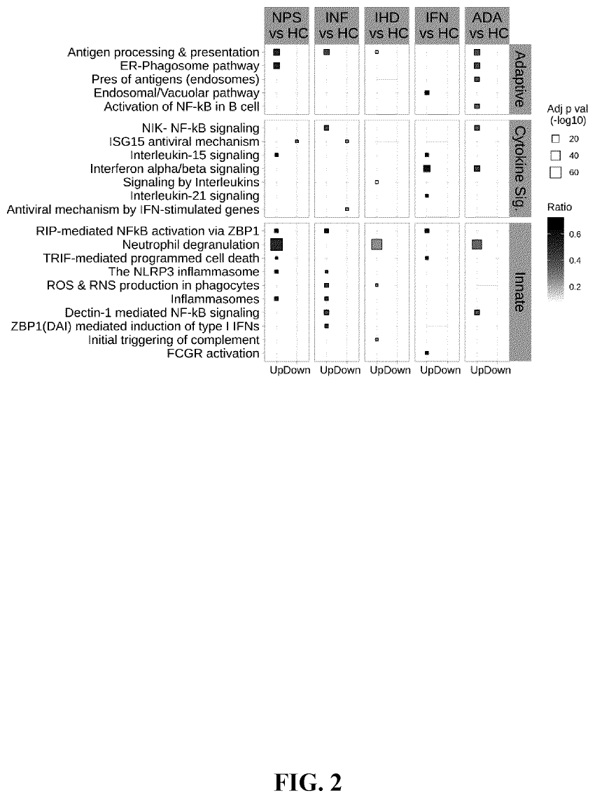

Patients belonged to one of five clusters, with each cluster representing a mechanistically-distinct endotype. We characterized the dominant biological mechanisms of each endotype by comparison to a set of 4 healthy controls (FIG. 2). Specifically, differential expression analysis was performed followed by over-representation (enrichment) analysis of up- and down-regulated pathways using the Reactome pathway database. Based on this, clusters 1 to 5 were named Neutrophilic-Suppressive (NPS), Inflammatory (INF), Innate Host Defence (IHD), Interferon (IFN) and Adaptive (ADA) endotypes, each based on several enriched pathways.

The NPS endotype showed a large dysregulation of gene expression when compared to healthy controls (5,341 total; 2,573 up-regulated; 2,768 down-regulated). Upregulated genes were related to aspects of the immune system pathways, particularly neutrophil degranulation, IL-15 signaling, TRIF-mediated programmed cell death and adaptive immune pathways (FIG. 2). The INF endotype also showed a large dysregulation of gene expression compared to healthy controls (3,830 total; 2,035 up-regulated; 1,795 down-regulated). Upregulated pathways were to some extent related to those seen in the NPS endotype, however, there was unique activation of the inflammatory NIK-NFκB signaling pathways and ROS/RNS production and reduced activation of neutrophil degranulation pathways.

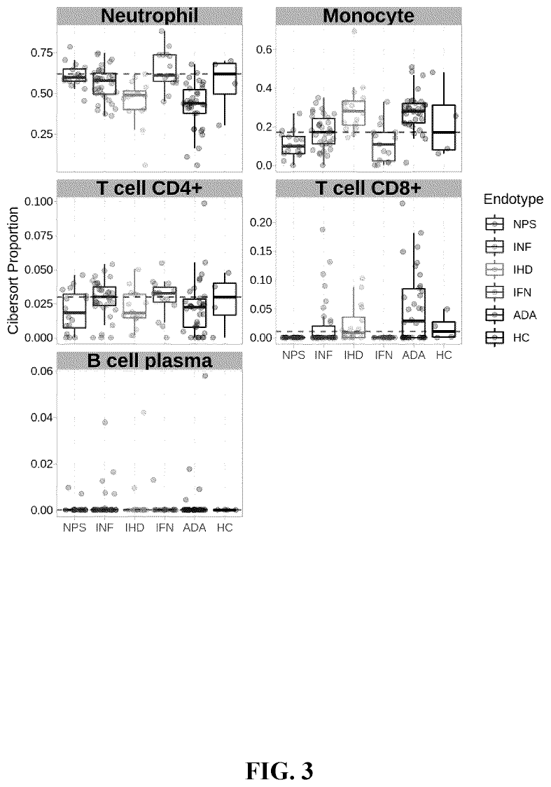

The IFN, ADA, and IHD endotypes collectively showed related gene expression trends, as indicated by principal component analysis, but also substantial differences. The IFN endotype showed 4,468 total (2,195 up-regulated; 2,273 down-regulated) dysregulated genes compared to healthy controls. The high expression of interferon-α, and -β signaling pathways was unique to this endotype. The ADA endotype showed substantial dysregulation of gene expression (3,227 total; 1,636 up-regulated; 1,591 down-regulated). The endotype was notable for upregulation of adaptive immune pathways. Furthermore, the endotype displayed the lowest number of neutrophils suggesting possible neutropenia and upregulation of lymphocytes (FIG. 3). Taken together, the IFN and ADA endotypes appeared to be the most immunocompetent or possibly less sick when compared to other endotypes. The IHD endotype showed the fewest dysregulated genes compared to the other endotypes (1,419 total; 721 up-regulated; 698 down-regulated). This endotype showed few enriched pathways, with the exception of neutrophil degranulation, complement cascade, and interleukin (IL) signaling.

A gene signature representing endotoxin tolerance/cellular reprogramming (CR; also referred to as the ET signature) that was predictive of the onset of severe sepsis and organ failure based on a retrospective meta-analysis of >600 patients and a modest clinical study of a cohort of 72 ER patients suspected of sepsis has been previously published (Pena O M, et al., 2014; see also: WO 2015/135071). The NPS, INF, and IFN endotypes showed similar fold changes with respect to the CR signature, but the NPS endotype had slightly higher fold changes, indicative of immunosuppression and poor outcomes.

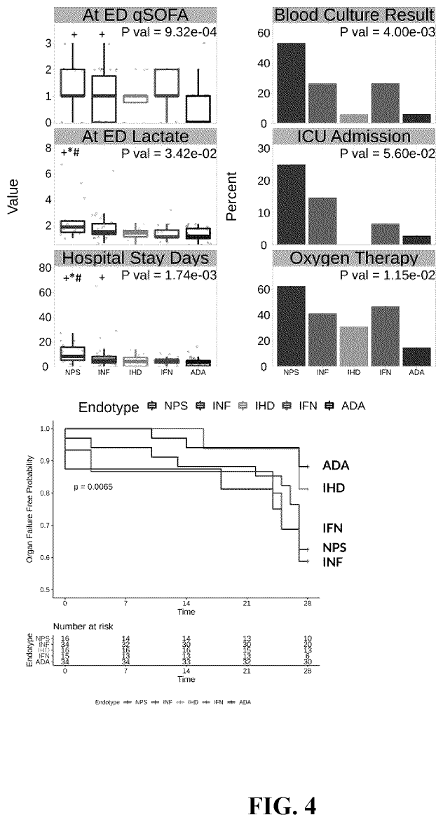

Based on clinical data (Table 2), the NPS and INF endotypes tended to be associated on average with more severe disease based on breathing difficulty (FIO₂) in the ER, hospital stay days, SOFA scores, blood culture and use of antibiotics, ICU admission, and increased risk of organ failure within 28 days of hospital admission (Table 2: FIG. 4). However, individual patients in each endotype had broadly different outcomes that could have been explained in part by the timeliness of appropriate treatment and other unknown variables. Using either Kruskal-Wallis or Chi square tests of significance, we determined endotypes were significantly associated with SOFA scores (p=0.00093), hospital stay duration (p=0.0017), ER FIO₂ (0.0077), blood culture (0.0040), blood lactate levels (0.034), and risk of organ failure within 28 days (0.0065). The clear associations between endotypes and clinical symptomology and outcomes indicated that the endotypes represent a useful tool to prognosticate patients, while their underlying mechanistic differences indicate the potential for personalized therapy.

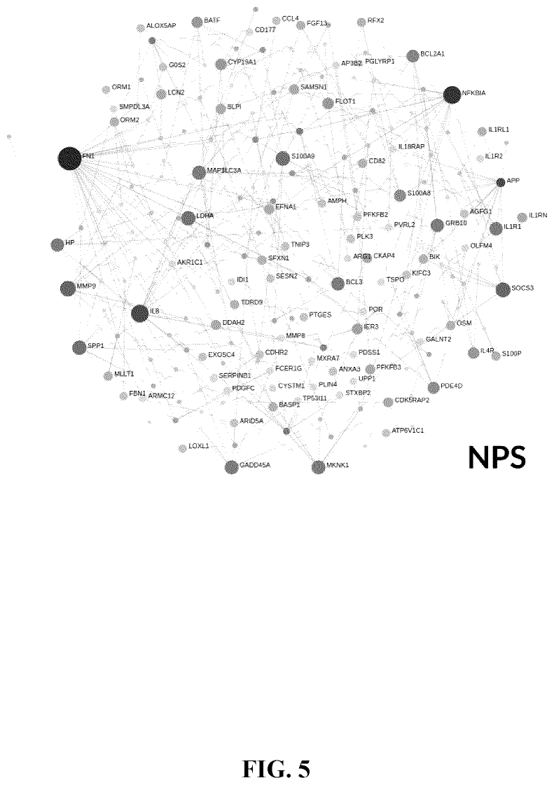

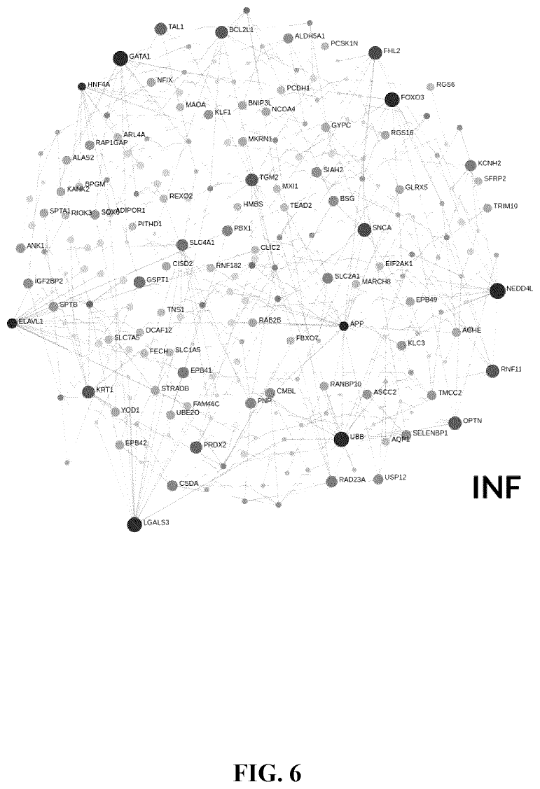

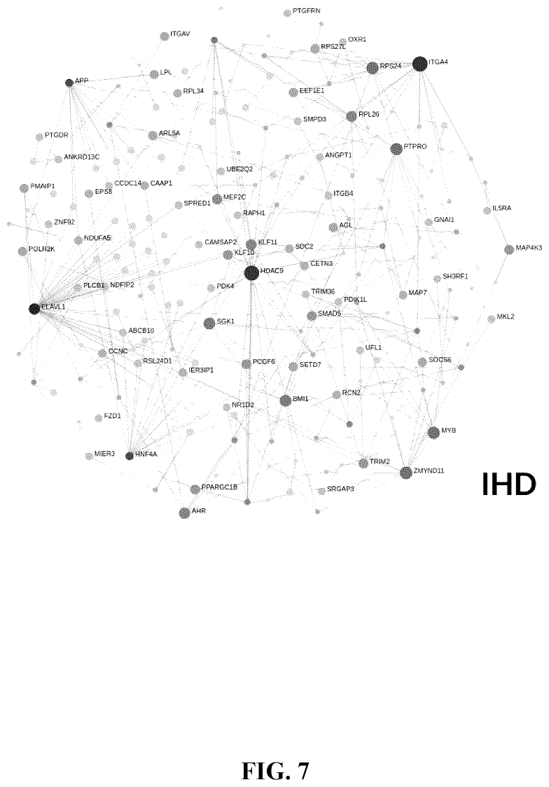

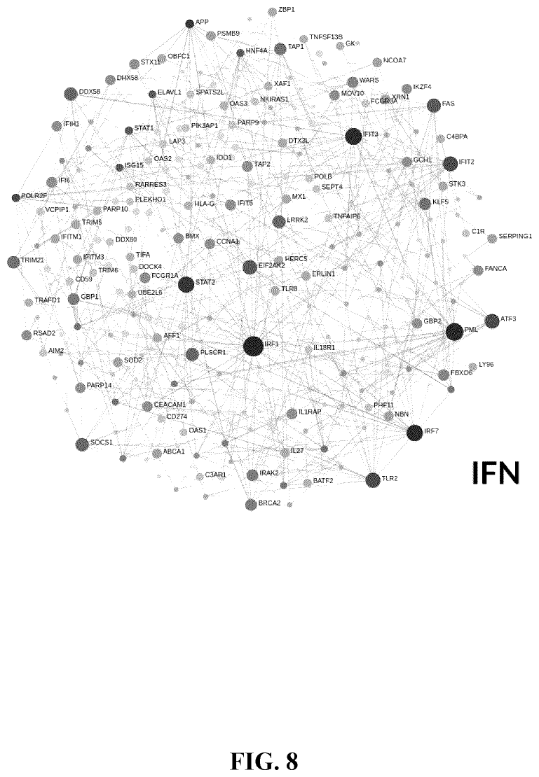

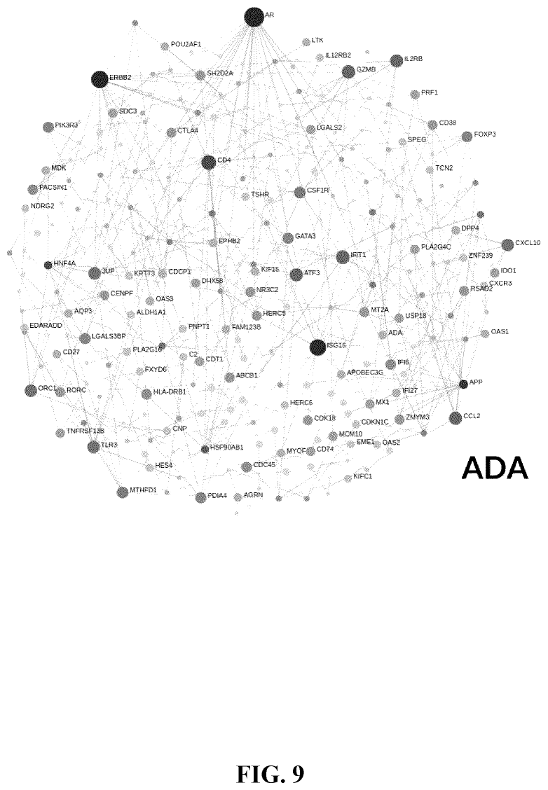

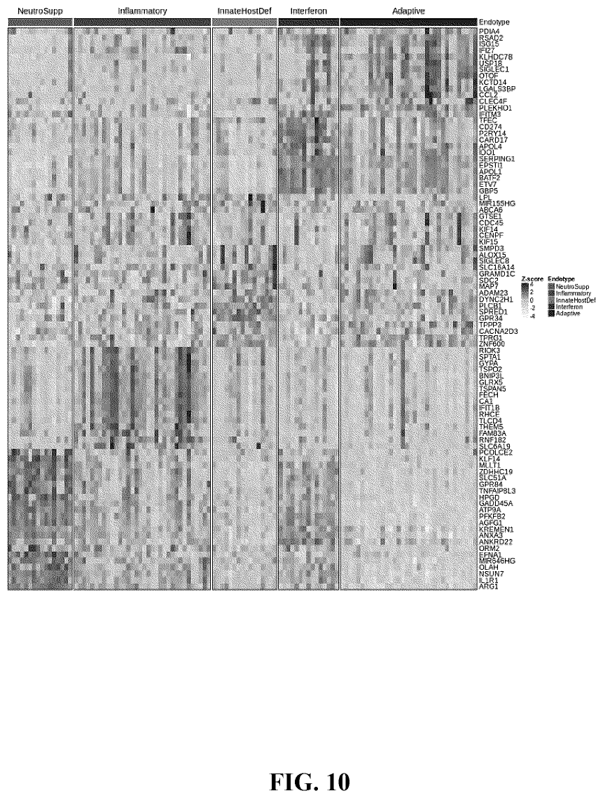

Subsequently gene expression signatures were identified by comparing global gene expression profiles between endotypes (top 200 differentially expressed genes when comparing each endotype to all others). This reflected the unique biological character of each endotype as revealed by plotting the gene expression differences onto protein:protein interaction (PPI) networks using NetworkAnalyst (FIGS. 5-9). The fact that, for each endotype, these unique genes form a coherent and well interconnected functional network indicates that they represent biologically meaningful clusters of genes, i.e. reflecting the underlying mechanisms of sepsis in the particular endotype. Darker coloured nodes (circles) represent genes from the signatures that are differentially expressed (DE), while light grey nodes are first order interacting and interconnecting nodes, while lines represent known (curated) functional interconnections from the database InnateDB (https://www.innatedb.ca/; Breuer et al., 2013). DE genes between endotypes were then used as input to a machine learning algorithm to obtain a signature that could be used to predict endotype status in a patient. Specifically, we derived a multinomial LASSO regularized regression to derive reduced gene sets to classify patients. This revealed that our signatures were very accurate in predicting endotypes with an Area under the receiver operating curve (AUC, a surrogate for accuracy, of 98%; Sensitivity of 80%; Specificity of 96%). There were 88 genes selected, which represents an effective signature to classify patients into endotypes (Table 3). These genes showed clear expression patterns with respect to the endotypes, indicating a moderate set of genes accurately differentiating each endotype (FIG. 10). Another 247 genes also showed clear expression patterns with respect to the endotypes (Table 4). In Tables 3 and 4, genes are bolded and arranged according to the endotype that they assist in classifying.

As can be clearly seen from Tables 3/4, most of these genes had high over-expression in one endotype (relative to individuals without sepsis) and either no increase or a decrease (i.e., negative fold changes) in expression in the other 4 endotypes. We examined for overlapping genes between our 88 gene signature and published literature on sepsis signatures. Generally, there was little overlap. Thus Maslove et al. (2012) described a 170 gene signature with only 2 overlapping genes (ARG1, ANXA3), Scicluna et al. (2017) described a 140 gene signature with only 9 overlapping genes [PLEKHO1 (oppositely regulated), APOL1, RIOK3, BNIP3L, GADD45A, PFKFB2 (not endotype specific), EPSTI1, SERPING1, GLRX5], while Sweeney et al. (2018) described a 33-gene signature with only 3 overlapping genes [PLEKHO1, GADD45A (oppositely regulated), ARG1]. Thus, the literature is ambiguous about genes PLEKHO1, GADD45A and PFKFB2. Furthermore, these studies looked at much later stage patients (already in the ICU) at which time sepsis is much easier to predict, and they also relied on microarray data which is considerably less accurate, and these studies were generally much smaller than ours. Critically it has been shown that at the time of first clinical presentation (in the ER) for every hour's delay in applying appropriate treatment there is a 7.6% increased risk of death from sepsis (Kumar A et al. Crit Care Med 2006; 34:1589-1596), so it is clear that these studies were looking at too late a time to provide meaningful clinical input that would impact strongly on treatment. Thus, it is perhaps not surprising that the endotypes described in those papers do not correspond in any simple fashion to the endotypes described in this patent application.

To try to reduce the size of signatures, we also tested whether the expression of pairs of genes from Tables 3/4 had diagnostic potential when predicting a specific endotype, compared to all others, by using logistic regression (e.g. 24 NPS DE genes led to 276 unique gene pairs tested). The data in Table 5 shows a broad range of gene pairs with excellent accuracy (expressed as Area under the Curve of Receiver Operating Characteristics, AUCROC or AUC), as well as testing Sensitivity (true positive rate) and Specificity (true negative rate). AUC helps one to visualize how well a machine learning classifier is performing, thus providing an estimate of accuracy. Sensitivity is the true positive rate (i.e., what proportion of the positive class got correctly classified) and Specificity is the true negative rate (i.e., what proportion of the negative class—in this case all other endotypes or rest—got correctly classified). The results are expressed as a fraction of 1 but can be considered equivalent to a percentage when multiplied by 100. Overall the range of AUC accuracy was 86.1-98.8%, with Sensitivity of 74.1-97.5%, and Specificity of 75.6-92.4%. Table 6 includes data from an expanded list of gene pairs that classify into specific endotypes when compared to all others. ROC/Accuracy, Sensitivity and Specificity are expressed as percent. It is thus clear from the results herein that these gene pairs, and predictably many of the genes in Tables 3/4 assessed as singles, pairs or other multiples, represent a highly effective method of classifying patients into endotypes, while they are still in the emergency ward.

(c) Different Mechanisms and Comparison with the Literature

Looking at the previous section, using consensus clustering we were able to test the hypothesis that robust mechanistically-distinct clusters exist within suspected sepsis patients. To confirm that each cluster represents clinically relevant-endotypes, we determined that the clusters were associated with clinical severity and outcomes. The endotype model stratified patients into one of five endotypes, each with a unique gene expression profile exhibiting diverse molecular responses. This has a very important implication. There are very few effective treatments for sepsis and to date our limited understanding of the mechanisms involved have limited the development of disease specific treatments. For example, more than 30 trials with different agents for suppressing the early hyper-inflammatory (cytokine storm) response in sepsis patients failed largely because of the different underlying mechanisms involved. To enable the development of personalized medicines for sepsis it is desirable to be able to understand the underlying mechanisms in subgroups (i.e., endotypes) of patients.

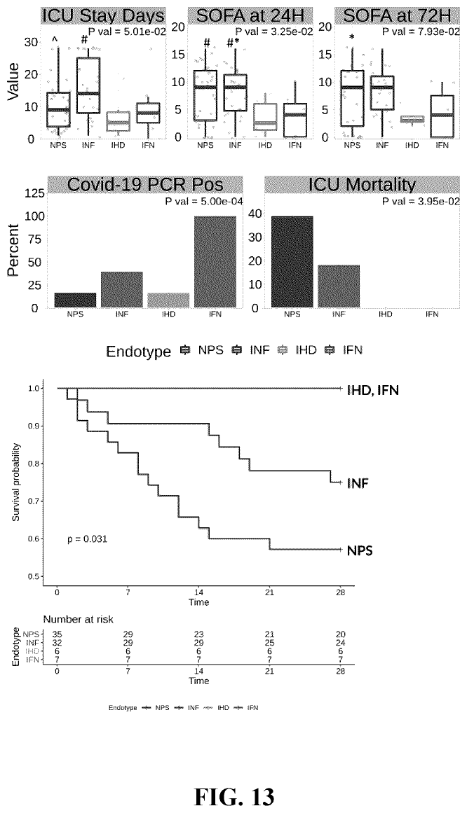

The five endotypes had diverse transcriptional profiles, with substantial heterogeneity observed in the innate, adaptive, and cytokine signaling pathways, and others (FIG. 2). The NPS and INF endotypes were associated with higher SOFA scores, longer hospital stays, and mortality among others (FIGS. 4, 13). In the ER cohort, the NPS and INF endotypes showed different cytokine signaling profiles and varying expression of the CR and inflammatory gene signatures, indicating the NPS endotype displayed a more immunosuppressive profile (FIG. 4). Studies indicate neutrophils do have paradoxical roles in sepsis, wherein their first-line host defences are beneficial, but when over-stimulated or reprogrammed contribute to organ dysfunction [Sônego F, et al. Frontiers in Immunology. 2016; 7:155. doi:10.3389/fimmu.2016.00155]. While not wishing to be limited by theory, this suggests neutrophil reprogramming may indeed be occurring, with the NPS and INF endotypes displaying varying states of reprogramming.

The other three endotypes demonstrated distinct and novel mechanisms, and tended to cluster on PCA while demonstrating significantly lower ER SOFA scores, and several other clinical parameters. Of these the ADA endotype was associated with substantially younger patients who showed down-regulation of the predictive CR signature, rapid resolution of SOFA scores, higher predicted levels of lymphocytes and upregulation of B-cell pathways, and was not identified in ICU patients. The IFN and ADA endotypes displayed the overall best prognoses, and less severe clinical symptomology (e.g., lower SOFA scores) and outcomes, cf. other endotypes.

The IFN endotype was particularly marked by an elevated expression of interferon signaling pathways. Intriguingly as shown below, in ICU patients, this endotype was associated with Covid-19 positivity. Thus, while not wishing to be limited by theory, the concerted interferon response could reflect a viral etiology [Li, H., et al. The Lancet, 2020; 395:1517-1520], or reflect strong inflammatory/anti-viral responses rather than immunosuppression that dominates in severe sepsis.

To the best of our knowledge, there are three studies which have explored endotypes in adult sepsis, and which have also analyzed associated clinical characteristics [Scicluna B P et al, 2017; Davenport E E, et al. 2016; Maslove D M, et al. 2012]. These studies looked at much later stage patients [already in the ICU] at which time sepsis is much easier to predict and relied on microarray data which is considerably less accurate and these studies were generally much smaller than ours. However, it has been shown that at the time of first clinical presentation [in the ER] for every hour's delay in applying appropriate treatment there is a 7.6% increased risk of death from sepsis, so it is clear that these studies were looking at too late a time to provide meaningful clinical input that would impact on treatment. Nonetheless, we were interested in comparing the endotypes our group identified to the ones previously published. Maslove et al. [2012] specifically profiled neutrophil gene expression to identify endotypes. They uncovered two endotypes, namely Subgroupl and Subgroup 2. Subgroup 1 was associated with higher severity scores, and increased expression of key inflammation pathways in neutrophils, specifically, cytokine signaling pathways and Toll-like receptor (TLR) signaling. This study is consistent with our findings, which showed an even earlier role of neutrophils. Davenport et al [2016] identified the Sepsis Response Signature 1 (SRS1) and Sepsis Response Signature 2 (SRS2) endotypes, with SRS1 associated with higher mortality. Similarly, the high mortality Mars1 endotype of Scicluna et al [2017], concluded on the basis of the reduced expression of TLR signaling, NFkB signaling, T cell receptor activity, and several metabolic pathways, that the endotype displayed hallmarks of immunosuppression. In our data, NPS, INF, and IHD endotypes all displayed hints of immunosuppression, and were evident in the ICU cohort. But most notably, the cellular underpinnings for these three endotypes are different from any previously described study. Whereas we identified neutrophils associated with immunosuppressed endotypes, the SRS and Mars endotypes were not associated with altered neutrophil proportions. The endotypes we identified deviate from several endotype models previously published by displaying a clear role of neutrophils in sepsis progression. Nevertheless, the evidence of immunosuppression in more severe patients is definite, regardless of cellular origin but the details seem to differ across these different studies and our study. This indicates endotypes characterizing suspected sepsis patients likely uncover some related patterns, which emphasizes the feasibility of identifying and prognosticating sepsis at first presentation to the ER and ICU, but also discrete differences suggesting that not all signatures have equal value.

(d) ICU Patients Including Those with Severe Covid-19 Infections Retained Endotypes

The presence of these mechanistically and clinically relevant sepsis endotypes were validated in a sub-cohort of 82 critically ill patients (Table 1) enrolled in the COLOBILI study (St. Michael's Hospital, Toronto). Patients had severe respiratory failure and suspected pulmonary sepsis on day-0/1 of ICU admission; of these PCR on nasopharyngeal and/or endotracheal tube aspirates confirmed SARS-CoV-2 RNA in 27 patients. This cohort demonstrated higher severity and poorer outcomes when compared to the ER cohorts (24% mortality cf. 14%).

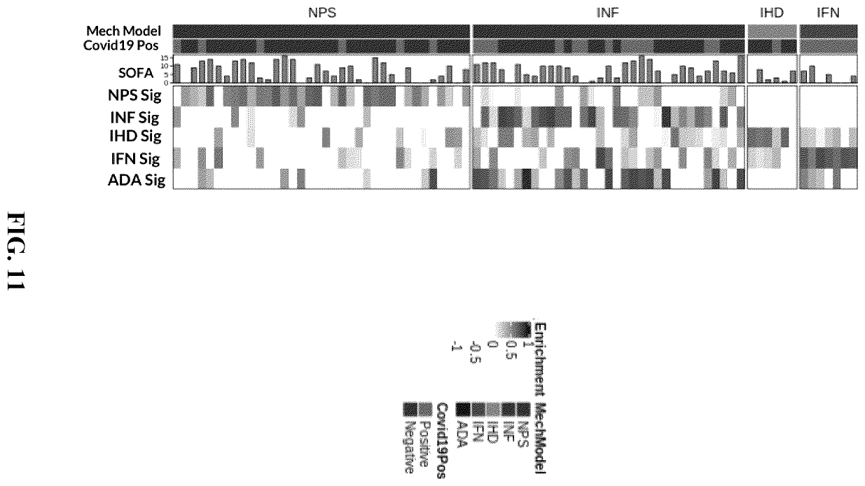

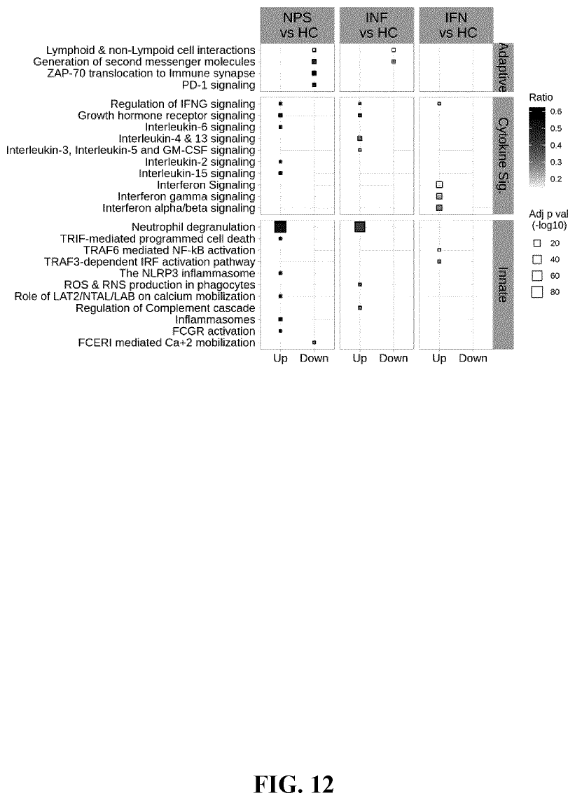

A Mechanistic endotype classifier was applied to predict endotype status using 88 genes from Table 3, and Gene-Set Variation Analysis was used to measure the enrichment of the five endotype signatures (FIG. 11). The model classified ICU patients into 4 endotypes with most (84%) fitting into the more severe NPS/INF endotypes. The ADA endotype was not identified, consistent with the observed downregulation of adaptive immune processes in later-stage sepsis patients. Interestingly, the IFN endotype was only found in 7/27 Covid-19 patients. The general trends in enriched pathways defining each endotype were recapitulated in the ICU (FIG. 12), when compared to the ER (FIG. 2), patients. In general, pathway trends were similar to those observed in the ER patients, with the exception of Neutrophil degranulation, which was enriched in NPS, INF, and IHD patients. While not wishing to be limited by theory, this may reflect the increased severity in ICU patients. The NPS and INF endotypes showed the worst prognosis, with higher 24-hour SOFA scores (mean 7-9±0.6; p=0-0035) (Table 7; FIG. 13), while the NPS endotype displayed substantially higher 28-day mortality when compared to the INF endotype. No patients from the IHD or IFN endotypes died. This indicated the IFN endotype reflected a robust/effective interferon response, while the IHD endotype might generally reflect less severe patients.

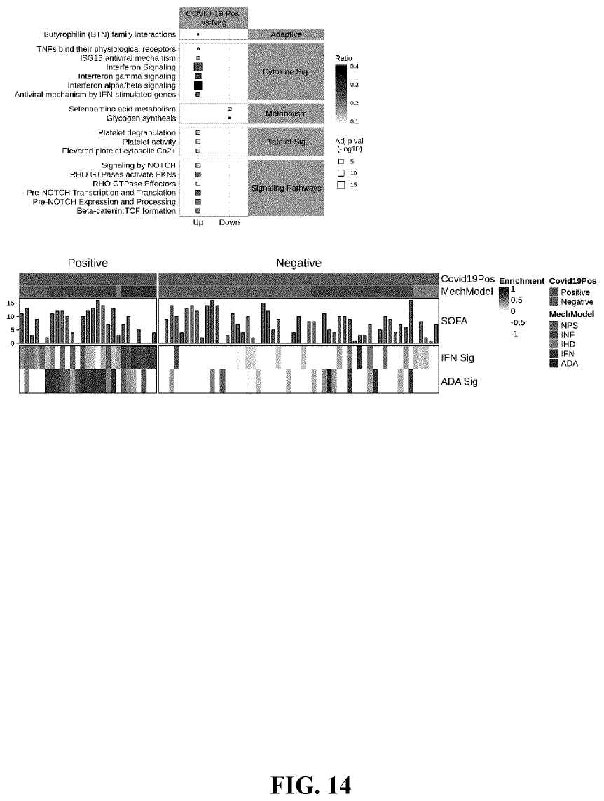

The patients in the ICU cohort were severely ill patients with suspicion of Covid-19. Final confirmation of Covid-19 positive infections was determined using multiple PCR analyses, resulting in a determination of 27 positive and 55 negative patients. Comparing gene expression profiles between SARS-CoV2 positive and negative patients demonstrated 1,221 DE genes (663 up; 558 down). As previously demonstrated [Sadanandam A et al. Cell Death Discov. 2020; 6, 141], interferon -α, -β, and -γ pathways, as well as NOTCH, RHO GTPases, WNT signaling pathways and platelet signaling pathways were upregulated in Covid-19 patients when compared to negative patients (FIG. 14, top left). Interferon pathways were also upregulated in non-Covid ICU patients, but Covid-19 positive patients demonstrated a relatively much larger increase in these and other anti-viral pathways. Importantly, Covid-19 patients grouped generally with other ICU patients in terms of endotype assignment (FIG. 14, right); this confirmed later stage Covid-19 patients generally display the same molecular responses as sepsis patients [Prescott H C, Girard T D. JAMA 2020; 324.8:739-740. doi:10.1001/jama.2020.14103]. Intriguingly the IFN endotype was identified only in Covid-19 patients and, although no patients were assigned to the Adaptive endotype by the endotype classifier, both the IFN and ADA endotype signatures were generally enriched in PCR-positive patients. While not wishing to be limited by theory, this likely reflected cellular immune system alterations in Covid-19 patients. Thus, it is evident that the molecular responses governing each endotype reflect markers of severity, and are generally applicable to patients with all-cause sepsis, including Covid-19 sepsis. Of note, no patients assigned to the IFN endotype died, which, while not wishing to be limited by theory, might suggest this endotype identified patients with viral infections moderated by effective anti-viral responses, and better prognoses.

An objective was to further validate our endotypes in ICU patients presenting with severe sepsis. Given the current pandemic, we had the unique opportunity to recruit ICU patients suspected of Covid-19. This allowed us to determine whether our endotypes were applicable to severe ICU patients, Covid-19, and more generally to sepsis patients with viral infections. We first examined the major gene expression differences between severe ICU patients and ER patients suspected of sepsis. It was evident that adaptive immune pathways were downregulated compared to the situation in suspected sepsis (ER) patients and healthy controls. It is discussed in the literature that severely sick septic patients typically display a suppressed adaptive immune system, or more generally immunosuppression, featuring T cell exhaustion, lymphocyte apoptosis, and diminished cytotoxicity [Hotchkiss et al. 2013]. When classifying patients into endotypes, we observed that there were no ADA endotype patients. Considering adaptive immune processes were downregulated, this explains the observation. Mortality was much more likely in the NPS and INF endotypes. Considering severely ill sepsis patients feature robust signals of immunosuppression, it seems likely that the NPS endotype captures this phenotype. Taken together, the cohort showed us that early signatures of SIRS and sepsis are applicable to severely ill patients collected within the first day of ICU admission and early changes appear to persist through early sepsis and SIRS to full blown sepsis.

The patients within the ICU cohort were suspected of Covid-19, and this constituted part of the inclusion criteria. Therefore, we also had the opportunity to explore differences between Covid-19 positive and negative patients, especially in the context of all cause sepsis endotypes we identified in ER patients. Functional enrichment showed Type I and II Interferon related pathways were upregulated in Covid-positive patients. This has been observed in previous literature exploring Covid-19 [Prescott H C, and Girard T D, 2020], and generally observed in viral infections given the role interferons play in curbing virus translation. Generally, the Covid-19 patients did not exclusively fall into one endotype, although most Covid-positive patients showed upregulation of the IFN (and ADA) signature. This may indicate the Interferon signature may be useful to identify viral infections. Many studies show that Covid-19 patients display evidence of excessive and dysfunctional neutrophils [Parackova Z et al. Cells. 2020; 9(10). doi:10.3390/cells9102206]. Considering the endotypes discovered reflect the same processes, it is evident the neutrophilic role in infection applies generally to severity and disease outcomes.

### (e) Supervised Analysis of SOFA-Based Severity Groups Displayed Signatures not Fully Captured by Endotypes

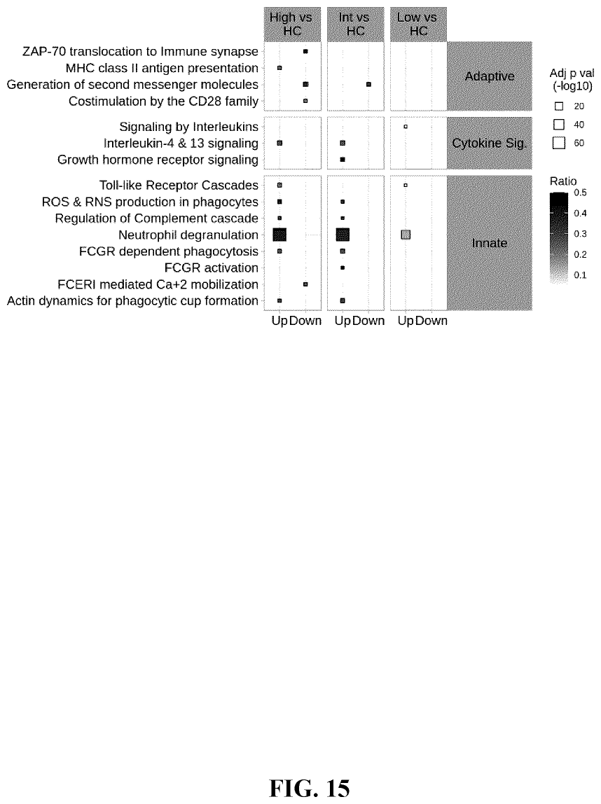

In general patients within the NPS and INF endotypes progressed to poorer outcomes when compared to the IHD, IFN, and ADA endotypes. However, for some patients the predicted prognoses were not accurate based on actual patient SOFA scores (an assessment of organ failure), ICU admission, and mortality. While not wishing to be limited by theory, this might reflect rapid treatments like antibiotics to prevent further progression, genetic background or existing conditions that could influence deterioration. We explored whether there were early gene expression differences between patients in High (SOFA scores ≥5), Intermediate (SOFA≥2 and <5), and Low (SOFA <2) severity groups (measured 24 hours after admission), representing signatures of severity. To capture the full range of severity observed, patients within the ER cohort and ICU cohort were included. Specifically, High severity patients (n=60) progressed to SOFA scores greater than or equal to five assuming baseline scores of zero; Intermediate severity patients progressed to SOFA scores between two and five (n=67); Low severity patients progressed to SOFA scores between zero and one (n=67). We identified DE genes by comparing each group to the healthy controls (n=9), followed by pathway enrichment (FIG. 15). There were 359 (336 up-regulated; 23 down-regulated), 2297 (1266 up-regulated; 1031 down-regulated), and 2068 (1333 up-regulated; 735 down-regulated) when comparing the Low, Intermediate, and High severity groups to healthy controls, respectively. The 157 genes that showed a pattern of differential expression in the more severe cf. less severe patients is shown in Table 8. A reduced 73 gene set was obtained by LASSO regularization (* in Table 8).

Using the various sets of DE genes, the hypothesis-based CR signature and an 8-gene sub-signature, we trained classification models predictive of severity group (Table 9). Specifically, logistic regression (with LASSO regularization) was used to predict High vs. Low (represented the extreme phenotypes) and High+Intermediate vs. Low severity groups. The models predicting High vs. Low severity groups performed quite well across the training and test sets, which did not include patients with Intermediate severity. The models predicting High/Intermediate patients vs. Low severity groups performed fairly and were comparable or better than models published by other groups that were trained on often questionable sepsis proxies like blood culture and clinician diagnoses rather than SOFA-based severity.

These data show that a six gene sub-signature, CCL4L2, GPR84, HRK, MMP8, GGT5, RASGRF1, selected from those genes in Table 8, was capable of accurately predicting severity as early as the first clinical presentation in the emergency room, and did this almost as effectively as the entire set of DE genes.

While the disclosure has been described with reference to what are presently considered to be the preferred examples, it is to be understood that the disclosure is not limited to the disclosed examples. To the contrary, the present disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### Full Citations for Documents Referred to in the Description

R C Bone et al., “Definitions for sepsis and organ failure and guidelines for the use of innovative therapies in sepsis” Chest 1992, 101:6, 1644-1655.

Breuer et al., “InnateDB: systems biology of innate immunity and beyond-recent updates and continuing curation” Nucleic Acids Res. 2013, 41:D1.
EE Davenport et al., “Genomic landscape of the individual host response and outcomes in sepsis: a prospective cohort study” The Lancet Respiratory Medicine, 2016, 4:4, 259-271.
A Fabregat et al., “The Reactome Pathway Knowledgebase” Nucleic Acids Res. 2018, 46:D1, D649-D655.
S Hänzelmann et al., “GSVA: gene set variation analysis for microarray and RNA-seq data” BMC Bioinformatics 2013, 14, 7.
R S Hotchkiss et al., “Immunosuppression in sepsis: a novel understanding of the disorder and a new therapeutic approach” Lancet Infect Dis. 2013, 13:3, 260-8.
LJ Kricka, “Nucleic acid detection technologies-labels, strategies, and formats” Clin Chem 1999, 45, 453-458.
A Kumar et al., “Duration of hypotension before initiation of effective antimicrobial therapy is the critical determinant of survival in human septic shock” Crit Care Med 2006, 34:6, 1589-96.
A H Lee et al., “Dynamic molecular changes during the first week of human life follow a robust developmental trajectory” Nat Commun. 2019, 10:1, 1092.
A Leligdowicz, and M A Matthay, “Heterogeneity in sepsis: new biological evidence with clinical applications” Critical Care 2019, 23, 80.
H Li et al., “SARS-CoV-2 and viral sepsis: observations and hypotheses” Lancet 2020, 395:10235, 1517-1520.
M I Love et al., “Moderated estimation of fold change and dispersion for RNA-seq data with DESeq2” Genome Biol. 2014, 15:12, 550.
J C Marshall, “Why have clinical trials in sepsis failed?” Trends Mol Med 2014 20:4, 195-203.
D M Maslove et al., “Identification of sepsis subtypes in critically ill adults using gene expression profiling” Crit Care 2012, 16:5, R183.
L McHugh et al., “A molecular host response assay to discriminate between sepsis and infection-negative systemic inflammation in critically ill patients: discovery and validation in independent cohorts” PLoSMed, 2015, 12:12, e1001916.
A M Newman et al., “Robust enumeration of cell subsets from tissue expression profiles” Nat Methods 2015, 12:5, 453-457.
Z Parackova et al., “Disharmonic inflammatory signatures in COVID-19: augmented neutrophils' but impaired monocytes' and dendritic cells' responsiveness” Cells 2020, 9:10, 2206.
O M Pena et al., “An endotoxin tolerance signature predicts sepsis and organ dysfunction at initial clinical presentation” EBioMedicine, 2014, 1:1, 64-71.
H C Prescott and TD Girard, “Recovery from severe COVID-19: Leveraging the lessons of survival from sepsis” JAMA 2020, 324:8, 739-740.
K E Rudd et al., “Global, regional, and national sepsis incidence and mortality, 1990-2017: analysis for the Global Burden of Disease Study” Lancet, 2020, 395:10219, 200-211.
A Sadanandam et al., “A blood transcriptome-based analysis of disease progression, immune regulation, and symptoms in coronavirus-infected patients” Cell Death Discov 2020, 6, 141.
B P Scicluna et al., “A molecular biomarker to diagnose community-acquired pneumonia on intensive care unit admission” Am J Respir Crit Care Med 2015, 192:7, 826-35.
B P Scicluna et al., “Classification of patients with sepsis according to blood genomic endotype: a prospective cohort study” Lancet Respir Med. 2017, 5:10, 816-826.
M Singer et al., “The Third International Consensus Definitions for Sepsis and Septic Shock (Sepsis-3)” JAMA 2016, 315:8, 801-810.
F Sônego et al., “Paradoxical Roles of the Neutrophil in Sepsis: Protective and Deleterious” Front Immunol 2016, 26:7, 155.
T E Sweeney et al., “A comprehensive time-course-based multicohort analysis of sepsis and sterile inflammation reveals a robust diagnostic gene set” Sci Transl Med 2015, 7:287, 287ra71.
T E Sweeney et al., “Unsupervised analysis of transcriptomics in bacterial sepsis across multiple datasets reveals three robust clusters” Crit Care Med. 2018, 46:6, 915-925.
R Tibshirani, “Regression shrinkage and selection via the lasso: a retrospective” J. Royal Statistical Soc. B 2011, 73, 273-282.
M D Wilkerson and D N Hayes, “ConsensusClusterPlus: a class discovery tool with confidence assessments and item tracking” Bioinformatics 2010, 26:12, 1572-1573.
G Zhou et al., “NetworkAnalyst 3.0: a visual analytics platform for comprehensive gene expression profiling and meta-analysis” Nucleic Acids Res 2019, 47:W1, W234-W241.

TABLE 1
Sepsis severity and outcomes of patients included
in the endotype discovery and validation cohorts*.

| | ER Cohort | | ICU Cohort | |
|---|---|---|---|---|
| Parameter | (N = 115) | | (N = 82) | |
| Age (years) | 59.9 ± 1.5 | | 61.7 ± 1.7 | (82) |
| Sex (% Female) | 43.5% | (50/115) | 30.5% | (25/82) |
| Location(s) | Groningen, | | Toronto, Canada | |
| | Netherlands (90%); | | (100%) | |
| | Vancouver, Canada (10%) | | | |
| Duration of illness | 4.2 ± 0.51 | | 6.6 ± 1.07 | (47) |
| before | | | | |
| ER/ICU arrival | | | | |
| (days) | | | | |
| ER qSOFA score | 0.9 ± 0.08 | | Not applicable | |
| ER/ICU 24 H SOFA | 2.0 ± 0.18 | | 7.7 ± 0.63 | (60) |
| score | | | | |
| ER/ICU 72 H SOFA | 1.0 ± 0.2 | | 7.8 ± 0.65 | (54) |
| score | | | | |
| Hospital/ICU | 6.2 ± 0.74 | | 11.8 ± 1.1 | (63) |
| stay (days) | | | | |
| Blood Culture | 21% | (24/112) | 14.3% | (9/63) |
| Positive (%) | | | | |
| ICU Admission | 9.6% | (11/115) | 98% | (80/82)** |
| Mortality | 13.9% | (16/115) | 24.4% | (20/82) |

| | | | |
|---|---|---|---|
| SARS-CoV-2 PCR | Not Available: | 32.9% | (27/82) |
| Positive | Pre-pandemic | | |

*The mean value, standard deviation, and total observations used are presented for numerical variables, i.e. mean ± SE (total observations when not equal to the size of the cohort). Categorical variables are presented as total positive observations, percent, and total observations, i.e. total % positives (of total observations).
**2 patients collected from ward.

TABLE 2
Clinical data of patients belonging to endotypes in the discovery ER cohorts*.

| | Mechanistic Endotypes | | | | | |
|---|---|---|---|---|---|---|
| | NPS | INF | IHD | IFN | ADA | P |
| Parameter | (N = 16) | (N = 34) | (N = 16) | (N = 15) | (N = 34) | Value |
| ER qSOFA | 1.3 ± 0.2 | 1.0 ± 0.1 | 0.9 ± 0.2 | 1.3 ± 0.2 | 0.5 ± 0.1 | 9.3e−4 |
| Hospital Stay (days) | 10.7 ± 1.9 | 8.3 ± 2.1 | 4.4 ± 1.1 | 4.7 ± 0.8 | 3.5 ± 0.6 | 1.7e−3 |
| | | (33) | | | | |
| Blood Culture Result | 53.3% | 26.5% | 6.2% | 26.7% | 6.2% | 0.0040 |
| | (8/15) | (9/34) | (1/16) | (4/15) | (2/32) | |
| ER FIO₂ (%) | 28.4 ± 5.0 | 26.7 ± 1.7 | 22.5 ± 1.0 | 23.4 ± 1.2 | 21.3 ± 0.3 | 0.0077 |
| | (15) | | (15) | | | |
| Sex, female | 81.2% | 41.2% | 31.2% | 20% | 44.1% | 0.0080 |
| | (13/16) | (14/34) | (5/16) | (3/15) | (15/34) | |
| Treatment - O₂ Therapy | 62.5% | 41.2% | 31.2% | 46.7% | 14.7% | 0.011 |
| | (10/16) | (14/34) | (5/16) | (7/15) | (5/34) | |
| Treatment - Antibiotics | 87.5% | 82.4% | 81.2% | 66.7% | 52.9% | 0.022 |
| | (14/16) | (28/34) | (13/16) | (10/15) | (18/34) | |
| ER Urea (mmol/L) | 8.5 ± 0.8 | 8.3 ± 1.3 | 10.5 ± 1.3 | 8.5 ± 0.85 | 6.8 ± 0.7 | 0.026 |
| | | (33) | | | | |
| ER Lactate | 2.5 ± 0.5 | 1.8 ± 0.2 | 1.4 ± 0.1 | 1.4 ± 0.2 | 1.3 ± 0.1 | 0.034 |
| | (14) | (26) | (12) | (12) | (20) | |
| ER Systolic (mmHg) | 113.1 ± 4.74 | 130.7 ± 4.09 | 137.6 ± 5.85 | 124.2 ± 6.83 | 129.6 ± 3.01 | 0.045 |
| ICU Admission | 25% | 14.7% | 0% (0/16) | 6.7% | 2.9% | 0.056 |
| | (4/16) | (5/34) | | (1/15) | (1/34) | |
| ER Respiratory Rate | 21.8 ± 1.5 | 22.8 ± 1.6 | 23.2 ± 1.4 | 23.8 ± 1.3 | 19.3 ± 0.9 | 0.061 |
| (breaths/min) | (16) | (33) | (15) | (14) | (28) | |
| Age | 60.1 ± 4.9 | 63.7 ± 3.0 | 66.2 ± 3.5 | 59.3 ± 6.0 | 53.4 ± 3.1 | 0.085 |
| ER MAP (mmHg) | 97.7 ± 5.0 | 109.5 ± 3.3 | 115.5 ± 4.8 | 104.5 ± 5.4 | 111.9 ± 2.9 | 0.089 |
| ER SOFA Score | 2.2 ± 0.4 | 2.4 ± 0.4 | 2.0 ± 0.5 | 1.9 ± 0.4 | 1.4 ± 0.3 | 0.15 |
| ER Diastolic (mmHg) | 73.6 ± 4.3 | 74.8 ± 2.3 | 76.3 ± 4.8 | 69.8 ± 3.7 | 80.4 ± 2.2 | 0.17 |
| ER Temperature (Celsius) | 37.9 ± 0.32 | 37.8 ± 0.17 | 37.5 ± 0.2 | 38.2 ± 0.24 | 37.8 ± 0.16 | 0.29 |
| Within 72 SOFA | 1.4 ± 0.56 | 1.3 ± 0.44 | 0.5 ± 0.18 | 1.1 ± 0.6 | 0.7 ± 0.31 | 0.29 |
| Readmit Within 28 Days | 26.7% | 17.6% | 18.8% | 0% | 24.2% | 0.36 |
| | (4/15) | (6/34) | (3/16) | (0/14) | (8/33) | |
| ER Creatinine (mg/dl) | 99.5 ± 10.9 | 94.5 ± 6.1 | 114.4 ± 10.8 | 117.9 ± 14.7 | 105.7 ± 19.0 | 0.36 |
| ER Aspartate amino- | 33 ± 4.5 | 38.6 ± 5.7 | 41.1 ± 10.7 | 30.5 ± 8.0 | 33.4 ± 3.96 | 0.37 |
| transferase (U/L) | | | (15) | | | |
| ER Alanine Amino | 32.7 ± 4.79 | 43.6 ± 6.08 | 31.6 ± 5.31 | 36.1 ± 11.31 | 36.6 ± 4.1 | 0.41 |
| transferase (IU/L) | | | (15) | | | |
| ER Alkaline phosphatase | 127.9 ± 16.0 | 169.5 ± 38.8 | 119.6 ± 19.0 | 90.9 ± 10.1 | 187.4 ± 52.3 | 0.45 |
| (U/L) | | (33) | (14) | | (32) | |
| Duration of Illness Prior to | 4.2 ± 1.1 | 4.1 ± 1.1 | 3.1 ± 0.5 | 3 ± 1.3 | 5.3 ± 1.1 | 0.47 |
| ED Arrival | | | | | | |
| ER Bilirubin (mg/dl) | 13.3 ± 1.5 | 15.8 ± 2.8 | 13.6 ± 2.1 | 14.1 ± 2.1 | 14.8 ± 3.4 | 0.55 |
| | | (33) | (14) | | (31) | |
| ER GGT Gamma-Glutamyl | 97.5 ± 20.4 | 165.6 ± 43.9 | 123.8 ± 64.0 | 81.8 ± 17.9 | 134.4 ± 33.9 | 0.55 |
| Trans-peptidase (IU/L) | | (33) | (14) | | (32) | |
| Readmit Within 6 Month | 38.5% | 38.7% | 20% | 21.4% | 25.8% | 0.58 |
| | (5/13) | (12/31) | (3/15) | (3/14) | (8/31) | |
| On Antibiotics Prior to ER | 25% | 32.4% | 25% | 13.3% | 26.5% | 0.77 |
| Arrival | (4/16) | (11/34) | (4/16) | (2/15) | (9/34) | |
| ER Altered Mental State | 18.8% | 17.6% | 12.5% | 20% | 8.8% | 0.81 |
| | (3/16) | (6/34) | (2/16) | (3/15) | (3/34) | |
| ER Heart Rate (beats/min) | 105.6 ± 5.5 | 103.8 ± 3.5 | 98.5 ± 3.7 | 104.3 ± 4.8 | 100.5 ± 2.9 | 0.87 |
| Mortality | 12.5% | 14.7% | 12.5% | 13.3% | 14.7% | 1 |
| | (2/16) | (5/34) | (2/16) | (2/15) | (5/34) | |

*The mean ± standard error, and total available observations for numerical variables (N only indicated for total available observations when not equal to total number of patients in the endotype). Categorical variables are presented as percent positive (total positive/total available observations). P values derived from Kruskal-Wallis and Chi square tests testing for significant differences between endotypes for numerical and categorical values, respectively.

TABLE 3
LASSO selected genes for endotype classification*.

| | | Fold Change (FC) | | | | |
|---|---|---|---|---|---|---|
| Gene | Description | NPS | INF | IHD | IFN | ADA |
| KLF14 | Kruppel like factor 14 | 14.45 | −1.56 | −3.39 | −3 | −16.31 |
| HPGD | 15-hydroxyprostaglandin dehydrogenase | 9.9 | −1.57 | −3.49 | −1.03 | −9.65 |
| PCOLCE2 | procollagen C-endopeptidase enhancer 2 | 9.21 | −1.39 | −3.01 | −1.35 | −11.56 |
| SLC51A | solute carrier family 51 subunit alpha | 8.87 | −1.35 | −7.55 | 1.17 | −17.26 |
| OLAH | oleoyl-ACP hydrolase | 8.8 | −1.43 | −4.18 | −1.07 | −11.82 |
| TNFAIP8L3 | TNF alpha induced protein 8 like 3 | 8.31 | −1.06 | −2.81 | −1.24 | −14.93 |
| EFNA1 | ephrin A1 | 8.1 | −1.37 | −2.21 | −1.87 | −3.9 |
| ZDHHC19 | Zinc finger DHHC-type palmitoyltransferase | 8.03 | −1.01 | −7.14 | 1.16 | −9.72 |
| GPR84 | G protein-coupled receptor 84 | 7.31 | 1.02 | −5.93 | −1.08 | −9.14 |
| ORM2 | orosomucoid 2 | 7.11 | 1.33 | −2.65 | −1.02 | −10.13 |
| ARG1 | arginase 1 | 5.4 | 1.05 | −1.74 | −1.09 | −7.91 |
| ATP9A | ATPase phospholipid transporting 9A (put.) | 4.68 | 1.21 | −2.61 | 1.14 | −7.49 |
| PFKFB2 | 6-phosphofructo-2-kinase | 4.22 | −1.01 | −2.65 | 1.81 | −9.23 |
| NSUN7 | NOP2/Sun RNA methyltransferase FM 7 | 4.01 | 1.12 | −2.3 | 1.28 | −5.47 |
| GADD45A | growth arrest & DNA damage inducible α | 3.89 | 1.22 | −1.93 | 1.09 | −5.64 |
| ANXA3 | annexin A3 | 3.27 | −1.05 | −2.61 | 2.05 | −3.99 |
| ILCR1 | interleukin 1 receptor type 1 | 3.18 | 1.1 | −1.62 | 1.18 | −3.35 |
| MLLT1 | MLLT1 super elongation complex subunit | 3.11 | −1.13 | −2.03 | 1.39 | −2.26 |
| MIR646HG | MIR646 host gene | 2.79 | 1.2 | −1.27 | 1.05 | −3.03 |
| AGFG1 | ArfGAP with FG repeats 1 | 2.73 | −1.07 | −1.51 | 1.58 | −2.84 |
| KREMEN1 | kringle containing transmembrane protein 1 | 1.92 | 1.03 | −1.81 | 1.84 | −2.06 |
| BNIP3L | BCL2 interacting protein 3 like | −1.47 | 3.19 | −1.55 | −1.81 | −2.29 |
| RIOK3 | RIO kinase 3 | −1.56 | 3.19 | −1.42 | −1.61 | −2.48 |
| TSPAN5 | tetraspanin 5 | −1.59 | 3.56 | −1.61 | −1.96 | −2.48 |
| GLRX5 | glutaredoxin 5 | −1.81 | 4.14 | −1.9 | −2.16 | −2.64 |
| TSPO2 | translocator protein 2 | −2.07 | 6.82 | −2.45 | −2.21 | −2.39 |
| TLCD4 | TLC domain containing 4 | −2.08 | 4.52 | −1.68 | −2.05 | −3.16 |
| SPTA1 | spectrin alpha, erythrocytic 1 | −2.7 | 3.66 | −1.16 | −1.82 | −2.51 |
| RHCE | Rh blood group CcEe antigens | −2.8 | 6.76 | −2.02 | −2.74 | −1.98 |
| THEM5 | thioesterase superfamily member 5 | −2.87 | 9.38 | −3.9 | −2.87 | −3.23 |
| FAM83A | family with sequence similarity 83 FM A | −3.09 | 9.87 | −3.72 | −18.01 | −11.3 |
| FECH | ferrochelatase | −3.2 | 6.85 | −2.09 | −4.08 | −3.66 |
| GYPA | glycophorin A (MNS blood group) | −4.37 | 10.03 | −1.32 | −5.01 | −2.92 |
| CA1 | carbonic anhydrase 1 | −4.81 | 10.56 | −2.95 | −5.16 | −7.89 |
| IFIT1B | IFN-induced prot, tetratricopeptide repeats | −5.14 | 7.39 | −3.25 | −3.29 | −2.7 |
| SLC6A19 | solute carrier family 6 member 19 | −5.99 | 27.78 | −2.6 | −9.18 | −10.2 |
| RNF182 | ring finger protein 182 | −4.19 | 7.2 | −1.9 | −2.16 | −4.34 |
| SDC2 | syndecan 2 | 1.37 | −1.28 | 2.81 | −2.28 | −1.72 |
| LPL | lipoprotein lipase | 1.33 | 1.46 | 4.57 | −1.35 | −2.68 |
| GRAMD1C | GRAM domain containing 1C | −1.01 | 1.08 | 1.8 | −1.27 | −1.5 |
| MAP7 | microtubule associated protein 7 | −1.03 | −1.56 | 1.84 | −1.08 | 1 |
| GPR34 | G protein-coupled receptor 34 | −1.03 | −1.3 | 2.68 | −1.2 | −1.57 |
| ABCA6 | ATP binding cassette subfamily A member 6 | −1.15 | 1.75 | 2.76 | −1.6 | −1.29 |
| CACNA2D3 | calcium voltage-gated channel aux. SU α2δ3 | −1.18 | −1.07 | 1.44 | −1.54 | 1.12 |
| SLC16A14 | solute carrier family 16 member 14 | −1.26 | −1.22 | 3.18 | −4.03 | −1.21 |
| PLCB1 | phospholipase C beta 1 | −1.3 | −1.12 | 1.63 | −1.72 | 1.14 |
| TPRG1 | tumor protein p63 regulated 1 | −1.37 | 1.03 | 1.28 | −1.66 | 1.19 |
| DYNC2H1 | dynein cytoplasmic 2 heavy chain 1 | −1.47 | −1.07 | 1.61 | −1.62 | 1.09 |
| MIR155HG | MIR155 host gene | −1.79 | −1.25 | 2.95 | −1.33 | −1.32 |
| ZNF600 | zinc finger protein 600 | −2.06 | −1.09 | 1.44 | −1.58 | 1.4 |
| ALOX15 | arachidonate 15-lipoxygenase | −2.36 | −1.52 | 3.73 | −3 | −1.05 |
| SPRED1 | sprouty related EVH1 domain containing 1 | −2.42 | −1.1 | 2.83 | −1.5 | −1.26 |
| TPPP3 | tubulin polymerization promoting prot. FM3 | −2.62 | −1.24 | 1.25 | −1.94 | 1.93 |
| ADAM23 | ADAM metallopeptidase domain 23 | −3.03 | 1.07 | 1.43 | −2.09 | 1.44 |
| SMPD3 | sphingomyelin phosphodiesterase 3 | −4.12 | −1.66 | 2.75 | −2.43 | 1.53 |
| SIGLEC8 | sialic acid binding Ig like lectin 8 | −5.01 | −2.2 | 3.58 | −3.16 | 1.51 |
| ANKRD22 | ankyrin repeat domain 22 | 1.28 | −1.54 | −4.83 | 4.49 | −1.92 |
| IFITM3 | interferon induced transmembrane protein 3 | 1.13 | −1.61 | −3.69 | 2.46 | 1.17 |
| PLEKHO1 | pleckstrin homology domain containing O1 | −1.64 | −1.22 | −1.25 | 1.03 | 1.63 |
| APOL1 | apolipoprotein L1 | −2.51 | −1.52 | −2.79 | 3.23 | 1.27 |
| TFEC | transcription factor (TF) EC | −2.51 | −1.49 | 1.22 | 4.95 | 1.32 |
| P2RY14 | purinergic receptor P2Y14 | −2.87 | −1.62 | −2.83 | 5.92 | −1.53 |
| BATF2 | basic leucine zipper ATF-like TF-2 | −2.89 | −2.02 | −5.53 | 4.84 | 1.26 |
| CARD17 | caspase recruitment domain FM 17 | −3.04 | −1.84 | −2.41 | 5.76 | −1.37 |
| EPSTI1 | epithelial stromal interaction 1 | −3.09 | −1.77 | −2.96 | 2.91 | 1.62 |
| ETV7 | ETS variant transcription factor 7 | −3.39 | −2.06 | −4.85 | 4.74 | 1.3 |
| SERPING1 | serpin family G member 1 | −5.44 | −2.56 | −6.36 | 4.15 | 1.87 |
| GBP5 | guanylate binding protein 5 | −7.65 | −1.72 | −2.81 | 4.59 | 1.16 |
| RSAD2 | radical S-adenosyl met. domain cont. 2 | −10.26 | −2.53 | −6.26 | 3.31 | 2.23 |
| IDO1 | indoleamine 2,3-dioxygenase 1 | −15.12 | −3.25 | −2.79 | 7.45 | 2.58 |
| APOL4 | apolipoprotein L4 | −16.66 | −1.72 | −3.5 | 4.46 | 1.53 |
| CD274 | CD274 molecule | −2.36 | −1.42 | −3.22 | 4.77 | −1.34 |
| PDIA4 | protein disulfide isomerase FM A 4 | −1.11 | −1.14 | −1.17 | −1.07 | 1.34 |
| KIF14 | kinesin family member 14 | −1.32 | 1.19 | −1.09 | −1.01 | 1.01 |
| CDC45 | cell division cycle 45 | −1.44 | −1.04 | −1.44 | −1.94 | 1.91 |
| GTSE1 | G2 and S-phase expressed 1 | −1.69 | 1.4 | −1.66 | −1.79 | 1.47 |
| CCL2 | C-C motif chemokine ligand 2 | −1.7 | −4.1 | −7.56 | −2.78 | 9.25 |
| KIF15 | kinesin family member 15 | −2.23 | 1.34 | −1.15 | −1.47 | 1.32 |
| CLEC4F | C-type lectin domain family 4 member F | −2.42 | −3.96 | 1.23 | 1.73 | 3.68 |
| LGALS3BP | galectin 3 binding protein | −2.8 | −3.12 | −3.45 | 1.33 | 4.16 |
| KLHDC7B | kelch domain containing 7B | −3.17 | −2.05 | −1.84 | −1.24 | 3.45 |
| KCTD14 | potassium channel tetramer domain cont. 14 | −4.09 | −7.29 | −6.69 | 1.46 | 5.74 |
| ISG15 | ISG15 ubiquitin like modifier | −5.14 | −3.77 | −9.93 | 2.3 | 3.47 |
| USP18 | ubiquitin specific peptidase 18 | −5.86 | −4.75 | −8.46 | 1.5 | 5.19 |
| IFI27 | interferon alpha inducible protein 27 | −5.92 | −1.58 | −31.09 | −1.02 | 4.01 |
| SIGLEC1 | sialic acid binding Ig like lectin 1 | −7.74 | −6.11 | −5.66 | 1.6 | 5.29 |
| OTOF | otoferlin | −22.51 | −18.01 | −17.31 | 1.22 | 13.7 |
| CENPF | centromere protein F | −1.33 | 1.46 | −1.39 | −1.48 | 1.09 |

*These 88 genes represent a reduced signature that can be more readily translated for clinical use.
FM = family member.
TF = transcription factor.

TABLE 4
Other markers based on highest gene expression in group of genes
maximally differentiating each endotype from each other endotype.

| | | Fold Change (FC) | | | | |
|---|---|---|---|---|---|---|
| Gene | Abbreviated Description | NPS | INF | IHD | IFN | ADA |
| HPGD | 15-hydroxyprostaglandin dehydrogenase | 9.9 | −1.57 | −3.49 | −1.03 | −9.65 |
| ADAMTS3 | ADAM metallopeptidase, thromb. type 1/3 | 6.55 | −1.04 | −2.43 | 1.18 | −16.67 |
| SEMA6B | semaphorin 6B | 5.68 | −1.04 | −2.93 | 1.4 | −7.97 |
| NECAB1 | N-terminal EF-hand Ca²⁺ binding protein 1 | 6.02 | 1.33 | −2.71 | −1.07 | −20.57 |
| CD177 | CD177 molecule | 5.66 | −1.08 | −5.06 | 1.83 | −13.12 |
| IL1R2 | interleukin 1 receptor type 2 | 5.92 | −1.06 | −5.13 | 1.35 | −9.6 |
| MMP9 | matrix metallopeptidase 9 | 5.68 | 1.11 | −3.17 | −1.23 | −5.94 |
| EXOSC4 | exosome component 4 | 5.45 | −1.25 | −3.69 | 1.13 | −3.5 |
| ENTPD7 | ectonucleoside tri-Pi diphosphohydrolase 7 | 5.27 | −1.16 | −2.07 | 1.32 | −6.03 |
| RGL4 | guanine nucl. dissociation stimulator like 4 | 5.13 | −1.17 | −2.71 | 1.28 | −4.42 |
| S100A12 | S100 calcium binding protein A12 | 5.08 | 1.01 | −2.8 | 1.27 | −5.02 |
| SPATCI | spermatogenesis and centriole associated 1 | 5.05 | −1.08 | −3.98 | 1.59 | −7.3 |
| DAAM2 | dishevelled assoc. activator morphogenesis 2 | 5.03 | −1.07 | −2.58 | 1.21 | −7.4 |
| PI3 | peptidase inhibitor 3 | 4.99 | 1.19 | −1.65 | −3.12 | −2.68 |
| OPLAH | 5-oxoprolinase, ATP-hydrolysing | 4.98 | −1.11 | −2.34 | −1.02 | −3.58 |
| SPP1 | secreted phosphoprotein 1 | 4.91 | −1.57 | −1.16 | 1.46 | −4.52 |
| PHF24 | PHD finger protein 24 | 4.81 | 1.16 | −1.78 | −1.24 | −4.88 |
| FGF13 | fibroblast growth factor 13 | 4.75 | −1.57 | −1.52 | 1.55 | −4.65 |
| XCR1 | X-C motif chemokine receptor 1 | 4.69 | −1.45 | −1.45 | 1.12 | −3.24 |
| CYP19A1 | cytochrome P450 family 19 subfam. A M1 | 4.6 | 1.21 | −1.68 | −1.05 | −7.26 |
| CYSTM1 | Cys-rich transmembrane module cont. 1 | 4.52 | −1.12 | −3.81 | 1.62 | −4.55 |
| MCEMP1 | mast cell expressed membrane protein 1 | 4.51 | −1 | −3.62 | 1.73 | −6.83 |
| GYG1 | glycogenin 1 | 4.34 | −1.03 | −2.85 | 1.49 | −4.72 |
| FFAR3 | free fatty acid receptor 3 | 4.25 | −1.41 | −5.23 | 1.47 | −2.13 |
| CA4 | carbonic anhydrase 4 | 4.24 | −1.02 | −2.56 | 1.14 | −3.58 |
| GRB10 | growth factor receptor bound protein 10 | 4.22 | 1.01 | −2.03 | 1.19 | −4.1 |
| S100P | S100 calcium binding protein P | 4.21 | 1.09 | −2.19 | 1.27 | −4.52 |
| GALNT14 | polypep N-acetylgalactosaminyltransferase | 4.12 | 1.13 | −2.6 | 1.14 | −3.78 |
| TMIGD3 | transmembrane and Ig domain containing 3 | 4.1 | −1.11 | −1.56 | 1.09 | −3.33 |
| ALDH1A2 | aldehyde dehydrogenase 1 FM-A2 | 4.01 | −1.13 | −2.26 | 1.17 | −2.25 |
| SYN2 | synapsin II | 3.91 | 1.01 | −2.7 | 1.31 | −3.23 |
| KCNMA1 | K⁺ Ca²⁺ -activated channel subfamily M α1 | 3.89 | −1.21 | −2.79 | 1.26 | −2.11 |
| FSTL4 | follistatin like 4 | 3.87 | 1.24 | −1.44 | −1.23 | −3.86 |
| IRAG1-AS1 | IRAGI antisense RNA 1 | 3.84 | 1.12 | −2.7 | 1.41 | −4.33 |
| PFKFB3 | 6-phosphofructo-2-kinase | 3.83 | 1 | −2.53 | 1.62 | −4.99 |
| PDGFC | platelet derived growth factor C | 3.82 | −1.05 | −1.2 | 1.06 | −4.07 |
| BTBD19 | BTB domain containing 19 | 3.78 | 1.06 | −3.69 | 1.54 | −3.94 |
| CST7 | cystatin F | 3.76 | −1.03 | −2.98 | 1.33 | −2.95 |
| ST6GALNAC3 | ST6 NAG α-2,6-sialyltransferase 3 | 3.71 | −1 | −1.47 | 1.19 | −3.97 |
| NSMCE1-DT | NSMCE1 divergent transcript | 3.71 | 1.15 | −2.03 | −1.19 | −2.54 |
| SOCS3 | suppressor of cytokine signaling 3 | 3.67 | −1.15 | −3.53 | 2.12 | −4.17 |
| PLK3 | polo like kinase 3 | 3.6 | −1 | −2.39 | 1.08 | −2.43 |
| ALPL | alkaline phos., biomineralization associated | 3.57 | 1.18 | −2.64 | 1.12 | −3.24 |
| PLIN5 | perilipin 5 | 3.56 | −1 | −2.54 | 1.26 | −2.81 |
| SHROOM4 | shroom family member 4 | 3.54 | 1.33 | −1.53 | 1.01 | −3.74 |
| KCNE1B | K⁺ voltage-gated channel SF E reg. SU1B | 3.48 | 1.09 | −3.28 | 1.31 | −2.71 |
| SLPI | secretory leukocyte peptidase inhibitor | 3.43 | −1.08 | −1.36 | −1.14 | −2.16 |
| ALOX5AP | arachidonate 5-lipoxygenase activating prot | 3.43 | −1.05 | −2.65 | 1.37 | −2.58 |
| TMEM120A | transmembrane protein 120A | 3.43 | −1.09 | −2.19 | 1.18 | −2.27 |
| IL1RN | interleukin 1 receptor antagonist | 3.4 | −1.56 | −4.62 | 1.31 | −1.95 |
| AKRIC1 | aldo-keto reductase family 1 member C1 | 3.38 | 1.12 | −2.09 | 1.93 | −5.34 |
| CD163L1 | CD163 molecule like 1 | 3.36 | −1.29 | −2 | 1.51 | −2.29 |
| GRAMDIA | GRAM domain containing 1A | 3.35 | 1 | −3.01 | 1.48 | −2.8 |
| PROK2 | prokineticin 2 | 3.35 | −1.06 | −1.83 | 1.52 | −3.4 |
| UPP1 | uridine phosphorylase 1 | 3.34 | −1.08 | −2.41 | 1.64 | −3.15 |
| ANKRD55 | ankyrin repeat domain 55 | 3.3 | 1.24 | −2.27 | 1.31 | −4.13 |
| TDRD9 | tudor domain containing 9 | 3.29 | 1.04 | −1.23 | 1.05 | −3.41 |
| CD82 | CD82 molecule | 3.26 | 1.03 | −2.02 | 1.14 | −2.47 |
| ECHDC3 | enoyl-CoA hydratase domain containing 3 | 3.24 | 1.03 | −1.72 | 1.4 | −3.51 |
| MKNK1 | MAPK interacting serine/threonine kinase 1 | 3.21 | −1.06 | −1.87 | 1.38 | −2.8 |
| POR | cytochrome p450 oxidoreductase | 3.2 | −1.09 | −2.18 | 1.44 | −2.57 |
| AMPH | amphiphysin | 3.19 | 1.04 | 1.03 | −1.56 | −2.67 |
| DGAT2 | diacylglycerol O-acyltransferase 2 | 3.18 | −1.04 | −1.92 | 1.24 | −2.44 |
| SPINK8 | serine peptidase inhibitor Kazal type 8 | 3.15 | 1.2 | −1.98 | 1.41 | −3.45 |
| BCL3 | BCL3 transcription coactivator | 3.14 | 1 | −2.49 | 1.2 | −2.15 |
| ROM1 | retinal outer segment membrane protein 1 | 3.14 | −1.1 | −1.67 | 1.38 | −2.45 |
| PLIN4 | perilipin 4 | 3.09 | 1.16 | −2.38 | 1.19 | −2.77 |
| SPDYA | speedy/RINGO cell cycle regulator FM A | 3.08 | 1.25 | −2.22 | 1.21 | −2.57 |
| MSRA | methionine sulfoxide reductase A | 3.07 | 1.06 | −1.82 | 1.01 | −2.21 |
| IL18RAP | interleukin 18 receptor accessory protein | 3.06 | −1.07 | −2.67 | 2 | −3.44 |
| IER3 | immediate early response 3 | 3.06 | 1.1 | −1.91 | 1.19 | −2.57 |
| RFX2 | regulatory factor X2 | 3 | 1.02 | −1.66 | 1.14 | −2.42 |
| TSPO | translocator protein | 3 | 1.01 | −1.83 | 1.18 | −2.25 |
| TENT5C | terminal nucleotidyltransferase 5C | −2.86 | 5.73 | −2.13 | −3.21 | −2.98 |
| TSPAN7 | tetraspanin 7 | −2.27 | 5.44 | −3.18 | −3 | −2.44 |
| KANK2 | KN motif and ankyrin repeat domains 2 | −2.49 | 5.36 | −1.9 | −2.76 | −3.13 |
| RAP1GAP | RAP1 GTPase activating protein | −2 | 5.29 | −3.4 | −5.54 | −2.29 |
| SLC14A1 | solute carrier Family 14/1 (Kidd blood gp) | −2.5 | 4.88 | −1.63 | −2.5 | −3.04 |
| HMBS | hydroxymethylbilane synthase | −1.63 | 4.83 | −1.9 | −2.04 | −1.88 |
| OSBP2 | oxysterol binding protein 2 | −1.75 | 4.79 | −2.45 | −3.31 | −2.57 |
| TFR2 | transferrin receptor 2 | −1.72 | 4.51 | −1.91 | −2.94 | −2.04 |
| TNS1 | tensin 1 | −2.08 | 4.51 | −1.82 | −2.53 | −2.64 |
| ALAS2 | 5′-aminolevulinate synthase 2 | −1.72 | 4.42 | −2.57 | −2.78 | −2.38 |
| ARHGEF37 | Rho guanine nucleotide exchange factor 37 | −1.98 | 4.35 | −1.71 | −1.94 | −2.89 |
| KCNH2 | K⁺ voltage-gated channel SFM H/2 | −1.62 | 4.29 | −1.9 | −3.39 | −2.81 |
| PTPRF | protein tyrosine phosphatase receptor type F | −1.85 | 4.29 | −1.62 | −1.73 | −1.67 |
| PRDX2 | peroxiredoxin 2 | −1.98 | 4.13 | −2.23 | −3.04 | −1.96 |
| ACKR1 | Aty. chemokine receptor 1 (Duffy blood gp) | −1.87 | 4.05 | −3.24 | −2.89 | −1.68 |
| RHAG | Rh associated glycoprotein | −2.62 | 3.5 | −1.56 | −1.94 | −2.22 |
| TMCC2 | transmembrane and coiled-coil domain F2 | −2.47 | 6.73 | −1.56 | −2.7 | −2.64 |
| DYRK3 | dual specificity Tyr phos. regulated kinase 3 | −2.33 | 5.39 | −1.61 | −2 | −2.2 |
| ITLN1 | intelectin 1 | −2.82 | 5.62 | −2.82 | −2.97 | −2.48 |
| KLHDC8A | kelch domain containing 8A | −1.74 | 3.79 | −1.74 | −2.48 | −2.18 |
| AHSP | alpha hemoglobin stabilizing protein | −2.64 | 6.31 | −3.21 | −2.99 | −3.07 |
| GYPB | glycophorin B (MNS blood group)] | −3.29 | 6.07 | −2.97 | −3.74 | −2.49 |
| YPEL4 | yippee like 4 | −1.83 | 2.71 | −1.32 | −1.67 | −1.96 |
| CTSE | cathepsin E | −1.66 | 3.68 | −2.23 | −1.98 | −3.18 |
| ACHE | acetylcholinesterase | −1.79 | 4.64 | −2.45 | −3.05 | −2.43 |
| KLF1 | Kruppel like factor 1 | −2 | 5.3 | −2.35 | −3.12 | −2.84 |
| XK | X-linked Kx blood group | −2.68 | 5.39 | −2.03 | −3.05 | −2.76 |
| LRRC2 | leucine rich repeat containing 2 | −2.38 | 6.2 | −2.18 | −3.35 | −3.47 |
| HEPACAM2 | HEPACAM family member 2 | −2.59 | 4.16 | −2.26 | −2.52 | −1.87 |
| MAOA | monoamine oxidase A | 1.44 | 3.6 | −1.87 | −3.67 | −4.5 |
| BPGM | bisphosphoglycerate mutase | −2.96 | 5.93 | −2.27 | −3.06 | −3.04 |
| SOX6 | SRY-box transcription factor 6 | −1.81 | 4.86 | −2.12 | −2.33 | −3.21 |
| BCAM | basal cell adhesion mol. (Lutheran blood gp) | −1.17 | 6.12 | −3.15 | −5.2 | −6.58 |
| ABCG2 | ATP bind. cassette FM G/2 (Junior bld gp) | −2.79 | 4.76 | −2.23 | −1.99 | −2.55 |
| HEMGN | hemogen | −2.36 | 5.09 | −2.19 | −2.65 | −2.8 |
| RIPOR3 | RIPOR family member 3 | −1.28 | 2.8 | −1.38 | −1.85 | −2.04 |
| RHD | Rh blood group D antigen | −1.73 | 4.94 | −2.65 | −2.84 | −2.71 |
| SLC6A9 | solute carrier family 6 member 9 | −2.66 | 5.96 | −2.32 | −4.31 | −3.78 |
| KRT1 | keratin 1 | −1.95 | 4.49 | −3.23 | −2.44 | −2.14 |
| TRIM10 | tripartite motif containing 10 | −2.31 | 3.96 | −1.72 | −1.94 | −2.28 |
| SELENOP | selenoprotein P | −2.17 | 3.1 | −1.11 | −2.01 | −1.92 |
| SLC4A1 | solute carrier FM 4/1 (Diego blood gp) | −2.33 | 5.04 | −2.03 | −2.74 | −2.8 |
| ERFE | erythroferrone | −1.32 | 4.31 | −1.9 | −5.73 | −3.13 |
| EPB42 | erythrocyte membrane protein band 4.2 | −2.08 | 4.99 | −2.64 | −2.99 | −2.47 |
| ANK1 | ankyrin 1 | −2.23 | 4.8 | −1.86 | −2.68 | −2.73 |
| SELENBP1 | selenium binding protein 1 | −1.93 | 4.99 | −2.48 | −2.84 | −2.8 |
| TMOD1 | tropomodulin 1 | −1.69 | 3.87 | −2.03 | −2.07 | −2.41 |
| SGIP1 | SH3GL interacting endocytic adaptor 1 | −1.91 | 3.8 | −2.35 | −2.47 | −1.82 |
| ATP1B2 | ATPase Na+/K+ transporting subunit beta 2 | −1.87 | 3.02 | −2.79 | −1.57 | −1.48 |
| DNAJC6 | DnaJ heat shock protein FM (Hsp40) C6 | −3.21 | 4.74 | −1.67 | −2.83 | −2.41 |
| CRIL | complement C3b/C4b receptor 1 like | −1.59 | 3.71 | −1.93 | −1.73 | −2.46 |
| KEL | Kell metallo-endopeptidase (Kell blood gp) | −1.93 | 4.87 | −2.66 | −3.13 | −3.09 |
| SNCA | synuclein alpha | −2.12 | 4.69 | −2.08 | −2.59 | −2.64 |
| SLC2A1 | solute carrier family 2 member 1 | −1.54 | 4.36 | −1.55 | −1.98 | −2.11 |
| SPTB | spectrin beta, erythrocytic | −1.89 | 4.85 | −2.05 | −2.68 | −2.98 |
| RFESD | Rieske Fe—S domain containing | −2.98 | 3.6 | −1.11 | −1.98 | −2.18 |
| SEC14L4 | SEC14 like lipid binding 4 | −1.86 | 4.71 | −1.98 | −2.43 | −2.84 |
| CA2 | carbonic anhydrase 2 | −2.41 | 4.03 | −1.45 | −2.11 | −2.44 |
| ACSL6 | acyl-CoA synthetase long chain FM 6 | −3.61 | 3.97 | −1.38 | −3.03 | −1.86 |
| GMPR | guanosine monophosphate reductase | −1.58 | 3.94 | −2.57 | −2.41 | −2.14 |
| C1orf116 | chromosome 1 open reading frame 116 | −1.78 | 3.94 | −1.82 | −2.18 | −2.37 |
| PGF | placental growth factor | −6.52 | 3.91 | −2.68 | −1.09 | −8.77 |
| SFRP2 | secreted frizzled related protein 2 | −1.53 | 3.9 | −2.08 | −1.85 | −2.47 |
| SLC6A8 | solute carrier family 6 member 8 | −1.57 | 3.88 | −1.91 | −2.78 | −2.25 |
| BCL2L1 | BCL2 like 1 | −1.53 | 3.88 | −2.17 | −2.34 | −2.32 |
| GSPT1 | G1 to S phase transition 1 | −1.93 | 3.8 | −1.77 | −2.16 | −2.22 |
| SLC1A5 | solute carrier family 1 member 5 | −2.05 | 3.79 | −1.86 | −2.67 | −1.89 |
| RGS16 | regulator of G protein signaling 16 | −1.4 | 3.79 | −1.6 | −1.7 | −2.32 |
| AQP1 | aquaporin 1 (Colton blood group) | −1.42 | 3.75 | −1.87 | −2.54 | −2.37 |
| BBOF1 | basal body orientation factor 1 | −1.64 | 3.75 | −1.68 | −2.02 | −2.6 |
| STRADB | STE20 related adaptor beta | −1.85 | 3.74 | −1.84 | −2.09 | −2.23 |
| RNF175 | ring finger protein 175 | −1.76 | 3.72 | −1.63 | −2.16 | −2.23 |
| CR1L | complement C3b/C4b receptor 1 like | −1.59 | 3.71 | −1.93 | −1.73 | −2.46 |
| MRC2 | mannose receptor C type 2 | −1.5 | 3.7 | −1.82 | −1.89 | −2.44 |
| ANKRD9 | ankyrin repeat domain 9 | −1.59 | 3.68 | −2.22 | −2.77 | −1.91 |
| MBNL3 | muscleblind like splicing regulator 3 | −1.92 | 3.64 | −1.55 | −2.12 | −2.29 |
| MXI1 | MAX interactor 1, dimerization protein | −1.63 | 3.64 | −1.62 | −2.21 | −2.42 |
| DCAF12 | DDB1 and CUL4 associated factor 12 | −1.65 | 3.63 | −1.68 | −1.95 | −2.45 |
| NFIX | nuclear factor I X | −1.58 | 3.61 | −2.18 | −1.9 | −2.13 |
| RFESD | Rieske Fe—S domain containing | −2.98 | 3.6 | −1.11 | −1.98 | −2.18 |
| RBM38 | RNA binding motif protein 38 | −1.39 | 3.6 | −1.72 | −2.43 | −2.41 |
| MYL4 | myosin light chain 4 | −1.51 | 3.59 | −2.73 | −1.83 | −2.03 |
| FRMD4A | FERM domain containing 4A | −1.94 | 3.55 | −1.57 | −2.06 | −2.05 |
| ARHGEF12 | Rho guanine nucleotide exchange factor 12 | −2.05 | 3.54 | −1.52 | −1.87 | −2.22 |
| PLEK2 | pleckstrin 2 | −1.32 | 3.54 | −1.85 | −1.9 | −2.64 |
| MARCHF8 | membrane associated ring-CH-type finger 8 | −1.81 | 3.52 | −1.72 | −1.82 | −2.22 |
| FAM210B | family with sequence similarity 210/B | −1.4 | 3.46 | −1.94 | −2.31 | −2.16 |
| TRIM58 | tripartite motif containing 58 | −1.37 | 3.43 | −1.84 | −2.23 | −2.23 |
| DPCD | Deleted in primary ciliary dyskinesia hom. | −1.96 | 3.41 | −1.78 | −2.48 | −1.72 |
| UBB | ubiquitin B | −1.52 | 3.41 | −2.23 | −1.74 | −2.11 |
| SMIM5 | small integral membrane protein 5 | −1.44 | 3.34 | −2.24 | −1.8 | −2.03 |
| CLIC2 | chloride intracellular channel 2 | −2.59 | 3.34 | −1.54 | −1.41 | −2.1 |
| MFSD2B | major facilitator superfam domain cont .- 2B | −1.27 | 3.3 | −1.96 | −2.41 | −2.04 |
| PBX1 | PBX homeobox 1 | −1.25 | 3.3 | −1.91 | −2.1 | −2.24 |
| ADD2 | adducin 2 | −1.69 | 3.29 | −1.37 | −2.34 | −1.18 |
| FAXDC2 | fatty acid hydroxylase domain containing 2 | −1.5 | 3.29 | −1.82 | −2.12 | −1.97 |
| ARL4A | ADP ribosylation factor like GTPase 4A | −1.77 | 3.28 | −1.46 | −1.3 | −2.72 |
| USP12 | ubiquitin specific peptidase 12 | −2.14 | 3.26 | −1.25 | −1.84 | −2.19 |
| EMID1 | EMI domain containing 1 | −1.13 | 3.25 | −1.51 | −2.38 | −2.41 |
| YBX3 | Y-box binding protein 3 | −1.54 | 3.24 | −1.69 | −2.09 | −2.02 |
| ISCA1 | iron-sulfur cluster assembly 1 | −1.68 | 3.24 | −1.29 | −2.07 | −2.27 |
| KLC3 | kinesin light chain 3 | −1.13 | 3.22 | −2.01 | −2.37 | −2.11 |
| KDM7A-DT | KDM7A divergent transcript | −1.39 | 3.16 | −1.77 | −1.94 | −2.07 |
| CTNNAL1 | catenin alpha like 1 | −1.38 | 3.16 | −1.57 | −2.28 | −2.07 |
| SLC7A5 | solute carrier family 7 member 5 | −1.02 | 3.16 | −1.91 | −2.26 | −2.35 |
| BLVRB | biliverdin reductase B | −1.46 | 3.14 | −2.26 | −1.61 | −1.92 |
| HBM | hemoglobin subunit mu | −1.31 | 3.13 | −3.22 | −1.97 | −1.64 |
| SIAH2 | siah E3 ubiquitin protein ligase 2 | −1.29 | 3.13 | −1.87 | −1.7 | −2.23 |
| RUNDC3A | RUN domain containing 3A | −1.34 | 3.12 | −2.64 | −2.45 | −1.54 |
| CISD2 | CDGSH iron sulfur domain 2 | −1.87 | 3.11 | −1.53 | −1.89 | −1.87 |
| PNP | purine nucleoside phosphorylase | −1.59 | 3.11 | −1.67 | −2.02 | −1.87 |
| DMTN | dematin actin binding protein | −1.31 | 3.11 | −1.96 | −2.02 | −1.94 |
| RGCC | regulator of cell cycle | −1.92 | 3.08 | −1.55 | −1.69 | −1.86 |
| TTC25 | tetratricopeptide repeat domain 25 | −1.32 | 3.08 | −2.13 | −1.68 | −1.92 |
| IGF2BP2 | insulin like growth factor 2 mRNA BP-2 | −1.41 | 3.08 | −1.65 | −2.1 | −1.95 |
| SLC22A23 | solute carrier family 22 member 23 | −1.41 | 3.04 | −1.54 | −1.7 | −1.23 |
| TAL1 | bHLH transcription factor 1, erythroid DF | −1.31 | 3.04 | −1.71 | −1.96 | −2.04 |
| NUDT4 | nudix hydrolase 4 | −1.58 | 3.03 | −1.19 | −2.06 | −2.24 |
| ATP1B2 | ATPase Na+/K+ transporting subunit beta 2 | −1.87 | 3.02 | −2.79 | −1.57 | −1.48 |
| ALDH5A1 | aldehyde dehydrogenase 5 FMA1 | −2.1 | 3.02 | −1.41 | −1.88 | −1.74 |
| PCDH1 | protocadherin 1 | −1.29 | 3.01 | −1.6 | −2.42 | −1.88 |
| PAGE2B | PAGE family member 2B | −1.18 | 3 | −2.1 | −1.61 | −2.09 |
| GPR82 | G protein-coupled receptor 82 | −1.23 | −1.37 | 2.17 | −2.27 | 1.12 |
| PRSS33 | serine protease 33 | −3.13 | −1.82 | 3.10 | −2.22 | 1.36 |
| IL5RA | interleukin 5 receptor subunit alpha | −2.87 | −1.31 | 2.62 | −1.65 | 1.14 |
| TRIM2 | tripartite motif containing 2 | −1.69 | −1.1 | 2.03 | −2.45 | 1.11 |
| TBC1D12 | TBC1 domain family member 12 | −1.87 | −1.39 | 2.35 | −1.33 | 1.03 |
| ADGRD1 | adhesion G protein-coupled receptor D1 | −2.34 | −1.01 | 2.34 | −2.32 | 1.01 |
| HDAC9 | histone deacetylase 9 | −2.38 | −1.42 | 2.26 | −1.56 | 1.28 |
| PTGFRN | prostaglandin F2 receptor inhibitor | −2.05 | −1.54 | 2.15 | −1.32 | 1.24 |
| PTGDR2 | prostaglandin D2 receptor 2 | −2.75 | −1.3 | 2.06 | −1.58 | 1.35 |
| ANGPT1 | angiopoietin 1 | −1.3 | 1.3 | 2.05 | −1.64 | −1.66 |
| KLHDC1 | kelch domain containing 1 | −1.73 | −1.48 | 2.03 | −2.01 | 1.3 |
| EXOC3L1 | exocyst complex component 3 like 1 | −7.85 | −2.5 | −3.3 | 3.09 | 2.11 |
| SEPTIN4 | septin 4 | −7.4 | −2.85 | −4.22 | 4.28 | 1.91 |
| IRF7 | interferon regulatory factor 7 | −1.45 | −2.18 | −3.75 | 3.23 | 1.4 |
| OAS1 | 2′-5′-oligoadenylate synthetase 1 | −4.35 | −2.39 | −3.36 | 3 | 2 |
| LY6E | lymphocyte antigen 6 family member E | −3.12 | −3.61 | −5.13 | 2.2 | 3.08 |
| LAMP3 | lysosomal associated membrane protein 3 | −11.7 | −2.67 | −7.9 | 3.49 | 2.24 |
| IFIT3 | IFN induced protein, tetratricopeptide repts 3 | −4.29 | −2.04 | −4.79 | 3.21 | 1.83 |
| IFI44L | interferon induced protein 44 like | −8.03 | −2.63 | −4.63 | 3.4 | 2.12 |
| TTC21A | tetratricopeptide repeat domain 21A | −2.44 | −1.89 | −1.84 | 1.15 | 2.65 |
| SAMD4A | sterile alpha motif domain containing 4A | −10.03 | −2.32 | −1.49 | 1.3 | 3.16 |
| SPATS2L | spermatogenesis associated serine rich 2 like | −4.28 | −2.49 | −3.13 | 2.29 | 2.51 |
| HERC5 | HECT&RLD, E3 ubiquitin protein ligase 5 | −6.12 | −2.09 | −5.47 | 2.85 | 2.17 |
| AGRN | agrin | −5.41 | −2.27 | −2.42 | 1.49 | 3.13 |
| DHX58 | DExH-box helicase 58 | −3.35 | −2.21 | −2.97 | 2.21 | 2.3 |
| TSHR | thyroid stimulating hormone receptor] | −1.88 | 1.01 | −1.19 | −3.88 | 2.18 |
| TNFRSF13B | TNF receptor superfamily member 13B | −1.09 | 1.04 | −1.49 | −1.62 | 2.06 |
| PARM1 | prostate androgen-reg. mucin-like protein 1 | −1.76 | −1.16 | −1.07 | −2.21 | 2.02 |
| FAM111B | family sequence similarity 111 member B | −2.92 | −1.37 | −1.39 | −1.53 | 2.81 |
| MCM10 | minichromosome maintenance 10 repln. IF | −2.35 | −1.13 | −1.72 | −1.51 | 2.4 |
| LAG3 | lymphocyte activating 3 | −2.68 | −1.07 | −1.94 | −1.3 | 2.2 |
| CD38 | CD38 molecule | −2.66 | −1.45 | −1.39 | −1.07 | 2.34 |
| IFNG-AS1 | IFNG antisense RNA 1 | −2.53 | 1.06 | −1.28 | −3.83 | 2.17 |
| CDT1 | chromatin licensing /DNA replication fact-1 | −2.02 | 1.13 | −1.44 | −2.17 | 2.31 |
| CDCA7 | cell division cycle associated 7 | −2.85 | −1.15 | −1.09 | −1.54 | 2.01 |
| EME1 | Essential meiotic structure-spec. endonucl-1 | −2.28 | −1.25 | −1.3 | −1.4 | 2.15 |
| CTLA4 | cytotoxic T-lymphocyte associated protein 4 | −2.48 | −1.13 | −1.13 | −1.56 | 1.99 |
| HES4 | hes family bHLH transcription factor 4 | −6.73 | −2.53 | −1.41 | 1.05 | 3.44 |
| PACSIN1 | PKC & casein kinase substrate in neurons 1 | −2.69 | −1.55 | −1.62 | −2.65 | 3.41 |
| IL12RB2 | interleukin 12 receptor subunit beta 2 | −4.46 | −1.4 | −2.12 | −1.59 | 3.23 |
| IL4I1 | interleukin 4 induced 1 | −2.28 | −1.82 | −2.7 | −1.12 | 3.17 |
| P2RY6 | pyrimidinergic receptor P2Y6 | −2.44 | −1.88 | −1.89 | −1.12 | 3.03 |
| KIF19 | kinesin family member 19 | −4.68 | −1.2 | −1.05 | −3.7 | 2.69 |
| TMPRSS3 | transmembrane serine protease 3 | −3.53 | −1.4 | −1.43 | −1.2 | 2.5 |

TABLE 5
Examples of diagnostic accuracy of pairs of endotype classifiers.

| | | Percent Accuracy | | |
|---|---|---|---|---|
| | | (AUC-ROC), Sensitivity and | | |
| | | Specificity of diagnosis | | |
| Comparison | Gene Pairs tested | AUC | Sensitivity | Specificity |
| NPS vs. Rest | GADD45A^{#}, EFNA1 | 98.8 | 96.0 | 90.6 |
| NPS vs. Rest | EFNA1, MIR646HG | 98.5 | 97.0 | 91.7 |
| NPS vs. Rest | MIR646HG, KLF14 | 98.4 | 93.4 | 89.8 |
| NPS vs. Rest | MLLT1, MIR646HG | 98.3 | 97.5 | 86.6 |
| NPS vs. Rest | ARG1*, MLLT1 | 97.7 | 90.9 | 88.3 |
| NPS vs. Rest | MLLT1, EFNA1 | 97.7 | 91.4 | 88.7 |
| NPS vs. Rest | MLLT1, NSUN7 | 97.7 | 92.4 | 81.2 |
| NPS vs. Rest | EFNA1, NSUN7 | 97.5 | 85.1 | 92.4 |
| NPS vs. Rest | SLC51A, EFNA1 | 97.4 | 89.8 | 90.5 |
| NPS vs. Rest | EFNA1, KLF14 | 97.4 | 88.0 | 92.7 |
| NPS vs. Rest | ZDHHC19, EFNA1 | 97.3 | 86.1 | 89.0 |
| NPS vs. Rest | EFNA1, AGFG1 | 97.3 | 87.5 | 90.3 |
| NPS vs. Rest | NSUN7, KLF14 | 97.3 | 95.4 | 91.8 |
| NPS vs. Rest | EFNA1, PFKFB2^{#} | 97.2 | 88.7 | 87.9 |
| NPS vs. Rest | MLLT1, KLF14 | 97.2 | 92.3 | 86.4 |
| INF vs. Rest | FECH*, TFEC | 91.3 | 83.0 | 83.0 |
| INF vs. Rest | TFEC, IFIT1B | 90.3 | 80.9 | 80.9 |
| INF vs. Rest | FECH*, RNF182 | 90.0 | 84.1 | 81.0 |
| INF vs. Rest | IFIT1B, FECH* | 89.9 | 81.6 | 79.2 |
| INF vs. Rest | FECH*, APOL4 | 89.4 | 82.5 | 79.5 |
| INF vs. Rest | FECH*, GYPA | 89.4 | 81.7 | 80.4 |
| INF vs. Rest | ITLN1, FECH* | 89.4 | 82.3 | 81.3 |
| INF vs. Rest | FECH*, THEM5 | 89.4 | 82.5 | 80.9 |
| INF vs. Rest | IFIT1B, CA1* | 89.4 | 82.2 | 80.9 |
| INF vs. Rest | RHAG, FECH* | 89.3 | 81.4 | 80.5 |
| INF vs. Rest | FECH*, FAM83A | 89.3 | 80.6 | 80.2 |
| INF vs. Rest | RHCE, FECH* | 89.3 | 79.1 | 80.4 |
| INF vs. Rest | TFEC, CA1* | 89.3 | 89.0 | 78.9 |
| INF vs. Rest | SPTA1, FECH* | 89.1 | 81.1 | 80.7 |
| IHD vs. Rest | MAP7, SPRED1 | 94.4 | 87.3 | 85.3 |
| IHD vs. Rest | SPRED1, GPR34 | 93.5 | 88.3 | 83.4 |
| IHD vs. Rest | IL5RA, SPRED1 | 92.6 | 82.0 | 81.5 |
| IHD vs. Rest | SPRED1, TPRG1 | 91.7 | 87.3 | 78.4 |
| IHD vs. Rest | HRK, SPRED1 | 91.6 | 80.3 | 81.2 |
| IHD vs. Rest | SPRED1, PLCB1 | 91.2 | 90.0 | 82.5 |
| IHD vs. Rest | TRIM2, SPRED1 | 90.7 | 82.3 | 80.7 |
| IHD vs. Rest | SIGLEC8, SPRED1 | 90.6 | 76.4 | 80.6 |
| IHD vs. Rest | SMPD3, SPRED1 | 90.5 | 78.7 | 78.8 |
| IHD vs. Rest | SPRED1, ZNF600 | 90.5 | 81.2 | 81.2 |
| IHD vs. Rest | SPRED1, SDC2 | 90.3 | 79.9 | 82.2 |
| IHD vs. Rest | MAP7, GPR34 | 89.9 | 86.8 | 80.2 |
| IHD vs. Rest | PRSS33, SPRED1 | 89.8 | 78.2 | 79 |
| IHD vs. Rest | SPRED1, DYNC2H1 | 89.6 | 82.4 | 79.8 |
| IHD vs. Rest | CACNA2D3, SPRED1 | 89.1 | 78.0 | 78.6 |
| IFN vs. Rest | ETV7, PLEKHO1* | 92.5 | 89.6 | 78.1 |
| IFN vs. Rest | IFITM3, ETV7 | 91.9 | 83.9 | 79.7 |
| IFN vs. Rest | ETV7, APOL1* | 91.7 | 89.4 | 79.9 |
| IFN vs. Rest | BATF2, ETV7 | 91.7 | 88.8 | 78.1 |
| IFN vs. Rest | PLEKHO1*, BATF2 | 91.7 | 89 | 78.6 |
| IFN vs. Rest | ETV7, EPSTI1* | 91.4 | 83.2 | 76.2 |
| IFN vs. Rest | USP18, EPSTI1* | 91.1 | 88.1 | 74.2 |
| IFN vs. Rest | EPSTI1*, BATF2 | 91.0 | 83.2 | 75.6 |
| IFN vs. Rest | IFITM3, BATF2 | 90.8 | 83.6 | 78.2 |
| IFN vs. Rest | ETV7, SEPTIN4 | 90.2 | 86.7 | 78.3 |
| IFN vs. Rest | ETV7, LAMP3 | 90.1 | 83.6 | 76.5 |
| IFN vs. Rest | SERPING1, BATF2 | 90 | 87.4 | 76.3 |
| IFN vs. Rest | LAMP3, BATF2 | 89.5 | 83.6 | 77 |
| IFN vs. Rest | LAMP3, SERPING1 | 87.5 | 81.3 | 76.8 |
| ADA vs. Rest | LGALS3BP, OTOF | 88.2 | 77.5 | 85.9 |
| ADA vs. Rest | LGALS3BP, IFI27 | 87.6 | 78.3 | 82.2 |
| ADA vs. Rest | LGALS3BP, KIF14 | 87.5 | 76.7 | 81.4 |
| ADA vs. Rest | LGALS3BP, CENPF | 87.1 | 78.7 | 83.2 |
| ADA vs. Rest | GTSE1, LGALS3BP | 86.9 | 75.9 | 83.6 |
| ADA vs. Rest | LGALS3BP, KCTD14 | 86.9 | 75.1 | 83.3 |
| ADA vs. Rest | LGALS3BP, PDIA4 | 86.9 | 76.1 | 83.9 |
| ADA vs. Rest | LGALS3BP, TSHR | 86.7 | 75.6 | 82.1 |
| ADA vs. Rest | LGALS3BP, PLAAT2 | 86.6 | 75.3 | 80.6 |
| ADA vs, Rest | OTOF, IFI27 | 86.6 | 75.8 | 86.0 |
| ADA vs. Rest | IGF1, LGALS3BP | 86.2 | 75.6 | 82.1 |
| ADA vs. Rest | CDC45, LGALS3BP | 86.2 | 75.2 | 83.0 |
| ADA vs. Rest | LGALS3BP, KIF15 | 86.2 | 75.6 | 83.2 |
| ADA vs. Rest | IGLL5, LGALS3BP | 86.2 | 76.7 | 80.4 |
| ADA vs. Rest | LGALS3BP, MIXL1 | 86.1 | 74.1 | 82.7 |

Genes with * in column 2 include instances where one member of the pair was reported in previous endotype papers, although never partnered with the other gene in the pair. Genes with ^{#} are where ambiguous relationships with endotypes were reported.

TABLE 6
Expanded list of Gene Pairs that classify into specific endotypes when compared to all others.

| Compare | Gene Pair | ROC | Sens | Spec | Compare | Gene Pair | ROC | Sens | Spec |
|---|---|---|---|---|---|---|---|---|---|
| NPS vs Rest | ATP9A/EPB41L4B | 90 | 86 | 78 | INF vs Rest | TSPO2/RHCE | 84 | 66 | 77 |
| NPS vs Rest | ATP9A/IL1R1 | 95 | 93 | 83 | INF vs Rest | TSPO2/THEM5 | 85 | 74 | 77 |
| NPS vs Rest | ATP9A/GADD45A | 95 | 96 | 84 | INF vs Rest | TSPO2/IFIT1B | 88 | 73 | 79 |
| NPS vs Rest | ATP9A/ARG1 | 97 | 95 | 86 | INF vs Rest | TSPO2/CARD17 | 81 | 69 | 73 |
| NPS vs Rest | ATP9A/PFKFB2 | 92 | 94 | 80 | INF vs Rest | CD274/TMCC2 | 83 | 69 | 74 |
| NPS vs Rest | ATP9A/MLLT1 | 95 | 91 | 80 | INF vs Rest | CD274/CA1 | 88 | 88 | 76 |
| NPS vs Rest | ATP9A/ANXA3 | 92 | 90 | 77 | INF vs Rest | CD274/DYRK3 | 80 | 68 | 74 |
| NPS vs Rest | ATP9A/GPR84 | 91 | 85 | 80 | INF vs Rest | CD274/FAM83A | 83 | 71 | 77 |
| NPS vs Rest | ATP9A/OLAH | 95 | 89 | 82 | INF vs Rest | CD274/TLCD4 | 84 | 73 | 74 |
| NPS vs Rest | ATP9A/ADAMTS3 | 92 | 87 | 83 | INF vs Rest | CD274/KLHDC8A | 77 | 72 | 67 |
| NPS vs Rest | ATP9A/PCOLCE2 | 92 | 87 | 83 | INF vs Rest | CD274/SPTA1 | 83 | 70 | 76 |
| NPS vs Rest | ATP9A/ZDHHC19 | 94 | 90 | 82 | INF vs Rest | CD274/TSPAN5 | 86 | 80 | 81 |
| NPS vs Rest | ATP9A/SLC51A | 93 | 90 | 82 | INF vs Rest | CD274/GYPA | 85 | 77 | 76 |
| NPS vs Rest | ATP9A/HPGD | 94 | 96 | 82 | INF vs Rest | CD274/ITLN1 | 81 | 73 | 74 |
| NPS vs Rest | ATP9A/SEMA6B | 90 | 87 | 78 | INF vs Rest | CD274/RNF182 | 84 | 77 | 79 |
| NPS vs Rest | ATP9A/EFNA1 | 97 | 88 | 89 | INF vs Rest | CD274/GLRX5 | 86 | 80 | 76 |
| NPS vs Rest | ATP9A/AGFG1 | 94 | 92 | 81 | INF vs Rest | CD274/RHCE | 85 | 72 | 78 |
| NPS vs Rest | ATP9A/NSUN7 | 96 | 97 | 82 | INF vs Rest | CD274/THEM5 | 83 | 72 | 78 |
| NPS vs Rest | ATP9A/TNFAIP8L3 | 91 | 87 | 80 | INF vs Rest | CD274/IFIT1B | 88 | 80 | 79 |
| NPS vs Rest | ATP9A/KREMEN1 | 90 | 90 | 76 | INF vs Rest | TMCC2/CA1 | 87 | 81 | 77 |
| NPS vs Rest | ATP9A/ORM2 | 91 | 94 | 79 | INF vs Rest | TMCC2/DYRK3 | 84 | 66 | 77 |
| NPS vs Rest | ATP9A/MIR646HG | 95 | 92 | 83 | INF vs Rest | TMCC2/FAM83A | 86 | 73 | 77 |
| NPS vs Rest | ATP9A/KLF14 | 96 | 94 | 87 | INF vs Rest | TMCC2/TLCD4 | 86 | 76 | 76 |
| NPS vs Rest | EPB41L4B/ILIR1 | 89 | 76 | 81 | INF vs Rest | TMCC2/ANKRD22 | 83 | 68 | 74 |
| NPS vs Rest | EPB41L4B/GADD45A | 93 | 87 | 82 | INF vs Rest | TMCC2/GBP5 | 82 | 70 | 74 |
| NPS vs Rest | EPB41L4B/ARG1 | 92 | 92 | 82 | INF vs Rest | TMCC2/KLHDC8A | 83 | 70 | 76 |
| NPS vs Rest | EPB41L4B/PFKFB2 | 88 | 82 | 75 | INF vs Rest | TMCC2/SPTA1 | 85 | 74 | 76 |
| NPS vs Rest | EPB41L4B/MLLT1 | 94 | 86 | 83 | INF vs Rest | TMCC2/TSPAN5 | 87 | 74 | 80 |
| NPS vs Rest | EPB41L4B/ANXA3 | 89 | 86 | 75 | INF vs Rest | TMCC2/GYPA | 87 | 75 | 76 |
| NPS vs Rest | EPB41L4B/GPR84 | 85 | 77 | 81 | INF vs Rest | TMCC2/P2RY14 | 82 | 68 | 74 |
| NPS vs Rest | EPB41L4B/OLAH | 87 | 69 | 80 | INF vs Rest | TMCC2/ITLN1 | 85 | 72 | 80 |
| NPS vs Rest | EPB41L4B/ADAMTS3 | 87 | 79 | 86 | INF vs Rest | TMCC2/RNF182 | 86 | 79 | 78 |
| NPS vs Rest | EPB41L4B/PCOLCE2 | 80 | 71 | 83 | INF vs Rest | TMCC2/GLRX5 | 86 | 75 | 77 |
| NPS vs Rest | EPB41L4B/ZDHHC19 | 89 | 78 | 82 | INF vs Rest | TMCC2/RHCE | 85 | 73 | 78 |
| NPS vs Rest | EPB41L4B/SLC51A | 92 | 85 | 83 | INF vs Rest | TMCC2/THEM5 | 86 | 74 | 79 |
| NPS vs Rest | EPB41L4B/HPGD | 88 | 71 | 79 | INF vs Rest | TMCC2/IFIT1B | 88 | 77 | 81 |
| NPS vs Rest | EPB41L4B/SEMA6B | 84 | 77 | 75 | INF vs Rest | TMCC2/CARD17 | 82 | 69 | 74 |
| NPS vs Rest | EPB41L4B/EFNA1 | 95 | 83 | 90 | INF vs Rest | CA1/DYRK3 | 87 | 83 | 76 |
| NPS vs Rest | EPB41L4B/AGFG1 | 92 | 87 | 80 | INF vs Rest | CA1/FAM83A | 88 | 85 | 77 |
| NPS vs Rest | EPB41L4B/NSUN7 | 94 | 95 | 82 | INF vs Rest | CA1/TLCD4 | 88 | 84 | 78 |
| NPS vs Rest | EPB41L4B/TNFAIP8L3 | 85 | 79 | 81 | INF vs Rest | CA1/ANKRD22 | 87 | 88 | 75 |
| NPS vs Rest | EPB41L4B/KREMEN1 | 81 | 77 | 72 | INF vs Rest | CA1/GBP5 | 87 | 88 | 76 |
| NPS vs Rest | EPB41L4B/MIR646HG | 90 | 84 | 80 | INF vs Rest | CA1/KLHDC8A | 87 | 84 | 76 |
| NPS vs Rest | EPB41L4B/KLF14 | 93 | 89 | 87 | INF vs Rest | CA1/SPTA1 | 88 | 83 | 78 |
| NPS vs Rest | IL1R1/GADD45A | 96 | 89 | 85 | INF vs Rest | CA1/TSPAN5 | 89 | 85 | 80 |
| NPS vs Rest | IL1R1/ARG1 | 93 | 82 | 84 | INF vs Rest | CA1/GYPA | 88 | 82 | 76 |
| NPS vs Rest | IL1R1/PFKFB2 | 92 | 80 | 81 | INF vs Rest | CA1/P2RY14 | 87 | 88 | 76 |
| NPS vs Rest | IL1R1/MLLT1 | 96 | 90 | 86 | INF vs Rest | CA1/ITLN1 | 87 | 82 | 77 |
| NPS vs Rest | IL1R1/ANXA3 | 93 | 87 | 83 | INF vs Rest | CA1/RNF182 | 89 | 82 | 78 |
| NPS vs Rest | IL1R1/GPR84 | 95 | 87 | 87 | INF vs Rest | CA1/GLRX5 | 88 | 85 | 78 |
| NPS vs Rest | IL1R1/OLAH | 92 | 73 | 86 | INF vs Rest | CA1/RHCE | 88 | 82 | 78 |
| NPS vs Rest | IL1R1/ADAMTS3 | 92 | 81 | 85 | INF vs Rest | CA1/THEM5 | 88 | 83 | 80 |
| NPS vs Rest | IL1R1/PCOLCE2 | 92 | 79 | 85 | INF vs Rest | CA1/IFIT1B | 89 | 82 | 81 |
| NPS vs Rest | IL1R1/ZDHHC19 | 95 | 83 | 86 | INF vs Rest | CA1/CARD17 | 87 | 88 | 74 |
| NPS vs Rest | IL1R1/SLC51A | 95 | 86 | 86 | INF vs Rest | DYRK3/FAM83A | 85 | 70 | 76 |
| NPS vs Rest | IL1R1/HPGD | 92 | 79 | 86 | INF vs Rest | DYRK3/TLCD4 | 84 | 71 | 77 |
| NPS vs Rest | IL1R1/SEMA6B | 94 | 89 | 84 | INF vs Rest | DYRK3/ANKRD22 | 80 | 68 | 75 |
| NPS vs Rest | IL1R1/EFNA1 | 96 | 78 | 92 | INF vs Rest | DYRK3/GBP5 | 79 | 68 | 73 |
| NPS vs Rest | IL1R1/AGFG1 | 94 | 90 | 85 | INF vs Rest | DYRK3/KLHDC8A | 81 | 65 | 74 |
| NPS vs Rest | IL1R1/NSUN7 | 95 | 90 | 84 | INF vs Rest | DYRK3/SPTA1 | 84 | 72 | 79 |
| NPS vs Rest | IL1R1/TNFAIP8L3 | 95 | 93 | 85 | INF vs Rest | DYRK3/TSPAN5 | 86 | 74 | 80 |
| NPS vs Rest | IL1R1/KREMEN1 | 91 | 87 | 82 | INF vs Rest | DYRK3/GYPA | 86 | 74 | 76 |
| NPS vs Rest | IL1R1/ORM2 | 91 | 85 | 83 | INF vs Rest | DYRK3/P2RY14 | 79 | 67 | 74 |
| NPS vs Rest | IL1R1/MIR646HG | 95 | 92 | 85 | INF vs Rest | DYRK3/ITLN1 | 84 | 70 | 79 |
| NPS vs Rest | IL1R1/KLF14 | 96 | 87 | 90 | INF vs Rest | DYRK3/RNF182 | 85 | 76 | 79 |
| NPS vs Rest | GADD45A/ARG1 | 96 | 95 | 83 | INF vs Rest | DYRK3/GLRX5 | 86 | 75 | 76 |
| NPS vs Rest | GADD45A/PFKFB2 | 94 | 93 | 80 | INF vs Rest | DYRK3/RHCE | 85 | 69 | 78 |
| NPS vs Rest | GADD45A/MLLT1 | 97 | 94 | 86 | INF vs Rest | DYRK3/THEM5 | 85 | 77 | 79 |
| NPS vs Rest | GADD45A/ANXA3 | 95 | 90 | 84 | INF vs Rest | DYRK3/IFIT1B | 88 | 75 | 80 |
| NPS vs Rest | GADD45A/GPR84 | 94 | 88 | 84 | INF vs Rest | DYRK3/CARD17 | 80 | 66 | 74 |
| NPS vs Rest | GADD45A/OLAH | 95 | 90 | 85 | INF vs Rest | FAM83A/TLCD4 | 88 | 78 | 78 |
| NPS vs Rest | GADD45A/ADAMTS3 | 95 | 93 | 86 | INF vs Rest | FAM83A/ANKRD22 | 83 | 70 | 77 |
| NPS vs Rest | GADD45A/PCOLCE2 | 93 | 85 | 84 | INF vs Rest | FAM83A/GBP5 | 82 | 70 | 77 |
| NPS vs Rest | GADD45A/ZDHHC19 | 95 | 89 | 87 | INF vs Rest | FAM83A/KLHDC8A | 84 | 70 | 76 |
| NPS vs Rest | GADD45A/SLC51A | 95 | 92 | 87 | INF vs Rest | FAM83A/SPTA1 | 87 | 75 | 78 |
| NPS vs Rest | GADD45A/HPGD | 95 | 88 | 85 | INF vs Rest | FAM83A/TSPAN5 | 88 | 79 | 81 |
| NPS vs Rest | GADD45A/SEMA6B | 93 | 87 | 83 | INF vs Rest | FAM83A/GYPA | 87 | 77 | 78 |
| NPS vs Rest | GADD45A/EFNA1 | 99 | 96 | 91 | INF vs Rest | FAM83A/P2RY14 | 83 | 69 | 77 |
| NPS vs Rest | GADD45A/AGFG1 | 95 | 88 | 83 | INF vs Rest | FAM83A/ITLN1 | 85 | 80 | 83 |
| NPS vs Rest | GADD45A/NSUN7 | 97 | 99 | 86 | INF vs Rest | FAM83A/RNF182 | 88 | 80 | 78 |
| NPS vs Rest | GADD45A/TNFAIP8L3 | 94 | 86 | 85 | INF vs Rest | FAM83A/GLRX5 | 87 | 79 | 76 |
| NPS vs Rest | GADD45A/KREMEN1 | 94 | 93 | 84 | INF vs Rest | FAM83A/RHCE | 86 | 75 | 78 |
| NPS vs Rest | GADD45A/ORM2 | 96 | 94 | 84 | INF vs Rest | FAM83A/THEM5 | 86 | 75 | 80 |
| NPS vs Rest | GADD45A/MIR646HG | 97 | 97 | 85 | INF vs Rest | FAM83A/IFIT1B | 89 | 79 | 82 |
| NPS vs Rest | GADD45A/KLF14 | 95 | 93 | 90 | INF vs Rest | FAM83A/CARD17 | 83 | 71 | 77 |
| NPS vs Rest | ARG1/PFKFB2 | 94 | 94 | 80 | INF vs Rest | TLCD4/ANKRD22 | 84 | 71 | 74 |
| NPS vs Rest | ARG1/MLLT1 | 98 | 91 | 88 | INF vs Rest | TLCD4/GBP5 | 84 | 73 | 74 |
| NPS vs Rest | ARG1/ANXA3 | 94 | 86 | 83 | INF vs Rest | TLCD4/KLHDC8A | 84 | 78 | 73 |
| NPS vs Rest | ARG1/GPR84 | 96 | 89 | 87 | INF vs Rest | TLCD4/SPTA1 | 85 | 74 | 78 |
| NPS vs Rest | ARG1/OLAH | 94 | 86 | 83 | INF vs Rest | TLCD4/TSPAN5 | 87 | 73 | 79 |
| NPS vs Rest | ARG1/ADAMTS3 | 95 | 90 | 87 | INF vs Rest | TLCD4/GYPA | 87 | 74 | 76 |
| NPS vs Rest | ARG1/PCOLCE2 | 93 | 86 | 84 | INF vs Rest | TLCD4/P2RY14 | 84 | 72 | 75 |
| NPS vs Rest | ARG1/ZDHHC19 | 95 | 98 | 87 | INF vs Rest | TLCD4/ITLN1 | 86 | 75 | 77 |
| NPS vs Rest | ARG1/SLC51A | 96 | 91 | 86 | INF vs Rest | TLCD4/RNF182 | 87 | 79 | 79 |
| NPS vs Rest | ARG1/HPGD | 94 | 81 | 86 | INF vs Rest | TLCD4/GLRX5 | 87 | 76 | 76 |
| NPS vs Rest | ARG1/SEMA6B | 96 | 91 | 87 | INF vs Rest | TLCD4/RHCE | 86 | 74 | 78 |
| NPS vs Rest | ARG1/EFNA1 | 97 | 80 | 92 | INF vs Rest | TLCD4/THEM5 | 88 | 80 | 80 |
| NPS vs Rest | ARG1/AGFG1 | 96 | 88 | 86 | INF vs Rest | TLCD4/IFIT1B | 89 | 79 | 79 |
| NPS vs Rest | ARG1/NSUN7 | 96 | 95 | 86 | INF vs Rest | TLCD4/CARD17 | 84 | 72 | 74 |
| NPS vs Rest | ARG1/TNFAIP8L3 | 95 | 86 | 87 | INF vs Rest | ANKRD22/KLHDC8A | 77 | 67 | 68 |
| NPS vs Rest | ARG1/KREMEN1 | 95 | 89 | 85 | INF vs Rest | ANKRD22/SPTA1 | 83 | 69 | 74 |
| NPS vs Rest | ARG1/ORM2 | 93 | 81 | 84 | INF vs Rest | ANKRD22/TSPAN5 | 86 | 75 | 78 |
| NPS vs Rest | ARG1/MIR646HG | 97 | 98 | 87 | INF vs Rest | ANKRD22/GYPA | 85 | 75 | 76 |
| NPS vs Rest | ARG1/KLF14 | 96 | 95 | 89 | INF vs Rest | ANKRD22/ITLN1 | 81 | 75 | 74 |
| NPS vs Rest | PFKFB2/MLLT1 | 95 | 95 | 83 | INF vs Rest | ANKRD22/RNF182 | 83 | 78 | 78 |
| NPS vs Rest | PFKFB2/ANXA3 | 92 | 90 | 77 | INF vs Rest | ANKRD22/GLRX5 | 86 | 80 | 75 |
| NPS vs Rest | PFKFB2/GPR84 | 92 | 87 | 79 | INF vs Rest | ANKRD22/RHCE | 84 | 74 | 78 |
| NPS vs Rest | PFKFB2/OLAH | 92 | 84 | 81 | INF vs Rest | ANKRD22/THEM5 | 83 | 71 | 77 |
| NPS vs Rest | PFKFB2/ADAMTS3 | 91 | 86 | 81 | INF vs Rest | ANKRD22/IFIT1B | 88 | 78 | 81 |
| NPS vs Rest | PFKFB2/PCOLCE2 | 90 | 84 | 79 | INF vs Rest | GBP5/KLHDC8A | 77 | 71 | 68 |
| NPS vs Rest | PFKFB2/ZDHHC19 | 94 | 89 | 83 | INF vs Rest | GBP5/SPTA1 | 82 | 70 | 76 |
| NPS vs Rest | PFKFB2/SLC51A | 93 | 87 | 83 | INF vs Rest | GBP5/TSPAN5 | 86 | 78 | 79 |
| NPS vs Rest | PFKFB2/HPGD | 91 | 87 | 80 | INF vs Rest | GBP5/GYPA | 85 | 79 | 75 |
| NPS vs Rest | PFKFB2/SEMA6B | 89 | 81 | 78 | INF vs Rest | GBP5/ITLN1 | 81 | 74 | 74 |
| NPS vs Rest | PFKFB2/EFNA1 | 97 | 89 | 88 | INF vs Rest | GBP5/RNF182 | 84 | 74 | 77 |
| NPS vs Rest | PFKFB2/AGFG1 | 92 | 92 | 80 | INF vs Rest | GBP5/GLRX5 | 86 | 80 | 75 |
| NPS vs Rest | PFKFB2/NSUN7 | 95 | 97 | 81 | INF vs Rest | GBP5/RHCE | 84 | 73 | 77 |
| NPS vs Rest | PFKFB2/TNFAIP8L3 | 91 | 86 | 80 | INF vs Rest | GBP5/THEM5 | 82 | 71 | 77 |
| NPS vs Rest | PFKFB2/KREMEN1 | 90 | 90 | 78 | INF vs Rest | GBP5/IFIT1B | 88 | 79 | 77 |
| NPS vs Rest | PFKFB2/ORM2 | 90 | 88 | 78 | INF vs Rest | KLHDC8A/SPTA1 | 82 | 75 | 73 |
| NPS vs Rest | PFKFB2/MIR646HG | 93 | 89 | 80 | INF vs Rest | KLHDC8A/TSPAN5 | 86 | 79 | 77 |
| NPS vs Rest | PFKFB2/KLF14 | 95 | 94 | 87 | INF vs Rest | KLHDC8A/GYPA | 86 | 75 | 74 |
| NPS vs Rest | MLLT1/ANXA3 | 94 | 84 | 82 | INF vs Rest | KLHDC8A/P2RY14 | 77 | 70 | 68 |
| NPS vs Rest | MLLT1/GPR84 | 94 | 82 | 83 | INF vs Rest | KLHDC8A/ITLN1 | 82 | 72 | 76 |
| NPS vs Rest | MLLT1/OLAH | 96 | 91 | 85 | INF vs Rest | KLHDC8A/RNF182 | 85 | 80 | 77 |
| NPS vs Rest | MLLT1/ADAMTS3 | 95 | 90 | 85 | INF vs Rest | KLHDC8A/GLRX5 | 86 | 78 | 75 |
| NPS vs Rest | MLLT1/PCOLCE2 | 93 | 78 | 85 | INF vs Rest | KLHDC8A/RHCE | 84 | 74 | 76 |
| NPS vs Rest | MLLT1/ZDHHC19 | 94 | 82 | 83 | INF vs Rest | KLHDC8A/THEM5 | 84 | 73 | 77 |
| NPS vs Rest | MLLT1/SLC51A | 95 | 81 | 84 | INF vs Rest | KLHDC8A/IFIT1B | 88 | 77 | 80 |
| NPS vs Rest | MLLT1/HPGD | 96 | 85 | 86 | INF vs Rest | KLHDC8A/CARD17 | 76 | 71 | 67 |
| NPS vs Rest | MLLT1/SEMA6B | 93 | 84 | 82 | INF vs Rest | SPTA1/TSPAN5 | 87 | 79 | 80 |
| NPS vs Rest | MLLT1/EFNA1 | 98 | 91 | 89 | INF vs Rest | SPTA1/GYPA | 86 | 72 | 78 |
| NPS vs Rest | MLLT1/AGFG1 | 95 | 86 | 85 | INF vs Rest | SPTA1/P2RY14 | 82 | 69 | 75 |
| NPS vs Rest | MLLT1/NSUN7 | 98 | 92 | 81 | INF vs Rest | SPTA1/ITLN1 | 85 | 77 | 79 |
| NPS vs Rest | MLLT1/TNFAIP8L3 | 93 | 80 | 83 | INF vs Rest | SPTA1/RNF182 | 86 | 77 | 79 |
| NPS vs Rest | MLLT1/KREMEN1 | 94 | 88 | 83 | INF vs Rest | SPTA1/GLRX5 | 86 | 78 | 78 |
| NPS vs Rest | MLLT1/ORM2 | 94 | 87 | 83 | INF vs Rest | SPTA1/RHCE | 86 | 72 | 77 |
| NPS vs Rest | MLLT1/MIR646HG | 98 | 98 | 87 | INF vs Rest | SPTA1/THEM5 | 87 | 74 | 81 |
| NPS vs Rest | MLLT1/KLF14 | 97 | 92 | 86 | INF vs Rest | SPTA1/IFIT1B | 88 | 81 | 82 |
| NPS vs Rest | ANXA3/GPR84 | 89 | 79 | 79 | INF vs Rest | SPTA1/CARD17 | 82 | 72 | 75 |
| NPS vs Rest | ANXA3/OLAH | 93 | 88 | 83 | INF vs Rest | TSPAN5/GYPA | 88 | 74 | 80 |
| NPS vs Rest | ANXA3/ADAMTS3 | 92 | 84 | 83 | INF vs Rest | TSPAN5/P2RY14 | 86 | 76 | 79 |
| NPS vs Rest | ANXA3/PCOLCE2 | 90 | 79 | 81 | INF vs Rest | TSPAN5/ITLN1 | 87 | 76 | 81 |
| NPS vs Rest | ANXA3/ZDHHC19 | 92 | 82 | 83 | INF vs Rest | TSPAN5/RNF182 | 89 | 79 | 79 |
| NPS vs Rest | ANXA3/SLC51A | 92 | 81 | 82 | INF vs Rest | TSPAN5/GLRX5 | 87 | 80 | 79 |
| NPS vs Rest | ANXA3/HPGD | 94 | 78 | 83 | INF vs Rest | TSPAN5/RHCE | 88 | 74 | 82 |
| NPS vs Rest | ANXA3/SEMA6B | 88 | 81 | 78 | INF vs Rest | TSPAN5/THEM5 | 88 | 80 | 79 |
| NPS vs Rest | ANXA3/EFNA1 | 96 | 83 | 89 | INF vs Rest | TSPAN5/IFIT1B | 89 | 77 | 81 |
| NPS vs Rest | ANXA3/AGFG1 | 93 | 82 | 83 | INF vs Rest | TSPAN5/CARD17 | 86 | 78 | 78 |
| NPS vs Rest | ANXA3/NSUN7 | 96 | 94 | 83 | INF vs Rest | GYPA/P2RY14 | 85 | 76 | 74 |
| NPS vs Rest | ANXA3/TNFAIP8L3 | 90 | 83 | 79 | INF vs Rest | GYPA/ITLN1 | 86 | 77 | 76 |
| NPS vs Rest | ANXA3/KREMEN1 | 89 | 80 | 75 | INF vs Rest | GYPA/RNF182 | 88 | 80 | 80 |
| NPS vs Rest | ANXA3/ORM2 | 88 | 77 | 76 | INF vs Rest | GYPA/GLRX5 | 87 | 75 | 75 |
| NPS vs Rest | ANXA3/MIR646HG | 95 | 94 | 81 | INF vs Rest | GYPA/RHCE | 87 | 74 | 78 |
| NPS vs Rest | ANXA3/KLF14 | 95 | 94 | 86 | INF vs Rest | GYPA/THEM5 | 88 | 77 | 79 |
| NPS vs Rest | GPR84/OLAH | 93 | 84 | 86 | INF vs Rest | GYPA/IFIT1B | 89 | 79 | 81 |
| NPS vs Rest | GPR84/ADAMTS3 | 90 | 76 | 84 | INF vs Rest | GYPA/CARD17 | 85 | 78 | 75 |
| NPS vs Rest | GPR84/PCOLCE2 | 87 | 77 | 83 | INF vs Rest | P2RY14/ITLN1 | 81 | 68 | 74 |
| NPS vs Rest | GPR84/ZDHHC19 | 91 | 84 | 82 | INF vs Rest | P2RY14/RNF182 | 83 | 74 | 78 |
| NPS vs Rest | GPR84/SLC51A | 91 | 75 | 82 | INF vs Rest | P2RY14/GLRX5 | 86 | 80 | 76 |
| NPS vs Rest | GPR84/HPGD | 93 | 86 | 83 | INF vs Rest | P2RY14/RHCE | 84 | 70 | 77 |
| NPS vs Rest | GPR84/SEMA6B | 85 | 80 | 79 | INF vs Rest | P2RY14/THEM5 | 82 | 70 | 77 |
| NPS vs Rest | GPR84/EFNA1 | 97 | 88 | 90 | INF vs Rest | P2RY14/IFIT1B | 88 | 78 | 79 |
| NPS vs Rest | GPR84/AGFG1 | 93 | 83 | 83 | INF vs Rest | ITLN1/RNF182 | 87 | 81 | 81 |
| NPS vs Rest | GPR84/NSUN7 | 97 | 99 | 84 | INF vs Rest | ITLN1/GLRX5 | 87 | 75 | 76 |
| NPS vs Rest | GPR84/TNFAIP8L3 | 87 | 76 | 81 | INF vs Rest | ITLN1/RHCE | 86 | 74 | 79 |
| NPS vs Rest | GPR84/KREMEN1 | 86 | 78 | 76 | INF vs Rest | ITLN1/THEM5 | 84 | 76 | 79 |
| NPS vs Rest | GPR84/ORM2 | 86 | 78 | 79 | INF vs Rest | ITLN1/IFIT1B | 88 | 78 | 81 |
| NPS vs Rest | GPR84/MIR646HG | 95 | 90 | 83 | INF vs Rest | ITLN1/CARD17 | 81 | 74 | 75 |
| NPS vs Rest | GPR84/KLF14 | 93 | 90 | 88 | INF vs Rest | RNF182/GLRX5 | 89 | 75 | 79 |
| NPS vs Rest | OLAH/ADAMTS3 | 92 | 78 | 84 | INF vs Rest | RNF182/RHCE | 88 | 80 | 81 |
| NPS vs Rest | OLAH/PCOLCE2 | 90 | 74 | 86 | INF vs Rest | RNF182/THEM5 | 87 | 78 | 82 |
| NPS vs Rest | OLAH/ZDHHC19 | 94 | 77 | 85 | INF vs Rest | RNF182/IFIT1B | 89 | 84 | 81 |
| NPS vs Rest | OLAH/SLC51A | 95 | 86 | 86 | INF vs Rest | RNF182/CARD17 | 84 | 77 | 79 |
| NPS vs Rest | OLAH/HPGD | 92 | 75 | 84 | INF vs Rest | GLRX5/RHCE | 87 | 76 | 78 |
| NPS vs Rest | OLAH/SEMA6B | 93 | 89 | 82 | INF vs Rest | GLRX5/THEM5 | 87 | 79 | 77 |
| NPS vs Rest | OLAH/EFNA1 | 96 | 83 | 90 | INF vs Rest | GLRX5/IFIT1B | 88 | 79 | 80 |
| NPS vs Rest | OLAH/AGFG1 | 94 | 88 | 84 | INF vs Rest | GLRX5/CARD17 | 86 | 80 | 75 |
| NPS vs Rest | OLAH/NSUN7 | 95 | 91 | 85 | INF vs Rest | RHCE/THEM5 | 87 | 75 | 80 |
| NPS vs Rest | OLAH/TNFAIP8L3 | 93 | 84 | 86 | INF vs Rest | RHCE/IFIT1B | 89 | 75 | 81 |
| NPS vs Rest | OLAH/KREMEN1 | 91 | 80 | 84 | INF vs Rest | RHCE/CARD17 | 84 | 72 | 79 |
| NPS vs Rest | OLAH/ORM2 | 89 | 75 | 83 | INF vs Rest | THEM5/IFIT1B | 89 | 78 | 82 |
| NPS vs Rest | OLAH/MIR646HG | 96 | 94 | 89 | INF vs Rest | THEM5/CARD17 | 82 | 72 | 78 |
| NPS vs Rest | OLAH/KLF14 | 95 | 94 | 90 | INF vs Rest | IFIT1B/CARD17 | 88 | 78 | 76 |
| NPS vs Rest | ADAMTS3/PCOLCE2 | 87 | 77 | 84 | IHD Endotype | | | | |
| NPS vs Rest | ADAMTS3/ZDHHC19 | 92 | 85 | 85 | IHD vs Rest | IL5RA/TRIM2 | 78 | 61 | 74 |
| NPS vs Rest | ADAMTS3/SLC51A | 93 | 86 | 86 | IHD vs Rest | IL5RA/SPRED1 | 93 | 82 | 82 |
| NPS vs Rest | ADAMTS3/HPGD | 92 | 78 | 84 | IHD vs Rest | IL5RA/GPR34 | 82 | 73 | 73 |
| NPS vs Rest | ADAMTS3/SEMA6B | 89 | 79 | 83 | IHD vs Rest | IL5RA/PLCB1 | 80 | 71 | 73 |
| NPS vs Rest | ADAMTS3/EFNA1 | 96 | 82 | 91 | IHD vs Rest | IL5RA/DYNC2H1 | 76 | 62 | 72 |
| NPS vs Rest | ADAMTS3/AGFG1 | 93 | 87 | 86 | IHD vs Rest | SMPD3/TRIM2 | 76 | 62 | 71 |
| NPS vs Rest | ADAMTS3/NSUN7 | 95 | 93 | 86 | IHD vs Rest | SMPD3/MAP7 | 75 | 69 | 71 |
| NPS vs Rest | ADAMTS3/TNFAIP8L3 | 90 | 77 | 85 | IHD vs Rest | SMPD3/SPRED1 | 91 | 79 | 79 |
| NPS vs Rest | ADAMTS3/KREMEN1 | 88 | 82 | 80 | IHD vs Rest | SMPD3/GPR34 | 81 | 71 | 75 |
| NPS vs Rest | ADAMTS3/ORM2 | 89 | 78 | 81 | IHD vs Rest | SMPD3/PLCB1 | 80 | 72 | 72 |
| NPS vs Rest | ADAMTS3/MIR646HG | 93 | 84 | 85 | IHD vs Rest | SMPD3/DYNC2H1 | 76 | 62 | 70 |
| NPS vs Rest | ADAMTS3/KLF14 | 94 | 89 | 88 | IHD vs Rest | PRSS33/SPRED1 | 90 | 78 | 79 |
| NPS vs Rest | PCOLCE2/ZDHHC19 | 90 | 78 | 85 | IHD vs Rest | PRSS33/GPR34 | 80 | 74 | 74 |
| NPS vs Rest | PCOLCE2/SLC51A | 91 | 74 | 85 | IHD vs Rest | PRSS33/PLCB1 | 79 | 67 | 71 |
| NPS vs Rest | PCOLCE2/HPGD | 89 | 74 | 83 | IHD vs Rest | SIGLEC8/TRIM2 | 77 | 63 | 73 |
| NPS vs Rest | PCOLCE2/SEMA6B | 85 | 77 | 81 | IHD vs Rest | SIGLEC8/MAP7 | 76 | 69 | 73 |
| NPS vs Rest | PCOLCE2/EFNA1 | 96 | 79 | 92 | IHD vs Rest | SIGLEC8/SPRED1 | 91 | 76 | 81 |
| NPS vs Rest | PCOLCE2/AGFG1 | 92 | 82 | 84 | IHD vs Rest | SIGLEC8/GPR34 | 81 | 74 | 75 |
| NPS vs Rest | PCOLCE2/NSUN7 | 96 | 92 | 84 | IHD vs Rest | SIGLEC8/PLCB1 | 80 | 68 | 72 |
| NPS vs Rest | PCOLCE2/TNFAIP8L3 | 85 | 75 | 83 | IHD vs Rest | SIGLEC8/DYNC2H1 | 76 | 66 | 72 |
| NPS vs Rest | PCOLCE2/KREMEN1 | 89 | 77 | 81 | IHD vs Rest | TRIM2/HRK | 76 | 64 | 70 |
| NPS vs Rest | PCOLCE2/ORM2 | 86 | 71 | 80 | IHD vs Rest | TRIM2/MAP7 | 88 | 80 | 79 |
| NPS vs Rest | PCOLCE2/MIR646HG | 95 | 86 | 87 | IHD vs Rest | TRIM2/CACNA2D3 | 79 | 70 | 71 |
| NPS vs Rest | PCOLCE2/KLF14 | 92 | 88 | 88 | IHD vs Rest | TRIM2/SPRED1 | 91 | 82 | 81 |
| NPS vs Rest | ZDHHC19/SLC51A | 92 | 76 | 83 | IHD vs Rest | TRIM2/SDC2 | 79 | 68 | 73 |
| NPS vs Rest | ZDHHC19/HPGD | 93 | 84 | 85 | IHD vs Rest | TRIM2/GPR82 | 78 | 66 | 71 |
| NPS vs Rest | ZDHHC19/SEMA6B | 90 | 84 | 82 | IHD vs Rest | TRIM2/GPR34 | 85 | 78 | 75 |
| NPS vs Rest | ZDHHC19/EFNA1 | 97 | 86 | 89 | IHD vs Rest | TRIM2/GRAMD1C | 76 | 66 | 70 |
| NPS vs Rest | ZDHHC19/AGFG1 | 95 | 89 | 85 | IHD vs Rest | TRIM2/PLCB1 | 84 | 81 | 76 |
| NPS vs Rest | ZDHHC19/NSUN7 | 96 | 100 | 87 | IHD vs Rest | TRIM2/DYNC2H1 | 80 | 63 | 73 |
| NPS vs Rest | ZDHHC19/TNFAIP8L3 | 91 | 82 | 84 | IHD vs Rest | TRIM2/TPRG1 | 79 | 71 | 71 |
| NPS vs Rest | ZDHHC19/KREMEN1 | 91 | 82 | 83 | IHD vs Rest | TRIM2/ZNF600 | 80 | 68 | 72 |
| NPS vs Rest | ZDHHC19/ORM2 | 91 | 86 | 81 | IHD vs Rest | ADAM23/MAP7 | 79 | 72 | 72 |
| NPS vs Rest | ZDHHC19/MIR646HG | 96 | 94 | 83 | IHD vs Rest | ADAM23/SPRED1 | 89 | 86 | 76 |
| NPS vs Rest | ZDHHC19/KLF14 | 95 | 89 | 88 | IHD vs Rest | ADAM23/GPR34 | 80 | 77 | 73 |
| NPS vs Rest | SLC51A/HPGD | 95 | 87 | 88 | IHD vs Rest | ADAM23/PLCB1 | 77 | 74 | 70 |
| NPS vs Rest | SLC51A/SEMA6B | 91 | 74 | 82 | IHD vs Rest | HRK/MAP7 | 77 | 64 | 75 |
| NPS vs Rest | SLC51A/EFNA1 | 97 | 90 | 91 | IHD vs Rest | HRK/SPRED1 | 92 | 80 | 81 |
| NPS vs Rest | SLC51A/AGFG1 | 95 | 83 | 85 | IHD vs Rest | HRK/GPR34 | 83 | 73 | 75 |
| NPS vs Rest | SLC51A/NSUN7 | 96 | 94 | 87 | IHD vs Rest | HRK/PLCB1 | 79 | 69 | 72 |
| NPS vs Rest | SLC51A/TNFAIP8L3 | 91 | 75 | 84 | IHD vs Rest | HRK/DYNC2H1 | 75 | 64 | 72 |
| NPS vs Rest | SLC51A/KREMEN1 | 92 | 80 | 82 | IHD vs Rest | HRK/ZNF600 | 77 | 64 | 70 |
| NPS vs Rest | SLC51A/ORM2 | 93 | 77 | 82 | IHD vs Rest | MAP7/CACNA2D3 | 76 | 64 | 71 |
| NPS vs Rest | SLC51A/MIR646HG | 96 | 94 | 84 | IHD vs Rest | MAP7/BAALC | 77 | 70 | 67 |
| NPS vs Rest | SLC51A/KLF14 | 96 | 90 | 90 | IHD vs Rest | MAP7/SPRED1 | 94 | 87 | 85 |
| NPS vs Rest | HPGD/SEMA6B | 91 | 75 | 83 | IHD vs Rest | MAP7/GPR82 | 85 | 68 | 78 |
| NPS vs Rest | HPGD/EFNA1 | 97 | 76 | 91 | IHD vs Rest | MAP7/GPR34 | 90 | 87 | 80 |
| NPS vs Rest | HPGD/AGFG1 | 94 | 86 | 84 | IHD vs Rest | MAP7/GRAMD1C | 75 | 65 | 74 |
| NPS vs Rest | HPGD/NSUN7 | 96 | 86 | 85 | IHD vs Rest | MAP7/PLCB1 | 86 | 76 | 77 |
| NPS vs Rest | HPGD/TNFAIP8L3 | 93 | 82 | 84 | IHD vs Rest | MAP7/DYNC2H1 | 84 | 72 | 79 |
| NPS vs Rest | HPGD/KREMEN1 | 92 | 82 | 82 | IHD vs Rest | MAP7/TPRG1 | 80 | 71 | 73 |
| NPS vs Rest | HPGD/ORM2 | 93 | 89 | 86 | IHD vs Rest | MAP7/ZNF600 | 86 | 74 | 76 |
| NPS vs Rest | HPGD/MIR646HG | 97 | 95 | 88 | IHD vs Rest | CACNA2D3/SPRED1 | 89 | 78 | 79 |
| NPS vs Rest | HPGD/KLF14 | 95 | 95 | 89 | IHD vs Rest | CACNA2D3/GPR34 | 82 | 73 | 74 |
| NPS vs Rest | SEMA6B/EFNA1 | 95 | 80 | 88 | IHD vs Rest | CACNA2D3/PLCB1 | 80 | 75 | 71 |
| NPS vs Rest | SEMA6B/AGFG1 | 91 | 83 | 81 | IHD vs Rest | CACNA2D3/DYNC2H1 | 75 | 64 | 71 |
| NPS vs Rest | SEMA6B/NSUN7 | 96 | 95 | 81 | IHD vs Rest | CACNA2D3/ZNF600 | 77 | 80 | 68 |
| NPS vs Rest | SEMA6B/TNFAIP8L3 | 86 | 76 | 79 | IHD vs Rest | ALOX15/SPRED1 | 89 | 75 | 79 |
| NPS vs Rest | SEMA6B/KREMEN1 | 85 | 78 | 73 | IHD vs Rest | ALOX15/GPR34 | 81 | 72 | 74 |
| NPS vs Rest | SEMA6B/ORM2 | 84 | 79 | 77 | IHD vs Rest | ALOX15/PLCB1 | 78 | 67 | 73 |
| NPS vs Rest | SEMA6B/MIR646HG | 92 | 82 | 80 | IHD vs Rest | BAALC/SPRED1 | 89 | 84 | 79 |
| NPS vs Rest | SEMA6B/KLF14 | 93 | 81 | 86 | IHD vs Rest | BAALC/GPR34 | 81 | 83 | 73 |
| NPS vs Rest | EFNA1/AGFG1 | 97 | 88 | 90 | IHD vs Rest | BAALC/PLCB1 | 78 | 79 | 68 |
| NPS vs Rest | EFNA1/NSUN7 | 98 | 85 | 92 | IHD vs Rest | SPRED1/SDC2 | 90 | 80 | 82 |
| NPS vs Rest | EFNA1/TNFAIP8L3 | 96 | 88 | 92 | IHD vs Rest | SPRED1/GPR82 | 89 | 76 | 79 |
| NPS vs Rest | EFNA1/KREMEN1 | 95 | 85 | 90 | IHD vs Rest | SPRED1/GPR34 | 94 | 88 | 83 |
| NPS vs Rest | EFNA1/ORM2 | 94 | 83 | 90 | IHD vs Rest | SPRED1/GRAMD1C | 89 | 83 | 80 |
| NPS vs Rest | EFNA1/MIR646HG | 99 | 97 | 92 | IHD vs Rest | SPRED1/PLCB1 | 91 | 90 | 83 |
| NPS vs Rest | EFNA1/KLF14 | 97 | 88 | 93 | IHD vs Rest | SPRED1/DYNC2H1 | 90 | 82 | 80 |
| NPS vs Rest | AGFG1/NSUN7 | 96 | 95 | 82 | IHD vs Rest | SPRED1/TPRG1 | 92 | 87 | 78 |
| NPS vs Rest | AGFG1/TNFAIP8L3 | 93 | 85 | 84 | IHD vs Rest | SPRED1/ZNF600 | 91 | 81 | 81 |
| NPS vs Rest | AGFG1/KREMEN1 | 93 | 88 | 83 | IHD vs Rest | SDC2/GPR34 | 84 | 69 | 79 |
| NPS vs Rest | AGFG1/ORM2 | 93 | 82 | 82 | IHD vs Rest | SDC2/PLCB1 | 81 | 75 | 73 |
| NPS vs Rest | AGFG1/MIR646HG | 95 | 93 | 87 | IHD vs Rest | SDC2/DYNC2H1 | 77 | 58 | 74 |
| NPS vs Rest | AGFG1/KLF14 | 97 | 95 | 89 | IHD vs Rest | SDC2/ZNF600 | 81 | 77 | 76 |
| NPS vs Rest | NSUN7/TNFAIP8L3 | 96 | 97 | 87 | IHD vs Rest | GPR82/GPR34 | 80 | 71 | 76 |
| NPS vs Rest | NSUN7/KREMEN1 | 95 | 91 | 80 | IHD vs Rest | GPR82/GRAMD1C | 76 | 68 | 69 |
| NPS vs Rest | NSUN7/ORM2 | 95 | 93 | 82 | IHD vs Rest | GPR82/PLCB1 | 84 | 79 | 73 |
| NPS vs Rest | NSUN7/MIR646HG | 96 | 97 | 84 | IHD vs Rest | GPR82/DYNC2H1 | 77 | 69 | 73 |
| NPS vs Rest | NSUN7/KLF14 | 97 | 95 | 92 | IHD vs Rest | GPR82/TPRG1 | 77 | 65 | 68 |
| NPS vs Rest | TNFAIP8L3/KREMEN1 | 89 | 80 | 79 | IHD vs Rest | GPR82/ZNF600 | 78 | 69 | 70 |
| NPS vs Rest | TNFAIP8L3/ORM2 | 88 | 76 | 82 | IHD vs Rest | GPR34/GRAMD1C | 81 | 75 | 74 |
| NPS vs Rest | TNFAIP8L3/MIR646HG | 94 | 85 | 84 | IHD vs Rest | GPR34/PLCB1 | 88 | 87 | 78 |
| NPS vs Rest | TNFAIP8L3/KLF14 | 93 | 84 | 87 | IHD vs Rest | GPR34/DYNC2H1 | 83 | 70 | 81 |
| NPS vs Rest | KREMEN1/ORM2 | 82 | 84 | 69 | IHD vs Rest | GPR34/TPRG1 | 83 | 81 | 78 |
| NPS vs Rest | KREMEN1/MIR646HG | 93 | 85 | 79 | IHD vs Rest | GPR34/ZNF600 | 84 | 80 | 79 |
| NPS vs Rest | KREMEN1/KLF14 | 94 | 85 | 86 | IHD vs Rest | GRAMD1C/PLCB1 | 77 | 70 | 70 |
| NPS vs Rest | ORM2/MIR646HG | 91 | 82 | 80 | IHD vs Rest | GRAMDIC/DYNC2H1 | 75 | 63 | 71 |
| NPS vs Rest | ORM2/KLF14 | 94 | 86 | 88 | IHD vs Rest | GRAMD1C/ZNF600 | 76 | 75 | 69 |
| NPS vs Rest | MIR646HG/KLF14 | 98 | 93 | 90 | IHD vs Rest | PLCB1/DYNC2H1 | 83 | 76 | 77 |
| INF Endotype | | | | | IHD vs Rest | PLCB1/TPRG1 | 82 | 83 | 70 |
| INF vs Rest | FECH/APOL4 | 89 | 83 | 80 | IHD vs Rest | PLCB1/ZNF600 | 83 | 78 | 73 |
| INF vs Rest | FECH/RIOK3 | 89 | 79 | 79 | IHD vs Rest | DYNC2H1/TPRG1 | 78 | 63 | 72 |
| INF vs Rest | FECH/BNIP3L | 89 | 83 | 81 | IHD vs Rest | DYNC2H1/ZNF600 | 79 | 65 | 77 |
| INF vs Rest | FECH/TFEC | 91 | 83 | 83 | IFN Endotype | | | | |
| INF vs Rest | FECH/RHAG | 89 | 81 | 81 | IFN vs Rest | ETV7/PLEKHO1 | 93 | 90 | 78 |
| INF vs Rest | FECH/TSPO2 | 89 | 79 | 81 | IFN vs Rest | ETV7/LAMP3 | 90 | 84 | 77 |
| INF vs Rest | FECH/CD274 | 89 | 82 | 81 | IFN vs Rest | ETV7/APOL1 | 92 | 89 | 80 |
| INF vs Rest | FECH/TMCC2 | 89 | 79 | 79 | IFN vs Rest | ETV7/SEPTIN4 | 90 | 87 | 78 |
| INF vs Rest | FECH/CA1 | 90 | 83 | 79 | IFN vs Rest | ETV7/EPSTI1 | 91 | 83 | 76 |
| INF vs Rest | FECH/DYRK3 | 89 | 80 | 80 | IFN vs Rest | ETV7/RSAD2 | 89 | 82 | 76 |
| INF vs Rest | FECH/FAM83A | 89 | 81 | 80 | IFN vs Rest | ETV7/IFITM3 | 92 | 84 | 80 |
| INF vs Rest | FECH/TLCD4 | 89 | 78 | 81 | IFN vs Rest | ETV7/SERPING1 | 90 | 88 | 78 |
| INF vs Rest | FECH/ANKRD22 | 89 | 82 | 79 | IFN vs Rest | ETV7/CLEC4F | 90 | 88 | 76 |
| INF vs Rest | FECH/GBP5 | 89 | 83 | 79 | IFN vs Rest | ETV7/TPPP3 | 90 | 87 | 77 |
| INF vs Rest | FECH/KLHDC8A | 89 | 83 | 78 | IFN vs Rest | ETV7/LY6E | 89 | 85 | 76 |
| INF vs Rest | FECH/SPTA1 | 89 | 81 | 81 | IFN vs Rest | ETV7/BATF2 | 92 | 89 | 78 |
| INF vs Rest | FECH/TSPAN5 | 89 | 81 | 82 | IFN vs Rest | ETV7/EXOC3L1 | 90 | 84 | 76 |
| INF vs Rest | FECH/GYPA | 89 | 82 | 80 | IFN vs Rest | ETV7/HES4 | 89 | 84 | 76 |
| INF vs Rest | FECH/P2RY14 | 89 | 82 | 80 | IFN vs Rest | PLEKHO1/LAMP3 | 81 | 75 | 75 |
| INF vs Rest | FECH/ITLN1 | 89 | 82 | 81 | IFN vs Rest | PLEKHO1/APOL1 | 91 | 85 | 80 |
| INF vs Rest | FECH/RNF182 | 90 | 84 | 81 | IFN vs Rest | PLEKHO1/SEPTIN4 | 82 | 73 | 75 |
| INF vs Rest | FECH/GLRX5 | 89 | 81 | 79 | IFN vs Rest | PLEKHO1/EPSTI1 | 88 | 84 | 71 |
| INF vs Rest | FECH/RHCE | 89 | 79 | 80 | IFN vs Rest | PLEKHO1/RSAD2 | 76 | 69 | 66 |
| INF vs Rest | FECH/THEM5 | 89 | 83 | 81 | IFN vs Rest | PLEKHO1/IFITM3 | 82 | 78 | 72 |
| INF vs Rest | FECH/IFIT1B | 90 | 82 | 79 | IFN vs Rest | PLEKHO1/SERPING1 | 89 | 81 | 78 |
| INF vs Rest | FECH/CARD17 | 88 | 82 | 79 | IFN vs Rest | PLEKHO1/BATF2 | 92 | 89 | 79 |
| INF vs Rest | APOL4/RIOK3 | 84 | 68 | 77 | IFN vs Rest | PLEKHO1/EXOC3L1 | 78 | 70 | 71 |
| INF vs Rest | APOL4/BNIP3L | 86 | 75 | 77 | IFN vs Rest | LAMP3/APOL1 | 89 | 85 | 80 |
| INF vs Rest | APOL4/RHAG | 83 | 70 | 72 | IFN vs Rest | LAMP3/SEPTIN4 | 84 | 74 | 77 |
| INF vs Rest | APOL4/TSPO2 | 81 | 72 | 71 | IFN vs Rest | LAMP3/EPSTI1 | 87 | 82 | 73 |
| INF vs Rest | APOL4/TMCC2 | 83 | 75 | 73 | IFN vs Rest | LAMP3/RSAD2 | 81 | 76 | 71 |
| INF vs Rest | APOL4/CA1 | 88 | 87 | 75 | IFN vs Rest | LAMP3/IFITM3 | 85 | 79 | 75 |
| INF vs Rest | APOL4/DYRK3 | 81 | 73 | 73 | IFN vs Rest | LAMP3/SERPING1 | 88 | 81 | 77 |
| INF vs Rest | APOL4/FAM83A | 82 | 68 | 77 | IFN vs Rest | LAMP3/CLEC4F | 81 | 80 | 73 |
| INF vs Rest | APOL4/TLCD4 | 84 | 74 | 76 | IFN vs Rest | LAMP3/TPPP3 | 81 | 76 | 73 |
| INF vs Rest | APOL4/KLHDC8A | 78 | 74 | 67 | IFN vs Rest | LAMP3/LY6E | 81 | 74 | 74 |
| INF vs Rest | APOL4/SPTA1 | 83 | 71 | 77 | IFN vs Rest | LAMP3/BATF2 | 90 | 84 | 77 |
| INF vs Rest | APOL4/TSPAN5 | 86 | 78 | 78 | IFN vs Rest | LAMP3/EXOC3L1 | 81 | 76 | 72 |
| INF vs Rest | APOL4/GYPA | 85 | 79 | 74 | IFN vs Rest | LAMP3/HES4 | 86 | 80 | 76 |
| INF vs Rest | APOL4/ITLN1 | 82 | 74 | 74 | IFN vs Rest | APOL1/SEPTIN4 | 88 | 86 | 80 |
| INF vs Rest | APOL4/RNF182 | 84 | 74 | 78 | IFN vs Rest | APOL1/EPSTI1 | 91 | 84 | 78 |
| INF vs Rest | APOL4/GLRX5 | 86 | 79 | 76 | IFN vs Rest | APOL1/RSAD2 | 89 | 81 | 79 |
| INF vs Rest | APOL4/RHCE | 84 | 71 | 75 | IFN vs Rest | APOL1/IFITM3 | 91 | 82 | 81 |
| INF vs Rest | APOL4/THEM5 | 83 | 75 | 76 | IFN vs Rest | APOL1/SERPING1 | 90 | 88 | 80 |
| INF vs Rest | APOL4/IFIT1B | 88 | 79 | 77 | IFN vs Rest | APOL1/CLEC4F | 89 | 87 | 80 |
| INF vs Rest | RIOK3/BNIP3L | 86 | 73 | 79 | IFN vs Rest | APOL1/TPPP3 | 89 | 82 | 78 |
| INF vs Rest | RIOK3/TFEC | 87 | 77 | 80 | IFN vs Rest | APOL1/LY6E | 88 | 82 | 80 |
| INF vs Rest | RIOK3/RHAG | 85 | 68 | 79 | IFN vs Rest | APOL1/BATF2 | 91 | 89 | 79 |
| INF vs Rest | RIOK3/TSPO2 | 84 | 67 | 78 | IFN vs Rest | APOL1/EXOC3L1 | 89 | 81 | 80 |
| INF vs Rest | RIOK3/CD274 | 82 | 70 | 77 | IFN vs Rest | APOL1/HES4 | 88 | 82 | 80 |
| INF vs Rest | RIOK3/TMCC2 | 86 | 69 | 79 | IFN vs Rest | SEPTIN4/EPSTI1 | 88 | 84 | 72 |
| INF vs Rest | RIOK3/CA1 | 88 | 79 | 79 | IFN vs Rest | SEPTIN4/RSAD2 | 82 | 78 | 72 |
| INF vs Rest | RIOK3/DYRK3 | 83 | 70 | 81 | IFN vs Rest | SEPTIN4/IFITM3 | 87 | 79 | 79 |
| INF vs Rest | RIOK3/FAM83A | 86 | 74 | 78 | IFN vs Rest | SEPTIN4/SERPING1 | 87 | 80 | 76 |
| INF vs Rest | RIOK3/TLCD4 | 85 | 71 | 78 | IFN vs Rest | SEPTIN4/CLEC4F | 82 | 79 | 74 |
| INF vs Rest | RIOK3/ANKRD22 | 82 | 68 | 78 | IFN vs Rest | SEPTIN4/TPPP3 | 83 | 78 | 75 |
| INF vs Rest | RIOK3/GBP5 | 83 | 67 | 77 | IFN vs Rest | SEPTIN4/LY6E | 81 | 76 | 75 |
| INF vs Rest | RIOK3/KLHDC8A | 83 | 66 | 74 | IFN vs Rest | SEPTIN4/BATF2 | 89 | 86 | 77 |
| INF vs Rest | RIOK3/SPTA1 | 84 | 71 | 81 | IFN vs Rest | SEPTIN4/EXOC3L1 | 83 | 79 | 73 |
| INF vs Rest | RIOK3/TSPAN5 | 86 | 75 | 79 | IFN vs Rest | SEPTIN4/HES4 | 83 | 75 | 76 |
| INF vs Rest | RIOK3/GYPA | 86 | 70 | 77 | IFN vs Rest | EPSTI1/RSAD2 | 87 | 84 | 70 |
| INF vs Rest | RIOK3/P2RY14 | 82 | 66 | 78 | IFN vs Rest | EPSTI1/IFITM3 | 89 | 85 | 75 |
| INF vs Rest | RIOK3/ITLN1 | 85 | 70 | 79 | IFN vs Rest | EPSTI1/SERPING1 | 89 | 85 | 75 |
| INF vs Rest | RIOK3/RNF182 | 86 | 76 | 79 | IFN vs Rest | EPSTI1/CLEC4F | 87 | 84 | 71 |
| INF vs Rest | RIOK3/GLRX5 | 86 | 75 | 75 | IFN vs Rest | EPSTI1/TPPP3 | 88 | 87 | 73 |
| INF vs Rest | RIOK3/RHCE | 85 | 70 | 79 | IFN vs Rest | EPSTI1/LY6E | 91 | 88 | 73 |
| INF vs Rest | RIOK3/THEM5 | 86 | 73 | 80 | IFN vs Rest | EPSTI1/BATF2 | 91 | 83 | 76 |
| INF vs Rest | RIOK3/IFIT1B | 88 | 74 | 80 | IFN vs Rest | EPSTI1/EXOC3L1 | 87 | 86 | 69 |
| INF vs Rest | RIOK3/CARD17 | 82 | 68 | 75 | IFN vs Rest | EPSTI1/HES4 | 89 | 86 | 76 |
| INF vs Rest | BNIP3L/TFEC | 89 | 80 | 81 | IFN vs Rest | RSAD2/IFITM3 | 83 | 80 | 73 |
| INF vs Rest | BNIP3L/RHAG | 87 | 75 | 79 | IFN vs Rest | RSAD2/SERPING1 | 86 | 81 | 74 |
| INF vs Rest | BNIP3L/TSPO2 | 87 | 72 | 78 | IFN vs Rest | RSAD2/CLEC4F | 77 | 72 | 66 |
| INF vs Rest | BNIP3L/CD274 | 86 | 77 | 77 | IFN vs Rest | RSAD2/TPPP3 | 79 | 83 | 67 |
| INF vs Rest | BNIP3L/TMCC2 | 87 | 75 | 78 | IFN vs Rest | RSAD2/LY6E | 80 | 75 | 69 |
| INF vs Rest | BNIP3L/CA1 | 88 | 80 | 78 | IFN vs Rest | RSAD2/BATF2 | 89 | 80 | 75 |
| INF vs Rest | BNIP3L/DYRK3 | 86 | 73 | 77 | IFN vs Rest | RSAD2/EXOC3L1 | 78 | 74 | 67 |
| INF vs Rest | BNIP3L/FAM83A | 88 | 75 | 79 | IFN vs Rest | RSAD2/HES4 | 81 | 73 | 70 |
| INF vs Rest | BNIP3L/TLCD4 | 87 | 72 | 79 | IFN vs Rest | IFITM3/SERPING1 | 89 | 83 | 77 |
| INF vs Rest | BNIP3L/ANKRD22 | 86 | 76 | 75 | IFN vs Rest | IFITM3/CLEC4F | 82 | 80 | 72 |
| INF vs Rest | BNIP3L/GBP5 | 86 | 76 | 75 | IFN vs Rest | IFITM3/TPPP3 | 86 | 86 | 73 |
| INF vs Rest | BNIP3L/KLHDC8A | 86 | 78 | 77 | IFN vs Rest | IFITM3/LY6E | 81 | 76 | 72 |
| INF vs Rest | BNIP3L/SPTA1 | 87 | 76 | 80 | | | | | |
| INF vs Rest | BNIP3L/TSPAN5 | 87 | 77 | 81 | IFN vs Rest | IFITM3/EXOC3L1 | 83 | 80 | 73 |
| INF vs Rest | BNIP3L/GYPA | 88 | 74 | 78 | IFN vs Rest | IFITM3/HES4 | 82 | 80 | 69 |
| INF vs Rest | BNIP3L/P2RY14 | 86 | 76 | 77 | IFN vs Rest | SERPING1/CLEC4F | 87 | 83 | 75 |
| INF vs Rest | BNIP3L/ITLN1 | 87 | 75 | 80 | IFN vs Rest | SERPING1/TPPP3 | 89 | 90 | 76 |
| INF vs Rest | BNIP3L/RNF182 | 89 | 77 | 79 | IFN vs Rest | SERPING1/LY6E | 87 | 83 | 76 |
| INF vs Rest | BNIP3L/GLRX5 | 87 | 76 | 77 | IFN vs Rest | SERPING1/BATF2 | 90 | 87 | 76 |
| INF vs Rest | BNIP3L/RHCE | 88 | 75 | 80 | IFN vs Rest | SERPING1/EXOC3L1 | 87 | 84 | 74 |
| INF vs Rest | BNIP3L/THEM5 | 89 | 77 | 80 | IFN vs Rest | SERPING1/HES4 | 88 | 84 | 74 |
| INF vs Rest | BNIP3L/IFIT1B | 89 | 78 | 81 | IFN vs Rest | CLEC4F/BATF2 | 88 | 87 | 75 |
| INF vs Rest | BNIP3L/CARD17 | 86 | 76 | 76 | IFN vs Rest | CLEC4F/EXOC3L1 | 80 | 77 | 70 |
| INF vs Rest | TFEC/RHAG | 84 | 72 | 76 | IFN vs Rest | TPPP3/BATF2 | 89 | 86 | 75 |
| INF vs Rest | TFEC/TSPO2 | 82 | 69 | 75 | IFN vs Rest | TPPP3/EXOC3L1 | 82 | 74 | 71 |
| INF vs Rest | TFEC/TMCC2 | 84 | 71 | 75 | IFN vs Rest | LY6E/BATF2 | 88 | 81 | 76 |
| INF vs Rest | TFEC/CA1 | 89 | 89 | 79 | IFN vs Rest | LY6E/EXOC3L1 | 80 | 74 | 72 |
| INF vs Rest | TFEC/DYRK3 | 83 | 74 | 74 | IFN vs Rest | BATF2/EXOC3L1 | 89 | 85 | 76 |
| INF vs Rest | TFEC/FAM83A | 84 | 73 | 78 | IFN vs Rest | BATF2/HES4 | 88 | 84 | 76 |
| INF vs Rest | TFEC/TLCD4 | 87 | 76 | 79 | IFN vs Rest | EXOC3L1/HES4 | 85 | 78 | 75 |
| INF vs Rest | TFEC/KLHDC8A | 79 | 70 | 70 | ADA Endotype | | | | |
| INF vs Rest | TFEC/SPTA1 | 85 | 71 | 79 | ADA vs Rest | IGF1/LGALS3BP | 86 | 76 | 82 |
| INF vs Rest | TFEC/TSPAN5 | 88 | 80 | 82 | ADA vs Rest | IGF1/OTOF | 82 | 71 | 83 |
| INF vs Rest | TFEC/GYPA | 88 | 84 | 77 | ADA vs Rest | TNFRSF17/LGALS3BP | 86 | 73 | 82 |
| INF vs Rest | TFEC/ITLN1 | 83 | 70 | 78 | ADA vs Rest | TNFRSF17/OTOF | 83 | 68 | 82 |
| INF vs Rest | TFEC/RNF182 | 84 | 72 | 78 | ADA vs Rest | GTSE1/LGALS3BP | 87 | 76 | 84 |
| INF vs Rest | TFEC/GLRX5 | 89 | 81 | 80 | ADA vs Rest | GTSE1/OTOF | 82 | 69 | 85 |
| INF vs Rest | TFEC/RHCE | 87 | 71 | 82 | ADA vs Rest | CDC45/LGALS3BP | 86 | 75 | 83 |
| INF vs Rest | TFEC/THEM5 | 84 | 71 | 80 | ADA vs Rest | CDC45/OTOF | 83 | 69 | 85 |
| INF vs Rest | TFEC/IFIT1B | 90 | 81 | 81 | ADA vs Rest | CAVI/LGALS3BP | 85 | 74 | 81 |
| INF vs Rest | RHAG/TSPO2 | 83 | 71 | 75 | ADA vs Rest | CAVI/OTOF | 81 | 70 | 82 |
| INF vs Rest | RHAG/CD274 | 82 | 70 | 73 | ADA vs Rest | LGALS3BP/GPRC5D | 86 | 77 | 82 |
| INF vs Rest | RHAG/TMCC2 | 84 | 73 | 76 | ADA vs Rest | LGALS3BP/OTOF | 88 | 78 | 86 |
| INF vs Rest | RHAG/CA1 | 87 | 86 | 77 | ADA vs Rest | LGALS3BP/SDC1 | 86 | 73 | 82 |
| INF vs Rest | RHAG/DYRK3 | 83 | 71 | 75 | ADA vs Rest | LGALS3BP/CENPF | 87 | 79 | 83 |
| INF vs Rest | RHAG/FAM83A | 85 | 73 | 77 | ADA vs Rest | LGALS3BP/KIF14 | 88 | 77 | 81 |
| INF vs Rest | RHAG/TLCD4 | 85 | 77 | 78 | ADA vs Rest | LGALS3BP/PLAAT2 | 87 | 75 | 81 |
| INF vs Rest | RHAG/ANKRD22 | 82 | 69 | 73 | ADA vs Rest | LGALS3BP/KCTD14 | 87 | 75 | 83 |
| INF vs Rest | RHAG/GBP5 | 82 | 72 | 72 | ADA vs Rest | LGALS3BP/PDIA4 | 87 | 76 | 84 |
| INF vs Rest | RHAG/KLHDC8A | 83 | 75 | 75 | ADA vs Rest | LGALS3BP/SLC16A14 | 85 | 75 | 81 |
| INF vs Rest | RHAG/SPTA1 | 85 | 70 | 77 | ADA vs Rest | LGALS3BP/KIF15 | 86 | 76 | 83 |
| INF vs Rest | RHAG/TSPAN5 | 87 | 76 | 80 | ADA vs Rest | LGALS3BP/TSHR | 87 | 76 | 82 |
| INF vs Rest | RHAG/GYPA | 86 | 73 | 76 | ADA vs Rest | LGALS3BP/IFI27 | 88 | 78 | 82 |
| INF vs Rest | RHAG/P2RY14 | 81 | 71 | 73 | ADA vs Rest | LGALS3BP/MIXL1 | 86 | 74 | 83 |
| INF vs Rest | RHAG/ITLN1 | 85 | 73 | 78 | ADA vs Rest | LGALS3BP/KLHL14 | 86 | 77 | 82 |
| INF vs Rest | RHAG/RNF182 | 86 | 79 | 79 | ADA vs Rest | LGALS3BP/MIR155HG | 85 | 73 | 82 |
| INF vs Rest | RHAG/GLRX5 | 86 | 76 | 77 | ADA vs Rest | LGALS3BP/IGLL5 | 86 | 77 | 80 |
| INF vs Rest | RHAG/RHCE | 85 | 76 | 78 | ADA vs Rest | GPRC5D/OTOF | 82 | 71 | 84 |
| INF vs Rest | RHAG/THEM5 | 86 | 74 | 81 | ADA vs Rest | OTOF/SDC1 | 81 | 71 | 82 |
| INF vs Rest | RHAG/IFIT1B | 89 | 79 | 81 | ADA vs Rest | OTOF/CENPF | 83 | 70 | 87 |
| INF vs Rest | RHAG/CARD17 | 82 | 72 | 71 | ADA vs Rest | OTOF/KIF14 | 83 | 74 | 82 |
| INF vs Rest | TSPO2/CD274 | 81 | 70 | 71 | ADA vs Rest | OTOF/PLAAT2 | 83 | 67 | 81 |
| INF vs Rest | TSPO2/TMCC2 | 83 | 66 | 76 | ADA vs Rest | OTOF/KCTD14 | 82 | 70 | 86 |
| INF vs Rest | TSPO2/CA1 | 87 | 82 | 76 | ADA vs Rest | OTOF/PDIA4 | 83 | 72 | 85 |
| INF vs Rest | TSPO2/DYRK3 | 82 | 66 | 75 | ADA vs Rest | OTOF/SLC16A14 | 82 | 69 | 84 |
| INF vs Rest | TSPO2/FAM83A | 85 | 76 | 78 | ADA vs Rest | OTOF/KIF15 | 82 | 69 | 84 |
| INF vs Rest | TSPO2/TLCD4 | 85 | 69 | 76 | ADA vs Rest | OTOF/TSHR | 83 | 70 | 82 |
| INF vs Rest | TSPO2/ANKRD22 | 81 | 67 | 70 | ADA vs Rest | OTOF/IFI27 | 87 | 76 | 86 |
| INF vs Rest | TSPO2/GBP5 | 80 | 70 | 70 | ADA vs Rest | OTOF/MIXL1 | 82 | 71 | 82 |
| INF vs Rest | TSPO2/KLHDC8A | 82 | 66 | 72 | ADA vs Rest | OTOF/KLHL14 | 81 | 68 | 84 |
| INF vs Rest | TSPO2/SPTA1 | 84 | 71 | 76 | ADA vs Rest | OTOF/MIR155HG | 81 | 69 | 83 |
| INF vs Rest | TSPO2/TSPAN5 | 86 | 73 | 80 | ADA vs Rest | OTOF/IGLL5 | 83 | 70 | 83 |
| INF vs Rest | TSPO2/GYPA | 85 | 72 | 76 | ADA vs Rest | CENPF/KCTD14 | 76 | 61 | 75 |
| INF vs Rest | TSPO2/P2RY14 | 80 | 68 | 72 | ADA vs Rest | KIF14/KCTD14 | 77 | 58 | 75 |
| INF vs Rest | TSPO2/ITLN1 | 83 | 67 | 77 | ADA vs Rest | PLAAT2/KCTD14 | 76 | 66 | 76 |
| INF vs Rest | TSPO2/RNF182 | 87 | 73 | 79 | ADA vs Rest | KCTD14/PDIA4 | 77 | 63 | 76 |
| INF vs Rest | TSPO2/GLRX5 | 86 | 77 | 76 | ADA vs Rest | KCTD14/TSHR | 75 | 62 | 77 |
| | | | | | ADA vs Rest | KCTD14/KLHL14 | 75 | 66 | 73 |

TABLE 7
Severity and outcomes of the endotypes in the ICU cohort.

| | Mechanistic Endotypes | | | | | | | | |
|---|---|---|---|---|---|---|---|---|---|
| | NPS | | INF | | IHD | | IFN | | |
| Parameter | (N = 36) | | (N = 33) | | (N = 6) | | (N = 7) | | P Val |
| Covid-19 PCR | 16.7% | (6/36) | 39.4% | (13/33) | 16.7% | (1/6) | 100% | (7/7) | 5.0e−4 |
| Positivity | | | | | | | | | |
| Mortality | 45.7% | (16/35) | 25.9% | (7/27) | 0% | (0/5) | 0% | (0/6) | 2.5e−2 |
| within 28 Days | | | | | | | | | |
| SOFA 24 H post | 7.6 ± 0.9 | (34) | 8.2 ± 0.78 | (32) | 3.5 ± 1.34 | (6) | 3.7 ± 1.49 | (7) | 3.3e−2 |
| ICU admission | | | | | | | | | |
| ICU Mortality | 38.9% | (14/36) | 18.2% | (6/33) | 0% | (0/6) | 0% | (0/5) | 3.4-e−2 |
| ICU Stay Days | 10.4 ± 1.29 | (36) | 15.2 ± 1.63 | (33) | 6.8 ± 2.7 | (6) | 9.7 ± 3.43 | (7) | 5.0e−2 |
| SOFA 48 H post | 7.5 ± 0.98 | (31) | 8.4 ± 0.75 | (30) | 3.5 ± 0.87 | (4) | 4.1 ± 1.7 | (7) | 7.9e−2 |
| admission | | | | | | | | | |
| SOFA at ICU | 8.4 ± 0.9 | (36) | 7.9 ± 0.64 | (33) | 4.2 ± 1.7 | (6) | 5 ± 1.66 | (7) | 9.3e−2 |
| admission | | | | | | | | | |

The mean value ± standard error is presented for numerical variables with the total available observations/patient numbers recorded in brackets. Categorical variables are presented as percent positive (% total positive/total available observations). P values are derived from Wilcoxon and Chi squared tests testing for significant differences between endotypes for numerical and categorical values, respectively.

TABLE 8
Gene set for classifying patients into severity groups.

| | | Fold Change | | | |
|---|---|---|---|---|---|
| | | High | Int. vs. | High + Int. | High |
| Gene Name | Description | VS. Low | Low | vs. Low | vs. Int. |
| ABCA13 | ATP binding cassette | 1.67 | −1.56 | −1.06 | 2.62 |
| | subfamily A member 13 | | | | |
| ADAMTS2 | ADAM metallopeptidase | 1.1 | 1.73 | 1.52 | −1.57 |
| | thrombospondin T1 M2 | | | | |
| ADAMTS3 | ADAM metallopeptidase | 2.99 | 1.24 | 1.88 | 2.41 |
| | thrombospondin T1 M3 | | | | |
| AK5* | adenylate kinase 5 | −1.52 | −1.37 | −1.42 | −1.11 |
| ANKRD22* | ankyrin repeat domain 22 | 2.48 | 1.1 | 1.41 | 2.25 |
| ANKRD34B | ankyrin repeat domain 34B | 1.68 | −1.05 | 1.17 | 1.78 |
| ANLN | anillin actin binding protein | 2.03 | −1.48 | 1.03 | 3.01 |
| AQP1 | aquaporin 1 (Colton blood group) | 1.66 | −1.01 | 1.19 | 1.68 |
| ARG1 | arginase 1 | 1.11 | 1.31 | 1.25 | −1.18 |
| ARHGAP44 | Rho GTPase activating protein 44 | −4 | −2.44 | −2.36 | −1.64 |
| ARHGEF17* | Rho guanine nucleotide | 1.64 | 1.46 | 1.53 | 1.12 |
| | exchange factor 17 | | | | |
| ASPM* | assembly factor for spindle | 1.69 | −1.91 | −1.19 | 3.23 |
| | microtubules | | | | |
| ATP1B2* | ATPase Na+/K+ | 1.96 | 1.09 | 1.37 | 1.79 |
| | transporting subunit beta 2 | | | | |
| AURKA* | aurora kinase A | 1.63 | −1.09 | 1.14 | 1.78 |
| AZU1 | azurocidin 1 | 1.89 | −1.56 | −1.02 | 2.95 |
| BAIAP3* | BAI1 associated protein 3 | −2.46 | −1.25 | −1.57 | −1.97 |
| BPI | bactericidal permeability | 2.17 | −1.07 | 1.31 | 2.34 |
| | increasing protein | | | | |
| C1orf226* | chromosome 1 open reading | 1.66 | 1.29 | 1.42 | 1.28 |
| | frame 226 | | | | |
| CACNB4* | calcium voltage-gated | −1.63 | −1.15 | −1.31 | −1.41 |
| | channel auxiliary SU β4 | | | | |
| CCL4L2*^{#} | C—C motif chemokine | 40.32 | 5.19 | 1 | 7.77 |
| | ligand 4 like 2 | | | | |
| CCN3* | cellular communication | −2.35 | −1.6 | −1.82 | −1.47 |
| | network factor 3 | | | | |
| CCNA1 | cyclin A1 | 1.56 | 1.82 | 1.72 | −1.17 |
| CD177* | CD177 molecule | 1.85 | 1.38 | 1.51 | 1.34 |
| CD24* | CD24 molecule | 1.82 | −1.38 | 1.04 | 2.51 |
| CDK1 | cyclin dependent kinase 1 | 2.1 | −1.52 | 1.05 | 3.19 |
| CDKN3 | cyclin dependent kinase | 1.99 | −1.25 | 1.16 | 2.5 |
| | inhibitor 3 | | | | |
| CEACAM6 | CEA cell adhesion molecule 6 | 2.06 | −1.43 | 1.06 | 2.96 |
| CEACAM8 | CEA cell adhesion molecule 8 | 1.99 | −1.4 | 1.09 | 2.8 |
| CENPA* | centromere protein A | 1.99 | −1.3 | 1.15 | 2.59 |
| CFH* | complement factor H | 1.79 | 1.13 | 1.35 | 1.58 |
| CHDH* | choline dehydrogenase | 1.75 | −1.25 | 1.07 | 2.19 |
| CHIT1* | chitinase 1 | 1.39 | 1.11 | 1.2 | 1.24 |
| CKAP2L* | cytoskeleton associated | 2.23 | −1.33 | 1.19 | 2.96 |
| | protein 2 like | | | | |
| CLEC4C* | C-type lectin domain family | −2.3 | −1.13 | −1.42 | −2.04 |
| | 4 member C | | | | |
| CLEC4F | C-type lectin domain family | −2.53 | −1.78 | −2.03 | −1.43 |
| | 4 member F | | | | |
| CLNK | cytokine dependent | −2.59 | −1.58 | −1.78 | −1.64 |
| | hematopoietic cell linker | | | | |
| COL17A1 | collagen type XVII alpha | 2.1 | −1.25 | 1.16 | 2.62 |
| | 1 chain | | | | |
| CRISP2 | cysteine rich secretory | 2.48 | 1.76 | 1.98 | 1.41 |
| | protein 2 | | | | |
| CRISP3 | cysteine rich secretory | 1.97 | 1.44 | 1.61 | 1.37 |
| | protein 3 | | | | |
| CTSE | cathepsin E | 1.59 | 1.33 | 1.41 | 1.2 |
| CTSG | cathepsin G | 2.03 | −1.8 | −1.07 | 3.66 |
| CYP19A1 | cytochrome P450 family | 2.38 | 1.08 | 1.38 | 2.2 |
| | 19 SF A member 1 | | | | |
| CYYR1 | cysteine and tyrosine | 1.47 | 1.3 | 1.36 | 1.13 |
| | rich 1 | | | | |
| DEFA4 | defensin alpha 4 | 2.27 | −1.58 | 1.06 | 3.59 |
| DENND2C* | DENN domain containing 2C | 1.71 | −1.24 | 1.08 | 2.13 |
| DEPDC1 | DEP domain containing 1 | 2.05 | −2.01 | −1.11 | 4.11 |
| DGKK | diacylglycerol kinase kappa | −1.66 | 1 | −1.24 | −1.67 |
| DLC1 | DLC1 Rho GTPase activating | 1.82 | 1.13 | 1.4 | 1.62 |
| | protein | | | | |
| DLGAP5* | DLG associated protein 5 | 2.25 | −1.51 | 1.07 | 3.39 |
| DNAH10* | dynein axonemal heavy chain | 1.81 | 1.06 | 1.32 | 1.71 |
| | 10 | | | | |
| DOC2B | double C2 domain beta | 1.84 | −1.29 | 1.1 | 2.37 |
| DSP* | desmoplakin | −2.48 | −1.73 | −2.05 | −1.43 |
| ELANE | elastase, neutrophil expressed | 2.39 | −1.33 | 1.17 | 3.18 |
| ERG | ETS transcription factor ERG | 1.87 | −1.13 | 1.21 | 2.12 |
| FAM20A* | Golgi associated secretory | 1.58 | 1.07 | 1.21 | 1.47 |
| | pathway pseudokinase | | | | |
| FAM83A | family with sequence similarity | 2.73 | 1.8 | 1.66 | 1.51 |
| | 83 member A | | | | |
| FBN1* | fibrillin 1 | 1.5 | −1.44 | −1.13 | 2.16 |
| FFAR3 | free fatty acid receptor 3 | 2.02 | 1.42 | 1.62 | 1.42 |
| G0S2* | G0/G1 switch 2 | 2.4 | 1.42 | 1.79 | 1.69 |
| GGT5*^{#} | gamma-glutamyltransferase 5 | 3.14 | 1.68 | 2.18 | 1.87 |
| GLB1L2* | galactosidase beta 1 like 2 | −1.52 | −1.6 | −1.57 | 1.05 |
| GJB6 | gap junction protein beta 6 | 1.96 | 1.27 | 1.5 | 1.55 |
| GPR84*^{#} | G protein-coupled receptor 84 | 2.93 | 1.81 | 2.27 | 1.62 |
| GRAMD1C* | GRAM domain containing 1C | −1.97 | −1.46 | −1.61 | −1.35 |
| GYPA | glycophorin A (MNS blood group) | 1.61 | −1.64 | −1.07 | 2.64 |
| HBM* | hemoglobin subunit mu | 2.17 | 1.41 | 1.67 | 1.54 |
| HMGB3* | high mobility group box 3 | 1.53 | −1.1 | 1.1 | 1.69 |
| HP* | haptoglobin | 2.4 | 1.52 | 1.8 | 1.58 |
| HPGD | 15-hydroxyprostaglandin | 1.32 | 1.24 | 1.27 | 1.06 |
| | dehydrogenase | | | | |
| HRK*^{#} | harakiri, BCL2 interacting | −4.84 | −1.94 | −2.75 | −2.49 |
| | protein | | | | |
| IGLL1 | immunoglobulin lambda like | 2.53 | 1.04 | 1.53 | 2.43 |
| | polypeptide 1 | | | | |
| IL1R2 | interleukin 1 receptor type 2 | 1.25 | 1.17 | 1.19 | 1.07 |
| IL1RL1 | interleukin 1 receptor like 1 | 1.4 | −1.03 | 1.03 | 1.45 |
| INHBA | inhibin subunit beta A | 2.14 | −1.52 | 1.08 | 3.25 |
| IQGAP3* | IQ motif containing GTPase | 2.63 | −1.12 | 1.39 | 2.94 |
| | activating protein 3 | | | | |
| ITGA7 | integrin subunit alpha 7 | 1.56 | −1.25 | 1.04 | 1.96 |
| ITGB4* | integrin subunit beta 4 | −2.36 | −2.18 | −2.23 | −1.08 |
| KIF15* | kinesin family member 15 | 1.65 | −1.68 | −1.12 | 2.78 |
| KIF20A | kinesin family member 20A | 2.01 | −1.37 | 1.07 | 2.74 |
| KLF14 | Kruppel like factor 14 | 1.56 | 1.22 | 1.34 | 1.28 |
| LAMB3* | laminin subunit beta 3 | 1.98 | 1.4 | 1.58 | 1.41 |
| LCN2* | lipocalin 2 | 2.35 | −1.2 | 1.14 | 2.81 |
| LGR4 | Leu rich repeat cont. G | 2.03 | −1.09 | 1.24 | 2.22 |
| | protein-coupled receptor 4 | | | | |
| LPL* | lipoprotein lipase | −1 | 1.65 | 1.89 | −1.65 |
| LTF* | lactotransferrin | 2.56 | −1.04 | 1.38 | 2.65 |
| MAFG* | MAF bZIP transcription | 1.42 | 1.27 | 1.32 | 1.12 |
| | factor G | | | | |
| MERTK* | MER proto-oncogene, tyrosine | 1.75 | 1.1 | 1.27 | 1.59 |
| | kinase | | | | |
| METTL7B | methyltransferase like 7B | 1.58 | 1.12 | 1.26 | 1.41 |
| MMP8*^{#} | matrix metallopeptidase 8 | 4.37 | −1.06 | 1.61 | 4.63 |
| MMP9* | matrix metallopeptidase 9 | 1.45 | 1.36 | 1.39 | 1.06 |
| MPO | myeloperoxidase | 2.05 | −1.37 | 1.09 | 2.82 |
| MRC1* | mannose receptor C-type 1 | 1.95 | 1.81 | 2.45 | 1.08 |
| MROCKI | cis-regulating promoter of | 2.2 | −1.16 | 1.48 | 2.55 |
| | cytokines inflammation | | | | |
| MS4A3 | membrane spanning 4-domains A3 | 1.91 | −1.65 | −1.03 | 3.15 |
| MS4A4A* | membrane spanning 4-domains A4A | 2.01 | 1.18 | 1.39 | 1.71 |
| NECAB1 | N-terminal EF-hand calcium | 1.51 | −1.33 | 1.01 | 2 |
| | binding protein 1 | | | | |
| NEIL3 | nei like DNA glycosylase 3 | 2 | −1.67 | 1 | 3.36 |
| NEK2 | NIMA related kinase 2 | 1.73 | −1.7 | −1.06 | 2.94 |
| NRXN2* | neurexin 2 | −1.93 | −1.1 | −1.34 | −1.75 |
| NUF2* | NUF2 component NDC80 kinetochore | 1.51 | −1.68 | −1.15 | 2.53 |
| | complex | | | | |
| OLAH | oleoyl-ACP hydrolase | 1.42 | 1.52 | 1.49 | −1.08 |
| OLFM4 | olfactomedin 4 | 2.26 | −1.04 | 1.35 | 2.34 |
| OLIG2 | oligodendrocyte transcription | 1.28 | −1.84 | −1.35 | 2.36 |
| | factor 2 | | | | |
| PCOLCE2 | procollagen C-endopeptidase | 2.5 | −1.16 | 1.3 | 2.89 |
| | enhancer 2 | | | | |
| PCSK9 | proprotein convertase subtilisin/ | 1.43 | 1.01 | 1.13 | 1.42 |
| | kexin type 9 | | | | |
| PHF24* | PHD finger protein 24 | −2.85 | 1.13 | −1.3 | −3.23 |
| PIGR | polymeric immunoglobulin receptor | −5.12 | −3.66 | −2.27 | −1.4 |
| PLAAT2 | phospholipase A and acyltransferase | 1.36 | 1.47 | 1.45 | −1.08 |
| | 2 | | | | |
| PPARG | peroxisome proliferator | 1.72 | 1.49 | 1.57 | 1.15 |
| | activated receptor gamma | | | | |
| PRTN3 | proteinase 3 | 2.65 | −1.43 | 1.16 | 3.78 |
| PTGES* | prostaglandin E synthase | 1.69 | 1.59 | 1.62 | 1.06 |
| PYCR1* | pyrroline-5-carboxylate | 1.72 | −1.01 | 1.25 | 1.74 |
| | reductase 1 | | | | |
| RAB3IL1* | RAB3A interacting protein like 1 | 1.4 | −1.04 | 1.1 | 1.46 |
| RASGRF1^{#} | Ras protein specific guanine | -4.85 | −1.17 | −2.77 | -4.15 |
| | nucleotide RF1 | | | | |
| RETN* | resistin | 1.97 | 1.41 | 1.56 | 1.39 |
| RHCE | Rh blood group CcEe antigens | 1.63 | −1.29 | 1.07 | 2.1 |
| RIPOR3 | RIPOR family member 3 | 1.82 | −1.11 | 1.22 | 2.01 |
| RPGRIP1* | RPGR interacting protein 1 | −2.22 | −1.41 | −1.61 | −1.57 |
| RRM2* | ribonucleotide reductase regulatory | 2.29 | −1.38 | 1.12 | 3.16 |
| | subunit M2 | | | | |
| S100A12 | S100 calcium binding protein A12 | 1.89 | 1.12 | 1.35 | 1.68 |
| S100A8 | S100 calcium binding protein A8 | 1.63 | 1.03 | 1.21 | 1.59 |
| SCN8A* | sodium voltage-gated channel | 2.02 | −1.26 | 1.19 | 2.55 |
| | alpha subunit 8 | | | | |
| SEMA6B | semaphorin 6B | 1.96 | 2.35 | 1.63 | −1.2 |
| SERPINB10* | serpin family B member 10 | 2.28 | −1.14 | 1.29 | 2.6 |
| SIGLEC8 | sialic acid binding Ig like | 1.22 | −2.3 | −1.55 | 2.8 |
| | lectin 8 | | | | |
| SIL1* | SIL1 nucleotide exchange factor | 1.31 | 1.19 | 1.23 | 1.1 |
| SLC16A1* | solute carrier family 16 member 1 | 1.55 | −1.3 | 1.04 | 2.02 |
| SLC28A3 | solute carrier family 28 member 3 | 2.06 | 1.11 | 1.42 | 1.86 |
| SLC39A8* | solute carrier family 39 member 8 | 2.64 | 1.39 | 1.81 | 1.9 |
| SLC4A10* | solute carrier family 4 member 10 | −2.64 | −1.51 | −1.83 | −1.75 |
| SLC51A | solute carrier family 51 subunit | 1.72 | 1.02 | 1.21 | 1.68 |
| | alpha | | | | |
| SLC6A19* | solute carrier family 6 member 19 | 2.5 | 1.87 | 2.06 | 1.34 |
| SLC8A3* | solute carrier family 8 member A3 | −1.93 | −1.51 | −1.66 | −1.28 |
| SLCO4A1 | solute carrier organic anion | 1.95 | 1.07 | 1.33 | 1.83 |
| | transporter | | | | |
| | FM4A1 | | | | |
| SMIM1* | small integral membrane protein 1 | 1.73 | 1.49 | 1.58 | 1.16 |
| | (Vel blood gp) | | | | |
| SMPDL3A | sphingomyelin phosphodiesterase | 2.03 | 1.12 | 1.34 | 1.82 |
| | acid like 3A | | | | |
| SPATC1* | spermatogenesis and centriole | 2.33 | 1.44 | 1.7 | 1.62 |
| | associated 1 | | | | |
| SPOP* | speckle type BTB/POZ protein | −1.14 | −1.06 | −1.08 | −1.08 |
| SSBP2* | single stranded DNA binding | −1.34 | −1.21 | −1.25 | −1.11 |
| | protein 2 | | | | |
| TCN1 | transcobalamin 1 | 2.12 | −1.1 | 1.28 | 2.34 |
| TCTEX1D1* | Tctex1 domain containing 1 | 2.39 | −1.14 | 1.31 | 2.73 |
| TDRD9 | tudor domain containing 9 | 1.68 | 1.04 | 1.22 | 1.61 |
| TEAD2* | TEA domain transcription | −2.22 | −1.38 | −1.6 | −1.6 |
| | factor 2 | | | | |
| TFRC | transferrin receptor | 1.47 | −1.39 | −1.01 | 2.05 |
| THBS1 | thrombospondin 1 | 1.94 | 1.21 | 1.45 | 1.6 |
| TIMP3 | TIMP metallopeptidase inhibitor | 1.33 | −1.39 | −1.1 | 1.85 |
| | 3 | | | | |
| TLN2* | talin 2 | 1.51 | 1.16 | 1.29 | 1.3 |
| TMEM255A* | transmembrane protein 255A | −2.07 | −1.32 | −1.61 | −1.56 |
| TMEM45A* | transmembrane protein 45A | 1.14 | 1.05 | 1.07 | 1.09 |
| TNFAIP8L3 | TNF alpha induced protein 8 | 2.28 | 1.22 | 1.58 | 1.87 |
| | like 3 | | | | |
| TNIP3 | TNFAIP3 interacting protein 3 | 1.75 | 1.07 | 1.34 | 1.63 |
| TROAP | trophinin associated protein | 1.75 | −1.15 | 1.14 | 2.02 |
| TTK | TTK protein kinase | 2 | −1.46 | 1.06 | 2.92 |
| VSIG4 | V-set and immunoglobulin | 1.32 | 2.06 | 1.9 | −1.56 |
| | domain containing 4 | | | | |
| WNT3 | Wnt family member 3 | −2.92 | −1.03 | −1.4 | −2.83 |
| YPEL4 | yippee like 4 | 1.64 | −1.24 | 1.12 | 2.03 |
| ZDHHC19 | zinc finger DHHC-type | 1.73 | 1.03 | 1.2 | 1.68 |
| | palmitoyltransferase 19 | | | | |

These 157 genes demonstrate differential expression between patients from different severity groups. Int. means intermediate. A reduced 73 gene set used for classifying patients into High vs. Low severity groups is indicated by * in column 1; these represent highly accurate and discriminative genes. A reduced signature presented in Table 8 is indicated by ^{#}.

TABLE 9
Severity model performance for three outcome measures
predicting impending severity and mortality.

| | | Cross Validation |
|---|---|---|
| | | Accuracy (AUC)/ |
| | | Sensitivity/ |
| | | Specificity |
| Comparison | Gene Set | (N = 194) |
| High vs. | High vs. Low all DE Genes | 85%; 76%; 76% |
| Low | Reduced High vs Low DE Signature^{a} | 80%; 72%; 71% |
| | Hypothesis Based Signature^{b} | 77%; 73%; 73% |
| High + | High vs. Low DE Genes | 79%; 65%; 75% |
| Intermediate | Reduced High vs Low DE Signature^{a} | 70%; 70%; 69% |
| vs. Low | Hypothesis Based Signature^{b} | 74%; 69%; 68% |

The AUC, sensitivity, and specificity of the models, and the machine learning algorithm used is provided. DE = differentially expressed.
^{a}CCL4L2, GPR84, HRK, MMP8, GGT5, RASGRF1
^{b}ADAMTS2, RETN, MMP8, G0S2, CYP19A1, OLAH, SLC6A19, TNFAIP8L3

What is claimed is:

1. A method for classifying a subject into a sepsis mechanistic endotype selected from neutrophilic-suppressive (NPS), inflammatory (INF), innate host defense (IHD), interferon (IFN) and adaptive (ADA) endotypes, the method comprising:
(a) determining, in a biological sample from the subject, a level of expression for each of a plurality of genes, to provide a sample gene signature; and
(b) comparing the sample gene signature with a reference gene signature to determine whether the subject has the sepsis mechanistic endotype,
wherein the sample gene signature and reference gene signature comprise an NPS endotype sub-signature, an INF endotype sub-signature, an IHD endotype sub-signature, an IFN endotype sub-signature, an ADA endotype sub-signature or combinations thereof,
wherein the NPS endotype sub-signature comprises genes selected from the group consisting of AGFG1, ARG1, ATP9A, ANXA3, EFNA1, GADD45A, GPR84, HPGD, IL1R1, KLF14, KREMEN1, MIR646HG, MLLT1, NSUN7, OLAH, ORM2, PCOLCE2, PFKFB2, SLC51A, TNFAIP8L3, ZDHHC19, ADAMTS3, AKR1C1, ALDH1A2, ALOX5AP, ALPL, AMPH, ANKRD55, BCL3, BTBD19, CA4, CD163L1, CD177, CD82, CST7, CYP19A1, CYSTM1, DAAM2, DGAT2, ECHDC3, ENTPD7, EXOSC4, FFAR3, FGF13, FSTL4, GALNT14, GRAMD1A, GRB10, GYG1, HPGD, IER3, IL18RAP, IL1R2, IL1RN, IRAG1-AS1, KCNE1B, KCNMA1, MCEMP1, MKNK1, MMP9, MSRA, NECAB1, NSMCE1-DT, OPLAH, PDGFC, PFKFB3, PHF24, PI3, PLIN4, PLIN5, PLK3, POR, PROK2, RFX2, RGL4, ROM1, S100A12, S100P, SEMA6B, SHROOM4, SLP1, SOCS3, SPATC1, SPDYA, SPINK8, SPP1, ST6GALNAC3, SYN2, TDRD9, TMEM120A, TMIGD3, TSPO, UPP1, and XCR1;
wherein the INF endotype sub-signature comprises genes selected from the group consisting of: BNIP3L, CA1, FAM83A, FECH, GLRX5, GYPA, IFIT1B, RHCE, RIOK3, RNF182, SLC6A19, SPTA1, THEM5, TLCD4, TSPAN5, TSPO2, ABCG2, ACHE, ACKR1, ACSL6, ADD2, AHSP, ALAS2, ALDH5A1, ANK1, ANKRD9, AQP1, ARHGEF12, ARHGEF37, ARL4A, ATP1B2, ATP1B2, BBOF1, BCAM, BCL2L1, BLVRB, BPGM, Clorf116, CA2, CISD2, CLIC2, CR1L, CR1L, CTNNAL1, CTSE, DCAF12, DMTN, DNAJC6, DPCD, DYRK3, EMID1, EPB42, ERFE, FAM210B, FAXDC2, FRMD4A, GMPR, GSPT1, GYPB, HBM, HEMGN, HEPACAM2, HMBS, IGF2BP2, ISCA1, ITLN1, KANK2, KCNH2, KDM7A-DT, KEL, KLC3, KLF1, KLHDC8A, KRT1, LRRC2, MAOA, MARCHF8, MBNL3, MFSD2B, MRC2, MXI1, MYL4, NFIX, NUDT4, OSBP2, PAGE2B, PBX1, PCDH1, PGF, PLEK2, PNP, PRDX2, PTPRF, RAPIGAP, RBM38, RFESD, RFESD, RGCC, RGS16, RHAG, RHD, RIPOR3, RNF175, RUNDC3A, SEC14L4, SELENBP1, SELENOP, SFRP2, SGIP1, SIAH2, SLC14A1, SLC1A5, SLC22A23, SLC2A1, SLC4A1, SLC6A8, SLC6A9, SLC7A5, SMIM5, SNCA, SOX6, SPTB, STRADB, TAL1, TENT5C, TFR2, TMCC2, TMOD1, TNS1, TRIM10, TRIM58, TSPAN7, TTC25, UBB, USP12, XK, YBX3, and YPEL4;
wherein the IHD endotype sub-signature comprises genes selected from the group consisting of: ABCA6, ADAM23, ALOX15, CACNA2D3, DYNC2H1, GPR34, GRAMD1C, LPL, MAP7, MIR155HG, PLCB1, SDC2, SIGLEC8, SPRED1, SLC16A14, SMPD3, TPPP3, TPRG1, ZNF600, ADGRD1, ANGPT1, GPR82, HDAC9, IL5RA, KLHDC1, PRSS33, PTGDR2, PTGFRN, TBC1D12, and TRIM2;
wherein the IFN endotype sub-signature comprises genes selected from the group consisting of: ANKRD22, APOL1, APOL4, BATF2, CARD17, CD274, EPSTI1, ETV7, GBP5, IDO1, IFITM3, P2RY14, PLEKHO1, RSAD2, SERPING1, TFEC, EXOC3L1, IRF7, OAS1, SEPTIN4, LY6E, and LAMP3; and
wherein the ADA endotype sub-signature comprises genes selected from the group consisting of: CCL2, CDC45, CENPF, CLEC4F, GTSE1, IFI27, ISG15, KCTD14, KIF14, KIF15, KLHDC7B, LGALS3BP, OTOF, PDIA4, SIGLEC1, USP18, AGRN, CD38, CDCA7, CDT1, CTLA4, DHX58, EME1, FAM111B, HES4, IFI44L, IFIT3, IFNG-AS1, IL12RB2, IL4I1, KIF19, LAG3, MCM10, P2RY6, PACSIN1, PARM1, SAMD4A, SPATS2L, HERC5, TMPRSS3, TNFRSF13B, TSHR, and TTC21A.

2. The method of claim 1, wherein the reference gene signature represents a standard level of expression of the genes comprised therein and a difference between a sample endotype sub-signature and a reference endotype sub-signature indicates that the subject has the sepsis mechanistic endotype corresponding to that sub-signature.

3. The method of claim 1 or 2, wherein the sample gene signature and the reference gene signature comprise the NPS endotype sub-signature, the INF endotype sub-signature, the IHD endotype sub-signature, the IFN endotype sub-signature, and the ADA endotype sub-signature.

4. The method of any one of claims 1 to 3, wherein when the NPS endotype sub-signature comprises genes selected from the group consisting of AGFG1, ARG1, ATP9A, ANXA3, EFNA1, GADD45A, GPR84, HPGD, IL1R1, KLF14, KREMEN1, MIR646HG, MLLT1, NSUN7, OLAH, ORM2, PCOLCE2, PFKFB2, SLC51A, TNFAIP8L3, and ZDHHC19.

5. The method of claim 4, wherein the NPS endotype sub-signature comprises: AGFG1, ARG1, ATP9A, ANXA3, EFNA1, GADD45A, GPR84, HPGD, IL1R1, KLF14, KREMEN1, MIR646HG, MLLT1, NSUN7, OLAH, ORM2, PCOLCE2, PFKFB2, SLC51A, TNFAIP8L3, and ZDHHC19.

6. The method of any one of claims 1 to 5, wherein the INF endotype sub-signature comprises genes selected from the group consisting of: BNIP3L, CA1, FAM83A, FECH, GLRX5, GYPA, IFIT1B, RHCE, RIOK3, RNF182, SLC6A19, SPTA1, THEM5, TLCD4, TSPAN5, and TSPO2.

7. The method of claim 6, wherein the INF endotype sub-signature comprises: BNIP3L, CA1, FAM83A, FECH, GLRX5, GYPA, IFIT1B, RHCE, RIOK3, RNF182, SLC6A19, SPTA1, THEM5, TLCD4, TSPAN5, and TSPO2.

8. The method of any one of claims 1 to 7, wherein the IHD endotype sub-signature comprises genes selected from the group consisting of ABCA6, ADAM23, ALOX15, CACNA2D3, DYNC2H1, GPR34, GRAMD1C, LPL, MAP7, MIR155HG, PLCB1, SDC2, SIGLEC8, SPRED1, SLC16A14, SMPD3, TPPP3, TPRG1, and ZNF600.

9. The method of claim 8, wherein the IHD endotype sub-signature comprises: ABCA6, ADAM23, ALOX15, CACNA2D3, DYNC2H1, GPR34, GRAMD1C, LPL, MAP7, MIR155HG, PLCB1, SDC2, SIGLEC8, SPRED1, SLC16A14, SMPD3, TPPP3, TPRG1, and ZNF600.

10. The method of any one of claims 1 to 9, wherein the IFN endotype sub-signature comprises genes selected from the group consisting of: ANKRD22, APOL1, APOL4, BATF2, CARD17, CD274, EPSTI1, ETV7, GBP5, IDO1, IFITM3, P2RY14, PLEKHO1, RSAD2, SERPING1, and TFEC.

11. The method of claim 10, wherein the IFN endotype sub-signature comprises: ANKRD22, APOL1, APOL4, BATF2, CARD17, CD274, EPSTI1, ETV7, GBP5, IDO1, IFITM3, P2RY14, PLEKHO1, RSAD2, SERPING1, and TFEC.

12. The method of any one of claims 1 to 11, wherein the ADA endotype sub-signature comprises genes selected from the group consisting of: CCL2, CDC45, CENPF, CLEC4F, GTSE1, IF127, ISG15, KCTD14, KIF14, KIF15, KLHDC7B, LGALS3BP, OTOF, PDIA4, SIGLEC1, and USP18.

13. The method of claim 12, wherein the ADA endotype sub-signature comprises: CCL2, CDC45, CENPF, CLEC4F, GTSE1, IF127, ISG15, KCTD14, KIF14, KIF15, KLHDC7B, LGALS3BP, OTOF, PDIA4, SIGLEC1 and USP18.

14. A method for classifying a subject into a sepsis mechanistic endotype selected from neutrophilic-suppressive (NPS), inflammatory (INF), innate host defense (IHD), interferon (IFN) and adaptive (ADA) endotypes, the method comprising:
(a) determining, in a biological sample from the subject, a level of expression for each of a plurality of genes, to provide a sample gene signature; and
(b) comparing the sample gene signature with a reference gene signature to determine whether the subject has the sepsis mechanistic endotype,
wherein the sample gene signature and reference gene signature comprise anNPS endotype signature pair, an INF endotype signature pair, an IHD endotype signature pair, an IFN endotype signature pair, an ADA endotype signature pair or combinations thereof,
wherein the NPS endotype signature pair is selected from: GADD45A/EFNA1, EFNA1/MIR646HG, MIR646HG/KLF14, MLLT1/MIR646HG, ARG1/MLLT1, MLLT1/EFNA1, MLLT1/NSUN7, EFNA1/NSUN7, SLC51A/EFNA1, EFNA1/KLF14, ZDHHC19/EFNA1, EFNA1/AGFG1, NSUN7/KLF14, EFNA1/PFKFB2, MLLT1/KLF14, ADAMTS3/PCOLCE2, ADAMTS3/ZDHHC19, ADAMTS3/SLC51A, ADAMTS3/HPGD, ADAMTS3/SEMA6B, ADAMTS3/EFNA1, ADAMTS3/AGFG1, ADAMTS3/NSUN7, ADAMTS3/TNFAIP8L3, ADAMTS3/KREMEN1, ADAMTS3/ORM2, ADAMTS3/MIR646HG, ADAMTS3/KLF14, AGFG1/NSUN7, AGFG1/TNFAIP8L3, AGFG1/KREMEN1, AGFG1/ORM2, AGFG1/MIR646HG, AGFG1/KLF14, ANXA3/GPR84, ANXA3/OLAH, ANXA3/ADAMTS3, ANXA3/PCOLCE2, ANXA3/ZDHHC19, ANXA3/SLC51A, ANXA3/HPGD, ANXA3/SEMA6B, ANXA3/EFNA1, ANXA3/AGFG1, ANXA3/NSUN7, ANXA3/TNFAIP8L3, ANXA3/KREMEN1, ANXA3/ORM2, ANXA3/MIR646HG, ANXA3/KLF14, ARG1/PFKFB2, ARG1/MLLT1, ARG1/ANXA3, ARG1/GPR84, ARG1/OLAH, ARG1/ADAMTS3, ARG1/PCOLCE2, ARG1/ZDHHC19, ARG1/SLC51A, ARG1/HPGD, ARG1/SEMA6B, ARG1/EFNA1, ARG1/AGFG1, ARG1/NSUN7, ARG1/TNFAIP8L3, ARG1/KREMEN1, ARG1/ORM2, ARG1/MIR646HG, ARG1/KLF14, ATP9A/EPB41L4B, ATP9A/IL1R1, ATP9A/GADD45A, ATP9A/ARG1, ATP9A/PFKFB2, ATP9A/MLLT1, ATP9A/ANXA3, ATP9A/GPR84, ATP9A/OLAH, ATP9A/ADAMTS3, ATP9A/PCOLCE2, ATP9A/ZDHHC19, ATP9A/SLC51A, ATP9A/HPGD, ATP9A/SEMA6B, ATP9A/EFNA1, ATP9A/AGFG1, ATP9A/NSUN7, ATP9A/TNFAIP8L3, ATP9A/KREMEN1, ATP9A/ORM2, ATP9A/MIR646HG, ATP9A/KLF14, EFNA1/AGFG1, EFNA1/NSUN7, EFNA1/TNFAIP8L3, EFNA1/KREMEN1, EFNA1/ORM2, EFNA1/MIR646HG, EFNA1/KLF14, EPB41L4B/IL1R1, EPB41L4B/GADD45A, EPB41L4B/ARG1, EPB41L4B/PFKFB2, EPB41L4B/MLLT1, EPB41L4B/ANXA3, EPB41L4B/GPR84, EPB41L4B/OLAH, EPB41L4B/ADAMTS3, EPB41L4B/PCOLCE2, EPB41L4B/ZDHHC19, EPB41L4B/SLC51A, EPB41L4B/HPGD, EPB41L4B/SEMA6B, EPB41L4B/EFNA1, EPB41L4B/AGFG1, EPB41L4B/NSUN7, EPB41L4B/TNFAIP8L3, EPB41L4B/KREMEN1, EPB41L4B/MIR646HG, EPB41L4B/KLF14, GADD45A/ARG1, GADD45A/PFKFB2, GADD45A/MLLT1, GADD45A/ANXA3, GADD45A/GPR84, GADD45A/OLAH, GADD45A/ADAMTS3, GADD45A/PCOLCE2, GADD45A/ZDHHC19, GADD45A/SLC51A, GADD45A/HPGD, GADD45A/SEMA6B, GADD45A/EFNA1, GADD45A/AGFG1, GADD45A/NSUN7, GADD45A/TNFAIP8L3, GADD45A/KREMEN1, GADD45A/ORM2, GADD45A/MIR646HG, GADD45A/KLF14, GPR84/OLAH, GPR84/ADAMTS3, GPR84/PCOLCE2, GPR84/ZDHHC19, GPR84/SLC51A, GPR84/HPGD, GPR84/SEMA6B, GPR84/EFNA1, GPR84/AGFG1, GPR84/NSUN7, GPR84/TNFAIP8L3, GPR84/KREMEN1, GPR84/ORM2, GPR84/MIR646HG, GPR84/KLF14, HPGD/SEMA6B, HPGD/EFNA1, HPGD/AGFG1, HPGD/NSUN7, HPGD/TNFAIP8L3, HPGD/KREMEN1, HPGD/ORM2, HPGD/MIR646HG, HPGD/KLF14, IL1R1/GADD45A, IL1R1/ARG1, IL1R1/PFKFB2, IL1R1/MLLT1, IL1R1/ANXA3, IL1R1/GPR84, IL1R1/OLAH, IL1R1/ADAMTS3, IL1R1/PCOLCE2, IL1R1/ZDHHC19, IL1R1/SLC51A, IL1R1/HPGD, IL1R1/SEMA6B, IL1R1/EFNA1, IL1R1/AGFG1, IL1R1/NSUN7, IL1R1/TNFAIP8L3, IL1R1/KREMEN1, IL1R1/ORM2, IL1R1/MIR646HG, IL1R1/KLF14, KREMEN1/ORM2, KREMEN1/MIR646HG, KREMEN1/KLF14, MIR646HG/KLF14, MLLT1/ANXA3, MLLT1/GPR84, MLLT1/OLAH, MLLT1/ADAMTS3, MLLT1/PCOLCE2, MLLT1/ZDHHC19, MLLT1/SLC51A, MLLT1/HPGD, MLLT1/SEMA6B, MLLT1/EFNA1, MLLT1/AGFG1, MLLT1/NSUN7, MLLT1/TNFAIP8L3, MLLT1/KREMEN1, MLLT1/ORM2, MLLT1/MIR646HG, MLLT1/KLF14, NSUN7/TNFAIP8L3, NSUN7/KREMEN1, NSUN7/ORM2, NSUN7/MIR646HG, NSUN7/KLF14, OLAH/ADAMTS3, OLAH/PCOLCE2, OLAH/ZDHHC19, OLAH/SLC51A, OLAH/HPGD, OLAH/SEMA6B, OLAH/EFNA1, OLAH/AGFG1, OLAH/NSUN7, OLAH/TNFAIP8L3, OLAH/KREMEN1, OLAH/ORM2, OLAH/MIR646HG, OLAH/KLF14, ORM2/MIR646HG, ORM2/KLF14, PCOLCE2/ZDHHC19, PCOLCE2/SLC51A, PCOLCE2/HPGD, PCOLCE2/SEMA6B, PCOLCE2/EFNA1, PCOLCE2/AGFG1, PCOLCE2/NSUN7, PCOLCE2/TNFAIP8L3, PCOLCE2/KREMEN1, PCOLCE2/ORM2, PCOLCE2/MIR646HG, PCOLCE2/KLF14, PFKFB2/MLLT1, PFKFB2/ANXA3, PFKFB2/GPR84, PFKFB2/OLAH, PFKFB2/ADAMTS3, PFKFB2/PCOLCE2, PFKFB2/ZDHHC19, PFKFB2/SLC51A, PFKFB2/HPGD, PFKFB2/SEMA6B, PFKFB2/EFNA1, PFKFB2/AGFG1, PFKFB2/NSUN7, PFKFB2/TNFAIP8L3, PFKFB2/KREMEN1, PFKFB2/ORM2, PFKFB2/MIR646HG, PFKFB2/KLF14, SEMA6B/EFNA1, SEMA6B/AGFG1, SEMA6B/NSUN7, SEMA6B/TNFAIP8L3, SEMA6B/KREMEN1, SEMA6B/ORM2, SEMA6B/MIR646HG, SEMA6B/KLF14, SLC51A/HPGD, SLC51A/SEMA6B, SLC51A/EFNA1, SLC51A/AGFG1, SLC51A/NSUN7, SLC51A/TNFAIP8L3, SLC51A/KREMEN1, SLC51A/ORM2, SLC51A/MIR646HG, SLC51A/KLF14, TNFAIP8L3/KREMEN1, TNFAIP8L3/ORM2, TNFAIP8L3/MIR646HG, TNFAIP8L3/KLF14, ZDHHC19/SLC51A, ZDHHC19/HPGD, ZDHHC19/SEMA6B, ZDHHC19/EFNA1, ZDHHC19/AGFG1, ZDHHC19/NSUN7, ZDHHC19/TNFAIP8L3, ZDHHC19/KREMEN1, ZDHHC19/ORM2, ZDHHC19/MIR646HG, and ZDHHC19/KLF14;
wherein the INF endotype signature pair is selected from: FECH/TFEC, TFEC/IFIT1B, FECH/RNF182, IFIT1B/FECH, FECH/APOL4, FECH/GYPA, ITLN1/FECH, FECH/THEM5, IFIT1B/CA1, RHAG/FECH, FECH/FAM83A, RHCE/FECH, TFEC/CA1, SPTA1/FECH, ANKRD22/GLRX5, ANKRD22/GYPA, ANKRD22/IFIT1B, ANKRD22/ITLN1, ANKRD22/KLHDC8A, ANKRD22/RHCE, ANKRD22/RNF182, ANKRD22/SPTA1, ANKRD22/THEM5, ANKRD22/TSPAN5, APOL4/BNIP3L, APOL4/CA1, APOL4/DYRK3, APOL4/FAM83A, APOL4/GLRX5, APOL4/GYPA, APOL4/IFIT1B, APOL4/ITLN1, APOL4/KLHDC8A, APOL4/RHAG, APOL4/RHCE, APOL4/RIOK3, APOL4/RNF182, APOL4/SPTA1, APOL4/THEM5, APOL4/TLCD4, APOL4/TMCC2, APOL4/TSPAN5, APOL4/TSPO2, BNIP3L/ANKRD22, BNIP3L/CA1, BNIP3L/CARD17, BNIP3L/CD274, BNIP3L/DYRK3, BNIP3L/FAM83A, BNIP3L/GBP5, BNIP3L/GLRX5, BNIP3L/GYPA, BNIP3L/IFIT1B, BNIP3L/ITLN1, BNIP3L/KLHDC8A, BNIP3L/P2RY14, BNIP3L/RHAG, BNIP3L/RHCE, BNIP3L/RNF182, BNIP3L/SPTA1, BNIP3L/TFEC, BNIP3L/THEM5, BNIP3L/TLCD4, BNIP3L/TMCC2, BNIP3L/TSPAN5, BNIP3L/TSPO2, CA1/ANKRD22, CA1/CARD17, CA1/DYRK3, CA1/FAM83A, CA1/GBP5, CA1/GLRX5, CA1/GYPA, CA1/IFIT1B, CA1/ITLN1, CA1/KLHDC8A, CA1/P2RY14, CA1/RHCE, CA1/RNF182, CA1/SPTA1, CA1/THEM5, CA1/TLCD4, CA1/TSPAN5, CD274/CA1, CD274/DYRK3, CD274/FAM83A, CD274/GLRX5, CD274/GYPA, CD274/IFIT1B, CD274/ITLN1, CD274/KLHDC8A, CD274/RHCE, CD274/RNF182, CD274/SPTA1, CD274/THEM5, CD274/TLCD4, CD274/TMCC2, CD274/TSPAN5, DYRK3/ANKRD22, DYRK3/CARD17, DYRK3/FAM83A, DYRK3/GBP5, DYRK3/GLRX5, DYRK3/GYPA, DYRK3/IFIT1B, DYRK3/ITLN1, DYRK3/KLHDC8A, DYRK3/P2RY14, DYRK3/RHCE, DYRK3/RNF182, DYRK3/SPTA1, DYRK3/THEM5, DYRK3/TLCD4, DYRK3/TSPAN5, FAM83A/ANKRD22, FAM83A/CARD17, FAM83A/GBP5, FAM83A/GLRX5, FAM83A/GYPA, FAM83A/IFIT1B, FAM83A/ITLN1, FAM83A/KLHDC8A, FAM83A/P2RY14, FAM83A/RHCE, FAM83A/RNF182, FAM83A/SPTA1, FAM83A/THEM5, FAM83A/TLCD4, FAM83A/TSPAN5, FECH/ANKRD22, FECH/APOL4, FECH/BNIP3L, FECH/CA1, FECH/CARD17, FECH/CD274, FECH/DYRK3, FECH/FAM83A, FECH/GBP5, FECH/GLRX5, FECH/GYPA, FECH/IFIT1B, FECH/ITLN1, FECH/KLHDC8A, FECH/P2RY14, FECH/RHAG, FECH/RHCE, FECH/RIOK3, FECH/RNF182, FECH/SPTA1, FECH/TFEC, FECH/THEM5, FECH/TLCD4, FECH/TMCC2, FECH/TSPAN5, FECH/TSPO2, GBP5/GLRX5, GBP5/GYPA, GBP5/IFIT1B, GBP5/ITLN1, GBP5/KLHDC8A, GBP5/RHCE, GBP5/RNF182, GBP5/SPTA1, GBP5/THEM5, GBP5/TSPAN5, GLRX5/CARD17, GLRX5/IFIT1B, GLRX5/RHCE, GLRX5/THEM5, GYPA/CARD17, GYPA/GLRX5, GYPA/IFIT1B, GYPA/ITLN1, GYPA/P2RY14, GYPA/RHCE, GYPA/RNF182, GYPA/THEM5, IFIT1B/CARD17, ITLN1/CARD17, ITLN1/GLRX5, ITLN1/IFIT1B, ITLN1/RHCE, ITLN1/RNF182, ITLN1/THEM5, KLHDC8A/CARD17, KLHDC8A/GLRX5, KLHDC8A/GYPA, KLHDC8A/IFIT1B, KLHDC8A/ITLN1, KLHDC8A/P2RY14, KLHDC8A/RHCE, KLHDC8A/RNF182, KLHDC8A/SPTA1, KLHDC8A/THEM5, KLHDC8A/TSPAN5, P2RY14/GLRX5, P2RY14/IFIT1B, P2RY14/ITLN1, P2RY14/RHCE, P2RY14/RNF182, P2RY14/THEM5, RHAG/ANKRD22, RHAG/CA1, RHAG/CARD17, RHAG/CD274, RHAG/DYRK3, RHAG/FAM83A, RHAG/GBP5, RHAG/GLRX5, RHAG/GYPA, RHAG/IFIT1B, RHAG/ITLN1, RHAG/KLHDC8A, RHAG/P2RY14, RHAG/RHCE, RHAG/RNF182, RHAG/SPTA1, RHAG/THEM5, RHAG/TLCD4, RHAG/TMCC2, RHAG/TSPAN5, RHAG/TSPO2, RHCE/CARD17, RHCE/IFIT1B, RHCE/THEM5, RIOK3/ANKRD22, RIOK3/BNIP3L, RIOK3/CA1, RIOK3/CARD17, RIOK3/CD274, RIOK3/DYRK3, RIOK3/FAM83A, RIOK3/GBP5, RIOK3/GLRX5, RIOK3/GYPA, RIOK3/IFIT1B, RIOK3/ITLN1, RIOK3/KLHDC8A, RIOK3/P2RY14, RIOK3/RHAG, RIOK3/RHCE, RIOK3/RNF182, RIOK3/SPTA1, RIOK3/TFEC, RIOK3/THEM5, RIOK3/TLCD4, RIOK3/TMCC2, RIOK3/TSPAN5, RIOK3/TSPO2, RNF182/CARD17, RNF182/GLRX5, RNF182/IFIT1B, RNF182/RHCE, RNF182/THEM5, SPTA1/CARD17, SPTA1/GLRX5, SPTA1/GYPA, SPTA1/IFIT1B, SPTA1/ITLN1, SPTA1/P2RY14, SPTA1/RHCE, SPTA1/RNF182, SPTA1/THEM5, SPTA1/TSPAN5, TFEC/CA1, TFEC/DYRK3, TFEC/FAM83A, TFEC/GLRX5, TFEC/GYPA, TFEC/IFIT1B, TFEC/ITLN1, TFEC/KLHDC8A, TFEC/RHAG, TFEC/RHCE, TFEC/RNF182, TFEC/SPTA1, TFEC/THEM5, TFEC/TLCD4, TFEC/TMCC2, TFEC/TSPAN5, TFEC/TSPO2, THEM5/CARD17, THEM5/IFIT1B, TLCD4/ANKRD22, TLCD4/CARD17, TLCD4/GBP5, TLCD4/GLRX5, TLCD4/GYPA, TLCD4/IFIT1B, TLCD4/ITLN1, TLCD4/KLHDC8A, TLCD4/P2RY14, TLCD4/RHCE, TLCD4/RNF182, TLCD4/SPTA1, TLCD4/THEM5, TLCD4/TSPAN5, TMCC2/ANKRD22, TMCC2/CA1, TMCC2/CARD17, TMCC2/DYRK3, TMCC2/FAM83A, TMCC2/GBP5, TMCC2/GLRX5, TMCC2/GYPA, TMCC2/IFIT1B, TMCC2/ITLN1, TMCC2/KLHDC8A, TMCC2/P2RY14, TMCC2/RHCE, TMCC2/RNF182, TMCC2/SPTA1, TMCC2/THEM5, TMCC2/TLCD4, TMCC2/TSPAN5, TSPAN5/CARD17, TSPAN5/GLRX5, TSPAN5/GYPA, TSPAN5/IFIT1B, TSPAN5/ITLN1, TSPAN5/P2RY14, TSPAN5/RHCE, TSPAN5/RNF182, TSPAN5/THEM5, TSPO2/ANKRD22, TSPO2/CA1, TSPO2/CARD17, TSPO2/CD274, TSPO2/DYRK3, TSPO2/FAM83A, TSPO2/GBP5, TSPO2/GLRX5, TSPO2/GYPA, TSPO2/IFIT1B, TSPO2/ITLN1, TSPO2/KLHDC8A, TSPO2/P2RY14, TSPO2/RHCE, TSPO2/RNF182, TSPO2/SPTA1, TSPO2/THEM5, TSPO2/TLCD4, TSPO2/TMCC2, and TSPO2/TSPAN5;
wherein the IHD endotype signature pair is selected from: MAP7/SPRED1, SPRED1/GPR34, IL5RA/SPRED1, SPRED1/TPRG1, HRK/SPRED1, SPRED1/PLCB1, TRIM2/SPRED1, SIGLEC8/SPRED1, SMPD3/SPRED1, SPRED1/ZNF600, SPRED1/SDC2, MAP7/GPR34, PRSS33/SPRED1, SPRED1/DYNC2H1, CACNA2D3/SPRED1, ADAM23/GPR34, ADAM23/MAP7, ADAM23/PLCB1, ADAM23/SPRED1, ALOX15/GPR34, ALOX15/PLCB1, ALOX15/SPRED1, BAALC/GPR34, BAALC/PLCB1, BAALC/SPRED1, CACNA2D3/DYNC2H1, CACNA2D3/GPR34, CACNA2D3/PLCB1, CACNA2D3/SPRED1, CACNA2D3/ZNF600, GPR34/DYNC2H1, GPR34/GRAMD1C, GPR34/PLCB1, GPR34/TPRG1, GPR34/ZNF600, GPR82/DYNC2H1, GPR82/GPR34, GPR82/GRAMD1C, GPR82/PLCB1, GPR82/TPRG1, GPR82/ZNF600, GRAMD1C/DYNC2H1, GRAMD1C/PLCB1, GRAMD1C/ZNF600, HRK/DYNC2H1, HRK/GPR34, HRK/MAP7, HRK/PLCB1, HRK/SPRED1, HRK/ZNF600, IL5RA/DYNC2H1, IL5RA/GPR34, IL5RA/PLCB1, IL5RA/SPRED1, IL5RA/TRIM2, MAP7/BAALC, MAP7/CACNA2D3, MAP7/DYNC2H1, MAP7/GPR34, MAP7/GPR82, MAP7/GRAMD1C, MAP7/PLCB1, MAP7/SPRED1, MAP7/TPRG1, MAP7/ZNF600, PLCB1/DYNC2H1, PLCB1/TPRG1, PLCB1/ZNF600, PRSS33/GPR34, PRSS33/PLCB1, PRSS33/SPRED1, SDC2/DYNC2H1, SDC2/GPR34, SDC2/PLCB1, SDC2/ZNF600, SIGLEC8/DYNC2H1, SIGLEC8/GPR34, SIGLEC8/MAP7, SIGLEC8/PLCB1, SIGLEC8/SPRED1, SIGLEC8/TRIM2, SMPD3/DYNC2H1, SMPD3/GPR34, SMPD3/MAP7, SMPD3/PLCB1, SMPD3/SPRED1, SMPD3/TRIM2, SPRED1/DYNC2H1, SPRED1/GPR34, SPRED1/GPR82, SPRED1/GRAMD1C, SPRED1/PLCB1, SPRED1/SDC2, SPRED1/TPRG1, SPRED1/ZNF600, TRIM2/CACNA2D3, TRIM2/DYNC2H1, TRIM2/GPR34, TRIM2/GPR82, TRIM2/GRAMD1C, TRIM2/HRK, TRIM2/MAP7, TRIM2/PLCB1, TRIM2/SDC2, TRIM2/SPRED1, TRIM2/TPRG1, and TRIM2/ZNF600;
wherein the IFN endotype signature pair is selected from: ETV7/PLEKHO1, IFITM3/ETV7, ETV7/APOL1, BATF2/ETV7, PLEKHO1/BATF2, ETV7/EPSTI1, EPSTI1/BATF2, IFITM3/BATF2, USP18/EPSTI1, ETV7/SEPTIN4, ETV7/LAMP3, SERPING1/BATF2, LAMP3/BATF2, LAMP3/SERPING1, APOL1/BATF2, APOL1/CLEC4F, APOL1/EPSTI1, APOL1/EXOC3L1, APOL1/HES4, APOL1/IFITM3, APOL1/LY6E, APOL1/RSAD2, APOL1/SEPTIN4, APOL1/SERPING1, APOL1/TPPP3, BATF2/EXOC3L1, BATF2/HES4, CLEC4F/BATF2, CLEC4F/EXOC3L1, EPSTI1/BATF2, EPSTI1/CLEC4F, EPSTI1/EXOC3L1, EPSTI1/HES4, EPSTI1/IFITM3, EPSTI1/LY6E, EPSTI1/RSAD2, EPSTI1/SERPING1, EPSTI1/TPPP3, ETV7/APOL1, ETV7/BATF2, ETV7/CLEC4F, ETV7/EPSTI1, ETV7/EXOC3L1, ETV7/HES4, ETV7/IFITM3, ETV7/LAMP3, ETV7/LY6E, ETV7/PLEKHO1, ETV7/RSAD2, ETV7/SEPTIN4, ETV7/SERPING1, ETV7/TPPP3, EXOC3L1/HES4, IFITM3/SERPING1, IFITM3/CLEC4F, IFITM3/TPPP3, IFITM3/LY6E, IFITM3/EXOC3L1, IFITM3/HES4, LAMP3/APOL1, LAMP3/BATF2, LAMP3/CLEC4F, LAMP3/EPSTI1, LAMP3/EXOC3L1, LAMP3/HES4, LAMP3/IFITM3, LAMP3/LY6E, LAMP3/RSAD2, LAMP3/SEPTIN4, LAMP3/SERPING1, LAMP3/TPPP3, LY6E/BATF2, LY6E/EXOC3L1, PLEKHO1/APOL1, PLEKHO1/BATF2, PLEKHO1/EPSTI1, PLEKHO1/EXOC3L1, PLEKHO1/IFITM3, PLEKHO1/LAMP3, PLEKHO1/RSAD2, PLEKHO1/SEPTIN4, PLEKHO1/SERPING1, RSAD2/BATF2, RSAD2/CLEC4F, RSAD2/EXOC3L1, RSAD2/HES4, RSAD2/IFITM3, RSAD2/LY6E, RSAD2/SERPING1, RSAD2/TPPP3, SEPTIN4/BATF2, SEPTIN4/CLEC4F, SEPTIN4/EPSTI1, SEPTIN4/EXOC3L1, SEPTIN4/HES4, SEPTIN4/IFITM3, SEPTIN4/LY6E, SEPTIN4/RSAD2, SEPTIN4/SERPING1, SEPTIN4/TPPP3, SERPING1/BATF2, SERPING1/CLEC4F, SERPING1/EXOC3L1, SERPING1/HES4, SERPING1/LY6E, SERPING1/TPPP3, TPPP3/BATF2, and TPPP3/EXOC3L1; and
wherein the ADA endotype signature pair is selected from: LGALS3BP/OTOF, LGALS3BP/IF127, LGALS3BP/KIF14, LGALS3BP/CENPF, GTSE1/LGALS3BP, LGALS3BP/KCTD14, LGALS3BP/PDIA4, LGALS3BP/TSHR, LGALS3BP/PLAAT2, OTOF/IF127, IGF1/LGALS3BP, CDC45/LGALS3BP, LGALS3BP/KIF15, LGALS3BP/IGLL5, LGALS3BP/MIXL, CAV1/LGALS3BP, CAV1/OTOF, CDC45/LGALS3BP, CDC45/OTOF, CENPF/KCTD14, GPRC5D/OTOF, GTSE1/LGALS3BP, GTSE1/OTOF, IGF1/LGALS3BP, IGF1/OTOF, KCTD14/KLHL14, KCTD14/PDIA4, KCTD14/TSHR, KIF14/KCTD14, LGALS3BP/CENPF, LGALS3BP/GPRC5D, LGALS3BP/IFI27, LGALS3BP/IGLL5, LGALS3BP/KCTD14, LGALS3BP/KIF14, LGALS3BP/KIF15, LGALS3BP/KLHL14, LGALS3BP/MIR155HG, LGALS3BP/MIXL1, LGALS3BP/OTOF, LGALS3BP/PDIA4, LGALS3BP/PLAAT2, LGALS3BP/SDC1, LGALS3BP/SLC16A14, LGALS3BP/TSHR, OTOF/CENPF, OTOF/IFI27, OTOF/IGLL5, OTOF/KCTD14, OTOF/KIF14, OTOF/KIF15, OTOF/KLHL14, OTOF/MIR155HG, OTOF/MIXL1, OTOF/PDIA4, OTOF/PLAAT2, OTOF/SDC1, OTOF/SLC16A14, OTOF/TSHR, PLAAT2/KCTD14, TNFRSF17/LGALS3BP, and TNFRSF17/OTOF.

15. The method of claim 14, wherein the reference gene signature represents a standard level of expression of the genes comprised therein and a difference between a sample endotype signature pair and a reference endotype signature pair indicates that the subject has the sepsis mechanistic endotype corresponding to that signature pair.

16. The method of claim 14 or 15, wherein the sample gene signature and the reference gene signature comprise the NPS endotype signature pair, the INF endotype signature pair, the IHD endotype signature pair, the IFN endotype signature pair, and the ADA endotype signature pair.

17. The method of any one of claims 14 to 16, wherein the NPS endotype signature pair is selected from: GADD45A/EFNA1, EFNA1/MIR646HG, MIR646HG/KLF14, MLLT1/MIR646HG, ARG1/MLLT1, MLLT1/EFNA1, MLLT1/NSUN7, EFNA1/NSUN7, SLC51A/EFNA1, EFNA1/KLF14, ZDHHC19/EFNA1, EFNA1/AGFG1, NSUN7/KLF14, EFNA1/PFKFB2, and MLLT1/KLF14.

18. The method of any one of claims 14 to 17, wherein the INF endotype signature pair is selected from: FECH/TFEC, TFEC/IFIT1B, FECH/RNF182, IFIT1B/FECH, FECH/APOL4, FECH/GYPA, ITLN1/FECH, FECH/THEM5, IFIT1B/CA1, RHAG/FECH, FECH/FAM83A, RHCE/FECH, TFEC/CA1, and SPTA1/FECH.

19. The method of any one of claims 14 to 18, wherein the IHD endotype signature pair is selected from: MAP7/SPRED1, SPRED1/GPR34, IL5RA/SPRED1, SPRED1/TPRG1, HRK/SPRED1, SPRED1/PLCB1, TRIM2/SPRED1, SIGLEC8/SPRED1, SMPD3/SPRED1, SPRED1/ZNF600, SPRED1/SDC2, MAP7/GPR34, PRSS33/SPRED1, SPRED1/DYNC2H1, and CACNA2D3/SPRED1.

20. The method of any one of claims 14 to 19, wherein the IFN endotype signature pair is selected from: ETV7/PLEKHO1, IFITM3/ETV7, ETV7/APOL1, BATF2/ETV7, PLEKHO1/BATF2, ETV7/EPSTI1, EPSTI1/BATF2, IFITM3/BATF2, USP18/EPSTI1, ETV7/SEPTIN4, ETV7/LAMP3, SERPING1/BATF2, LAMP3/BATF2, and LAMP3/SERPING1.

21. The method of any one of claims 14 to 20, wherein the ADA endotype signature pair is selected from: LGALS3BP/OTOF, LGALS3BP/IF127, LGALS3BP/KIF14, LGALS3BP/CENPF, GTSE1/LGALS3BP, LGALS3BP/KCTD14, LGALS3BP/PDIA4, LGALS3BP/TSHR, LGALS3BP/PLAAT2, OTOF/IF127, IGF1/LGALS3BP, CDC45/LGALS3BP, LGALS3BP/KIF15, LGALS3BP/IGLL5, and LGALS3BP/MIXL1.

22. A method for predicting severity of sepsis in a subject, wherein the severity of the sepsis is selected from high severity sepsis, intermediate severity sepsis and low severity sepsis, the method comprising:
(a) determining, in a biological sample from the subject, a level of expression for each of a plurality of genes, to provide a sample gene signature; and
(b) comparing the sample gene signature with a reference gene signature to predict the severity of the sepsis in the subject,
wherein high severity sepsis means a sequential organ failure assessment (SOFA) score of greater than or equal to 5, intermediate severity sepsis means a SOFA score of greater than or equal to 2 but less than 5, and low severity sepsis means a SOFA score of less than 2; and
wherein the plurality of genes is selected from the group consisting of ABCA13, ADAMTS2, ADAMTS3, AK5, ANKRD22, ANKRD34B, ANLN, AQP1, ARG1, ARHGAP44, ARHGEF17, ASPM, ATP1B2, AURKA, AZUl, BAIAP3, BPI, Clorf226, CACNB4, CCL4L2, CCN3, CCNA1, CD177, CD24, CDK1, CDKN3, CEACAM6, CEACAM8, CENPA, CFH, CHDH, CHITI, CKAP2L, CLEC4C, CLEC4F, CLNK, COL17A1, CRISP2, CRISP3, CTSE, CTSG, CYP19A1, CYYR1, DEFA4, DENND2C, DEPDC1, DGKK, DLC1, DLGAP5, DNAH10, DOC2B, DSP, ELANE, ERG, FAM20A, FAM83A, FBN1, FFAR3, GOS2, GGT5, GLB1L2, GJB6, GPR84, GRAMD1C, GYPA, HBM, HMGB3, HP, HPGD, HRK, IGLL1, IL1R2, IL1RL1, INHBA, IQGAP3, ITGA7, ITGB4, KIF15, KIF20A, KLF14, LAMB3, LCN2, LGR4, LPL, LTF, MAFG, MERTK, METTL7B, MMP8, MMP9, MPO, MRC1, MROCKI, MS4A3, MS4A4A, NECAB1, NEIL3, NEK2, NRXN2, NUF2, OLAH, OLFM4, OLIG2, PCOLCE2, PCSK9, PHF24, PIGR, PLAAT2, PPARG, PRTN3, PTGES, PYCRI, RAB3IL1, RASGRF1, RETN, RHCE, RIPOR3, RPGRIP1, RRM2, S100A12, S100A8, SCN8A, SEMA6B, SERPINB10, SIGLEC8, SIL1, SLC16A1, SLC28A3, SLC39A8, SLC4A10, SLC51A, SLC6A19, SLC8A3, SLCO4A1, SMIMI, SMPDL3A, SPATC1, SPOP, SSBP2, TCN1, TCTEX1D1, TDRD9, TEAD2, TFRC, THBS1, TIMP3, TLN2, TMEM255A, TMEM45A, TNFAIP8L3, TNIP3, TROAP, TTK, VSIG4, WNT3, YPEL4, and ZDHHC19.

23. The method of claim 22, wherein the plurality of genes comprises CCL4L2, GPR84, HRK, MMP8, GGT5, and RASGRF1.

24. The method of claim 22, wherein the plurality of genes is CCL4L2, GPR84, HRK, MMP8, GGT5, and RASGRF1.

25. The method of any one of claims 1 to 24, wherein determining the level of expression comprises detecting nucleic acids encoded by each of the plurality of genes.

26. The method of claim 25, wherein determining the level of expression comprises one or more of a polymerase chain reaction (PCR) amplification method, a non-PCR based amplification method, reverse transcriptase-(RT) PCR, Q-beta replicase amplification, ligase chain reaction, signal amplification (Ampliprobe), light cycling, differential display, Northern analysis, hybridization, microarray analysis, DNA sequencing, RNA sequencing (RNA-Seq), MassArray analysis and MALDI-TOF mass spectrometry.

27. The method of claim 26, wherein determining the level of expression comprises a polymerase chain reaction (PCR) amplification method.

28. The method of any one of claims 25 to 27, wherein determining the level of expression comprises RNA sequencing (RNA-Seq).

29. The method according to any one of claims 1 to 28, wherein the biological sample comprises sputum, blood, nasal brushings, throat swabs, urine, amniotic fluid, plasma, serum, saliva, semen, bone marrow, tissue or fine needle biopsy samples, stool, bronchoalveolar lavage fluid, cerebrospinal fluid, peritoneal fluid, pleural fluid, skin, or cells therefrom.

30. The method according to any one of claims 1 to 28, wherein the biological sample comprises blood.

31. The method according to any one of claims 1 to 30, wherein the biological sample has been obtained from the subject prior to admission in an intensive care unit.

32. The method according to any one of claims 1 to 30, wherein the biological sample has been obtained from the subject at first clinical presentation.

33. The method according to any one of claims 1 to 30, wherein the biological sample has been obtained from the subject within the first day after entry into an intensive care unit.

34. A use of one or more therapies that act specifically against a mechanism associated with a sepsis mechanistic endotype, for treatment of sepsis in a subject classified as having the sepsis mechanistic endotype by a method as defined in any one of claims 1 to 21 and 25 to 33, as dependent on any one of claims 1 to 21.

35. One or more therapies that act specifically against a mechanism associated with a sepsis mechanistic endotype for use to treat sepsis in a subject classified as having the sepsis mechanistic endotype by a method as defined in any one of claims 1 to 21 and 25 to 33, as dependent on any one of claims 1 to 21.

36. A use of an effective amount of one or more antibiotics for treatment of sepsis in a subject predicted as having high or intermediate severity sepsis by a method as defined in any one of claims 22 to 24 and claims 25 to 33, as dependent on any one of claims 22 to 24.

37. The use of claim 36, wherein the one or more antibiotics is one or a combination of a glycopeptide, a cephalosporin, a beta-lactam, a beta-lactamase inhibitor, a carbapenem, a quinolone, a fluoroquinolone, an aminoglycoside, a macrolide and a monobactam.

38. One or more antibiotics for use to treat sepsis in a subject predicted as having high or intermediate severity sepsis by a method as defined in any one of claims 22 to 24 and claims 25 to 33, as dependent on any one of claims 22 to 24.

39. The one or more antibiotics for the use of claim 38, wherein the one or more antibiotics is one or a combination of a glycopeptide, a cephalosporin, a beta-lactam, a beta-lactamase inhibitor, a carbapenem, a quinolone, a fluoroquinolone, an aminoglycoside, a macrolide and a monobactam.

40. A kit:
(a) for classifying a subject into a sepsis mechanistic endotype selected from neutrophilic-suppressive (NPS), inflammatory (INF), innate host defense (IHD), interferon (IFN) and adaptive (ADA) endotypes, the kit comprising gene specific reagents, each of the gene specific reagents capable of detecting an expression product of:
(i) a respective one of a plurality of genes or complement thereof in an NPS endotype sub-signature, an INF endotype sub-signature, an IHD endotype sub-signature, an IFN endotype sub-signature, an ADA endotype sub-signature or combinations thereof, wherein the NPS endotype sub-signature, the INF endotype sub-signature, the IHD endotype sub-signature, the IFN endotype sub-signature, the ADA endotype sub-signature or combinations thereof are as defined in any one of claims 1 and 4 to 13; or
(ii) a respective one of a plurality of genes or complement thereof in an NPS endotype signature pair, an INF endotype signature pair, an IHD endotype signature pair, an IFN endotype signature pair, an ADA endotype signature pair or combinations thereof, wherein the NPS endotype signature pair, the INF endotype signature pair, the IHD endotype signature pair, the IFN endotype signature pair, the ADA endotype signature pair or combinations thereof are as defined in any one of claims 14 and 16 to 21; or
(b) for predicting severity of sepsis in a subject, wherein the severity of the sepsis is selected from high severity sepsis, intermediate severity sepsis and low severity sepsis, wherein high severity sepsis means a sequential organ failure assessment (SOFA) score of greater than or equal to 5, intermediate severity sepsis means a SOFA score of greater than or equal to 2 but less than 5, and low severity sepsis means a SOFA score ofless than 2, the kit comprising gene specific reagents, each of the gene specific reagents capable of detecting an expression product of a respective one of a plurality of genes as defined in any one of claims 22 to 24 or complement thereof; and
optionally instructions for use.

41. A method for identifying a candidate agent for the treatment of sepsis in a subject classified as having a sepsis mechanistic endotype selected from neutrophilic-suppressive (NPS), inflammatory (INF), innate host defense (IHD), interferon (IFN) and adaptive (ADA) endotypes, the method comprising: (a) contacting a cell having the sepsis endotype with a test agent, (b) determining the level of expression for each of a plurality of genes in the cell to provide an expression signature; (c) comparing the expression signature with a reference signature, wherein the reference signature represents the level of expression of the plurality of genes in a normal cell; and (d) selecting the test agent as a candidate agent for treatment of the sepsis when the expression signature substantially corresponds with the reference signature, wherein the expression signature and reference signature comprise:
(a) an NPS endotype sub-signature for an NPS endotype cell, an INF endotype sub-signature for an INF endotype cell, an IHD endotype sub-signature for an IHD endotype cell, an IFN endotype sub-signature for an IFN endotype cell and an ADA endotype sub-signature for an ADA endotype cell, wherein the NPS endotype sub-signature, the INF endotype sub-signature, the IHD endotype sub-signature, the IFN endotype sub-signature, the ADA endotype sub-signature or combinations thereof are as defined in any one of claims 1 and 4 to 13; or
(b) an NPS endotype signature pair for an NPS endotype cell, an INF endotype signature pair for an INF endotype cell, an IHD endotype signature pair for an IHD endotype cell, an IFN endotype signature pair for an IFN endotype cell, and an ADA endotype signature pair for an ADA endotype cell, wherein the NPS endotype signature pair, the INF endotype signature pair, the IHD endotype signature pair, the IFN endotype signature pair, the ADA endotype signature pair or combinations thereof are as defined in any one of claims 14 and 16 to 21.
